SEQUENCE LISTING (1324 sequences)

SEQ ID NO: 1 - DNA, 22 bp, homo sapiens
aacagcactgcgtcatgcttga

SEQ ID NO: 2 - DNA, 22 bp, homo sapiens
catgattcagataggaacgaag

SEQ ID NO: 3 - DNA, 22 bp, homo sapiens
cagataggaacgaagctgaaac

SEQ ID NO: 4 - DNA, 22 bp, homo sapiens
tttgaactcaccgaacatttct

SEQ ID NO: 5 - DNA, 22 bp, homo sapiens
agccctgaggccccacctgccc

SEQ ID NO: 6 - DNA, 22 bp, homo sapiens
tcagggtcagccggtgcctggc

SEQ ID NO: 7 - DNA, 22 bp, homo sapiens
ctcctcagcctcctctttcaga

SEQ ID NO: 8 - DNA, 22 bp, homo sapiens
tctcttggtcaatggcactctt

SEQ ID NO: 9 - DNA, 22 bp, homo sapiens
gcaggcattgtcctcatcctcg

SEQ ID NO: 10 - DNA, 22 bp, homo sapiens
tcactctgtgtcctcaaacctc

SEQ ID NO: 11 - DNA, 22 bp, homo sapiens
cctgaacaaaggctgagccagc

SEQ ID NO: 12 - DNA, 22 bp, homo sapiens
aacaccagcacgacacctccga

SEQ ID NO: 13 - DNA, 22 bp, homo sapiens
gaaggagaagacgctggggttg

SEQ ID NO: 14 - DNA, 22 bp, homo sapiens
caggccttgtgccgatggcact

SEQ ID NO: 15 - DNA, 22 bp, homo sapiens
cggtagggctccaggtctaaag

SEQ ID NO: 16 - DNA, 22 bp, homo sapiens
acccctacagcctgaggaagct

SEQ ID NO: 17 - DNA, 22 bp, homo sapiens
gagctcctgcagtcggatgagc

SEQ ID NO: 18 - DNA, 22 bp, homo sapiens
aaccgagtggaactggggtatg

SEQ ID NO: 19 - DNA, 22 bp, homo sapiens
tcggggtagaggttcacgtaga

SEQ ID NO: 20 - DNA, 22 bp, homo sapiens
gcaaatgcagcgcttcccctcc

SEQ ID NO: 21 - DNA, 22 bp, homo sapiens
cagcaatcaataaagcaaacat

SEQ ID NO: 22 - DNA, 22 bp, homo sapiens
ctcttcacatggatctcagaaa

SEQ ID NO: 23 - DNA, 22 bp, homo sapiens
ggttaattcctgtctcacaaat

SEQ ID NO: 24 - DNA, 22 bp, homo sapiens
cacgggagcacttggatcgcat

SEQ ID NO: 25 - DNA, 22 bp, homo sapiens
cttttggaggatggtggtggga

SEQ ID NO: 26 - DNA, 22 bp, homo sapiens
agttttaagtacttttttgctg

SEQ ID NO: 27 - DNA, 22 bp, homo sapiens
ttaaaaaaaaaaacaacaccac

SEQ ID NO: 28 - DNA, 22 bp, homo sapiens
aaaaaaaaacaacaccacccac

SEQ ID NO: 29 - DNA, 22 bp, homo sapiens
acgcccaagagatatcgggggc

SEQ ID NO: 30 - DNA, 22 bp, homo sapiens
ttgtatgattagtgcacagaat

SEQ ID NO: 31 - DNA, 22 bp, homo sapiens
agtgcacagaatttctgtgccc

SEQ ID NO: 32 - DNA, 22 bp, homo sapiens
atccaatcctcctagagctccc

SEQ ID NO: 33 - DNA, 22 bp, homo sapiens
ctggaaatcaccggcaaagtct

SEQ ID NO: 34 - DNA, 22 bp, homo sapiens
gatgaggtcagcgacaccgaca

SEQ ID NO: 35 - DNA, 22 bp, homo sapiens
cagtggcatcatcctgcagtct

SEQ ID NO: 36 - DNA, 22 bp, homo sapiens
ggaccacccctatgagaagccc

SEQ ID NO: 37 - DNA, 22 bp, homo sapiens
ccaggaggagtgtgagatggag

SEQ ID NO: 38 - DNA, 22 bp, homo sapiens
acacagccatgggtcagggggc

SEQ ID NO: 39 - DNA, 22 bp, homo sapiens
tgaatttgggggcaaccaacgt

SEQ ID NO: 40 - DNA, 22 bp, homo sapiens
tgggtcagtttcgaaccaggct

SEQ ID NO: 41 - DNA, 22 bp, homo sapiens
gtataccttaggacatgaagat

SEQ ID NO: 42 - DNA, 22 bp, homo sapiens
acacccccaaggctccagggtc

SEQ ID NO: 43 - DNA, 22 bp, homo sapiens
tgctatacccagaaggttcggg

SEQ ID NO: 44 - DNA, 22 bp, homo sapiens
gtcgctctgcacccgggtgatg

SEQ ID NO: 45 - DNA, 22 bp, homo sapiens
caaagctgcacgtggtaaaaaa

SEQ ID NO: 46 - DNA, 22 bp, homo sapiens
ggtcctcgaacgtgtgtgccgc

SEQ ID NO: 47 - DNA, 22 bp, homo sapiens
aaggccgcggtggcgaaggcgt

SEQ ID NO: 48 - DNA, 22 bp, homo sapiens
caaccccgttactcaggccatg

SEQ ID NO: 49 - DNA, 22 bp, homo sapiens
taaggatccctcggataccgga

SEQ ID NO: 50 - DNA, 22 bp, homo sapiens
tttagggggaagaaaggaagaa

SEQ ID NO: 51 - DNA, 22 bp, homo sapiens
tgagggtcagcgtgtttacagc

SEQ ID NO: 52 - DNA, 22 bp, homo sapiens
acagtcgcaatcgcttccacga

SEQ ID NO: 53 - DNA, 22 bp, homo sapiens
tttcgactgacgtcaggaacta

SEQ ID NO: 54 - DNA, 22 bp, homo sapiens
ttattgagaagcctatgaaaga

SEQ ID NO: 55 - DNA, 22 bp, homo sapiens
atttccttgatataaccaattg

SEQ ID NO: 56 - DNA, 22 bp, homo sapiens
tttccttgatataaccaattgc

SEQ ID NO: 57 - DNA, 22 bp, homo sapiens
gaaccacgaggcaagaaaaaca

SEQ ID NO: 58 - DNA, 22 bp, homo sapiens
gtgaccttgtcgttagttactg

SEQ ID NO: 59 - DNA, 22 bp, homo sapiens
tctgtgatctgttaatgtggaa

SEQ ID NO: 60 - DNA, 22 bp, homo sapiens
acttagagttagtcatttctgg

SEQ ID NO: 61 - DNA, 22 bp, homo sapiens
tgtccagtagcacttttaacct

SEQ ID NO: 62 - DNA, 22 bp, homo sapiens
ctttcttcgatctgcctgaata

SEQ ID NO: 63 - DNA, 22 bp, homo sapiens
cataggaaaatggatctgtaaa

SEQ ID NO: 64 - DNA, 22 bp, homo sapiens
aagtcgagaaggccttgcacag

SEQ ID NO: 65 - DNA, 22 bp, homo sapiens
atacgtagaatgattcaaaagt

SEQ ID NO: 66 - DNA, 22 bp, homo sapiens
taagtcatcttgattcagttgg

SEQ ID NO: 67 - DNA, 22 bp, homo sapiens
acattctgtaagatagtttttg

SEQ ID NO: 68 - DNA, 22 bp, homo sapiens
atgtacacattctgttctaaaa

SEQ ID NO: 69 - DNA, 22 bp, homo sapiens
tgccgagcactgtctaaaagac

SEQ ID NO: 70 - DNA, 22 bp, homo sapiens
agtaaattattgggaaactata

SEQ ID NO: 71 - DNA, 22 bp, homo sapiens
ttccctactccacatttttctt

SEQ ID NO: 72 - DNA, 22 bp, homo sapiens
tggtacgactcgtgggatgttg

SEQ ID NO: 73 - DNA, 22 bp, homo sapiens
gtttataaactatgtgagttat

SEQ ID NO: 74 - DNA, 22 bp, homo sapiens
ccatgcaagtacgagtattaaa

SEQ ID NO: 75 - DNA, 22 bp, homo sapiens
attgttgcataaatatctggtt

SEQ ID NO: 76 - DNA, 22 bp, homo sapiens
ttccaggcaacggcaaagaccc

SEQ ID NO: 77 - DNA, 22 bp, homo sapiens
cagaggggagcgaaatgctgca

SEQ ID NO: 78 - DNA, 22 bp, homo sapiens
cgagggtcgaccgcgatgcctg

SEQ ID NO: 79 - DNA, 22 bp, homo sapiens
cctggctggtgcccagacctcg

SEQ ID NO: 80 - DNA, 22 bp, homo sapiens
acccgcagcgcggcctcaatgc

SEQ ID NO: 81 - DNA, 22 bp, homo sapiens
actcactcacccgactcctccc

SEQ ID NO: 82 - DNA, 22 bp, homo sapiens
aatctctaggagccttgtggct

SEQ ID NO: 83 - DNA, 22 bp, homo sapiens
cttgtttccatgaagcgcagga

SEQ ID NO: 84 - DNA, 22 bp, homo sapiens
tcaggtcctaggtgactgcgca

SEQ ID NO: 85 - DNA, 22 bp, homo sapiens
cccactgactaagcctggacgt

SEQ ID NO: 86 - DNA, 22 bp, homo sapiens
gtgagatcatactgcacgacca

SEQ ID NO: 87 - DNA, 22 bp, homo sapiens
tttcatccaaggaatcttaaat

SEQ ID NO: 88 - DNA, 22 bp, homo sapiens
aaagatgatggcaggaagggcc

SEQ ID NO: 89 - DNA, 22 bp, homo sapiens
caatagcccccagtgtcttctg

SEQ ID NO: 90 - DNA, 22 bp, homo sapiens
aagcttgtaggcgacctcaggc

SEQ ID NO: 91 - DNA, 22 bp, homo sapiens
tgtctaaccccggcactagaag

SEQ ID NO: 92 - DNA, 22 bp, homo sapiens
caggaacggatcgaggagtttc

SEQ ID NO: 93 - DNA, 22 bp, homo sapiens
agctccatatagtgctcccggg

SEQ ID NO: 94 - DNA, 22 bp, homo sapiens
aagtcatgctgacgatgaccac

SEQ ID NO: 95 - DNA, 22 bp, homo sapiens
tattgacgtggctctgaaggcg

SEQ ID NO: 96 - DNA, 22 bp, homo sapiens
caaagagtctttttcctggaag

SEQ ID NO: 97 - DNA, 22 bp, homo sapiens
aaagagtctttttcctggaaga

SEQ ID NO: 98 - DNA, 22 bp, homo sapiens
aataaatgaatgtttagtgtga

SEQ ID NO: 99 - DNA, 22 bp, homo sapiens
ggcagggttaatgagtccagga

SEQ ID NO: 100 - DNA, 22 bp, homo sapiens
cccagggtttaggacacatctg

SEQ ID NO: 101 - DNA, 22 bp, homo sapiens
tgcaaactcaggtctactttgg

SEQ ID NO: 102 - DNA, 22 bp, homo sapiens
acaacacagagggaaagtgata

SEQ ID NO: 103 - DNA, 22 bp, homo sapiens
accagcagcccggagacccaca

SEQ ID NO: 104 - DNA, 22 bp, homo sapiens
tcacgtggtttcggtggccgag

SEQ ID NO: 105 - DNA, 22 bp, homo sapiens
agaaaacagaaaggtgtccctc

SEQ ID NO: 106 - DNA, 22 bp, homo sapiens
ataatataagcgaaaatcaaga

SEQ ID NO: 107 - DNA, 22 bp, homo sapiens
agccaggctaacgagataatca

SEQ ID NO: 108 - DNA, 22 bp, homo sapiens
gttcctgtaggaaagaacaaag

SEQ ID NO: 109 - DNA, 22 bp, homo sapiens
actgaattagcaggaagaaaag

SEQ ID NO: 110 - DNA, 22 bp, homo sapiens
ttctgattcctctatctccatg

SEQ ID NO: 111 - DNA, 22 bp, homo sapiens
tctttcagggcggtcttgtcgt

SEQ ID NO: 112 - DNA, 22 bp, homo sapiens
cccggccctcagtggcagggga

SEQ ID NO: 113 - DNA, 22 bp, homo sapiens
agaataggcatcgttattcaag

SEQ ID NO: 114 - DNA, 22 bp, homo sapiens
aacagcagagccttagtgatat

SEQ ID NO: 115 - DNA, 22 bp, homo sapiens
aaatacatttctctaacacaag

SEQ ID NO: 116 - DNA, 22 bp, homo sapiens
actgcctcttcccaccaagtag

SEQ ID NO: 117 - DNA, 22 bp, homo sapiens
acccctgactcatgctgggtcg

SEQ ID NO: 118 - DNA, 22 bp, homo sapiens
atcctattcggtttggacttgt

SEQ ID NO: 119 - DNA, 22 bp, homo sapiens
aacaacaacaacaaaaaaaggc

SEQ ID NO: 120 - DNA, 22 bp, homo sapiens
gtgggagaaataacttccaaat

SEQ ID NO: 121 - DNA, 22 bp, homo sapiens
acaacattacataggcagaatt

SEQ ID NO: 122 - DNA, 22 bp, homo sapiens
agttgacccttgtttgtaatca

SEQ ID NO: 123 - DNA, 22 bp, homo sapiens
ttcccattccaacgtttctccg

SEQ ID NO: 124 - DNA, 22 bp, homo sapiens
cttttcagaatatattagaata

SEQ ID NO: 125 - DNA, 22 bp, homo sapiens
tagtgagaactgaggaaagaaa

SEQ ID NO: 126 - DNA, 22 bp, homo sapiens
cacacaccctcgcacgtgcatg

SEQ ID NO: 127 - DNA, 22 bp, homo sapiens
cctggggattagcagcttgatc

SEQ ID NO: 128 - DNA, 22 bp, homo sapiens
aggactccatgcgaggctccat

SEQ ID NO: 129 - DNA, 22 bp, homo sapiens
tacagcttctggcaggccgtgc

SEQ ID NO: 130 - DNA, 22 bp, homo sapiens
cagctctatgaatatctacaaa

SEQ ID NO: 131 - DNA, 22 bp, homo sapiens
tcttctattgtctctttcttgt

SEQ ID NO: 132 - DNA, 22 bp, homo sapiens
gtgaagacttacgatccttctg

SEQ ID NO: 133 - DNA, 22 bp, homo sapiens
ttatattttcaatgtgattact

SEQ ID NO: 134 - DNA, 22 bp, homo sapiens
agagacgctgttcccttgaggg

SEQ ID NO: 135 - DNA, 22 bp, homo sapiens
ctctgggttcaattaagaaggt

SEQ ID NO: 136 - DNA, 22 bp, homo sapiens
ctgggttcaattaagaaggtta

SEQ ID NO: 137 - DNA, 22 bp, homo sapiens
cctttttttttttctttccagg

SEQ ID NO: 138 - DNA, 22 bp, homo sapiens
aagggtcgagtggtggaaaata

SEQ ID NO: 139 - DNA, 22 bp, homo sapiens
aaataaaccaagcctattacat

SEQ ID NO: 140 - DNA, 22 bp, homo sapiens
atgaagattaattggtgaccaa

SEQ ID NO: 141 - DNA, 22 bp, homo sapiens
actgcacataagtttttgtttc

SEQ ID NO: 142 - DNA, 22 bp, homo sapiens
tttgtttcttgttctctcagtg

SEQ ID NO: 143 - DNA, 22 bp, homo sapiens
actttttgctttgcagctcaga

SEQ ID NO: 144 - DNA, 22 bp, homo sapiens
aaaagactcaagtatgcatcat

SEQ ID NO: 145 - DNA, 22 bp, homo sapiens
tagttggtaccgttttctcccc

SEQ ID NO: 146 - DNA, 22 bp, homo sapiens
ggcatagatttaaagaatacaa

SEQ ID NO: 147 - DNA, 22 bp, homo sapiens
ctccagataaccgacagcacac

SEQ ID NO: 148 - DNA, 22 bp, homo sapiens
aatcctaaaaatagtaatgtgt

SEQ ID NO: 149 - DNA, 22 bp, homo sapiens
cacattagatgtagaactgtgg

SEQ ID NO: 150 - DNA, 22 bp, homo sapiens
gtgattttgtttattgtaactc

SEQ ID NO: 151 - DNA, 22 bp, homo sapiens
gtgccccaagtatgttgagaca

SEQ ID NO: 152 - DNA, 22 bp, homo sapiens
ctgaatttttttccttaatgcc

SEQ ID NO: 153 - DNA, 22 bp, homo sapiens
aatcgactgacatagcagaaag

SEQ ID NO: 154 - DNA, 22 bp, homo sapiens
gggagccttagctgaaaggcag

SEQ ID NO: 155 - DNA, 22 bp, homo sapiens
caggcttacaggccggttccag

SEQ ID NO: 156 - DNA, 22 bp, homo sapiens
ctgaagtgactagggtttttaa

SEQ ID NO: 157 - DNA, 22 bp, homo sapiens
aatggtccaaggtcaccaataa

SEQ ID NO: 158 - DNA, 22 bp, homo sapiens
ttacagttacagcagatgaatg

SEQ ID NO: 159 - DNA, 22 bp, homo sapiens
ggcggaagttctcttgccacat

SEQ ID NO: 160 - DNA, 22 bp, homo sapiens
cttgccacattagagtcggtcc

SEQ ID NO: 161 - DNA, 22 bp, homo sapiens
caccaaactactttgctttaca

SEQ ID NO: 162 - DNA, 22 bp, homo sapiens
ccgtcaggtgcaggctcaacac

SEQ ID NO: 163 - DNA, 22 bp, homo sapiens
caagagcccaacgaaagtgatc

SEQ ID NO: 164 - DNA, 22 bp, homo sapiens
ttccagtctctcggtgtggcct

SEQ ID NO: 165 - DNA, 22 bp, homo sapiens
cgggcacaggcctggtgctaca

SEQ ID NO: 166 - DNA, 22 bp, homo sapiens
ttttcatctctccagggggaaa

SEQ ID NO: 167 - DNA, 22 bp, homo sapiens
atccacttttcgtagtgtgtta

SEQ ID NO: 168 - DNA, 22 bp, homo sapiens
agtgcatgattgtggtagggtg

SEQ ID NO: 169 - DNA, 22 bp, homo sapiens
aattcaaggacgaatattttca

SEQ ID NO: 170 - DNA, 22 bp, homo sapiens
taaacgtatctctggtaatgag

SEQ ID NO: 171 - DNA, 22 bp, homo sapiens
aaaaacaaggtagtatcacaat

SEQ ID NO: 172 - DNA, 22 bp, homo sapiens
catctgtttatttttaagaggt

SEQ ID NO: 173 - DNA, 22 bp, homo sapiens
catcagtgacaggtacacacct

SEQ ID NO: 174 - DNA, 22 bp, homo sapiens
caagccactgtataaggtttca

SEQ ID NO: 175 - DNA, 22 bp, homo sapiens
ctgagagtagtcgactattaca

SEQ ID NO: 176 - DNA, 22 bp, homo sapiens
tccttttaataagagaactaat

SEQ ID NO: 177 - DNA, 22 bp, homo sapiens
aactttataaacggtaaaactg

SEQ ID NO: 178 - DNA, 22 bp, homo sapiens
cattggatttgcgaacaaagta

SEQ ID NO: 179 - DNA, 22 bp, homo sapiens
cactcataaatataataaagac

SEQ ID NO: 180 - DNA, 22 bp, homo sapiens
atttagttttgataagaagaaa

SEQ ID NO: 181 - DNA, 22 bp, homo sapiens
cagatatccttcgatactttat

SEQ ID NO: 182 - DNA, 22 bp, homo sapiens
tagttatgtattagtgcaagct

SEQ ID NO: 183 - DNA, 22 bp, homo sapiens
tatatctaaatccactgtgtat

SEQ ID NO: 184 - DNA, 22 bp, homo sapiens
tgaaagagtacgacctacatat

SEQ ID NO: 185 - DNA, 22 bp, homo sapiens
gggcaataaacaacttccaaag

SEQ ID NO: 186 - DNA, 22 bp, homo sapiens
gttaagaaggaaaagaccacca

SEQ ID NO: 187 - DNA, 22 bp, homo sapiens
gagtgatctttgttccattgta

SEQ ID NO: 188 - DNA, 22 bp, homo sapiens
atggtaaaggtaagaaagaagg

SEQ ID NO: 189 - DNA, 22 bp, homo sapiens
aggataaccatgtgaggacttg

SEQ ID NO: 190 - DNA, 22 bp, homo sapiens
tttccctgcagcagtgcgaggt

SEQ ID NO: 191 - DNA, 22 bp, homo sapiens
tgggaattgccgacaggatgca

SEQ ID NO: 192 - DNA, 22 bp, homo sapiens
aaggctgtggccaagtatcttc

SEQ ID NO: 193 - DNA, 22 bp, homo sapiens
ttcgattcaactgtccaacaaa

SEQ ID NO: 194 - DNA, 22 bp, homo sapiens
ggagtgtaatgccatacctgtt

SEQ ID NO: 195 - DNA, 22 bp, homo sapiens
agtgagatgttaatagctataa

SEQ ID NO: 196 - DNA, 22 bp, homo sapiens
aaggaagatgaggaggggaaag

SEQ ID NO: 197 - DNA, 22 bp, homo sapiens
tccttggcctgggtagagtggc

SEQ ID NO: 198 - DNA, 22 bp, homo sapiens
tggtgcccatattagccaggga

SEQ ID NO: 199 - DNA, 22 bp, homo sapiens
aaacgctttcgcgccgctcaga

SEQ ID NO: 200 - DNA, 22 bp, homo sapiens
cgctttcgcgccgctcagaaga

SEQ ID NO: 201 - DNA, 22 bp, homo sapiens
cgtgcgcagtgcggtttggcct

SEQ ID NO: 202 - DNA, 22 bp, homo sapiens
cagaagtttaacggccacgacg

SEQ ID NO: 203 - DNA, 22 bp, homo sapiens
attaagctgaaaatcaccaaaa

SEQ ID NO: 204 - DNA, 22 bp, homo sapiens
gtgtctaagatctcaagtcctt

SEQ ID NO: 205 - DNA, 22 bp, homo sapiens
gtttgcttgacctgtcagagtg

SEQ ID NO: 206 - DNA, 22 bp, homo sapiens
ctggagctcagatcgcagcaag

SEQ ID NO: 207 - DNA, 22 bp, homo sapiens
tgttctttgcacctctctctcc

SEQ ID NO: 208 - DNA, 22 bp, homo sapiens
cgagtgttcccgtacatggagg

SEQ ID NO: 209 - DNA, 22 bp, homo sapiens
tggtgaaaattccctttaaaaa

SEQ ID NO: 210 - DNA, 22 bp, homo sapiens
ggcctgtttgccgtctgtgaca

SEQ ID NO: 211 - DNA, 22 bp, homo sapiens
tgtgtgctcacatcttgtgttc

SEQ ID NO: 212 - DNA, 22 bp, homo sapiens
aatatagattataaaaactact

SEQ ID NO: 213 - DNA, 22 bp, homo sapiens
ctttgaaaagccaagtgactct

SEQ ID NO: 214 - DNA, 22 bp, homo sapiens
aagaaaggcacaaaattctttt

SEQ ID NO: 215 - DNA, 22 bp, homo sapiens
gcttacttgtccgctaggtaac

SEQ ID NO: 216 - DNA, 22 bp, homo sapiens
aaatctttagtacttttttcag

SEQ ID NO: 217 - DNA, 22 bp, homo sapiens
atcagactaacatgaacacaga

SEQ ID NO: 218 - DNA, 22 bp, homo sapiens
aaagaatcatagtcaatctcta

SEQ ID NO: 219 - DNA, 22 bp, homo sapiens
ttaggtcaaacggaggtttaat

SEQ ID NO: 220 - DNA, 22 bp, homo sapiens
gagtaaaccatactgaatagca

SEQ ID NO: 221 - DNA, 22 bp, homo sapiens
tgtgaagagatggcttgtttcc

SEQ ID NO: 222 - DNA, 22 bp, homo sapiens
tacaataatgaagcatggctga

SEQ ID NO: 223 - DNA, 22 bp, homo sapiens
tacagcaacttgctatatactg

SEQ ID NO: 224 - DNA, 22 bp, homo sapiens
ctgaaaaaccatctgaaaagcg

SEQ ID NO: 225 - DNA, 22 bp, homo sapiens
tatttggtttcattgtaatgat

SEQ ID NO: 226 - DNA, 22 bp, homo sapiens
gttcatctgtactatgttggtg

SEQ ID NO: 227 - DNA, 22 bp, homo sapiens
agtcaggtaatatacataagga

SEQ ID NO: 228 - DNA, 22 bp, homo sapiens
agtaaccccagcgagaagggaa

SEQ ID NO: 229 - DNA, 22 bp, homo sapiens
agcagtttgctgaatgacccct

SEQ ID NO: 230 - DNA, 22 bp, homo sapiens
gtggccaagcagagcactgccc

SEQ ID NO: 231 - DNA, 22 bp, homo sapiens
cccagcatctccgtgagcagtg

SEQ ID NO: 232 - DNA, 22 bp, homo sapiens
tgggaaaagaggaggatctcag

SEQ ID NO: 233 - DNA, 22 bp, homo sapiens
ttgctggtagacgtgtctttta

SEQ ID NO: 234 - DNA, 22 bp, homo sapiens
gaatacatccacgacttcaaca

SEQ ID NO: 235 - DNA, 22 bp, homo sapiens
aagagcaggaccgcagtggacg

SEQ ID NO: 236 - DNA, 22 bp, homo sapiens
catctttcgccgttcttcatag

SEQ ID NO: 237 - DNA, 22 bp, homo sapiens
tactgaaagcaatggagccaga

SEQ ID NO: 238 - DNA, 22 bp, homo sapiens
ccagaactgagcaccttgtcac

SEQ ID NO: 239 - DNA, 22 bp, homo sapiens
gctcagaagaatgagcaagggg

SEQ ID NO: 240 - DNA, 22 bp, homo sapiens
accagaaaggcgacatgaatcc

SEQ ID NO: 241 - DNA, 22 bp, homo sapiens
caccaactccctttgccagaag

SEQ ID NO: 242 - DNA, 22 bp, homo sapiens
aattccatgccgaccacaggtt

SEQ ID NO: 243 - DNA, 22 bp, homo sapiens
gccccagagtgggcatccccgt

SEQ ID NO: 244 - DNA, 22 bp, homo sapiens
ggcggtctgcagcctcagattc

SEQ ID NO: 245 - DNA, 22 bp, homo sapiens
ggctgtaatcccagtttgccta

SEQ ID NO: 246 - DNA, 22 bp, homo sapiens
agaacggggaagtccgggtctc

SEQ ID NO: 247 - DNA, 22 bp, homo sapiens
agcaaatcattcgattgtggaa

SEQ ID NO: 248 - DNA, 22 bp, homo sapiens
aggagaggcccactggtacggc

SEQ ID NO: 249 - DNA, 22 bp, homo sapiens
tggtcgactttcccctcaacaa

SEQ ID NO: 250 - DNA, 22 bp, homo sapiens
agcccctggacggtgtgatgtt

SEQ ID NO: 251 - DNA, 22 bp, homo sapiens
gaaaacatcaagtcatccacat

SEQ ID NO: 252 - DNA, 22 bp, homo sapiens
acctgctccttcagaaaatact

SEQ ID NO: 253 - DNA, 22 bp, homo sapiens
ctttgaaatgtacagacaataa

SEQ ID NO: 254 - DNA, 22 bp, homo sapiens
aaatcactatactgtcaagcaa

SEQ ID NO: 255 - DNA, 22 bp, homo sapiens
gcacttgtgcacttggaaagca

SEQ ID NO: 256 - DNA, 22 bp, homo sapiens
gttatgagcgatctggatatga

SEQ ID NO: 257 - DNA, 22 bp, homo sapiens
tgtcataaaaggattgctctct

SEQ ID NO: 258 - DNA, 22 bp, homo sapiens
atttagagcccatcatctgtaa

SEQ ID NO: 259 - DNA, 22 bp, homo sapiens
tcataggaatacggtcacagaa

SEQ ID NO: 260 - DNA, 22 bp, homo sapiens
gtcaagtagccgttgatgcaca

SEQ ID NO: 261 - DNA, 22 bp, homo sapiens
cagcttcccaaagcaaagcatg

SEQ ID NO: 262 - DNA, 22 bp, homo sapiens
caggcccacagcactaccgcgg

SEQ ID NO: 263 - DNA, 22 bp, homo sapiens
agaacttccaagggagagaagt

SEQ ID NO: 264 - DNA, 22 bp, homo sapiens
cagatgtgacctcatggcagag

SEQ ID NO: 265 - DNA, 22 bp, homo sapiens
ctcctggaagaactcagtaagt

SEQ ID NO: 266 - DNA, 22 bp, homo sapiens
tttgttgcagaatggaaaaagg

SEQ ID NO: 267 - DNA, 22 bp, homo sapiens
tccctacccaacccagccaagg

SEQ ID NO: 268 - DNA, 22 bp, homo sapiens
acccagccaaggggcaccttaa

SEQ ID NO: 269 - DNA, 22 bp, homo sapiens
gtggctccgccgatgttcattc

SEQ ID NO: 270 - DNA, 22 bp, homo sapiens
ccttgctgtctcgtttacaagt

SEQ ID NO: 271 - DNA, 22 bp, homo sapiens
ggacctgtcaaaatccttacct

SEQ ID NO: 272 - DNA, 22 bp, homo sapiens
atggtctggagcctgaaggcag

SEQ ID NO: 273 - DNA, 22 bp, homo sapiens
gacagagttctcggagccggct

SEQ ID NO: 274 - DNA, 22 bp, homo sapiens
gttctcggagccggctcagagc

SEQ ID NO: 275 - DNA, 22 bp, homo sapiens
tggccagaaaagttgttccctc

SEQ ID NO: 276 - DNA, 22 bp, homo sapiens
ttccctcgaaggtgggtttctt

SEQ ID NO: 277 - DNA, 22 bp, homo sapiens
ttttctctaaccctctagaaaa

SEQ ID NO: 278 - DNA, 22 bp, homo sapiens
atgtgtctcagctgcatggcca

SEQ ID NO: 279 - DNA, 22 bp, homo sapiens
ggatcttcaggctctacgtgag

SEQ ID NO: 280 - DNA, 22 bp, homo sapiens
aggctctacgtgagcataggct

SEQ ID NO: 281 - DNA, 22 bp, homo sapiens
gagaccttgacccctcttctaa

SEQ ID NO: 282 - DNA, 22 bp, homo sapiens
ctctctccctccccagctgctg

SEQ ID NO: 283 - DNA, 22 bp, homo sapiens
ccattctctttgccattggggt

SEQ ID NO: 284 - DNA, 22 bp, homo sapiens
cctgccaatccccggccccacc

SEQ ID NO: 285 - DNA, 22 bp, homo sapiens
atggacgagcacgcccggggga

SEQ ID NO: 286 - DNA, 22 bp, homo sapiens
tgaatgaaggttgcagaaatgg

SEQ ID NO: 287 - DNA, 22 bp, homo sapiens
ccaatattaaggactataaaac

SEQ ID NO: 288 - DNA, 22 bp, homo sapiens
agttgaagaacgttgttccaaa

SEQ ID NO: 289 - DNA, 22 bp, homo sapiens
atataaagtctatgtactccag

SEQ ID NO: 290 - DNA, 22 bp, homo sapiens
tttcaacatgaataaatttaaa

SEQ ID NO: 291 - DNA, 22 bp, homo sapiens
ggggccgctttatcagtgatgg

SEQ ID NO: 292 - DNA, 22 bp, homo sapiens
gtccaatttcaatatgtactct

SEQ ID NO: 293 - DNA, 22 bp, homo sapiens
tcttcaatgccgtctcagctat

SEQ ID NO: 294 - DNA, 22 bp, homo sapiens
ccatttcaacactattagactt

SEQ ID NO: 295 - DNA, 22 bp, homo sapiens
actattttatcgtaacaactaa

SEQ ID NO: 296 - DNA, 22 bp, homo sapiens
aaagactgaataatctcctttt

SEQ ID NO: 297 - DNA, 22 bp, homo sapiens
cttatccaggagaatcaagagc

SEQ ID NO: 298 - DNA, 22 bp, homo sapiens
agattctgtacattgttaacct

SEQ ID NO: 299 - DNA, 22 bp, homo sapiens
ttcagcctactcgtgctgctga

SEQ ID NO: 300 - DNA, 22 bp, homo sapiens
cggctccggggggcacggggct

SEQ ID NO: 301 - DNA, 22 bp, homo sapiens
aaggtttttatttgttggtttg

SEQ ID NO: 302 - DNA, 22 bp, homo sapiens
gaagaaccgatgatgagccctg

SEQ ID NO: 303 - DNA, 22 bp, homo sapiens
ctgaggggagccggagttgggc

SEQ ID NO: 304 - DNA, 22 bp, homo sapiens
aaatgacgctcaagttttcctg

SEQ ID NO: 305 - DNA, 22 bp, homo sapiens
cagcagggtccctagtccctgg

SEQ ID NO: 306 - DNA, 22 bp, homo sapiens
tcaccccccccccgcctgtctc

SEQ ID NO: 307 - DNA, 22 bp, homo sapiens
accccccccccgcctgtctccc

SEQ ID NO: 308 - DNA, 22 bp, homo sapiens
tatggcccagctgtctgttttt

SEQ ID NO: 309 - DNA, 22 bp, homo sapiens
gttattagcacaagccttaaga

SEQ ID NO: 310 - DNA, 22 bp, homo sapiens
gtaaaatgtgacgtaaaaatta

SEQ ID NO: 311 - DNA, 22 bp, homo sapiens
aggtgtgcacttattttgaaag

SEQ ID NO: 312 - DNA, 22 bp, homo sapiens
ttgttaaatgctcacttaatgg

SEQ ID NO: 313 - DNA, 22 bp, homo sapiens
caggtaggatatgaatgctcag

SEQ ID NO: 314 - DNA, 22 bp, homo sapiens
tagttagtaaaccattatttta

SEQ ID NO: 315 - DNA, 22 bp, homo sapiens
gttttaagatgcctaccacagt

SEQ ID NO: 316 - DNA, 22 bp, homo sapiens
cacagttccttacagatagcac

SEQ ID NO: 317 - DNA, 22 bp, homo sapiens
atattatttttgccattttttc

SEQ ID NO: 318 - DNA, 22 bp, homo sapiens
tagttttgtaatcctgtatcct

SEQ ID NO: 319 - DNA, 22 bp, homo sapiens
ttaaaagaagatacatgtttta

SEQ ID NO: 320 - DNA, 22 bp, homo sapiens
aatatttgacaaatttttactg

SEQ ID NO: 321 - DNA, 22 bp, homo sapiens
tatggttctgaaaaaaaaaata

SEQ ID NO: 322 - DNA, 22 bp, homo sapiens
tcctggtttgcatacctttttg

SEQ ID NO: 323 - DNA, 22 bp, homo sapiens
acacacgtagaacaggttagcc

SEQ ID NO: 324 - DNA, 22 bp, homo sapiens
ccgggcagagagagggtaggag

SEQ ID NO: 325 - DNA, 22 bp, homo sapiens
tccgttttgcattgtacttgcc

SEQ ID NO: 326 - DNA, 22 bp, homo sapiens
tccatcacttccagtgttgggt

SEQ ID NO: 327 - DNA, 22 bp, homo sapiens
cttggggggccgcttacctcct

SEQ ID NO: 328 - DNA, 22 bp, homo sapiens
cagtgagatcaatttctcatcc

SEQ ID NO: 329 - DNA, 22 bp, homo sapiens
tccgcggggagcgtgacagcag

SEQ ID NO: 330 - DNA, 22 bp, homo sapiens
gaggttcaggacgagctgcttc

SEQ ID NO: 331 - DNA, 22 bp, homo sapiens
cgggctcgtgggctgcactgtc

SEQ ID NO: 332 - DNA, 22 bp, homo sapiens
tttcagagccatcacgcctggc

SEQ ID NO: 333 - DNA, 22 bp, homo sapiens
tgcactttttgtgctcttgggg

SEQ ID NO: 334 - DNA, 22 bp, homo sapiens
ggaaactgtggcatgttcacgt

SEQ ID NO: 335 - DNA, 22 bp, homo sapiens
actgaagaggtccaagaaagga

SEQ ID NO: 336 - DNA, 22 bp, homo sapiens
ttacctgttccatagctcctgg

SEQ ID NO: 337 - DNA, 22 bp, homo sapiens
atcgttctggtagggaaaactg

SEQ ID NO: 338 - DNA, 22 bp, homo sapiens
acaccatccttggagaggaaat

SEQ ID NO: 339 - DNA, 22 bp, homo sapiens
ttcctaatttactgtgatttgt

SEQ ID NO: 340 - DNA, 22 bp, homo sapiens
tttgtgccccatgaggtttgtt

SEQ ID NO: 341 - DNA, 22 bp, homo sapiens
tgccgtggaacataatatgcag

SEQ ID NO: 342 - DNA, 22 bp, homo sapiens
tataaacatttacttacacttt

SEQ ID NO: 343 - DNA, 22 bp, homo sapiens
gactgcggcaggttggacacta

SEQ ID NO: 344 - DNA, 22 bp, homo sapiens
agaagattggagaaggcttgta

SEQ ID NO: 345 - DNA, 22 bp, homo sapiens
cgggagtccatcaagttggtga

SEQ ID NO: 346 - DNA, 22 bp, homo sapiens
aaaaggcttggcgctgggcagt

SEQ ID NO: 347 - DNA, 22 bp, homo sapiens
atggcatacatcgagcggaaga

SEQ ID NO: 348 - DNA, 22 bp, homo sapiens
ccaatcccgtaagaagtgaaca

SEQ ID NO: 349 - DNA, 22 bp, homo sapiens
ttatttggactgtaccgctggc

SEQ ID NO: 350 - DNA, 22 bp, homo sapiens
aagcctgaaatgactacttaca

SEQ ID NO: 351 - DNA, 22 bp, homo sapiens
gtaaatgaactggttcttggag

SEQ ID NO: 352 - DNA, 22 bp, homo sapiens
ggaggatgccaccaggaacata

SEQ ID NO: 353 - DNA, 22 bp, homo sapiens
ggcggcatcgccggaggcaggg

SEQ ID NO: 354 - DNA, 22 bp, homo sapiens
aaaaagaaaatctttacaatta

SEQ ID NO: 355 - DNA, 22 bp, homo sapiens
gctaaggagctggctgcggagg

SEQ ID NO: 356 - DNA, 22 bp, homo sapiens
gatgaggctctcggtggcaggt

SEQ ID NO: 357 - DNA, 22 bp, homo sapiens
cccaccggggtgtcactgcggg

SEQ ID NO: 358 - DNA, 22 bp, homo sapiens
ctaggattccacacagactggc

SEQ ID NO: 359 - DNA, 22 bp, homo sapiens
ttacatcaatggcatatgaccg

SEQ ID NO: 360 - DNA, 22 bp, homo sapiens
ctgacccggctgtctttctgtg

SEQ ID NO: 361 - DNA, 22 bp, homo sapiens
gctgccctgctcaagctgtcct

SEQ ID NO: 362 - DNA, 22 bp, homo sapiens
gtggtcattaccctgccattca

SEQ ID NO: 363 - DNA, 22 bp, homo sapiens
ggaagtaatggagaagaggtga

SEQ ID NO: 364 - DNA, 22 bp, homo sapiens
ccagagcctgcgaagacaagag

SEQ ID NO: 365 - DNA, 22 bp, homo sapiens
ctctggaaaacctgttcaatct

SEQ ID NO: 366 - DNA, 22 bp, homo sapiens
atccgtctctacgctgctatca

SEQ ID NO: 367 - DNA, 22 bp, homo sapiens
atttatactacggggagaaaga

SEQ ID NO: 368 - DNA, 22 bp, homo sapiens
ataagaatttacttcaagaaat

SEQ ID NO: 369 - DNA, 22 bp, homo sapiens
cttaaaaaaaaatcctgctata

SEQ ID NO: 370 - DNA, 22 bp, homo sapiens
aataaaacaaataaacaaatta

SEQ ID NO: 371 - DNA, 22 bp, homo sapiens
gtttcctctggaaaaaaaaaaa

SEQ ID NO: 372 - DNA, 22 bp, homo sapiens
ttaaagcaataattagtgaaat

SEQ ID NO: 373 - DNA, 22 bp, homo sapiens
gattctatcatcctggagtctt

SEQ ID NO: 374 - DNA, 22 bp, homo sapiens
tatatgactggcggacgggtca

SEQ ID NO: 375 - DNA, 22 bp, homo sapiens
tgactggcggacgggtcatgag

SEQ ID NO: 376 - DNA, 22 bp, homo sapiens
agatatagatatatgcaatgcg

SEQ ID NO: 377 - DNA, 22 bp, homo sapiens
ctctatgcccaactggaaatat

SEQ ID NO: 378 - DNA, 22 bp, homo sapiens
gagagaccaaacggaagagtcc

SEQ ID NO: 379 - DNA, 22 bp, homo sapiens
atgtttgccctggtgtgctgct

SEQ ID NO: 380 - DNA, 22 bp, homo sapiens
ggtactcatgggcttgatttga

SEQ ID NO: 381 - DNA, 22 bp, homo sapiens
cccaaagcaagttggactaaat

SEQ ID NO: 382 - DNA, 22 bp, homo sapiens
gtgcactgactgggggtgctga

SEQ ID NO: 383 - DNA, 22 bp, homo sapiens
agacagcaaaagaacagagaga

SEQ ID NO: 384 - DNA, 22 bp, homo sapiens
cctctaagggcgttttcctcag

SEQ ID NO: 385 - DNA, 22 bp, homo sapiens
gtggctcctccggtcaccaggg

SEQ ID NO: 386 - DNA, 22 bp, homo sapiens
ggataactcaccgcgctcatga

SEQ ID NO: 387 - DNA, 22 bp, homo sapiens
taagaaataaaccaacctcctc

SEQ ID NO: 388 - DNA, 22 bp, homo sapiens
atgttgagtgacgccaagatgc

SEQ ID NO: 389 - DNA, 22 bp, homo sapiens
atgcagaccacgacatggccac

SEQ ID NO: 390 - DNA, 22 bp, homo sapiens
tggggcagaagcgggcaacatc

SEQ ID NO: 391 - DNA, 22 bp, homo sapiens
gatattaactcgctgacctttg

SEQ ID NO: 392 - DNA, 22 bp, homo sapiens
caattggctagcggggctcagt

SEQ ID NO: 393 - DNA, 22 bp, homo sapiens
ttgtttaacatttcttctggga

SEQ ID NO: 394 - DNA, 22 bp, homo sapiens
aattttactgtctccaaatggg

SEQ ID NO: 395 - DNA, 22 bp, homo sapiens
ccaaaagggccgtgaacagcca

SEQ ID NO: 396 - DNA, 22 bp, homo sapiens
acaggaccctgcatcccacccc

SEQ ID NO: 397 - DNA, 22 bp, homo sapiens
aggattctaccgcagcagaggc

SEQ ID NO: 398 - DNA, 22 bp, homo sapiens
actcagagagggtcttgttgca

SEQ ID NO: 399 - DNA, 22 bp, homo sapiens
gtaacagcacccgcttagcagg

SEQ ID NO: 400 - DNA, 22 bp, homo sapiens
cattgtttgtagggagacagcc

SEQ ID NO: 401 - DNA, 22 bp, homo sapiens
gtagtgcttagggtagacgatg

SEQ ID NO: 402 - DNA, 22 bp, homo sapiens
caaaacattcagaacaaagtta

SEQ ID NO: 403 - DNA, 22 bp, homo sapiens
atttgcctagaaaaaaaaaaaa

SEQ ID NO: 404 - DNA, 22 bp, homo sapiens
agcaagcttcaaaagtggtgaa

SEQ ID NO: 405 - DNA, 22 bp, homo sapiens
ggtagagtagagcaggggtgag

SEQ ID NO: 406 - DNA, 22 bp, homo sapiens
ggaaagaaactatccacggacc

SEQ ID NO: 407 - DNA, 22 bp, homo sapiens
tatgtgaatggtagaaactttg

SEQ ID NO: 408 - DNA, 22 bp, homo sapiens
cagcaagctcacgtgcaggcac

SEQ ID NO: 409 - DNA, 22 bp, homo sapiens
acctcagattggggcgtggcga

SEQ ID NO: 410 - DNA, 22 bp, homo sapiens
agctaccacaggatcaaaacag

SEQ ID NO: 411 - DNA, 22 bp, homo sapiens
tattggggttgccattttgtga

SEQ ID NO: 412 - DNA, 22 bp, homo sapiens
aagaatgtgtagtaattaataa

SEQ ID NO: 413 - DNA, 22 bp, homo sapiens
tatttatacagcgtgtatattt

SEQ ID NO: 414 - DNA, 22 bp, homo sapiens
atttatacagcgtgtatatttc

SEQ ID NO: 415 - DNA, 22 bp, homo sapiens
ttacaaacactaagccagctcc

SEQ ID NO: 416 - DNA, 22 bp, homo sapiens
ttgcccttttgatttattatct

SEQ ID NO: 417 - DNA, 22 bp, homo sapiens
agtaagtctactaaaaagccta

SEQ ID NO: 418 - DNA, 22 bp, homo sapiens
gcacttttgtggaggacactca

SEQ ID NO: 419 - DNA, 22 bp, homo sapiens
atttgtggatatgtaggagtct

SEQ ID NO: 420 - DNA, 22 bp, homo sapiens
tcagccaccctcagtagtgctg

SEQ ID NO: 421 - DNA, 22 bp, homo sapiens
gaggattcaacatgggaatcac

SEQ ID NO: 422 - DNA, 22 bp, homo sapiens
cagcatctttggggtgatggta

SEQ ID NO: 423 - DNA, 22 bp, homo sapiens
agctggtttctgttgatcggtg

SEQ ID NO: 424 - DNA, 22 bp, homo sapiens
ccagtgcccccgtaactcactg

SEQ ID NO: 425 - DNA, 22 bp, homo sapiens
ttcctttgcaaggtatccatgg

SEQ ID NO: 426 - DNA, 22 bp, homo sapiens
aacctggtagccgtacagtctt

SEQ ID NO: 427 - DNA, 22 bp, homo sapiens
acttccgggtacgcctcctctt

SEQ ID NO: 428 - DNA, 22 bp, homo sapiens
tgggaatgcagatggctggaca

SEQ ID NO: 429 - DNA, 22 bp, homo sapiens
ccacctgcaagcgccactttat

SEQ ID NO: 430 - DNA, 22 bp, homo sapiens
ccaggacagctgtctctgagtg

SEQ ID NO: 431 - DNA, 22 bp, homo sapiens
ggatccggcaggtatgagtgct

SEQ ID NO: 432 - DNA, 22 bp, homo sapiens
cactccttcaaggtcagcaact

SEQ ID NO: 433 - DNA, 22 bp, homo sapiens
ttctgattctgcgtttcctatt

SEQ ID NO: 434 - DNA, 22 bp, homo sapiens
aattgcattacaagacaaaggt

SEQ ID NO: 435 - DNA, 22 bp, homo sapiens
ggtaaatgcacactgagaagaa

SEQ ID NO: 436 - DNA, 22 bp, homo sapiens
tgagttctgaagtccaaagtaa

SEQ ID NO: 437 - DNA, 22 bp, homo sapiens
ctccatatttgggggtgcagat

SEQ ID NO: 438 - DNA, 22 bp, homo sapiens
gccagctctgagaatgagagac

SEQ ID NO: 439 - DNA, 22 bp, homo sapiens
caactcagcattagcccagcct

SEQ ID NO: 440 - DNA, 22 bp, homo sapiens
cgcaggagttccgataagtgaa

SEQ ID NO: 441 - DNA, 22 bp, homo sapiens
gtgcaggagagctgcaagaggg

SEQ ID NO: 442 - DNA, 22 bp, homo sapiens
caggcctatcccttcctatcct

SEQ ID NO: 443 - DNA, 22 bp, homo sapiens
tgtatctgttaaattataatag

SEQ ID NO: 444 - DNA, 22 bp, homo sapiens
ctcaccaaagatgtaaagttgc

SEQ ID NO: 445 - DNA, 22 bp, homo sapiens
ttcactattcttatttgcctct

SEQ ID NO: 446 - DNA, 22 bp, homo sapiens
tgatctctagaaagcgatctga

SEQ ID NO: 447 - DNA, 22 bp, homo sapiens
aaatccagaggtatcatgaaac

SEQ ID NO: 448 - DNA, 22 bp, homo sapiens
aagtaagtaaagcagtaaacaa

SEQ ID NO: 449 - DNA, 22 bp, homo sapiens
cgaaactaagagccatttttct

SEQ ID NO: 450 - DNA, 22 bp, homo sapiens
gaagttcagaagtggactaacc

SEQ ID NO: 451 - DNA, 22 bp, homo sapiens
cttttttttttttctttaaaca

SEQ ID NO: 452 - DNA, 22 bp, homo sapiens
ttcaaccttctgattggggctg

SEQ ID NO: 453 - DNA, 22 bp, homo sapiens
tccgttttcatactgctcattc

SEQ ID NO: 454 - DNA, 22 bp, homo sapiens
gatgggggagaaaaaaaaaaag

SEQ ID NO: 455 - DNA, 22 bp, homo sapiens
catcttttaagttgataacacc

SEQ ID NO: 456 - DNA, 22 bp, homo sapiens
acactagttactcccagaaatc

SEQ ID NO: 457 - DNA, 22 bp, homo sapiens
ttaaacatgaggcacacacagc

SEQ ID NO: 458 - DNA, 22 bp, homo sapiens
ctgctttcagcatcagccctcc

SEQ ID NO: 459 - DNA, 22 bp, homo sapiens
tttaagaggaaagtcccaagtc

SEQ ID NO: 460 - DNA, 22 bp, homo sapiens
tactggcacatcgggaggagaa

SEQ ID NO: 461 - DNA, 22 bp, homo sapiens
agaaaaggtataattttttaaa

SEQ ID NO: 462 - DNA, 22 bp, homo sapiens
gcttttgtcatccccatcctgg

SEQ ID NO: 463 - DNA, 22 bp, homo sapiens
aaatagacgaggtcctgtcgca

SEQ ID NO: 464 - DNA, 22 bp, homo sapiens
ttgaatacctgtttagtatcgt

SEQ ID NO: 465 - DNA, 22 bp, homo sapiens
ttttggattccatctagcatgg

SEQ ID NO: 466 - DNA, 22 bp, homo sapiens
agggagggcttgtagttaggtt

SEQ ID NO: 467 - DNA, 22 bp, homo sapiens
atgactctaagccctctgtccc

SEQ ID NO: 468 - DNA, 22 bp, homo sapiens
cgctgcggtcgcacagctcgca

SEQ ID NO: 469 - DNA, 22 bp, homo sapiens
cacgggggcagcgctctggtgt

SEQ ID NO: 470 - DNA, 22 bp, homo sapiens
gaggcacctcggtcatggagtg

SEQ ID NO: 471 - DNA, 22 bp, homo sapiens
aatttgctctatttgcagaagc

SEQ ID NO: 472 - DNA, 22 bp, homo sapiens
acaaggacatagctgttcccta

SEQ ID NO: 473 - DNA, 22 bp, homo sapiens
gagagcctgccgtttaaagtat

SEQ ID NO: 474 - DNA, 22 bp, homo sapiens
tttgtgtcggtggttatgacaa

SEQ ID NO: 475 - DNA, 22 bp, homo sapiens
gacagcagcggcgaccctgatg

SEQ ID NO: 476 - DNA, 22 bp, homo sapiens
tttcttcccgatcaagatcaga

SEQ ID NO: 477 - DNA, 22 bp, homo sapiens
gcttgcttctgtcatcgaaatc

SEQ ID NO: 478 - DNA, 22 bp, homo sapiens
tctgaagcatacctgcaaacaa

SEQ ID NO: 479 - DNA, 22 bp, homo sapiens
agggcaggataggagtaagaga

SEQ ID NO: 480 - DNA, 22 bp, homo sapiens
tagcgctccccggcagccctgg

SEQ ID NO: 481 - DNA, 22 bp, homo sapiens
cctgccaagaacagagagacac

SEQ ID NO: 482 - DNA, 22 bp, homo sapiens
acaaaggtacatacatacctga

SEQ ID NO: 483 - DNA, 22 bp, homo sapiens
aaaaaaaaaaaagttggcatga

SEQ ID NO: 484 - DNA, 22 bp, homo sapiens
taaaatgatcacattttccaaa

SEQ ID NO: 485 - DNA, 22 bp, homo sapiens
gtctgcctccacggatagtgaa

SEQ ID NO: 486 - DNA, 22 bp, homo sapiens
gtcctatgcaacaataacacag

SEQ ID NO: 487 - DNA, 22 bp, homo sapiens
tttctttgtctagatggattcc

SEQ ID NO: 488 - DNA, 22 bp, homo sapiens
atgagtatgttccatgcaatat

SEQ ID NO: 489 - DNA, 22 bp, homo sapiens
ccagatcactccttcaccctcc

SEQ ID NO: 490 - DNA, 22 bp, homo sapiens
attttttttttttctggtagga

SEQ ID NO: 491 - DNA, 22 bp, homo sapiens
ttatttgatgacctggcaactt

SEQ ID NO: 492 - DNA, 22 bp, homo sapiens
ttcaaacttcagttcctatctg

SEQ ID NO: 493 - DNA, 22 bp, homo sapiens
ctagcaacatttatggttatag

SEQ ID NO: 494 - DNA, 22 bp, homo sapiens
ctttcaacttaccaaaatcaat

SEQ ID NO: 495 - DNA, 22 bp, homo sapiens
tttttttttttaataggtatga

SEQ ID NO: 496 - DNA, 22 bp, homo sapiens
tagtaatattttcaaaattata

SEQ ID NO: 497 - DNA, 22 bp, homo sapiens
taatgcccattgtgtattgata

SEQ ID NO: 498 - DNA, 22 bp, homo sapiens
gtgtgctgcggcgagtgcttcc

SEQ ID NO: 499 - DNA, 22 bp, homo sapiens
tgattaattacggcacagacat

SEQ ID NO: 500 - DNA, 22 bp, homo sapiens
atactggccaacagttttagag

SEQ ID NO: 501 - DNA, 22 bp, homo sapiens
ttccacacggagtcccttctca

SEQ ID NO: 502 - DNA, 22 bp, homo sapiens
tgacaatatttgataaaacttt

SEQ ID NO: 503 - DNA, 22 bp, homo sapiens
agacatgggaatcactctgcat

SEQ ID NO: 504 - DNA, 22 bp, homo sapiens
tttatgtagtattgaacatttt

SEQ ID NO: 505 - DNA, 22 bp, homo sapiens
cgtagttggcatagatatctat

SEQ ID NO: 506 - DNA, 22 bp, homo sapiens
tagaggttctcgagtggagtaa

SEQ ID NO: 507 - DNA, 22 bp, homo sapiens
gattttttttttatttaactaa

SEQ ID NO: 508 - DNA, 22 bp, homo sapiens
agcagggtgtgaggaatttcac

SEQ ID NO: 509 - DNA, 22 bp, homo sapiens
tgagcaaaactactctggataa

SEQ ID NO: 510 - DNA, 22 bp, homo sapiens
aataaaggttagcacatagtaa

SEQ ID NO: 511 - DNA, 22 bp, homo sapiens
catagtaagtgctcattaagta

SEQ ID NO: 512 - DNA, 22 bp, homo sapiens
ttccacagtcatggtaaagttc

SEQ ID NO: 513 - DNA, 22 bp, homo sapiens
agtgtaatttattaaggggttt

SEQ ID NO: 514 - DNA, 22 bp, homo sapiens
atttattaaggggtttacaaat

SEQ ID NO: 515 - DNA, 22 bp, homo sapiens
taaggggtttacaaatatgttt

SEQ ID NO: 516 - DNA, 22 bp, homo sapiens
aggtgggtatccgcttttccct

SEQ ID NO: 517 - DNA, 22 bp, homo sapiens
agagacccaaaccctaggtctg

SEQ ID NO: 518 - DNA, 22 bp, homo sapiens
ctgcaccccacactagacacct

SEQ ID NO: 519 - DNA, 22 bp, homo sapiens
actccatatctcaagttctcca

SEQ ID NO: 520 - DNA, 22 bp, homo sapiens
cgttcctagggaaaaaaaaaaa

SEQ ID NO: 521 - DNA, 22 bp, homo sapiens
gaactactacattgggaacatg

SEQ ID NO: 522 - DNA, 22 bp, homo sapiens
caaagattttctggttacttac

SEQ ID NO: 523 - DNA, 22 bp, homo sapiens
taaaaaagcatatgtaaagtac

SEQ ID NO: 524 - DNA, 22 bp, homo sapiens
ctattcatttcgtaccctaaac

SEQ ID NO: 525 - DNA, 22 bp, homo sapiens
ccagcttcctcggacaacctgg

SEQ ID NO: 526 - DNA, 22 bp, homo sapiens
cccacgctgagagtcgtcttag

SEQ ID NO: 527 - DNA, 22 bp, homo sapiens
tttcttaccctccatcttctgc

SEQ ID NO: 528 - DNA, 22 bp, homo sapiens
agctacacacacgagattatac

SEQ ID NO: 529 - DNA, 22 bp, homo sapiens
cacatcaggcactggaactatc

SEQ ID NO: 530 - DNA, 22 bp, homo sapiens
aagtggcactcgagtgctgttt

SEQ ID NO: 531 - DNA, 22 bp, homo sapiens
cagcaagttcgtccagcaactt

SEQ ID NO: 532 - DNA, 22 bp, homo sapiens
aaggtcctcttctaagtcctcc

SEQ ID NO: 533 - DNA, 22 bp, homo sapiens
aacaatcgacaggctagaagtt

SEQ ID NO: 534 - DNA, 22 bp, homo sapiens
tcacaatgatctgatgctgggg

SEQ ID NO: 535 - DNA, 22 bp, homo sapiens
catttgctggtaagttattgta

SEQ ID NO: 536 - DNA, 22 bp, homo sapiens
acttttacttatagctttattt

SEQ ID NO: 537 - DNA, 22 bp, homo sapiens
ggaggttatctgtagagacagt

SEQ ID NO: 538 - DNA, 22 bp, homo sapiens
cctggtgaaattaggtcttctt

SEQ ID NO: 539 - DNA, 22 bp, homo sapiens
taactggttctggagaatctgg

SEQ ID NO: 540 - DNA, 22 bp, homo sapiens
gtataggtaatcctcctgggcc

SEQ ID NO: 541 - DNA, 22 bp, homo sapiens
gtcggggagatccctggtcaag

SEQ ID NO: 542 - DNA, 22 bp, homo sapiens
gttgcggatttggcgaaagtta

SEQ ID NO: 543 - DNA, 22 bp, homo sapiens
gggcaaacacataagccgctat

SEQ ID NO: 544 - DNA, 22 bp, homo sapiens
gttccatatcaatcagccagct

SEQ ID NO: 545 - DNA, 22 bp, homo sapiens
accccccatgcggaagatatgg

SEQ ID NO: 546 - DNA, 22 bp, homo sapiens
aactcgtgattgtcggggggaa

SEQ ID NO: 547 - DNA, 22 bp, homo sapiens
acatctgtttgagataacttgg

SEQ ID NO: 548 - DNA, 22 bp, homo sapiens
ggaatacagtgaacactttcca

SEQ ID NO: 549 - DNA, 22 bp, homo sapiens
ttatgaaaagatgagcacagca

SEQ ID NO: 550 - DNA, 22 bp, homo sapiens
tcaagtgagtacccctctccca

SEQ ID NO: 551 - DNA, 22 bp, homo sapiens
aatatccgtcagcagacgcctg

SEQ ID NO: 552 - DNA, 22 bp, homo sapiens
tttcacgagaatagaatgtggc

SEQ ID NO: 553 - DNA, 22 bp, homo sapiens
ggctccagtcagtggttttttt

SEQ ID NO: 554 - DNA, 22 bp, homo sapiens
tcaccaagaaagaggtaaaccc

SEQ ID NO: 555 - DNA, 22 bp, homo sapiens
tcgaggaaggagctgtgggagt

SEQ ID NO: 556 - DNA, 22 bp, homo sapiens
ctcgtacccgacgtctcctacg

SEQ ID NO: 557 - DNA, 22 bp, homo sapiens
ctggatgtcaacgagggcccag

SEQ ID NO: 558 - DNA, 22 bp, homo sapiens
taaatgtttaaatatacacatg

SEQ ID NO: 559 - DNA, 22 bp, homo sapiens
tacacacgccctggtaagcctt

SEQ ID NO: 560 - DNA, 22 bp, homo sapiens
atagcaacaggaaaaaaaaaaa

SEQ ID NO: 561 - DNA, 22 bp, homo sapiens
cgcggccatggcgcgcgcttac

SEQ ID NO: 562 - DNA, 22 bp, homo sapiens
gtgattcagcagcatcaccggc

SEQ ID NO: 563 - DNA, 22 bp, homo sapiens
ggtcagtgccattccacagctg

SEQ ID NO: 564 - DNA, 22 bp, homo sapiens
tagaaacagacaccacatactt

SEQ ID NO: 565 - DNA, 22 bp, homo sapiens
gtgtcctggtacgactggggca

SEQ ID NO: 566 - DNA, 22 bp, homo sapiens
tgccccaagcgaaaacagaaaa

SEQ ID NO: 567 - DNA, 22 bp, homo sapiens
aagctctcctggagcaaatatg

SEQ ID NO: 568 - DNA, 22 bp, homo sapiens
gagagggtctccttcctatgcg

SEQ ID NO: 569 - DNA, 22 bp, homo sapiens
cagctgtgctctcgtcccctgc

SEQ ID NO: 570 - DNA, 22 bp, homo sapiens
gctcatcttcctaccgagtgct

SEQ ID NO: 571 - DNA, 22 bp, homo sapiens
ttctgaaacgccgcggatcaat

SEQ ID NO: 572 - DNA, 22 bp, homo sapiens
agctctgacctccatgttcttc

SEQ ID NO: 573 - DNA, 22 bp, homo sapiens
cgtggaaaaaacgaaacaaaag

SEQ ID NO: 574 - DNA, 22 bp, homo sapiens
atccaagagctcctaccaggca

SEQ ID NO: 575 - DNA, 22 bp, homo sapiens
tccatgtccccgggcctgtaag

SEQ ID NO: 576 - DNA, 22 bp, homo sapiens
aagtatcaagcgttcattcagt

SEQ ID NO: 577 - DNA, 22 bp, homo sapiens
tcccataagagaattggctttg

SEQ ID NO: 578 - DNA, 22 bp, homo sapiens
gtaacgaggctcccttttgttt

SEQ ID NO: 579 - DNA, 22 bp, homo sapiens
caaagctcccattggtgtaatt

SEQ ID NO: 580 - DNA, 22 bp, homo sapiens
atccttacctgaacctctgtct

SEQ ID NO: 581 - DNA, 22 bp, homo sapiens
attgctagggccacacatttat

SEQ ID NO: 582 - DNA, 22 bp, homo sapiens
tcttcagcaagctgtcttactt

SEQ ID NO: 583 - DNA, 22 bp, homo sapiens
cttactcttatttgatccacaa

SEQ ID NO: 584 - DNA, 22 bp, homo sapiens
ctaaggaaagagaaacagcttt

SEQ ID NO: 585 - DNA, 22 bp, homo sapiens
ttattatactagcagatccttt

SEQ ID NO: 586 - DNA, 22 bp, homo sapiens
aagaaaaagagggggtaggtgg

SEQ ID NO: 587 - DNA, 22 bp, homo sapiens
aagagggggtaggtggaaacaa

SEQ ID NO: 588 - DNA, 22 bp, homo sapiens
gtcttaaaattcatgacaacca

SEQ ID NO: 589 - DNA, 22 bp, homo sapiens
caggacctgggcgtgaagaact

SEQ ID NO: 590 - DNA, 22 bp, homo sapiens
ccgtgtcctccggtgtgtgtga

SEQ ID NO: 591 - DNA, 22 bp, homo sapiens
gtgtgaggccaagctcctgggg

SEQ ID NO: 592 - DNA, 22 bp, homo sapiens
gggaagacaggggaatggcatt

SEQ ID NO: 593 - DNA, 22 bp, homo sapiens
ttgggtaaatccaccttaatca

SEQ ID NO: 594 - DNA, 22 bp, homo sapiens
cgtggctctgtgcagatattga

SEQ ID NO: 595 - DNA, 22 bp, homo sapiens
cccatcccccacgcagctggca

SEQ ID NO: 596 - DNA, 22 bp, homo sapiens
gccacagggatgggcccatctc

SEQ ID NO: 597 - DNA, 22 bp, homo sapiens
ggaccggctggtgaacgagaag

SEQ ID NO: 598 - DNA, 22 bp, homo sapiens
cagtctcatcaaccagaaatag

SEQ ID NO: 599 - DNA, 22 bp, homo sapiens
ttgggtaagttcctgtatgtca

SEQ ID NO: 600 - DNA, 22 bp, homo sapiens
accaccccaaagaattccttct

SEQ ID NO: 601 - DNA, 22 bp, homo sapiens
tagttgaaatattatattgagg

SEQ ID NO: 602 - DNA, 22 bp, homo sapiens
aatattatattgaggggtcttt

SEQ ID NO: 603 - DNA, 22 bp, homo sapiens
tgatttgtgtacactgttggtt

SEQ ID NO: 604 - DNA, 22 bp, homo sapiens
aaaggagctgttgccgcacaag

SEQ ID NO: 605 - DNA, 22 bp, homo sapiens
gccgtggtccacggtacttgaa

SEQ ID NO: 606 - DNA, 22 bp, homo sapiens
gtggacctgtccccacgatgca

SEQ ID NO: 607 - DNA, 22 bp, homo sapiens
cctgcacccgccgtcattcctc

SEQ ID NO: 608 - DNA, 22 bp, homo sapiens
aaggaatatatcgatggcctgg

SEQ ID NO: 609 - DNA, 22 bp, homo sapiens
ttcctgtgaaatctgcgtggct

SEQ ID NO: 610 - DNA, 22 bp, homo sapiens
catctggtcaccgtggtggctt

SEQ ID NO: 611 - DNA, 22 bp, homo sapiens
ttagaaattcaccctctttccc

SEQ ID NO: 612 - DNA, 22 bp, homo sapiens
cacagcaccctgccctactcac

SEQ ID NO: 613 - DNA, 22 bp, homo sapiens
ttgtccgtccgcgggttcctgg

SEQ ID NO: 614 - DNA, 22 bp, homo sapiens
ggcacaggcaggctcagagcag

SEQ ID NO: 615 - DNA, 22 bp, homo sapiens
gcagactcagacgcatccccag

SEQ ID NO: 616 - DNA, 22 bp, homo sapiens
cgagcaagggcaggcaagttac

SEQ ID NO: 617 - DNA, 22 bp, homo sapiens
cagcccttggactgaggcaggc

SEQ ID NO: 618 - DNA, 22 bp, homo sapiens
tacagcttacacgggaaatcac

SEQ ID NO: 619 - DNA, 22 bp, homo sapiens
agaaggaagattggctgttact

SEQ ID NO: 620 - DNA, 22 bp, homo sapiens
atcaaggagcagagaagttctg

SEQ ID NO: 621 - DNA, 22 bp, homo sapiens
ctccctagaagagaagcaacaa

SEQ ID NO: 622 - DNA, 22 bp, homo sapiens
tggtggcgtgggaagctcttgc

SEQ ID NO: 623 - DNA, 22 bp, homo sapiens
gggaccttggcccaaacatcag

SEQ ID NO: 624 - DNA, 22 bp, homo sapiens
gcagtgctcacgccccggaaag

SEQ ID NO: 625 - DNA, 22 bp, homo sapiens
gggactgcgtcccagagatggg

SEQ ID NO: 626 - DNA, 22 bp, homo sapiens
ttgactcttccgcaggtagggg

SEQ ID NO: 627 - DNA, 22 bp, homo sapiens
gcagcccatgggtgggactctg

SEQ ID NO: 628 - DNA, 22 bp, homo sapiens
aggagaggaagaagggaccagc

SEQ ID NO: 629 - DNA, 22 bp, homo sapiens
ccttaagaagggataaaagtga

SEQ ID NO: 630 - DNA, 22 bp, homo sapiens
agaacctcatccgtcagagaag

SEQ ID NO: 631 - DNA, 22 bp, homo sapiens
tgcattgaatgcgggaaattct

SEQ ID NO: 632 - DNA, 22 bp, homo sapiens
gtgccaggtacgtgggaacctt

SEQ ID NO: 633 - DNA, 22 bp, homo sapiens
gacctgaagacgcagctgtcac

SEQ ID NO: 634 - DNA, 22 bp, homo sapiens
gagtgtgttggggggcttgtga

SEQ ID NO: 635 - DNA, 22 bp, homo sapiens
tccccttagggtccgaaagatt

SEQ ID NO: 636 - DNA, 22 bp, homo sapiens
cctacatgcaggtgcacattca

SEQ ID NO: 637 - DNA, 22 bp, homo sapiens
gaaaagaagaccgcagtgccag

SEQ ID NO: 638 - DNA, 22 bp, homo sapiens
tttcaaagttgaaaaaccatcg

SEQ ID NO: 639 - DNA, 22 bp, homo sapiens
aaaaaccatcgcgtcatgtagg

SEQ ID NO: 640 - DNA, 22 bp, homo sapiens
ctctgtagtcacctgcttgcat

SEQ ID NO: 641 - DNA, 22 bp, homo sapiens
tttcttgtctcgcacaggcttt

SEQ ID NO: 642 - DNA, 22 bp, homo sapiens
gggtgagagcgcgaggctccgg

SEQ ID NO: 643 - DNA, 22 bp, homo sapiens
tcccttgtatccggcaggtggt

SEQ ID NO: 644 - DNA, 22 bp, homo sapiens
atacacgacaggcctgcagctc

SEQ ID NO: 645 - DNA, 22 bp, homo sapiens
gctcacaggcacgcacagggag

SEQ ID NO: 646 - DNA, 22 bp, homo sapiens
tagctagttgggtgatgaaggt

SEQ ID NO: 647 - DNA, 22 bp, homo sapiens
ccctctggggtggtccagccct

SEQ ID NO: 648 - DNA, 22 bp, homo sapiens
gagtagggcttctccccagagt

SEQ ID NO: 649 - DNA, 22 bp, homo sapiens
agccaccggctccccaggctga

SEQ ID NO: 650 - DNA, 22 bp, homo sapiens
agggcgatcccgcctggatgcg

SEQ ID NO: 651 - DNA, 22 bp, homo sapiens
ccgggcaaagacgtcatcctca

SEQ ID NO: 652 - DNA, 22 bp, homo sapiens
aacagcactgcatcatgcttga

SEQ ID NO: 653 - DNA, 22 bp, homo sapiens
catgattcagacaggaacgaag

SEQ ID NO: 654 - DNA, 22 bp, homo sapiens
cagataggaacaaagctgaaac

SEQ ID NO: 655 - DNA, 22 bp, homo sapiens
tttgaactcactgaacatttct

SEQ ID NO: 656 - DNA, 22 bp, homo sapiens
agccctgaggcgccacctgccc

SEQ ID NO: 657 - DNA, 22 bp, homo sapiens
tcagggtcagctggtgcctggc

SEQ ID NO: 658 - DNA, 22 bp, homo sapiens
ctcctcagccttctctttcaga

SEQ ID NO: 659 - DNA, 22 bp, homo sapiens
tctcttggtcagtggcactctt

SEQ ID NO: 660 - DNA, 22 bp, homo sapiens
gcaggcattgttctcatcctcg

SEQ ID NO: 661 - DNA, 22 bp, homo sapiens
tcactctgtgttctcaaacctc

SEQ ID NO: 662 - DNA, 22 bp, homo sapiens
cctgaacaaagcctgagccagc

SEQ ID NO: 663 - DNA, 22 bp, homo sapiens
aacaccagcacaacacctccga

SEQ ID NO: 664 - DNA, 22 bp, homo sapiens
gaaggagaagatgctggggttg

SEQ ID NO: 665 - DNA, 22 bp, homo sapiens
caggccttgtgtcgatggcact

SEQ ID NO: 666 - DNA, 22 bp, homo sapiens
cggtagggctctaggtctaaag

SEQ ID NO: 667 - DNA, 22 bp, homo sapiens
acccctacagcttgaggaagct

SEQ ID NO: 668 - DNA, 22 bp, homo sapiens
gagctcctgcaatcggatgagc

SEQ ID NO: 669 - DNA, 22 bp, homo sapiens
aaccgagtggagctggggtatg

SEQ ID NO: 670 - DNA, 22 bp, homo sapiens
tcggggtagagattcacgtaga

SEQ ID NO: 671 - DNA, 22 bp, homo sapiens
gcaaatgcagcacttcccctcc

SEQ ID NO: 672 - DNA, 22 bp, homo sapiens
cagcaatcaatgaagcaaacat

SEQ ID NO: 673 - DNA, 22 bp, homo sapiens
ctcttcacatgcatctcagaaa

SEQ ID NO: 674 - DNA, 22 bp, homo sapiens
ggttaattcctatctcacaaat

SEQ ID NO: 675 - DNA, 22 bp, homo sapiens
cacgggagcacctggatcgcat

SEQ ID NO: 676 - DNA, 22 bp, homo sapiens
cttttggaggaaggtggtggga

SEQ ID NO: 677 - DNA, 22 bp, homo sapiens
agttttaagtaattttttgctg

SEQ ID NO: 678 - DNA, 22 bp, homo sapiens
ttaaaaaaaaacacaacaccac

SEQ ID NO: 679 - DNA, 22 bp, homo sapiens
aaaaaaaaacaccaccacccac

SEQ ID NO: 680 - DNA, 22 bp, homo sapiens
acgcccaagagttatcgggggc

SEQ ID NO: 681 - DNA, 22 bp, homo sapiens
ttgtatgattaatgcacagaat

SEQ ID NO: 682 - DNA, 22 bp, homo sapiens
agtgcacagaacttctgtgccc

SEQ ID NO: 683 - DNA, 22 bp, homo sapiens
atccaatcctcttagagctccc

SEQ ID NO: 684 - DNA, 22 bp, homo sapiens
ctggaaatcactggcaaagtct

SEQ ID NO: 685 - DNA, 22 bp, homo sapiens
gatgaggtcagtgacaccgaca

SEQ ID NO: 686 - DNA, 22 bp, homo sapiens
cagtggcatcaacctgcagtct

SEQ ID NO: 687 - DNA, 22 bp, homo sapiens
ggaccacccctttgagaagccc

SEQ ID NO: 688 - DNA, 22 bp, homo sapiens
ccaggaggagtctgagatggag

SEQ ID NO: 689 - DNA, 22 bp, homo sapiens
acacagccatgcgtcagggggc

SEQ ID NO: 690 - DNA, 22 bp, homo sapiens
tgaatttggggacaaccaacgt

SEQ ID NO: 691 - DNA, 22 bp, homo sapiens
tgggtcagttttgaaccaggct

SEQ ID NO: 692 - DNA, 22 bp, homo sapiens
gtataccttagaacatgaagat

SEQ ID NO: 693 - DNA, 22 bp, homo sapiens
acacccccaagtctccagggtc

SEQ ID NO: 694 - DNA, 22 bp, homo sapiens
tgctatacccaaaaggttcggg

SEQ ID NO: 695 - DNA, 22 bp, homo sapiens
gtcgctctgcaaccgggtgatg

SEQ ID NO: 696 - DNA, 22 bp, homo sapiens
caaagctgcacatggtaaaaaa

SEQ ID NO: 697 - DNA, 22 bp, homo sapiens
ggtcctcgaacatgtgtgccgc

SEQ ID NO: 698 - DNA, 22 bp, homo sapiens
aaggccgcggtagcgaaggcgt

SEQ ID NO: 699 - DNA, 22 bp, homo sapiens
caaccccgttattcaggccatg

SEQ ID NO: 700 - DNA, 22 bp, homo sapiens
taaggatccctgggataccgga

SEQ ID NO: 701 - DNA, 22 bp, homo sapiens
tttagggggaaaaaaggaagaa

SEQ ID NO: 702 - DNA, 22 bp, homo sapiens
tgagggtcagcatgtttacagc

SEQ ID NO: 703 - DNA, 22 bp, homo sapiens
acagtcgcaattgcttccacga

SEQ ID NO: 704 - DNA, 22 bp, homo sapiens
tttcgactgacatcaggaacta

SEQ ID NO: 705 - DNA, 22 bp, homo sapiens
ttattgagaagtctatgaaaga

SEQ ID NO: 706 - DNA, 22 bp, homo sapiens
atttccttgatgtaaccaattg

SEQ ID NO: 707 - DNA, 22 bp, homo sapiens
tttccttgatacaaccaattgc

SEQ ID NO: 708 - DNA, 22 bp, homo sapiens
gaaccacgagggaagaaaaaca

SEQ ID NO: 709 - DNA, 22 bp, homo sapiens
gtgaccttgtcattagttactg

SEQ ID NO: 710 - DNA, 22 bp, homo sapiens
tctgtgatctgctaatgtggaa

SEQ ID NO: 711 - DNA, 22 bp, homo sapiens
acttagagttactcatttctgg

SEQ ID NO: 712 - DNA, 22 bp, homo sapiens
tgtccagtagcccttttaacct

SEQ ID NO: 713 - DNA, 22 bp, homo sapiens
ctttcttcgatgtgcctgaata

SEQ ID NO: 714 - DNA, 22 bp, homo sapiens
cataggaaaatcgatctgtaaa

SEQ ID NO: 715 - DNA, 22 bp, homo sapiens
aagtcgagaagtccttgcacag

SEQ ID NO: 716 - DNA, 22 bp, homo sapiens
atacgtagaatcattcaaaagt

SEQ ID NO: 717 - DNA, 22 bp, homo sapiens
taagtcatcttaattcagttgg

SEQ ID NO: 718 - DNA, 22 bp, homo sapiens
acattctgtaaaatagtttttg

SEQ ID NO: 719 - DNA, 22 bp, homo sapiens
atgtacacattttgttctaaaa

SEQ ID NO: 720 - DNA, 22 bp, homo sapiens
tgccgagcactctctaaaagac

SEQ ID NO: 721 - DNA, 22 bp, homo sapiens
agtaaattatttggaaactata

SEQ ID NO: 722 - DNA, 22 bp, homo sapiens
ttccctactccccatttttctt

SEQ ID NO: 723 - DNA, 22 bp, homo sapiens
tggtacgactcatgggatgttg

SEQ ID NO: 724 - DNA, 22 bp, homo sapiens
gtttataaactgtgtgagttat

SEQ ID NO: 725 - DNA, 22 bp, homo sapiens
ccatgcaagtatgagtattaaa

SEQ ID NO: 726 - DNA, 22 bp, homo sapiens
attgttgcatagatatctggtt

SEQ ID NO: 727 - DNA, 22 bp, homo sapiens
ttccaggcaacagcaaagaccc

SEQ ID NO: 728 - DNA, 22 bp, homo sapiens
cagaggggagcaaaatgctgca

SEQ ID NO: 729 - DNA, 22 bp, homo sapiens
cgagggtcgactgcgatgcctg

SEQ ID NO: 730 - DNA, 22 bp, homo sapiens
cctggctggtgtccagacctcg

SEQ ID NO: 731 - DNA, 22 bp, homo sapiens
acccgcagcgcagcctcaatgc

SEQ ID NO: 732 - DNA, 22 bp, homo sapiens
actcactcacctgactcctccc

SEQ ID NO: 733 - DNA, 22 bp, homo sapiens
aatctctaggaaccttgtggct

SEQ ID NO: 734 - DNA, 22 bp, homo sapiens
cttgtttccataaagcgcagga

SEQ ID NO: 735 - DNA, 22 bp, homo sapiens
tcaggtcctagatgactgcgca

SEQ ID NO: 736 - DNA, 22 bp, homo sapiens
cccactgactaggcctggacgt

SEQ ID NO: 737 - DNA, 22 bp, homo sapiens
gtgagatcatattgcacgacca

SEQ ID NO: 738 - DNA, 22 bp, homo sapiens
tttcatccaagaaatcttaaat

SEQ ID NO: 739 - DNA, 22 bp, homo sapiens
aaagatgatgggaggaagggcc

SEQ ID NO: 740 - DNA, 22 bp, homo sapiens
caatagcccccggtgtcttctg

SEQ ID NO: 741 - DNA, 22 bp, homo sapiens
aagcttgtaggggacctcaggc

SEQ ID NO: 742 - DNA, 22 bp, homo sapiens
tgtctaaccccagcactagaag

SEQ ID NO: 743 - DNA, 22 bp, homo sapiens
caggaacggattgaggagtttc

SEQ ID NO: 744 - DNA, 22 bp, homo sapiens
agctccatatactgctcccggg

SEQ ID NO: 745 - DNA, 22 bp, homo sapiens
aagtcatgctggcgatgaccac

SEQ ID NO: 746 - DNA, 22 bp, homo sapiens
tattgacgtggttctgaaggcg

SEQ ID NO: 747 - DNA, 22 bp, homo sapiens
caaagagtcttcttcctggaag

SEQ ID NO: 748 - DNA, 22 bp, homo sapiens
aaagagtctttctcctggaaga

SEQ ID NO: 749 - DNA, 22 bp, homo sapiens
aataaatgaatttttagtgtga

SEQ ID NO: 750 - DNA, 22 bp, homo sapiens
ggcagggttaacgagtccagga

SEQ ID NO: 751 - DNA, 22 bp, homo sapiens
cccagggtttaagacacatctg

SEQ ID NO: 752 - DNA, 22 bp, homo sapiens
tgcaaactcagatctactttgg

SEQ ID NO: 753 - DNA, 22 bp, homo sapiens
acaacacagagagaaagtgata

SEQ ID NO: 754 - DNA, 22 bp, homo sapiens
accagcagcccagagacccaca

SEQ ID NO: 755 - DNA, 22 bp, homo sapiens
tcacgtggttttggtggccgag

SEQ ID NO: 756 - DNA, 22 bp, homo sapiens
agaaaacagaacggtgtccctc

SEQ ID NO: 757 - DNA, 22 bp, homo sapiens
ataatataagcaaaaatcaaga

SEQ ID NO: 758 - DNA, 22 bp, homo sapiens
agccaggctaatgagataatca

SEQ ID NO: 759 - DNA, 22 bp, homo sapiens
gttcctgtagggaagaacaaag

SEQ ID NO: 760 - DNA, 22 bp, homo sapiens
actgaattagcgggaagaaaag

SEQ ID NO: 761 - DNA, 22 bp, homo sapiens
ttctgattcctttatctccatg

SEQ ID NO: 762 - DNA, 22 bp, homo sapiens
tctttcagggcagtcttgtcgt

SEQ ID NO: 763 - DNA, 22 bp, homo sapiens
cccggccctcattggcagggga

SEQ ID NO: 764 - DNA, 22 bp, homo sapiens
agaataggcattgttattcaag

SEQ ID NO: 765 - DNA, 22 bp, homo sapiens
aacagcagagctttagtgatat

SEQ ID NO: 766 - DNA, 22 bp, homo sapiens
aaatacatttccctaacacaag

SEQ ID NO: 767 - DNA, 22 bp, homo sapiens
actgcctcttcacaccaagtag

SEQ ID NO: 768 - DNA, 22 bp, homo sapiens
acccctgactcgtgctgggtcg

SEQ ID NO: 769 - DNA, 22 bp, homo sapiens
atcctattcggcttggacttgt

SEQ ID NO: 770 - DNA, 22 bp, homo sapiens
aacaacaacaaaaaaaaaaggc

SEQ ID NO: 771 - DNA, 22 bp, homo sapiens
gtgggagaaattacttccaaat

SEQ ID NO: 772 - DNA, 22 bp, homo sapiens
acaacattacacaggcagaatt

SEQ ID NO: 773 - DNA, 22 bp, homo sapiens
agttgacccttctttgtaatca

SEQ ID NO: 774 - DNA, 22 bp, homo sapiens
ttcccattccagcgtttctccg

SEQ ID NO: 775 - DNA, 22 bp, homo sapiens
cttttcagaatgtattagaata

SEQ ID NO: 776 - DNA, 22 bp, homo sapiens
tagtgagaactaaggaaagaaa

SEQ ID NO: 777 - DNA, 22 bp, homo sapiens
cacacaccctcacacgtgcatg

SEQ ID NO: 778 - DNA, 22 bp, homo sapiens
cctggggattaacagcttgatc

SEQ ID NO: 779 - DNA, 22 bp, homo sapiens
aggactccatgagaggctccat

SEQ ID NO: 780 - DNA, 22 bp, homo sapiens
tacagcttctgccaggccgtgc

SEQ ID NO: 781 - DNA, 22 bp, homo sapiens
cagctctatgagtatctacaaa

SEQ ID NO: 782 - DNA, 22 bp, homo sapiens
tcttctattgtttctttcttgt

SEQ ID NO: 783 - DNA, 22 bp, homo sapiens
gtgaagacttatgatccttctg

SEQ ID NO: 784 - DNA, 22 bp, homo sapiens
ttatattttcagtgtgattact

SEQ ID NO: 785 - DNA, 22 bp, homo sapiens
agagacgctgtgcccttgaggg

SEQ ID NO: 786 - DNA, 22 bp, homo sapiens
ctctgggttcagttaagaaggt

SEQ ID NO: 787 - DNA, 22 bp, homo sapiens
ctgggttcaatcaagaaggtta

SEQ ID NO: 788 - DNA, 22 bp, homo sapiens
cctttttttttctctttccagg

SEQ ID NO: 789 - DNA, 22 bp, homo sapiens
aagggtcgagttgtggaaaata

SEQ ID NO: 790 - DNA, 22 bp, homo sapiens
aaataaaccaaacctattacat

SEQ ID NO: 791 - DNA, 22 bp, homo sapiens
atgaagattaagtggtgaccaa

SEQ ID NO: 792 - DNA, 22 bp, homo sapiens
actgcacataaatttttgtttc

SEQ ID NO: 793 - DNA, 22 bp, homo sapiens
tttgtttcttggtctctcagtg

SEQ ID NO: 794 - DNA, 22 bp, homo sapiens
actttttgcttcgcagctcaga

SEQ ID NO: 795 - DNA, 22 bp, homo sapiens
aaaagactcaaatatgcatcat

SEQ ID NO: 796 - DNA, 22 bp, homo sapiens
tagttggtacctttttctcccc

SEQ ID NO: 797 - DNA, 22 bp, homo sapiens
ggcatagatttcaagaatacaa

SEQ ID NO: 798 - DNA, 22 bp, homo sapiens
ctccagataactgacagcacac

SEQ ID NO: 799 - DNA, 22 bp, homo sapiens
aatcctaaaaacagtaatgtgt

SEQ ID NO: 800 - DNA, 22 bp, homo sapiens
cacattagatgaagaactgtgg

SEQ ID NO: 801 - DNA, 22 bp, homo sapiens
gtgattttgttaattgtaactc

SEQ ID NO: 802 - DNA, 22 bp, homo sapiens
gtgccccaagtgtgttgagaca

SEQ ID NO: 803 - DNA, 22 bp, homo sapiens
ctgaattttttcccttaatgcc

SEQ ID NO: 804 - DNA, 22 bp, homo sapiens
aatcgactgacgtagcagaaag

SEQ ID NO: 805 - DNA, 22 bp, homo sapiens
gggagccttagttgaaaggcag

SEQ ID NO: 806 - DNA, 22 bp, homo sapiens
caggcttacagcccggttccag

SEQ ID NO: 807 - DNA, 22 bp, homo sapiens
ctgaagtgactggggtttttaa

SEQ ID NO: 808 - DNA, 22 bp, homo sapiens
aatggtccaagatcaccaataa

SEQ ID NO: 809 - DNA, 22 bp, homo sapiens
ttacagttacaccagatgaatg

SEQ ID NO: 810 - DNA, 22 bp, homo sapiens
ggcggaagttcccttgccacat

SEQ ID NO: 811 - DNA, 22 bp, homo sapiens
cttgccacattggagtcggtcc

SEQ ID NO: 812 - DNA, 22 bp, homo sapiens
caccaaactaccttgctttaca

SEQ ID NO: 813 - DNA, 22 bp, homo sapiens
ccgtcaggtgcgggctcaacac

SEQ ID NO: 814 - DNA, 22 bp, homo sapiens
caagagcccaatgaaagtgatc

SEQ ID NO: 815 - DNA, 22 bp, homo sapiens
ttccagtctcttggtgtggcct

SEQ ID NO: 816 - DNA, 22 bp, homo sapiens
cgggcacaggcgtggtgctaca

SEQ ID NO: 817 - DNA, 22 bp, homo sapiens
ttttcatctctgcagggggaaa

SEQ ID NO: 818 - DNA, 22 bp, homo sapiens
atccacttttcatagtgtgtta

SEQ ID NO: 819 - DNA, 22 bp, homo sapiens
agtgcatgattatggtagggtg

SEQ ID NO: 820 - DNA, 22 bp, homo sapiens
aattcaaggacaaatattttca

SEQ ID NO: 821 - DNA, 22 bp, homo sapiens
taaacgtatctttggtaatgag

SEQ ID NO: 822 - DNA, 22 bp, homo sapiens
aaaaacaaggtggtatcacaat

SEQ ID NO: 823 - DNA, 22 bp, homo sapiens
catctgtttatctttaagaggt

SEQ ID NO: 824 - DNA, 22 bp, homo sapiens
catcagtgacaagtacacacct

SEQ ID NO: 825 - DNA, 22 bp, homo sapiens
caagccactgtgtaaggtttca

SEQ ID NO: 826 - DNA, 22 bp, homo sapiens
ctgagagtagttgactattaca

SEQ ID NO: 827 - DNA, 22 bp, homo sapiens
tccttttaataggagaactaat

SEQ ID NO: 828 - DNA, 22 bp, homo sapiens
aactttataaatggtaaaactg

SEQ ID NO: 829 - DNA, 22 bp, homo sapiens
cattggatttgtgaacaaagta

SEQ ID NO: 830 - DNA, 22 bp, homo sapiens
cactcataaatgtaataaagac

SEQ ID NO: 831 - DNA, 22 bp, homo sapiens
atttagttttggtaagaagaaa

SEQ ID NO: 832 - DNA, 22 bp, homo sapiens
cagatatcctttgatactttat

SEQ ID NO: 833 - DNA, 22 bp, homo sapiens
tagttatgtatcagtgcaagct

SEQ ID NO: 834 - DNA, 22 bp, homo sapiens
tatatctaaatacactgtgtat

SEQ ID NO: 835 - DNA, 22 bp, homo sapiens
tgaaagagtacaacctacatat

SEQ ID NO: 836 - DNA, 22 bp, homo sapiens
gggcaataaaccacttccaaag

SEQ ID NO: 837 - DNA, 22 bp, homo sapiens
gttaagaaggagaagaccacca

SEQ ID NO: 838 - DNA, 22 bp, homo sapiens
gagtgatcttttttccattgta

SEQ ID NO: 839 - DNA, 22 bp, homo sapiens
atggtaaaggtcagaaagaagg

SEQ ID NO: 840 - DNA, 22 bp, homo sapiens
aggataaccatctgaggacttg

SEQ ID NO: 841 - DNA, 22 bp, homo sapiens
tttccctgcagtagtgcgaggt

SEQ ID NO: 842 - DNA, 22 bp, homo sapiens
tgggaattgccaacaggatgca

SEQ ID NO: 843 - DNA, 22 bp, homo sapiens
aaggctgtggcgaagtatcttc

SEQ ID NO: 844 - DNA, 22 bp, homo sapiens
ttcgattcaacagtccaacaaa

SEQ ID NO: 845 - DNA, 22 bp, homo sapiens
ggagtgtaatgacatacctgtt

SEQ ID NO: 846 - DNA, 22 bp, homo sapiens
agtgagatgttcatagctataa

SEQ ID NO: 847 - DNA, 22 bp, homo sapiens
aaggaagatgacgaggggaaag

SEQ ID NO: 848 - DNA, 22 bp, homo sapiens
tccttggcctgagtagagtggc

SEQ ID NO: 849 - DNA, 22 bp, homo sapiens
tggtgcccatactagccaggga

SEQ ID NO: 850 - DNA, 22 bp, homo sapiens
aaacgctttcgtgccgctcaga

SEQ ID NO: 851 - DNA, 22 bp, homo sapiens
cgctttcgcgctgctcagaaga

SEQ ID NO: 852 - DNA, 22 bp, homo sapiens
cgtgcgcagtgtggtttggcct

SEQ ID NO: 853 - DNA, 22 bp, homo sapiens
cagaagtttaatggccacgacg

SEQ ID NO: 854 - DNA, 22 bp, homo sapiens
attaagctgaagatcaccaaaa

SEQ ID NO: 855 - DNA, 22 bp, homo sapiens
gtgtctaagatttcaagtcctt

SEQ ID NO: 856 - DNA, 22 bp, homo sapiens
gtttgcttgacttgtcagagtg

SEQ ID NO: 857 - DNA, 22 bp, homo sapiens
ctggagctcaggtcgcagcaag

SEQ ID NO: 858 - DNA, 22 bp, homo sapiens
tgttctttgcatctctctctcc

SEQ ID NO: 859 - DNA, 22 bp, homo sapiens
cgagtgttcccatacatggagg

SEQ ID NO: 860 - DNA, 22 bp, homo sapiens
tggtgaaaatttcctttaaaaa

SEQ ID NO: 861 - DNA, 22 bp, homo sapiens
ggcctgtttgctgtctgtgaca

SEQ ID NO: 862 - DNA, 22 bp, homo sapiens
tgtgtgctcacttcttgtgttc

SEQ ID NO: 863 - DNA, 22 bp, homo sapiens
aatatagattacaaaaactact

SEQ ID NO: 864 - DNA, 22 bp, homo sapiens
ctttgaaaagctaagtgactct

SEQ ID NO: 865 - DNA, 22 bp, homo sapiens
aagaaaggcactaaattctttt

SEQ ID NO: 866 - DNA, 22 bp, homo sapiens
gcttacttgtctgctaggtaac

SEQ ID NO: 867 - DNA, 22 bp, homo sapiens
aaatctttagttcttttttcag

SEQ ID NO: 868 - DNA, 22 bp, homo sapiens
atcagactaacgtgaacacaga

SEQ ID NO: 869 - DNA, 22 bp, homo sapiens
aaagaatcataatcaatctcta

SEQ ID NO: 870 - DNA, 22 bp, homo sapiens
ttaggtcaaacagaggtttaat

SEQ ID NO: 871 - DNA, 22 bp, homo sapiens
gagtaaaccatgctgaatagca

SEQ ID NO: 872 - DNA, 22 bp, homo sapiens
tgtgaagagatagcttgtttcc

SEQ ID NO: 873 - DNA, 22 bp, homo sapiens
tacaataatgaggcatggctga

SEQ ID NO: 874 - DNA, 22 bp, homo sapiens
tacagcaacttcctatatactg

SEQ ID NO: 875 - DNA, 22 bp, homo sapiens
ctgaaaaaccaactgaaaagcg

SEQ ID NO: 876 - DNA, 22 bp, homo sapiens
tatttggtttcgttgtaatgat

SEQ ID NO: 877 - DNA, 22 bp, homo sapiens
gttcatctgtattatgttggtg

SEQ ID NO: 878 - DNA, 22 bp, homo sapiens
agtcaggtaatgtacataagga

SEQ ID NO: 879 - DNA, 22 bp, homo sapiens
agtaaccccagtgagaagggaa

SEQ ID NO: 880 - DNA, 22 bp, homo sapiens
agcagtttgctaaatgacccct

SEQ ID NO: 881 - DNA, 22 bp, homo sapiens
gtggccaagcaaagcactgccc

SEQ ID NO: 882 - DNA, 22 bp, homo sapiens
cccagcatctctgtgagcagtg

SEQ ID NO: 883 - DNA, 22 bp, homo sapiens
tgggaaaagagaaggatctcag

SEQ ID NO: 884 - DNA, 22 bp, homo sapiens
ttgctggtagatgtgtctttta

SEQ ID NO: 885 - DNA, 22 bp, homo sapiens
gaatacatccatgacttcaaca

SEQ ID NO: 886 - DNA, 22 bp, homo sapiens
aagagcaggactgcagtggacg

SEQ ID NO: 887 - DNA, 22 bp, homo sapiens
catctttcgccattcttcatag

SEQ ID NO: 888 - DNA, 22 bp, homo sapiens
tactgaaagcagtggagccaga

SEQ ID NO: 889 - DNA, 22 bp, homo sapiens
ccagaactgagtaccttgtcac

SEQ ID NO: 890 - DNA, 22 bp, homo sapiens
gctcagaagaaagagcaagggg

SEQ ID NO: 891 - DNA, 22 bp, homo sapiens
accagaaaggcaacatgaatcc

SEQ ID NO: 892 - DNA, 22 bp, homo sapiens
caccaactcccgttgccagaag

SEQ ID NO: 893 - DNA, 22 bp, homo sapiens
aattccatgccaaccacaggtt

SEQ ID NO: 894 - DNA, 22 bp, homo sapiens
gccccagagtgagcatccccgt

SEQ ID NO: 895 - DNA, 22 bp, homo sapiens
ggcggtctgcaacctcagattc

SEQ ID NO: 896 - DNA, 22 bp, homo sapiens
ggctgtaatcctagtttgccta

SEQ ID NO: 897 - DNA, 22 bp, homo sapiens
agaacggggaattccgggtctc

SEQ ID NO: 898 - DNA, 22 bp, homo sapiens
agcaaatcatttgattgtggaa

SEQ ID NO: 899 - DNA, 22 bp, homo sapiens
aggagaggcccgctggtacggc

SEQ ID NO: 900 - DNA, 22 bp, homo sapiens
tggtcgacttttccctcaacaa

SEQ ID NO: 901 - DNA, 22 bp, homo sapiens
agcccctggacagtgtgatgtt

SEQ ID NO: 902 - DNA, 22 bp, homo sapiens
gaaaacatcaaatcatccacat

SEQ ID NO: 903 - DNA, 22 bp, homo sapiens
acctgctcctttagaaaatact

SEQ ID NO: 904 - DNA, 22 bp, homo sapiens
ctttgaaatgtgcagacaataa

SEQ ID NO: 905 - DNA, 22 bp, homo sapiens
aaatcactatattgtcaagcaa

SEQ ID NO: 906 - DNA, 22 bp, homo sapiens
gcacttgtgcaattggaaagca

SEQ ID NO: 907 - DNA, 22 bp, homo sapiens
gttatgagcgacctggatatga

SEQ ID NO: 908 - DNA, 22 bp, homo sapiens
tgtcataaaagcattgctctct

SEQ ID NO: 909 - DNA, 22 bp, homo sapiens
atttagagcccgtcatctgtaa

SEQ ID NO: 910 - DNA, 22 bp, homo sapiens
tcataggaatatggtcacagaa

SEQ ID NO: 911 - DNA, 22 bp, homo sapiens
gtcaagtagccattgatgcaca

SEQ ID NO: 912 - DNA, 22 bp, homo sapiens
cagcttcccaaggcaaagcatg

SEQ ID NO: 913 - DNA, 22 bp, homo sapiens
caggcccacaggactaccgcgg

SEQ ID NO: 914 - DNA, 22 bp, homo sapiens
agaacttccaaaggagagaagt

SEQ ID NO: 915 - DNA, 22 bp, homo sapiens
cagatgtgaccgcatggcagag

SEQ ID NO: 916 - DNA, 22 bp, homo sapiens
ctcctggaagacctcagtaagt

SEQ ID NO: 917 - DNA, 22 bp, homo sapiens
tttgttgcagagtggaaaaagg

SEQ ID NO: 918 - DNA, 22 bp, homo sapiens
tccctacccaatccagccaagg

SEQ ID NO: 919 - DNA, 22 bp, homo sapiens
acccagccaagaggcaccttaa

SEQ ID NO: 920 - DNA, 22 bp, homo sapiens
gtggctccgccaatgttcattc

SEQ ID NO: 921 - DNA, 22 bp, homo sapiens
ccttgctgtcttgtttacaagt

SEQ ID NO: 922 - DNA, 22 bp, homo sapiens
ggacctgtcaatatccttacct

SEQ ID NO: 923 - DNA, 22 bp, homo sapiens
atggtctggaggctgaaggcag

SEQ ID NO: 924 - DNA, 22 bp, homo sapiens
gacagagttcttggagccggct

SEQ ID NO: 925 - DNA, 22 bp, homo sapiens
gttctcggagctggctcagagc

SEQ ID NO: 926 - DNA, 22 bp, homo sapiens
tggccagaaaaattgttccctc

SEQ ID NO: 927 - DNA, 22 bp, homo sapiens
ttccctcgaagatgggtttctt

SEQ ID NO: 928 - DNA, 22 bp, homo sapiens
ttttctctaactctctagaaaa

SEQ ID NO: 929 - DNA, 22 bp, homo sapiens
atgtgtctcagttgcatggcca

SEQ ID NO: 930 - DNA, 22 bp, homo sapiens
ggatcttcaggttctacgtgag

SEQ ID NO: 931 - DNA, 22 bp, homo sapiens
aggctctacgtcagcataggct

SEQ ID NO: 932 - DNA, 22 bp, homo sapiens
gagaccttgactcctcttctaa

SEQ ID NO: 933 - DNA, 22 bp, homo sapiens
ctctctccctctccagctgctg

SEQ ID NO: 934 - DNA, 22 bp, homo sapiens
ccattctctttaccattggggt

SEQ ID NO: 935 - DNA, 22 bp, homo sapiens
cctgccaatcctcggccccacc

SEQ ID NO: 936 - DNA, 22 bp, homo sapiens
atggacgagcatgcccggggga

SEQ ID NO: 937 - DNA, 22 bp, homo sapiens
tgaatgaaggtcgcagaaatgg

SEQ ID NO: 938 - DNA, 22 bp, homo sapiens
ccaatattaagtactataaaac

SEQ ID NO: 939 - DNA, 22 bp, homo sapiens
agttgaagaacattgttccaaa

SEQ ID NO: 940 - DNA, 22 bp, homo sapiens
atataaagtctgtgtactccag

SEQ ID NO: 941 - DNA, 22 bp, homo sapiens
tttcaacatgagtaaatttaaa

SEQ ID NO: 942 - DNA, 22 bp, homo sapiens
ggggccgctttgtcagtgatgg

SEQ ID NO: 943 - DNA, 22 bp, homo sapiens
gtccaatttcagtatgtactct

SEQ ID NO: 944 - DNA, 22 bp, homo sapiens
tcttcaatgccatctcagctat

SEQ ID NO: 945 - DNA, 22 bp, homo sapiens
ccatttcaacagtattagactt

SEQ ID NO: 946 - DNA, 22 bp, homo sapiens
actattttatcataacaactaa

SEQ ID NO: 947 - DNA, 22 bp, homo sapiens
aaagactgaatcatctcctttt

SEQ ID NO: 948 - DNA, 22 bp, homo sapiens
cttatccaggaaaatcaagagc

SEQ ID NO: 949 - DNA, 22 bp, homo sapiens
agattctgtacgttgttaacct

SEQ ID NO: 950 - DNA, 22 bp, homo sapiens
ttcagcctacttgtgctgctga

SEQ ID NO: 951 - DNA, 22 bp, homo sapiens
cggctccggggtgcacggggct

SEQ ID NO: 952 - DNA, 22 bp, homo sapiens
aaggtttttatctgttggtttg

SEQ ID NO: 953 - DNA, 22 bp, homo sapiens
gaagaaccgatcatgagccctg

SEQ ID NO: 954 - DNA, 22 bp, homo sapiens
ctgaggggagctggagttgggc

SEQ ID NO: 955 - DNA, 22 bp, homo sapiens
aaatgacgctccagttttcctg

SEQ ID NO: 956 - DNA, 22 bp, homo sapiens
cagcagggtccttagtccctgg

SEQ ID NO: 957 - DNA, 22 bp, homo sapiens
tcaccccccccacgcctgtctc

SEQ ID NO: 958 - DNA, 22 bp, homo sapiens
accccccccccccctgtctccc

SEQ ID NO: 959 - DNA, 22 bp, homo sapiens
tatggcccagcagtctgttttt

SEQ ID NO: 960 - DNA, 22 bp, homo sapiens
gttattagcactagccttaaga

SEQ ID NO: 961 - DNA, 22 bp, homo sapiens
gtaaaatgtgatgtaaaaatta

SEQ ID NO: 962 - DNA, 22 bp, homo sapiens
aggtgtgcactcattttgaaag

SEQ ID NO: 963 - DNA, 22 bp, homo sapiens
ttgttaaatgcacacttaatgg

SEQ ID NO: 964 - DNA, 22 bp, homo sapiens
caggtaggatacgaatgctcag

SEQ ID NO: 965 - DNA, 22 bp, homo sapiens
tagttagtaaatcattatttta

SEQ ID NO: 966 - DNA, 22 bp, homo sapiens
gttttaagatggctaccacagt

SEQ ID NO: 967 - DNA, 22 bp, homo sapiens
cacagttccttgcagatagcac

SEQ ID NO: 968 - DNA, 22 bp, homo sapiens
atattatttttaccattttttc

SEQ ID NO: 969 - DNA, 22 bp, homo sapiens
tagttttgtaaccctgtatcct

SEQ ID NO: 970 - DNA, 22 bp, homo sapiens
ttaaaagaagacacatgtttta

SEQ ID NO: 971 - DNA, 22 bp, homo sapiens
aatatttgacagatttttactg

SEQ ID NO: 972 - DNA, 22 bp, homo sapiens
tatggttctgagaaaaaaaata

SEQ ID NO: 973 - DNA, 22 bp, homo sapiens
tcctggtttgcgtacctttttg

SEQ ID NO: 974 - DNA, 22 bp, homo sapiens
acacacgtagaccaggttagcc

SEQ ID NO: 975 - DNA, 22 bp, homo sapiens
ccgggcagagacagggtaggag

SEQ ID NO: 976 - DNA, 22 bp, homo sapiens
tccgttttgcaatgtacttgcc

SEQ ID NO: 977 - DNA, 22 bp, homo sapiens
tccatcacttctagtgttgggt

SEQ ID NO: 978 - DNA, 22 bp, homo sapiens
cttggggggccacttacctcct

SEQ ID NO: 979 - DNA, 22 bp, homo sapiens
cagtgagatcagtttctcatcc

SEQ ID NO: 980 - DNA, 22 bp, homo sapiens
tccgcggggagtgtgacagcag

SEQ ID NO: 981 - DNA, 22 bp, homo sapiens
gaggttcaggatgagctgcttc

SEQ ID NO: 982 - DNA, 22 bp, homo sapiens
cgggctcgtggcctgcactgtc

SEQ ID NO: 983 - DNA, 22 bp, homo sapiens
tttcagagccaccacgcctggc

SEQ ID NO: 984 - DNA, 22 bp, homo sapiens
tgcactttttgggctcttgggg

SEQ ID NO: 985 - DNA, 22 bp, homo sapiens
ggaaactgtggtatgttcacgt

SEQ ID NO: 986 - DNA, 22 bp, homo sapiens
actgaagaggtgcaagaaagga

SEQ ID NO: 987 - DNA, 22 bp, homo sapiens
ttacctgttccgtagctcctgg

SEQ ID NO: 988 - DNA, 22 bp, homo sapiens
atcgttctggtggggaaaactg

SEQ ID NO: 989 - DNA, 22 bp, homo sapiens
acaccatccttcgagaggaaat

SEQ ID NO: 990 - DNA, 22 bp, homo sapiens
ttcctaatttattgtgatttgt

SEQ ID NO: 991 - DNA, 22 bp, homo sapiens
tttgtgccccacgaggtttgtt

SEQ ID NO: 992 - DNA, 22 bp, homo sapiens
tgccgtggaacttaatatgcag

SEQ ID NO: 993 - DNA, 22 bp, homo sapiens
tataaacatttgcttacacttt

SEQ ID NO: 994 - DNA, 22 bp, homo sapiens
gactgcggcagattggacacta

SEQ ID NO: 995 - DNA, 22 bp, homo sapiens
agaagattggaaaaggcttgta

SEQ ID NO: 996 - DNA, 22 bp, homo sapiens
cgggagtccattaagttggtga

SEQ ID NO: 997 - DNA, 22 bp, homo sapiens
aaaaggcttggtgctgggcagt

SEQ ID NO: 998 - DNA, 22 bp, homo sapiens
atggcatacattgagcggaaga

SEQ ID NO: 999 - DNA, 22 bp, homo sapiens
ccaatcccgtaggaagtgaaca

SEQ ID NO: 1000 - DNA, 22 bp, homo sapiens
ttatttggactataccgctggc

SEQ ID NO: 1001 - DNA, 22 bp, homo sapiens
aagcctgaaataactacttaca

SEQ ID NO: 1002 - DNA, 22 bp, homo sapiens
gtaaatgaactcgttcttggag

SEQ ID NO: 1003 - DNA, 22 bp, homo sapiens
ggaggatgccatcaggaacata

SEQ ID NO: 1004 - DNA, 22 bp, homo sapiens
ggcggcatcgctggaggcaggg

SEQ ID NO: 1005 - DNA, 22 bp, homo sapiens
aaaaagaaaatatttacaatta

SEQ ID NO: 1006 - DNA, 22 bp, homo sapiens
gctaaggagctcgctgcggagg

SEQ ID NO: 1007 - DNA, 22 bp, homo sapiens
gatgaggctcttggtggcaggt

SEQ ID NO: 1008 - DNA, 22 bp, homo sapiens
cccaccggggtatcactgcggg

SEQ ID NO: 1009 - DNA, 22 bp, homo sapiens
ctaggattccatacagactggc

SEQ ID NO: 1010 - DNA, 22 bp, homo sapiens
ttacatcaatgacatatgaccg

SEQ ID NO: 1011 - DNA, 22 bp, homo sapiens
ctgacccggctatctttctgtg

SEQ ID NO: 1012 - DNA, 22 bp, homo sapiens
gctgccctgctgaagctgtcct

SEQ ID NO: 1013 - DNA, 22 bp, homo sapiens
gtggtcattactctgccattca

SEQ ID NO: 1014 - DNA, 22 bp, homo sapiens
ggaagtaatggggaagaggtga

SEQ ID NO: 1015 - DNA, 22 bp, homo sapiens
ccagagcctgcaaagacaagag

SEQ ID NO: 1016 - DNA, 22 bp, homo sapiens
ctctggaaaacgtgttcaatct

SEQ ID NO: 1017 - DNA, 22 bp, homo sapiens
atccgtctctatgctgctatca

SEQ ID NO: 1018 - DNA, 22 bp, homo sapiens
atttatactacagggagaaaga

SEQ ID NO: 1019 - DNA, 22 bp, homo sapiens
ataagaatttagttcaagaaat

SEQ ID NO: 1020 - DNA, 22 bp, homo sapiens
cttaaaaaaaagtcctgctata

SEQ ID NO: 1021 - DNA, 22 bp, homo sapiens
aataaaacaaacaaacaaatta

SEQ ID NO: 1022 - DNA, 22 bp, homo sapiens
gtttcctctgggaaaaaaaaaa

SEQ ID NO: 1023 - DNA, 22 bp, homo sapiens
ttaaagcaatagttagtgaaat

SEQ ID NO: 1024 - DNA, 22 bp, homo sapiens
gattctatcatactggagtctt

SEQ ID NO: 1025 - DNA, 22 bp, homo sapiens
tatatgactggtggacgggtca

SEQ ID NO: 1026 - DNA, 22 bp, homo sapiens
tgactggcggatgggtcatgag

SEQ ID NO: 1027 - DNA, 22 bp, homo sapiens
agatatagatacatgcaatgcg

SEQ ID NO: 1028 - DNA, 22 bp, homo sapiens
ctctatgcccagctggaaatat

SEQ ID NO: 1029 - DNA, 22 bp, homo sapiens
gagagaccaaagggaagagtcc

SEQ ID NO: 1030 - DNA, 22 bp, homo sapiens
atgtttgccctagtgtgctgct

SEQ ID NO: 1031 - DNA, 22 bp, homo sapiens
ggtactcatggtcttgatttga

SEQ ID NO: 1032 - DNA, 22 bp, homo sapiens
cccaaagcaagctggactaaat

SEQ ID NO: 1033 - DNA, 22 bp, homo sapiens
gtgcactgactaggggtgctga

SEQ ID NO: 1034 - DNA, 22 bp, homo sapiens
agacagcaaaataacagagaga

SEQ ID NO: 1035 - DNA, 22 bp, homo sapiens
cctctaagggcattttcctcag

SEQ ID NO: 1036 - DNA, 22 bp, homo sapiens
gtggctcctccagtcaccaggg

SEQ ID NO: 1037 - DNA, 22 bp, homo sapiens
ggataactcactgcgctcatga

SEQ ID NO: 1038 - DNA, 22 bp, homo sapiens
taagaaataaaacaacctcctc

SEQ ID NO: 1039 - DNA, 22 bp, homo sapiens
atgttgagtgatgccaagatgc

SEQ ID NO: 1040 - DNA, 22 bp, homo sapiens
atgcagaccacaacatggccac

SEQ ID NO: 1041 - DNA, 22 bp, homo sapiens
tggggcagaagtgggcaacatc

SEQ ID NO: 1042 - DNA, 22 bp, homo sapiens
gatattaactcactgacctttg

SEQ ID NO: 1043 - DNA, 22 bp, homo sapiens
caattggctagtggggctcagt

SEQ ID NO: 1044 - DNA, 22 bp, homo sapiens
ttgtttaacatctcttctggga

SEQ ID NO: 1045 - DNA, 22 bp, homo sapiens
aattttactgtatccaaatggg

SEQ ID NO: 1046 - DNA, 22 bp, homo sapiens
ccaaaagggccatgaacagcca

SEQ ID NO: 1047 - DNA, 22 bp, homo sapiens
acaggaccctggatcccacccc

SEQ ID NO: 1048 - DNA, 22 bp, homo sapiens
aggattctaccacagcagaggc

SEQ ID NO: 1049 - DNA, 22 bp, homo sapiens
actcagagaggttcttgttgca

SEQ ID NO: 1050 - DNA, 22 bp, homo sapiens
gtaacagcacctgcttagcagg

SEQ ID NO: 1051 - DNA, 22 bp, homo sapiens
cattgtttgtaaggagacagcc

SEQ ID NO: 1052 - DNA, 22 bp, homo sapiens
gtagtgcttagagtagacgatg

SEQ ID NO: 1053 - DNA, 22 bp, homo sapiens
caaaacattcacaacaaagtta

SEQ ID NO: 1054 - DNA, 22 bp, homo sapiens
atttgcctagagaaaaaaaaaa

SEQ ID NO: 1055 - DNA, 22 bp, homo sapiens
agcaagcttcagaagtggtgaa

SEQ ID NO: 1056 - DNA, 22 bp, homo sapiens
ggtagagtagaacaggggtgag

SEQ ID NO: 1057 - DNA, 22 bp, homo sapiens
ggaaagaaactgtccacggacc

SEQ ID NO: 1058 - DNA, 22 bp, homo sapiens
tatgtgaatggcagaaactttg

SEQ ID NO: 1059 - DNA, 22 bp, homo sapiens
cagcaagctcatgtgcaggcac

SEQ ID NO: 1060 - DNA, 22 bp, homo sapiens
acctcagattgaggcgtggcga

SEQ ID NO: 1061 - DNA, 22 bp, homo sapiens
agctaccacagaatcaaaacag

SEQ ID NO: 1062 - DNA, 22 bp, homo sapiens
tattggggttgacattttgtga

SEQ ID NO: 1063 - DNA, 22 bp, homo sapiens
aagaatgtgtaataattaataa

SEQ ID NO: 1064 - DNA, 22 bp, homo sapiens
tatttatacagtgtgtatattt

SEQ ID NO: 1065 - DNA, 22 bp, homo sapiens
atttatacagcatgtatatttc

SEQ ID NO: 1066 - DNA, 22 bp, homo sapiens
ttacaaacactgagccagctcc

SEQ ID NO: 1067 - DNA, 22 bp, homo sapiens
ttgcccttttgttttattatct

SEQ ID NO: 1068 - DNA, 22 bp, homo sapiens
agtaagtctacaaaaaagccta

SEQ ID NO: 1069 - DNA, 22 bp, homo sapiens
gcacttttgtgaaggacactca

SEQ ID NO: 1070 - DNA, 22 bp, homo sapiens
atttgtggatacgtaggagtct

SEQ ID NO: 1071 - DNA, 22 bp, homo sapiens
tcagccacccttagtagtgctg

SEQ ID NO: 1072 - DNA, 22 bp, homo sapiens
gaggattcaacgtgggaatcac

SEQ ID NO: 1073 - DNA, 22 bp, homo sapiens
cagcatctttgaggtgatggta

SEQ ID NO: 1074 - DNA, 22 bp, homo sapiens
agctggtttctattgatcggtg

SEQ ID NO: 1075 - DNA, 22 bp, homo sapiens
ccagtgcccccataactcactg

SEQ ID NO: 1076 - DNA, 22 bp, homo sapiens
ttcctttgcaaagtatccatgg

SEQ ID NO: 1077 - DNA, 22 bp, homo sapiens
aacctggtagctgtacagtctt

SEQ ID NO: 1078 - DNA, 22 bp, homo sapiens
acttccgggtatgcctcctctt

SEQ ID NO: 1079 - DNA, 22 bp, homo sapiens
tgggaatgcaggtggctggaca

SEQ ID NO: 1080 - DNA, 22 bp, homo sapiens
ccacctgcaagtgccactttat

SEQ ID NO: 1081 - DNA, 22 bp, homo sapiens
ccaggacagctatctctgagtg

SEQ ID NO: 1082 - DNA, 22 bp, homo sapiens
ggatccggcagatatgagtgct

SEQ ID NO: 1083 - DNA, 22 bp, homo sapiens
cactccttcaaagtcagcaact

SEQ ID NO: 1084 - DNA, 22 bp, homo sapiens
ttctgattctgtgtttcctatt

SEQ ID NO: 1085 - DNA, 22 bp, homo sapiens
aattgcattaccagacaaaggt

SEQ ID NO: 1086 - DNA, 22 bp, homo sapiens
ggtaaatgcacgctgagaagaa

SEQ ID NO: 1087 - DNA, 22 bp, homo sapiens
tgagttctgaaatccaaagtaa

SEQ ID NO: 1088 - DNA, 22 bp, homo sapiens
ctccatatttgtgggtgcagat

SEQ ID NO: 1089 - DNA, 22 bp, homo sapiens
gccagctctgaaaatgagagac

SEQ ID NO: 1090 - DNA, 22 bp, homo sapiens
caactcagcatcagcccagcct

SEQ ID NO: 1091 - DNA, 22 bp, homo sapiens
cgcaggagttctgataagtgaa

SEQ ID NO: 1092 - DNA, 22 bp, homo sapiens
gtgcaggagagatgcaagaggg

SEQ ID NO: 1093 - DNA, 22 bp, homo sapiens
caggcctatcctttcctatcct

SEQ ID NO: 1094 - DNA, 22 bp, homo sapiens
tgtatctgttacattataatag

SEQ ID NO: 1095 - DNA, 22 bp, homo sapiens
ctcaccaaagacgtaaagttgc

SEQ ID NO: 1096 - DNA, 22 bp, homo sapiens
ttcactattctcatttgcctct

SEQ ID NO: 1097 - DNA, 22 bp, homo sapiens
tgatctctagacagcgatctga

SEQ ID NO: 1098 - DNA, 22 bp, homo sapiens
aaatccagaggcatcatgaaac

SEQ ID NO: 1099 - DNA, 22 bp, homo sapiens
aagtaagtaaaacagtaaacaa

SEQ ID NO: 1100 - DNA, 22 bp, homo sapiens
cgaaactaagaaccatttttct

SEQ ID NO: 1101 - DNA, 22 bp, homo sapiens
gaagttcagaactggactaacc

SEQ ID NO: 1102 - DNA, 22 bp, homo sapiens
cttttttttttctctttaaaca

SEQ ID NO: 1103 - DNA, 22 bp, homo sapiens
ttcaaccttctaattggggctg

SEQ ID NO: 1104 - DNA, 22 bp, homo sapiens
tccgttttcatgctgctcattc

SEQ ID NO: 1105 - DNA, 22 bp, homo sapiens
gatgggggagagaaaaaaaaag

SEQ ID NO: 1106 - DNA, 22 bp, homo sapiens
catcttttaagctgataacacc

SEQ ID NO: 1107 - DNA, 22 bp, homo sapiens
acactagttacgcccagaaatc

SEQ ID NO: 1108 - DNA, 22 bp, homo sapiens
ttaaacatgagacacacacagc

SEQ ID NO: 1109 - DNA, 22 bp, homo sapiens
ctgctttcagcgtcagccctcc

SEQ ID NO: 1110 - DNA, 22 bp, homo sapiens
tttaagaggaaggtcccaagtc

SEQ ID NO: 1111 - DNA, 22 bp, homo sapiens
tactggcacattgggaggagaa

SEQ ID NO: 1112 - DNA, 22 bp, homo sapiens
agaaaaggtatcattttttaaa

SEQ ID NO: 1113 - DNA, 22 bp, homo sapiens
gcttttgtcattcccatcctgg

SEQ ID NO: 1114 - DNA, 22 bp, homo sapiens
aaatagacgagctcctgtcgca

SEQ ID NO: 1115 - DNA, 22 bp, homo sapiens
ttgaatacctgcttagtatcgt

SEQ ID NO: 1116 - DNA, 22 bp, homo sapiens
ttttggattccttctagcatgg

SEQ ID NO: 1117 - DNA, 22 bp, homo sapiens
agggagggcttatagttaggtt

SEQ ID NO: 1118 - DNA, 22 bp, homo sapiens
atgactctaagacctctgtccc

SEQ ID NO: 1119 - DNA, 22 bp, homo sapiens
cgctgcggtcggacagctcgca

SEQ ID NO: 1120 - DNA, 22 bp, homo sapiens
cacgggggcagtgctctggtgt

SEQ ID NO: 1121 - DNA, 22 bp, homo sapiens
gaggcacctcgatcatggagtg

SEQ ID NO: 1122 - DNA, 22 bp, homo sapiens
aatttgctctacttgcagaagc

SEQ ID NO: 1123 - DNA, 22 bp, homo sapiens
acaaggacataactgttcccta

SEQ ID NO: 1124 - DNA, 22 bp, homo sapiens
gagagcctgccatttaaagtat

SEQ ID NO: 1125 - DNA, 22 bp, homo sapiens
tttgtgtcggtagttatgacaa

SEQ ID NO: 1126 - DNA, 22 bp, homo sapiens
gacagcagcggtgaccctgatg

SEQ ID NO: 1127 - DNA, 22 bp, homo sapiens
tttcttcccgaccaagatcaga

SEQ ID NO: 1128 - DNA, 22 bp, homo sapiens
gcttgcttctgccatcgaaatc

SEQ ID NO: 1129 - DNA, 22 bp, homo sapiens
tctgaagcatatctgcaaacaa

SEQ ID NO: 1130 - DNA, 22 bp, homo sapiens
agggcaggataagagtaagaga

SEQ ID NO: 1131 - DNA, 22 bp, homo sapiens
tagcgctccccagcagccctgg

SEQ ID NO: 1132 - DNA, 22 bp, homo sapiens
cctgccaagaagagagagacac

SEQ ID NO: 1133 - DNA, 22 bp, homo sapiens
acaaaggtacacacatacctga

SEQ ID NO: 1134 - DNA, 22 bp, homo sapiens
aaaaaaaaaaaggttggcatga

SEQ ID NO: 1135 - DNA, 22 bp, homo sapiens
taaaatgatcatattttccaaa

SEQ ID NO: 1136 - DNA, 22 bp, homo sapiens
gtctgcctccatggatagtgaa

SEQ ID NO: 1137 - DNA, 22 bp, homo sapiens
gtcctatgcaataataacacag

SEQ ID NO: 1138 - DNA, 22 bp, homo sapiens
tttctttgtctggatggattcc

SEQ ID NO: 1139 - DNA, 22 bp, homo sapiens
atgagtatgtttcatgcaatat

SEQ ID NO: 1140 - DNA, 22 bp, homo sapiens
ccagatcactctttcaccctcc

SEQ ID NO: 1141 - DNA, 22 bp, homo sapiens
attttttttttctctggtagga

SEQ ID NO: 1142 - DNA, 22 bp, homo sapiens
ttatttgatgatctggcaactt

SEQ ID NO: 1143 - DNA, 22 bp, homo sapiens
ttcaaacttcacttcctatctg

SEQ ID NO: 1144 - DNA, 22 bp, homo sapiens
ctagcaacattaatggttatag

SEQ ID NO: 1145 - DNA, 22 bp, homo sapiens
ctttcaacttatcaaaatcaat

SEQ ID NO: 1146 - DNA, 22 bp, homo sapiens
ttttttttttttataggtatga

SEQ ID NO: 1147 - DNA, 22 bp, homo sapiens
tagtaatatttgcaaaattata

SEQ ID NO: 1148 - DNA, 22 bp, homo sapiens
taatgcccattatgtattgata

SEQ ID NO: 1149 - DNA, 22 bp, homo sapiens
gtgtgctgcggtgagtgcttcc

SEQ ID NO: 1150 - DNA, 22 bp, homo sapiens
tgattaattacagcacagacat

SEQ ID NO: 1151 - DNA, 22 bp, homo sapiens
atactggccaatagttttagag

SEQ ID NO: 1152 - DNA, 22 bp, homo sapiens
ttccacacggaatcccttctca

SEQ ID NO: 1153 - DNA, 22 bp, homo sapiens
tgacaatattttataaaacttt

SEQ ID NO: 1154 - DNA, 22 bp, homo sapiens
agacatgggaaccactctgcat

SEQ ID NO: 1155 - DNA, 22 bp, homo sapiens
tttatgtagtactgaacatttt

SEQ ID NO: 1156 - DNA, 22 bp, homo sapiens
cgtagttggcagagatatctat

SEQ ID NO: 1157 - DNA, 22 bp, homo sapiens
tagaggttctcaagtggagtaa

SEQ ID NO: 1158 - DNA, 22 bp, homo sapiens
gatttttttttaatttaactaa

SEQ ID NO: 1159 - DNA, 22 bp, homo sapiens
agcagggtgtggggaatttcac

SEQ ID NO: 1160 - DNA, 22 bp, homo sapiens
tgagcaaaactgctctggataa

SEQ ID NO: 1161 - DNA, 22 bp, homo sapiens
aataaaggttaccacatagtaa

SEQ ID NO: 1162 - DNA, 22 bp, homo sapiens
catagtaagtgttcattaagta

SEQ ID NO: 1163 - DNA, 22 bp, homo sapiens
ttccacagtcacggtaaagttc

SEQ ID NO: 1164 - DNA, 22 bp, homo sapiens
agtgtaatttactaaggggttt

SEQ ID NO: 1165 - DNA, 22 bp, homo sapiens
atttattaaggagtttacaaat

SEQ ID NO: 1166 - DNA, 22 bp, homo sapiens
taaggggtttaaaaatatgttt

SEQ ID NO: 1167 - DNA, 22 bp, homo sapiens
aggtgggtatctgcttttccct

SEQ ID NO: 1168 - DNA, 22 bp, homo sapiens
agagacccaaatcctaggtctg

SEQ ID NO: 1169 - DNA, 22 bp, homo sapiens
ctgcaccccacgctagacacct

SEQ ID NO: 1170 - DNA, 22 bp, homo sapiens
actccatatcttaagttctcca

SEQ ID NO: 1171 - DNA, 22 bp, homo sapiens
cgttcctaggggaaaaaaaaaa

SEQ ID NO: 1172 - DNA, 22 bp, homo sapiens
gaactactacactgggaacatg

SEQ ID NO: 1173 - DNA, 22 bp, homo sapiens
caaagattttccggttacttac

SEQ ID NO: 1174 - DNA, 22 bp, homo sapiens
taaaaaagcatgtgtaaagtac

SEQ ID NO: 1175 - DNA, 22 bp, homo sapiens
ctattcatttcataccctaaac

SEQ ID NO: 1176 - DNA, 22 bp, homo sapiens
ccagcttcctcagacaacctgg

SEQ ID NO: 1177 - DNA, 22 bp, homo sapiens
cccacgctgagcgtcgtcttag

SEQ ID NO: 1178 - DNA, 22 bp, homo sapiens
tttcttacccttcatcttctgc

SEQ ID NO: 1179 - DNA, 22 bp, homo sapiens
agctacacacatgagattatac

SEQ ID NO: 1180 - DNA, 22 bp, homo sapiens
cacatcaggcagtggaactatc

SEQ ID NO: 1181 - DNA, 22 bp, homo sapiens
aagtggcactccagtgctgttt

SEQ ID NO: 1182 - DNA, 22 bp, homo sapiens
cagcaagttcgcccagcaactt

SEQ ID NO: 1183 - DNA, 22 bp, homo sapiens
aaggtcctcttgtaagtcctcc

SEQ ID NO: 1184 - DNA, 22 bp, homo sapiens
aacaatcgacaagctagaagtt

SEQ ID NO: 1185 - DNA, 22 bp, homo sapiens
tcacaatgatccgatgctgggg

SEQ ID NO: 1186 - DNA, 22 bp, homo sapiens
catttgctggtgagttattgta

SEQ ID NO: 1187 - DNA, 22 bp, homo sapiens
acttttacttacagctttattt

SEQ ID NO: 1188 - DNA, 22 bp, homo sapiens
ggaggttatctatagagacagt

SEQ ID NO: 1189 - DNA, 22 bp, homo sapiens
cctggtgaaatcaggtcttctt

SEQ ID NO: 1190 - DNA, 22 bp, homo sapiens
taactggttctagagaatctgg

SEQ ID NO: 1191 - DNA, 22 bp, homo sapiens
gtataggtaatactcctgggcc

SEQ ID NO: 1192 - DNA, 22 bp, homo sapiens
gtcggggagattcctggtcaag

SEQ ID NO: 1193 - DNA, 22 bp, homo sapiens
gttgcggatttcgcgaaagtta

SEQ ID NO: 1194 - DNA, 22 bp, homo sapiens
gggcaaacacacaagccgctat

SEQ ID NO: 1195 - DNA, 22 bp, homo sapiens
gttccatatcagtcagccagct

SEQ ID NO: 1196 - DNA, 22 bp, homo sapiens
accccccatgcagaagatatgg

SEQ ID NO: 1197 - DNA, 22 bp, homo sapiens
aactcgtgattatcggggggaa

SEQ ID NO: 1198 - DNA, 22 bp, homo sapiens
acatctgtttgtgataacttgg

SEQ ID NO: 1199 - DNA, 22 bp, homo sapiens
ggaatacagtggacactttcca

SEQ ID NO: 1200 - DNA, 22 bp, homo sapiens
ttatgaaaagacgagcacagca

SEQ ID NO: 1201 - DNA, 22 bp, homo sapiens
tcaagtgagtatccctctccca

SEQ ID NO: 1202 - DNA, 22 bp, homo sapiens
aatatccgtcaacagacgcctg

SEQ ID NO: 1203 - DNA, 22 bp, homo sapiens
tttcacgagaacagaatgtggc

SEQ ID NO: 1204 - DNA, 22 bp, homo sapiens
ggctccagtcaatggttttttt

SEQ ID NO: 1205 - DNA, 22 bp, homo sapiens
tcaccaagaaacaggtaaaccc

SEQ ID NO: 1206 - DNA, 22 bp, homo sapiens
tcgaggaaggaactgtgggagt

SEQ ID NO: 1207 - DNA, 22 bp, homo sapiens
ctcgtacccgatgtctcctacg

SEQ ID NO: 1208 - DNA, 22 bp, homo sapiens
ctggatgtcaatgagggcccag

SEQ ID NO: 1209 - DNA, 22 bp, homo sapiens
taaatgtttaactatacacatg

SEQ ID NO: 1210 - DNA, 22 bp, homo sapiens
tacacacgcccgggtaagcctt

SEQ ID NO: 1211 - DNA, 22 bp, homo sapiens
atagcaacagggaaaaaaaaaa

SEQ ID NO: 1212 - DNA, 22 bp, homo sapiens
cgcggccatggtgcgcgcttac

SEQ ID NO: 1213 - DNA, 22 bp, homo sapiens
gtgattcagcaccatcaccggc

SEQ ID NO: 1214 - DNA, 22 bp, homo sapiens
ggtcagtgccaatccacagctg

SEQ ID NO: 1215 - DNA, 22 bp, homo sapiens
tagaaacagacgccacatactt

SEQ ID NO: 1216 - DNA, 22 bp, homo sapiens
gtgtcctggtatgactggggca

SEQ ID NO: 1217 - DNA, 22 bp, homo sapiens
tgccccaagcggaaacagaaaa

SEQ ID NO: 1218 - DNA, 22 bp, homo sapiens
aagctctcctgaagcaaatatg

SEQ ID NO: 1219 - DNA, 22 bp, homo sapiens
gagagggtctctttcctatgcg

SEQ ID NO: 1220 - DNA, 22 bp, homo sapiens
cagctgtgctcccgtcccctgc

SEQ ID NO: 1221 - DNA, 22 bp, homo sapiens
gctcatcttcccaccgagtgct

SEQ ID NO: 1222 - DNA, 22 bp, homo sapiens
ttctgaaacgctgcggatcaat

SEQ ID NO: 1223 - DNA, 22 bp, homo sapiens
agctctgaccttcatgttcttc

SEQ ID NO: 1224 - DNA, 22 bp, homo sapiens
cgtggaaaaaatgaaacaaaag

SEQ ID NO: 1225 - DNA, 22 bp, homo sapiens
atccaagagcttctaccaggca

SEQ ID NO: 1226 - DNA, 22 bp, homo sapiens
tccatgtccccaggcctgtaag

SEQ ID NO: 1227 - DNA, 22 bp, homo sapiens
aagtatcaagctttcattcagt

SEQ ID NO: 1228 - DNA, 22 bp, homo sapiens
tcccataagaggattggctttg

SEQ ID NO: 1229 - DNA, 22 bp, homo sapiens
gtaacgaggctgccttttgttt

SEQ ID NO: 1230 - DNA, 22 bp, homo sapiens
caaagctcccactggtgtaatt

SEQ ID NO: 1231 - DNA, 22 bp, homo sapiens
atccttacctgcacctctgtct

SEQ ID NO: 1232 - DNA, 22 bp, homo sapiens
attgctagggcgacacatttat

SEQ ID NO: 1233 - DNA, 22 bp, homo sapiens
tcttcagcaagttgtcttactt

SEQ ID NO: 1234 - DNA, 22 bp, homo sapiens
cttactcttatctgatccacaa

SEQ ID NO: 1235 - DNA, 22 bp, homo sapiens
ctaaggaaagacaaacagcttt

SEQ ID NO: 1236 - DNA, 22 bp, homo sapiens
ttattatactaacagatccttt

SEQ ID NO: 1237 - DNA, 22 bp, homo sapiens
aagaaaaagagagggtaggtgg

SEQ ID NO: 1238 - DNA, 22 bp, homo sapiens
aagagggggtaagtggaaacaa

SEQ ID NO: 1239 - DNA, 22 bp, homo sapiens
gtcttaaaattgatgacaacca

SEQ ID NO: 1240 - DNA, 22 bp, homo sapiens
caggacctgggtgtgaagaact

SEQ ID NO: 1241 - DNA, 22 bp, homo sapiens
ccgtgtcctccagtgtgtgtga

SEQ ID NO: 1242 - DNA, 22 bp, homo sapiens
gtgtgaggccaggctcctgggg

SEQ ID NO: 1243 - DNA, 22 bp, homo sapiens
gggaagacaggagaatggcatt

SEQ ID NO: 1244 - DNA, 22 bp, homo sapiens
ttgggtaaatctaccttaatca

SEQ ID NO: 1245 - DNA, 22 bp, homo sapiens
cgtggctctgtacagatattga

SEQ ID NO: 1246 - DNA, 22 bp, homo sapiens
cccatcccccatgcagctggca

SEQ ID NO: 1247 - DNA, 22 bp, homo sapiens
gccacagggataggcccatctc

SEQ ID NO: 1248 - DNA, 22 bp, homo sapiens
ggaccggctggcgaacgagaag

SEQ ID NO: 1249 - DNA, 22 bp, homo sapiens
cagtctcatcagccagaaatag

SEQ ID NO: 1250 - DNA, 22 bp, homo sapiens
ttgggtaagtttctgtatgtca

SEQ ID NO: 1251 - DNA, 22 bp, homo sapiens
accaccccaaaaaattccttct

SEQ ID NO: 1252 - DNA, 22 bp, homo sapiens
tagttgaaatactatattgagg

SEQ ID NO: 1253 - DNA, 22 bp, homo sapiens
aatattatattaaggggtcttt

SEQ ID NO: 1254 - DNA, 22 bp, homo sapiens
tgatttgtgtatactgttggtt

SEQ ID NO: 1255 - DNA, 22 bp, homo sapiens
aaaggagctgtcgccgcacaag

SEQ ID NO: 1256 - DNA, 22 bp, homo sapiens
gccgtggtccatggtacttgaa

SEQ ID NO: 1257 - DNA, 22 bp, homo sapiens
gtggacctgtctccacgatgca

SEQ ID NO: 1258 - DNA, 22 bp, homo sapiens
cctgcacccgctgtcattcctc

SEQ ID NO: 1259 - DNA, 22 bp, homo sapiens
aaggaatatatagatggcctgg

SEQ ID NO: 1260 - DNA, 22 bp, homo sapiens
ttcctgtgaaacctgcgtggct

SEQ ID NO: 1261 - DNA, 22 bp, homo sapiens
catctggtcactgtggtggctt

SEQ ID NO: 1262 - DNA, 22 bp, homo sapiens
ttagaaattcatcctctttccc

SEQ ID NO: 1263 - DNA, 22 bp, homo sapiens
cacagcacccttccctactcac

SEQ ID NO: 1264 - DNA, 22 bp, homo sapiens
ttgtccgtccgtgggttcctgg

SEQ ID NO: 1265 - DNA, 22 bp, homo sapiens
ggcacaggcagactcagagcag

SEQ ID NO: 1266 - DNA, 22 bp, homo sapiens
gcagactcagatgcatccccag

SEQ ID NO: 1267 - DNA, 22 bp, homo sapiens
cgagcaagggccggcaagttac

SEQ ID NO: 1268 - DNA, 22 bp, homo sapiens
cagcccttggattgaggcaggc

SEQ ID NO: 1269 - DNA, 22 bp, homo sapiens
tacagcttacatgggaaatcac

SEQ ID NO: 1270 - DNA, 22 bp, homo sapiens
agaaggaagatcggctgttact

SEQ ID NO: 1271 - DNA, 22 bp, homo sapiens
atcaaggagcaaagaagttctg

SEQ ID NO: 1272 - DNA, 22 bp, homo sapiens
ctccctagaagggaagcaacaa

SEQ ID NO: 1273 - DNA, 22 bp, homo sapiens
tggtggcgtggaaagctcttgc

SEQ ID NO: 1274 - DNA, 22 bp, homo sapiens
gggaccttggctcaaacatcag

SEQ ID NO: 1275 - DNA, 22 bp, homo sapiens
gcagtgctcacaccccggaaag

SEQ ID NO: 1276 - DNA, 22 bp, homo sapiens
gggactgcgtcacagagatggg

SEQ ID NO: 1277 - DNA, 22 bp, homo sapiens
ttgactcttccacaggtagggg

SEQ ID NO: 1278 - DNA, 22 bp, homo sapiens
gcagcccatggatgggactctg

SEQ ID NO: 1279 - DNA, 22 bp, homo sapiens
aggagaggaagcagggaccagc

SEQ ID NO: 1280 - DNA, 22 bp, homo sapiens
ccttaagaaggaataaaagtga

SEQ ID NO: 1281 - DNA, 22 bp, homo sapiens
agaacctcatctgtcagagaag

SEQ ID NO: 1282 - DNA, 22 bp, homo sapiens
tgcattgaatgtgggaaattct

SEQ ID NO: 1283 - DNA, 22 bp, homo sapiens
gtgccaggtacatgggaacctt

SEQ ID NO: 1284 - DNA, 22 bp, homo sapiens
gacctgaagacacagctgtcac

SEQ ID NO: 1285 - DNA, 22 bp, homo sapiens
gagtgtgttggagggcttgtga

SEQ ID NO: 1286 - DNA, 22 bp, homo sapiens
tccccttagggcccgaaagatt

SEQ ID NO: 1287 - DNA, 22 bp, homo sapiens
cctacatgcagatgcacattca

SEQ ID NO: 1288 - DNA, 22 bp, homo sapiens
gaaaagaagactgcagtgccag

SEQ ID NO: 1289 - DNA, 22 bp, homo sapiens
tttcaaagttggaaaaccatcg

SEQ ID NO: 1290 - DNA, 22 bp, homo sapiens
aaaaaccatcgtgtcatgtagg

SEQ ID NO: 1291 - DNA, 22 bp, homo sapiens
ctctgtagtcaactgcttgcat

SEQ ID NO: 1292 - DNA, 22 bp, homo sapiens
tttcttgtctcacacaggcttt

SEQ ID NO: 1293 - DNA, 22 bp, homo sapiens
gggtgagagcgtgaggctccgg

SEQ ID NO: 1294 - DNA, 22 bp, homo sapiens
tcccttgtatctggcaggtggt

SEQ ID NO: 1295 - DNA, 22 bp, homo sapiens
atacacgacagacctgcagctc

SEQ ID NO: 1296 - DNA, 22 bp, homo sapiens
gctcacaggcatgcacagggag

SEQ ID NO: 1297 - DNA, 22 bp, homo sapiens
tagctagttggatgatgaaggt

SEQ ID NO: 1298 - DNA, 22 bp, homo sapiens
ccctctggggtagtccagccct

SEQ ID NO: 1299 - DNA, 22 bp, homo sapiens
gagtagggcttttccccagagt

SEQ ID NO: 1300 - DNA, 22 bp, homo sapiens
agccaccggcttcccaggctga

SEQ ID NO: 1301 - DNA, 22 bp, homo sapiens
agggcgatcccacctggatgcg

SEQ ID NO: 1302 - DNA, 22 bp, homo sapiens
ccgggcaaagatgtcatcctca

SEQ ID NO: 1303 - DNA, 18 bp, artificial sequence
  synthetic oligonucleotide
cagaccaccggttggaga

SEQ ID NO: 1304 - DNA, 23 bp, artificial sequence
  synthetic oligonucleotide
ccactcgtttctggtagttcttc

SEQ ID NO: 1305 - DNA, 18 bp, artificial sequence
  synthetic oligonucleotide
gcttggttgtcagcagca

SEQ ID NO: 1306 - DNA, 24 bp, artificial sequence
  synthetic oligonucleotide
tgcaattctcatggtagtgagttt

SEQ ID NO: 1307 - DNA, 20 bp, artificial sequence
  synthetic oligonucleotide
gggcagtgtcatgttggtag

SEQ ID NO: 1308 - DNA, 20 bp, artificial sequence
  synthetic oligonucleotide
cagccccaaagagggataat

SEQ ID NO: 1309 - DNA, 22 bp, artificial sequence
  synthetic oligonucleotide
gctttatgctgaagccctatga

SEQ ID NO: 1310 - DNA, 21 bp, artificial sequence
  synthetic oligonucleotide
tccaactctgcagacatttcc

SEQ ID NO: 1311 - DNA, 20 bp, artificial sequence
  synthetic oligonucleotide
ggttaatctccgcaggtcac

SEQ ID NO: 1312 - DNA, 20 bp, artificial sequence
  synthetic oligonucleotide
ctgcttgcagccttaaatga

SEQ ID NO: 1313 - DNA, 18 bp, artificial sequence
  synthetic oligonucleotide
cgatgccatcatgcaagt

SEQ ID NO: 1314 - DNA, 19 bp, artificial sequence
  synthetic oligonucleotide
acaccagcagccgtgtaac

SEQ ID NO: 1315 - DNA, 26 bp, artificial sequence
  synthetic oligonucleotide
aacaactgtgtaatcagatcaatgaa

SEQ ID NO: 1316 - DNA, 20 bp, artificial sequence
  synthetic oligonucleotide
tgcctatggcatgagacaag

SEQ ID NO: 1317 - DNA, 18 bp, artificial sequence
  synthetic oligonucleotide
tcgccagcaagacgttta

SEQ ID NO: 1318 - DNA, 18 bp, artificial sequence
  synthetic oligonucleotide
gccgcttcttaggggtct

SEQ ID NO: 1319 - DNA, 18 bp, artificial sequence
  synthetic oligonucleotide
tcacctccacgcacttcc

SEQ ID NO: 1320 - DNA, 20 bp, artificial sequence
  synthetic oligonucleotide
gggcctggtagacctcatct

SEQ ID NO: 1321 - DNA, 19 bp, artificial sequence
  synthetic oligonucleotide
agccacatcgctcagacac

SEQ ID NO: 1322 - DNA, 19 bp, artificial sequence
  synthetic oligonucleotide
gcccaatacgaccaaatcc

SEQ ID NO: 1323 - DNA, 27 bp, artificial sequence
  synthetic oligonucleotide
tgatagatccattcctatgactgtaga

SEQ ID NO: 1324 - DNA, 26 bp, artificial sequence
  synthetic oligonucleotide
caagacattctttccagttaaagttg

(19) United States
(12) Patent Application Publication
(10) Pub. No.: US 2019/0195859 A1
(43) Pub. Date: Jun. 27, 2019

(54) DETERMINING THE RISK OF SCOLIOSIS COMPRISING DETERMINING CELLULAR RESPONSE TO MECHANOSTIMULATION

(71) Applicant: CHU Sainte-Justine, Montreal (CA)

(72) Inventors: Alain Moreau, Montreal (CA); Niaz Oliazadeh, Vaudreuil-Dorion (CA); Kristen Fay Gorman, Chico, CA (US); Robert Eveleigh, Longueuil (CA); Guillaume Bourque, Montreal (CA)

(73) Assignee: CHU Sainte-Justine, Montreal, QC (CA)

(21) Appl. No.: 16/327,278

(22) PCT Filed: Aug. 23, 2017
(86) PCT No.: PCT/CA2017/050992

§ 371 Date: Feb. 21, 2019

Related U.S. Application Data
(60) Provisional application No. 62/378,297, filed on Aug. 23, 2016.

(51) Int. Cl. G01N 33/50; C12Q 1/6883

(52) U.S. Cl. CPC G01N 33/5005; C12Q 1/6883; G01N 2800/50; C12Q 2600/112; C12Q 2600/158; C12Q 2600/156

(57) ABSTRACT

Disclosed herein are novel molecular markers associated with idiopathic scoliosis (IS). Accordingly, the present invention concerns novel methods of identifying subjects at risk of developing IS or suffering from IS and of genotyping and classifying IS subjects into genetic and functional groups. Also provided are compositions, DNA chips and kits for applying the methods.

### CROSS REFERENCE TO RELATED APPLICATIONS

This application is a PCT application Serial No PCT/CA2017/* filed on Aug. 23, 2017 and published in English under PCT Article 21(2), which itself claims benefit of U.S. provisional application Ser. No. 62/378,297, filed on Aug. 23, 2016, which is incorporated herein in its entirety by reference.

### STATEMENT REGARDING FEDERALLY SPONSORED RESEARCH OR DEVELOPMENT

N.A

### FIELD OF THE INVENTION

The present invention relates to idiopathic scoliosis. More specifically, the present invention is concerned with molecular markers associated with IS and their use in the diagnosis, genotyping, classification and treatment of IS.

### REFERENCE TO SEQUENCE LISTING

This application contains a Sequence Listing in computer readable form (as an ASCII compliant text file) entitled “Seq_List_14033_161_ST25”, created on Aug. 21, 2017 having a size of 196 kilobytes. The content of the aforementioned file named Seq_List_14033_161_ST25 is hereby incorporated by reference in its entirety.

### BACKGROUND OF THE INVENTION

Primary cilia are antenna-like organelles that transmit chemical and mechanical signals from the pericellular environment.^{10,11} They are found in the cells of all human tissues (except blood), including bone, cartilage, tendons, and skeletal muscle (a comprehensive list of tissue types and cell lines with primary cilia can be found at: http://www.bowserlab.org/primarycilia/ciliumpage2.htm). In addition to functions linked to olfaction, photo and chemical sensation, recent studies have established a mechanosensory role for primary cilia in tissues, such as the kidney, liver, embryonic node, and bone structure (the mechanosensory role of cilia in bone is reviewed by Nguyen, et al., 2013).¹² As the most recent established role for cilia, mechanisms for mechanosensation are not yet entirely understood. For example, the involvement of calcium channels, in response to cilia bending following a fluid movement, is yet a matter of debate and might vary depending on the tissue examined.^{13,14}

As a mechanosensor in bone, the primary cilium can transduce fluid flow induced shear stress occurring within the canaliculi that interconnect osteocytes as well as strain-related mechanical stimuli in pre-osteoblasts.¹⁵ The load-induced fluid flow in bone canaliculi is recognized to play a role in maintaining bone homeostasis through bone resorption and formation cycles (i.e. bone tissue remodeling).¹⁶ Cilia mediate the transduction of this fluid flow to mesenchymal stem cells (MSCs), and is implicated in osteogenic gene expression and lineage commitment.¹⁷ Mechanical loading modulates the incidence and length of primary cilia in cells, such as chondrocytes, in which cilia direction affects the direction of growth in growth plates.¹⁸ Mechanical loading has also been shown to induce bone cell proliferation through a cilia-dependent mechanism.¹⁵ Interestingly, skeletal disorders are a common feature in several human ciliopathies, such as Jeune syndrome and short rib-polydactyly.¹⁹.

Idiopathic scoliosis (IS) is a complex pediatric syndrome that manifests primarily as an abnormal three-dimensional curvature of the spine. Eighty percent of all spinal curvatures are idiopathic, (MIM 181800) making IS the most prevalent form of spinal deformity. With a global incidence of 0.15% to 10% (depending on curve severity),¹ IS contributes significantly to the burden of musculoskeletal diseases on healthcare (http://www.boneandjointburden.org). Children with IS are born with a normal spine, and the abnormal curvature may begin at different points during growth, though adolescent onset is the most prevalent.² Idiopathic scoliosis is diagnosed by ruling out congenital defects and other causes of abnormal curvature, such as muscular dystrophies, tumors, or other syndromes.

The etiology of idiopathic scoliosis is unknown largely because of phenotypic and genetic heterogeneity. Curve magnitude is highly variable and the risk for severe curvature is not understood beyond the observed female bias. Although a genetic basis is accepted, genetic heterogeneity has been implicated in several familial studies,^{3,4} and numerous genome-wide association studies (GWAS) have detected different loci with small effects.⁵ Despite such genetic correlations, no clear biological mechanism for IS has emerged. It is likely that IS phenotypic heterogeneity is a consequence of genetic variations combined with biomechanical factors that are influenced by individual behavioral patterns. As a musculoskeletal syndrome, biomechanics are thought to have an important role in the IS deformity. Pathological stressors applied to a normal spine or normal forces on an already deformed spine have been studied for a role in curve predisposition and progression.⁶ For example, factors that contribute to spinal flexibility, sagittal balance, shear loading on the spine, and compressive or tension forces may contribute to the ‘column buckling’ phenotype associated with IS.⁷⁻⁹ Furthermore, therapeutic options available for IS, bracing and corrective surgery, approach the disease from a mechanical perspective, and successful outcomes depend on understanding the complex biomechanics of the spine.

Thus, there remains a need for the identification of new molecular markers associated with IS. There also remains a need for new ways to characterize, classify, diagnose and treat subjects suffering from IS or at risk of suffering from IS.

The present description refers to a number of documents, the content of which is herein incorporated by reference in their entirety.

### SUMMARY OF THE INVENTION

The present invention discloses evidence supporting an association between IS and mechanotransduction through the non-motile microtubule-based signaling organelle known as cilium. Applicant has found that numerous ciliary genes present a spinal curvature phenotype when knocked down in animal models, and that scoliosis is associated with many human ciliopathy syndromes.^{20,21} Additionally, the majority of confirmed IS associated genes are connected to cilia structure or function. Confocal images of primary osteoblast cultures, derived from bone fragments obtained intraoperatively at the time of the spine surgery, revealed that IS subjects have longer primary cilia and an increased density of cells with elongated cilia. Also, further studies have demonstrated the presence of an altered cellular response to mechanostimulation in cells from IS subjects. Furthermore, SKAT-O analysis of whole exomes has allowed to identify rare gene variants with a role in mechanotransduction in IS subjects.

Accordingly, there are provided novel molecular markers and alternative methods of identifying subjects at risk of developing IS or suffering from IS and of genotyping and classifying IS subjects into genetic and functional groups. Methods of the present invention can be used alone or with one or more previous methods to increase the specificity and/or sensitivity of risk prediction to improve subject's classification and to facilitate and improve the application of preventive treatment measures. Once a subject has been classified into one or more IS group, treatment and preventive measures can be adapted to his/her specific endophenotype/genotype.

More specifically, in an aspect, the present invention provides a method of determining the risk of or predisposition to developing a scoliosis comprising determining a cellular response to mechanostimulation in a cell sample from a subject, wherein an altered cellular response in said sample as compared to that in a control sample is indicative of an increased risk of developing a scoliosis.

In a further aspect, the present invention provides a method of determining the risk of or predisposition to developing a scoliosis comprising (i) determining the average length of cilia on the surface of cells in a cell sample from the subject; (ii) determining the number of cells with elongated cilia in a cell sample from the subject; (iii) determining the number of ciliated cells in a cell sample from the subject; or (iv) any combination of one of (i), (ii) and (iii), wherein an increase in the average length of cilia, an increase in the number of cells having elongated cilia or a decrease in the number of ciliated cells in the cell sample from the subject as compared to that in a control sample is indicative of an increased risk of or predisposition to developing a scoliosis.

In a further aspect, the present invention provides a method of determining the risk of or predisposition to developing a scoliosis comprising determining a cellular response to mechanostimulation of cells in a cell sample from a subject, wherein the determining comprises: (i) applying mechanostimulation to cells in a cell sample from the subject; and (ii) measuring the expression level of at least one mechanoresponsive gene, wherein the at least one mechanoresponsive gene is ITGB1; ITGB3, CTNNB1; POC5, BMP2, COX-2, RUNX2, CTNNB1 or any combination thereof; (iii) comparing the expression level measured in (b)(ii) to that of a control sample, wherein an altered expression level in said mechanoresponsive gene as compared to that of the control sample is indicative of an increased risk of or predisposition to developing a scoliosis. In embodiments, the above method is performed on cells having elongated cilia.

In embodiments, (i) determining the average length of cilia on the surface of cells in a cell sample from the subject; (ii) determining the number of cells with elongated cilia in a cell sample from the subject; (iii) determining the number of ciliated cells in a biological sample from the subject; (iv) determining a cellular response to mechanostimulation of cells in a cell sample from a subject; or (v) any combination of (i), (ii), (iii) and (iv), is assessed over time.

In a further aspect, the present invention provides a method of determining the risk of developing a scoliosis in a cell sample from a subject, the method comprising detecting the presence or absence of a polymorphic marker in at least one allele of at least one gene listed in Table 4 or substitute marker in linkage disequilibrium with the polymorphic marker. In embodiments, the polymorphic marker is a polynucleotide variant set forth in Table 6.

In another aspect, the present invention provides a method of genotyping a subject suffering from Idiopathic scoliosis or at risk of developing a scoliosis comprising determining in a cell sample from the subject the presence or absence of a polymorphic marker in at least one allele of at least one gene listed in Table 4 or a substitute marker in linkage disequilibrium with the polymorphic marker. In embodiments, the polymorphic marker is a polynucleotide variant set forth in Table 6.

In a further aspect, the present invention provides a method of classifying a subject (e.g., suffering from a scoliosis or at risk of developing a scoliosis) comprising performing one or more of the above-described methods and classifying the subject into an IS group.

In embodiments, the above-described methods comprise determining the presence or absence of at least two polymorphic markers. In embodiments, the methods comprise determining the presence or absence of at least two polymorphic markers in at least two genes. In embodiments, the above-described methods comprise determining the presence or absence of at least 3, 4, 5, 6, 7, 8, 9, 10, 20, 30, 40, 50, 60, 70, 80, 90, 100, 110, 120 or more polymorphic markers. In embodiments, the methods comprise determining the presence or absence of at least 3, 4, 5, 6, 7, 8, 9, 10, 20, 30, 40, 50, 60, 70, 80, 90, 100, 110, 120 or more polymorphic markers in at least 3, 4, 5, 6, 7, 8, 9, 10, 20, 30, 40, 50, 60, 70, 80, 90, 100, 110, 120 or more genes.

The above-described methods may be used alone or in combination with each other. Methods of the present invention may also be used in combination with known methods useful for determining the risk of or predisposition to developing a scoliosis; for genotyping a subject; and/or for classifying a subject into an IS group.

In a further aspect, the present invention provides a composition or kit comprising one or more reagent for detecting (a) the length of cilia at the surface of cells; (b) the number of cells with elongated cilia; (c) the number of ciliated cells; (d) the level of expression of at least one mechanoresponsive gene; and/or (e) the presence or absence of a polymorphic marker in at least one gene listed in Table 4 or a substitute marker in linkage disequilibrium therewith in a cell sample from a subject. In embodiments, the composition or kit further comprises the cell sample from the subject. In embodiments, the cell sample comprises cells which have been submitted to a mechanostimulation. In embodiments, the composition or kit comprises at least one oligonucleotide probe or primer for the specific detection of a polymorphic marker in a gene listed in Table 4. In embodiments, the polymorphic marker is a polynucleotide variant set forth in Table 6.

In a further aspect, the present invention provides a DNA chip comprising at least one oligonucleotide for detecting the presence or absence of a polymorphic marker in at least one gene listed in Table 4 and a substrate on which the oligonucleotide is immobilized. In embodiments, the polymorphic marker is a polynucleotide variant set forth in Table 6.

In a further aspect, the present invention provides oligonucleotide primers or probes for use in the above-described methods. In embodiments, the oligonucleotide is for the specific detection of a polymorphic marker of the present invention and comprises or consists of a nucleotide sequence having a variant at a position corresponding to that defined in Table 6. In embodiments, the variant is a risk variant defined in Table 6. In embodiments, the oligonucleotide primer or probe hybridizes to a reference or a variant polynucleotide sequence set forth in Table 6 or to its complementary sequence. In embodiments, the oligonucleotide primer or probe further comprises a label. In embodiments, the oligonucleotide primer or probe comprises or consists of a polynucleotide sequence set forth in Table 6 or the complement thereof. In embodiments, the oligonucleotide primer or probe consists of 10 to 100 nucleotides, preferably 10 to 60 nucleotides. In embodiments, the oligonucleotide primer or probe consists of at least 12 nucleotides.

In a further aspect, the present invention relates to the use of methods, compositions, oligonucleotide primers or probes, kits or DNA chips of the present invention for (i) determining the risk of or predisposition to developing a scoliosis; (ii) genotyping a subject; and (iii) classifying a subject into an IS group.

In embodiments, the above-mentioned mechanostimulation is fluid sheer stress. In embodiments, the level of sheer stress corresponds to a Womersley number of between about 5 and 18. In embodiments, the level of sheer stress corresponds to a Womersley number of about 8. In embodiments, mechanostimulation corresponds to an average sheer stress of about 1 Pa. In embodiments, mechanostimulation is applied at a frequency of between about 1 and about 3 Hz.

In embodiments, the at least one gene comprising a polymorphic marker (gene variant) comprises FEZF1, CDH13, FBXL2, TRIM13, CD1B, VAX1, CLASP1, SUGT1, MIPEP, FAM188A, TAF6, WHSC1, GPR158, HNRNPD, RUNX1T1, GRIK3, FUZ, LYN, DDX5, PODXL, ATP5B, PIGK, AL159977.1, BTN1A1, CDK11A, HIVEP1, HSD17B14, KCNMA1, PXDN, RAB31, RBM5, RNF149, SOD2, TOPBP1, ZCCHC14, ZNF323, or any combination thereof. In embodiments, the at least one gene comprises FEZF1, CDH13, FBXL2, TRIM13, CD1B, VAX1, CLASP1, SUGT1, MIPEP, FAM188A, TAF6, WHSC1, GPR158, HNRNPD, RUNX1T1, GRIK3, FUZ, LYN, DDX5, PODXL, ATP5B, PIGK, AL159977.1, or any combination thereof. In embodiments, the at least one gene comprises ATP5B, BTN1A1, CD1B, CDK11A, CLASP1, DDX5, FBXL2, HIVEP1, HSD17B14, KCNMA1, PXDN, RAB31, RBM5, RNF149, SOD2, SUGT1, TOPBP1, ZCCHC14, ZNF323 or any combination thereof. In embodiments the at least one gene comprises ATP5B, BTN1A1, CD1B, CDK11A, CLASP1, DDX5, FBXL2, HIVEP1, HSD17B14, KCNMA1, PXDN, RAB31, RBM5, RNF149, SOD2, SUGT1, TOPBP1, ZCCHC14 or ZNF323 or any combination thereof. In embodiments, the at least one gene comprises CDB1, CLASP1 and SUGT1.

In embodiments, the above-mentioned polymorphic marker is a polymorphic marker defined in Table 6. In embodiments, the polymorphic marker is a risk variant defined in Table 6.

In embodiments, the above-mention subject is a female. In embodiments, the subject is prediagnosed with a scoliosis (e.g., iodiopathic scoliosis). In embodiments, the subject has a family member which has been diagnosed with a scoliosis. In embodiments, the subject is a likely candidate for developing a scoliosis or for developing a more severe scoliosis.

In embodiments, the cell sample comprises bone cells. In embodiments, the cell sample comprises mesenchymal stem cells, myoblasts, preosteoblasts, osteoblasts, osteocytes and/or chondrocytes. In embodiments, the cell sample is a blood sample. In embodiments, the cell sample is a blood sample comprising PBMCs. In embodiments, the cell sample is a nucleic acid sample. In embodiments, the cell sample is a protein sample.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIGS. 1A-C show the morphology of primary cilia in osteoblasts from IS and controls. (A) Immunofluorescence micrographs of IS and control osteoblasts at 0, 24, 48, and 72 hours following serum-starvation. Cells were stained for acetylated α-Tubulin, F-Actin, and Hoescht. Long cilia (arrow) are visible in IS patients, at all time-points. (B) Elongated primary cilia appear more frequently in IS bone cells (4 IS vs. 4 controls assayed in duplicate, from 5×5 stitched tile images (50 fields) per sample). (C) Percentage of ciliated cells is not significantly different between IS and control cells (n≤1,000 count per individual). Error bars are constructed using 1 standard error from the mean. Statistical analysis was performed with t-test using JMP-11®, *P<0.005 (see Example 3);

FIG. 2 shows a similar growth rate among IS and control cells. There is no significant difference in the average cell number between control and IS patient cells at any given time point (n=8:4 IS vs. 4 controls). Plates were seeded with 100,000 cells per well, in triplicate, for each patient and control. Each error bar is constructed using 1 standard error from the mean (see Example 4);

FIGS. 3A-C show the biomechanical response profile of IS cells with elongated cilia. RT-qPCR was used to examine the effect of oscillatory fluid flow on gene expression after 4, 8, and 16 hours of stimulation. Gene expression in every sample has been normalized to two endogenous controls (GAPDH and HPRT). The 0 h of every sample has been defined as its calibrator. The graphs represent the fold changes at each time point, compared to the calibrator. For each gene, the two groups (control and IS) were compared at each time point using a pairwise t-test. In addition, for ITGB1 ((B)(ii)), CTNNB1 ((A(iii)) and POC5 (B(iii) the expression at 0 h for IS was compared to each of the other time points (4 h, 8 h and 16 h) using separate pairwise t-tests. For a post hoc Bonferroni analysis, the maximum number of comparisons per gene is 6, three comparisons per question (i.e. three comparisons per family of test). Even if we consider each gene as a family (i.e. six comparisons), using this formula (FWER=1−(1−α) M⁷⁵) the family-wise error rate (FWER) would be: 1−(1−0.05)6≈1−0.73≈0.26. Solving the Bonferroni (0.26/6) new α would be 0.043. CTNNB1 results at 4 (p=0.03) and 8 hours (p=0.008) will still be significant. It is the same case for POC5 4 h (p=0.01) but ITGB1 with a p=0.047 will not pass the test. Overall, the multiple test error is not significant in our analysis and it will not change the results. Genes were chosen based on the following characteristics: Biomechanically responsive genes in bone tissue: BMP2 ((A(i)), PTGS2 (COX2) ((B)(i)), RUNX2 ((C)(i)), SPP1 (OPN) ((A)(ii)); Role in mechanotransduction through cilia: ITGB1 ((B)(2)), ITGB3 ((C)(ii)); Indicator of Wnt pathway activity: CTNNB1 ((A)(iii)); or Implicated in an IS study: POC5 ((B)(iii)) and FUZ ((C)(iii)). Each error bar is constructed using 1 standard error from the mean. n=8, (4 IS vs. 4 controls) for all genes except CTNNB1 and FUZ, where n=4 (2 IS vs. 2 controls) (see Example 5);

FIG. 4 shows that the differentially affected molecules identified in FIG. 3 (Example 5) are connected through pathways linking ciliary mechanosensation to bone formation. The molecules shown herein to be differentially affected in IS (marked by an arrow) are related through multiple interconnected pathways, summarized in this figure. The results of gene expression studies reported herein are confirmed by expected responses through these pathways. For example, BMP2 expression directly affects RUNX2, which in turn affects COX2 expression;

FIGS. 5A-B show the mutation profile of IS patients tested in Examples 3-5 (FIG. 3). Patients used in the cellular assays were surveyed for variants (risk variants) in genes listed in Table 4 (significant genes from our SKAT-O analyses). Patients are listed as rows and each column is a gene. This heat map illustration shows the number of variants per patient for a given gene. Only genes with a total of more than 1 variant are listed. (A) KCNMA1, PXDN, RAB31, RBM5, RNF149, SOD2, SUGT1, TOPBP1, ZCCHC14 and ZNF323; and (B) ATPB5, BTN1A1, CDB1, CDK11A, CLASP1, DDX5, FBXL2, HIVEP1 and HSD17B14;

FIGS. 6A-C show the characterization of osteoblast cells. Osteoblasts were derived from bone fragments obtained intraoperatively. Alizarin red staining and expression of osteoblast markers were used to confirm that the cells are osteoblasts. (A) Mineralization was induced on a confluent monolayer of cells (in duplicate) by addition of ascorbic acid (50 μg/ml), beta-glycerophosphate (2.5 mM) and dexamethasone (10 nM). After 4 weeks of treatment, cells were fixed with formaldehyde and stained with Alizarin red. (B) In addition to the RT-qPCR performed in this study using osteoblast genes (RUNX2 and SPP1), RT-qPCR using Alkaline phosphatase (ALP) and Bone Sialoprotein II (BSP) as osteoblast markers was also performed. (C) Shows the sequence of the primers used for RT-PCR; and

FIG. 7 shows that elongated cilia found in IS cells are not microtubules. To validate the staining of cilia (FIG. 1), double immunostaining was performed on fixed IS osteoblasts using Anti-acetylated α-Tubulin and (A) anti-Ninein, as the basal body marker or (B) anti-IFT88 to stain the length of cilia. Lower parts of each panel (C and D) show the magnified version of the area framed in white rectangles from the upper part. Three different channels of staining are shown side by side the merged image. The images were captured on a Leica Confocal TCS-SP8 using ×63 (oil) objective and Maximum projections of full Z-stacks.

### DESCRIPTION OF ILLUSTRATIVE EMBODIMENTS

The primary cilium is an outward projecting antenna-like organelle with an important role in bone mechanotransduction. The capacity to sense mechanical stimuli can affect important cellular and molecular aspects of bone tissue. Idiopathic scoliosis (IS) is a complex pediatric disease of unknown cause, defined by abnormal spinal curvatures. As shown herein, a significant elongation of primary cilia in IS patient bone cells was established. Furthermore, IS subjects have an increase number of cells with elongated cilia. In response to mechanical stimulation, these cells differentially express osteogenic factors, mechanosensitive genes, and beta-catenin. Considering that numerous ciliary genes are associated with a scoliosis phenotype, among ciliopathies and knockout animal models, IS patients were expected to have an accumulation of rare variants (risk variants) in ciliary genes. Instead, the SKAT-O analysis of whole exomes presented herein showed enrichment among IS patients for rare variants in genes with a role in cellular mechanotransduction. Applicant's data indicates defective cilia in IS bone cells, which is likely linked to heterogeneous gene variants pertaining to cellular mechanotransduction.

The present invention is thus based on the identification of functional defects in cells from IS subjects and on the identification of novel molecular markers, and in particular novel SNPs in various genes involved in mechanotransduction. The present invention thus provides novel methods of determining the risk of developing IS (or of detecting a predisposition to or the presence of), of genotyping subjects (e.g., IS subjects or subjects at risk of developing a scoliosis) and of classifying subjects (e.g., IS subject or subjects at risk of developing a scoliosis). The present invention further provides methods for identifying novel therapeutic targets and means for improving the application of treatment and preventive measures.

### DEFINITIONS

For clarity, definitions of the following terms in the context of the present invention are provided.

The articles “a,” “an” and “the” are used herein to refer to one or to more than one (i.e., to at least one) of the grammatical object of the article.

The term “including” and “comprising” are used herein to mean, and are used interchangeably with, the phrases “including but not limited to” and “comprising but not limited to”.

The terms “such as” are used herein to mean, and is used interchangeably with, the phrase “such as but not limited to”.

Polymorphism. The genomic sequence within populations is not identical when individuals are compared. Rather, the genome exhibits sequence variability between individuals at many locations in the genome. Such variations in sequence are commonly referred to as polymorphisms, and there are many such sites within each genome. For example, the human genome exhibits sequence variations which occur on average every 500 base pairs. Thus, as used herein, a “polymorphism” refers to a variation in the sequence of nucleic acid (e.g., a gene sequence). Such variation include insertion, deletion, and substitutions in one or more nucleotides.

The most common sequence variation (or polymorphism) consist of base variations at a single base position in the genome, and such sequence variants, or polymorphisms, are commonly called Single Nucleotide Polymorphisms (“SNPs”). There are usually two possibilities (or two alleles) at each SNP site; the original allele and the mutated allele (although there may 3 or 4 possibilities for each SNP site). Due to natural genetic drift and possibly also selective pressure, the original mutation has resulted in a polymorphism characterized by a particular frequency of its alleles in any given population. There may also exists SNPs that vary between paired chromosomes in an individual. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). As used herein an SNP thus refers to a variation at a single nucleotide in a given nucleic acid sequence.

As noted above, many other types of sequence variants are found in the human genome, including microsatellites, insertions, deletions, inversions and copy number variations. A polymorphic microsatellite has multiple small repeats of bases (such as CA repeats, TG on the complimentary strand) at a particular site in which the number of repeat lengths varies in the general population.

In general terms, each version of the sequence with respect to the polymorphic site represents a specific allele of the polymorphic site. These sequence variants can all be referred to as polymorphisms, occurring at specific polymorphic sites characteristic of the sequence variant in question. In general terms, polymorphisms can comprise any number of specific alleles.

In some instances, reference is made to different alleles at a variant/polymorphic site without choosing a reference allele. Alternatively, a reference sequence can be referred to for a particular polymorphic site. The reference allele is sometimes referred to as the “wild-type” allele and refers herein to the allele from a “non-affected” or control/reference individual (e.g., an individual that does not display a trait or disease phenotype i.e., which does not suffer from a scoliosis or which has a lower risk of (or predisposition to) developing a scoliosis).

A “polymorphic marker”, also referred to as a “genetic marker” or “gene variant”, as described herein, refers to a variation (mutation or alteration) in a gene sequence that occurs in a given population. Each polymorphic marker/gene variant has at least two sequence variations (e.g., 2, 3, 4, 5, 6, 7, 8, or more sequence variations) characteristic of particular alleles at the polymorphic site. The marker/gene variant can comprise any allele of any variant type found in the genome, including variations in a single nucleotide (SNPs, microsatellites, insertions, deletions, duplications and translocations. The polymorphic marker/gene variant, if found in a transcribed region of the genome can be detected not only in genomic DNA but also in RNA. Polymorphic markers or gene variants of the present invention and identified in Table 6 are found in transcribed regions of the genome (were identified following exome sequencing). In addition, when the polymorphism/variant is found in the gene portion that is translated into a polypeptide or protein, the polymorphic marker/gene variant can be detected at the protein/polypeptide level.

The polymorphic marker/gene variant of the present invention and its specific sequence variation can be detected by various means such as by sequencing the nucleic acid or protein. Alternatively, when the polymorphism/variation affects the function of the gene or of its translated protein/polypeptide, the biological activity can be evaluated in order to identify which allele is present in the subject's sample. For example, if a particular risk allele (comprising a risk variant or combination of risk variants) affects the enzymatic activity of the protein, then, the presence of the allele or variant(s) can be assessed by performing an enzymatic test. Alternatively, if the risk allele (comprising a gene variant or combination of variants) affects the expression level of a polypeptide or nucleic acid, then, the presence of the variants(s) can be determined by assessing the expression level (e.g., Immunoassays, amplification assays, etc.) of such protein or nucleic acid and comparing it to a reference level in a control sample (e.g., sample from a subject not suffering from a scoliosis or at risk of developing a scoliosis).

An “allele” refers to the nucleotide sequence of a given locus (position) on a chromosome. A polymorphic marker allele thus refers to the composition (i.e., sequence) of the marker on a chromosome. Genomic DNA from an individual contains two alleles for any given polymorphic marker, representative of each copy of the marker on each chromosome. A “risk allele”, a “susceptibility allele” or a “predisposition allele” or a “risk variant” is nucleic acid sequence variation that is associated with an increased risk of (i.e. compared to a control/reference) or predisposition to suffering from a scoliosis. Conversely, a “protective allele” or “protective variant” is a sequence variation of a polymorphic marker that is associated with a lower risk of (i.e., compared to a control/reference) or predisposition to suffering from a scoliosis.

A “nucleic acid or gene associated with idiopathic scoliosis” as used herein is a nucleic acid (e.g., genomic DNA or RNA) that comprises a polymorphic marker/gene variant of the present invention, or any substitute marker in linkage disequilibrium therewith, which affects the risk of developing a scoliosis (e.g., a risk variant defined in Table 6). This nucleic acid may be of any length as long as it comprises the polymorphic region that is relevant to the determination of the presence or absence of susceptibility to scoliosis (e.g., the polymorphic markers or genes listed in Tables 4-6). For example, it can consist of a whole gene sequence (including the promoter or any other regulatory sequence) or of a fragment thereof. Similarly, a “polypeptide associated with idiopathic scoliosis” or a “protein associated with idiopathic scoliosis” refers to a protein or polypeptide which is encoded by a nucleic acid comprising a polymorphic marker of the present invention, or any marker in linkage disequilibrium therewith, which is associated with idiopathic scoliosis (e.g., comprising a risk variant defined in Table 6).

The term “sample” as used herein is any type of biological sample which may be used under the methods of the present invention. The term “cell sample” refers to a sample which originally comprised cells from the subject. For example, in certain embodiments, the “cell sample” is a sample in which it is possible to determine the average lengths of cilia on the surface of cells. Such cell sample thus comprises cells which normally have cilia. In embodiments, the cell sample allows for the detecting the presence or absence of a polymorphic marker (or gene variant) of the present invention (at the nucleic acid level or at the protein level) including but not limited to blood (including plasma and serum), urine, saliva, amniotic fluid, tissue biopsy etc. The sample may be a crude sample or a purified sample, it may be processed to a nucleic acid sample or a protein sample. In embodiments, the cell sample comprises bone cells. In embodiments, the cell sample comprises mesenchymal stem cells (MSC), chondrocytes, preosteoblasts, osteoblasts and/or osteocytes. In other embodiments, the cell sample is a blood sample (plasma or serum).

As used herein the terms “at risk of developing a scoliosis”, “predisposition to developing a scoliosis”, “at risk of developing IS”, and “predisposition to developing IS” refer to a genetic or metabolic predisposition of a subject to develop a scoliosis (i.e. spinal deformity) and/or a more severe scoliosis at a future time (i.e., curve progression of the spine). For instance, an increase of the Cobb's angle of a subject (e.g., from 40° to 50° or from 18° to 25°) is a “development” of a scoliosis. The terminology “a subject at risk of developing a scoliosis” includes asymptomatic subjects which are more likely than the general population to suffer in a future time of a scoliosis (i.e., a likely candidate for developing or suffering from a scoliosis) such as subjects (e.g., children) having at least one parent, sibling or family member suffering from a scoliosis. Among others, age (adolescence), gender and other family antecedent are factors that are known to contribute to the risk of developing a scoliosis and are used to evaluate the risk of developing a scoliosis. Also included in the terminology “a subject at risk of developing a scoliosis” are subjects already diagnosed with IS but which are at risk to develop a more severe scoliosis (i.e. curve progression).

As used herein the term “subject” is meant to refer to any mammal including human, mouse, rat, dog, chicken, cat, pig, monkey, horse, etc. In particular embodiments, it refers to a human (e.g., a child, adolescent (teenager) or adult which may benefit from any of the methods, compositions and kits of the present invention). In embodiments, the subject is a female. In embodiments, the subject has at least one family member which has been diagnosed with IS. In embodiments, the family member is a sibling.

As used herein the terminology “control sample” is meant to refer to a sample from which it is possible to make a suitable comparison under the methods of the present invention (e.g., to determine the risk of developing a scoliosis, to genotype subjects, classify/stratify subjects into a specific genetic or functional group, etc.). In embodiments, a “control sample” is a sample that does not originate from a subject known to have scoliosis or known to be a likely candidate for developing a scoliosis (e.g., idiopathic scoliosis (e.g., Infantile Idiopathic Scoliosis, Juvenile Idiopathic Scoliosis or Adolescent Idiopathic Scoliosis (AIS))). In the context of the present invention, “a control sample” also includes a “control value” or “reference signal” derived from one or more control samples from one or more subjects. In methods for classifying subjects or for determining the risk of developing a scoliosis in a subject that is pre-diagnosed with scoliosis, the sample may also come from the subject under scrutiny at an earlier stage of the disease or disorder. Preferably, the control sample is a cell of the same type (e.g., both the test sample and the reference sample(s) are e.g., lymphocytes, osteoblasts, myoblasts or chondrocytes) as that from the subject. Of course multiple control samples derived from different subjects can be used in the methods of the present invention. A control sample (or reference signal or control value) may correspond to a single control subject ((i.e., a normal healthy subject or a subject already classified in a given functional or genetic group) or may be derived from a group of control subjects (i.e., equivalent to the reference signal in control subjects).

### Methods of Determining the Risk of Developing Scoliosis, Methods of Genotyping and Methods of Classifying Subjects

In one aspect, the present invention provides a method of determining the risk of or predisposition to developing a scoliosis comprising determining the biomechanical profile of cells in a cell sample from a subject, wherein an altered biomechanical profile in the cell sample as compared to that in a control sample is indicative of an increased risk of or predisposition to developing a scoliosis.

In embodiments, determining the biochemical profile comprises (i) determining (e.g., measuring, detecting) the average length of cilia on the surface of cells in a cell sample from the subject; (ii) determining the number of cells with elongated cilia in a cell sample from the subject (iii) determining the number of ciliated cells in a cell sample from the subject; or (iv) any combination of (i), (ii) and (iii). An increase in the average length of cilia, an increase in the number of cells with elongated cilia or a decrease in the number of ciliated cells in the cell sample from the subject as compared to that in a control sample is indicative of an increased risk of or predisposition to developing a scoliosis.

In embodiments, determining the biochemical profile comprises measuring a response to mechanostimulation of cells in a cell sample from a subject, comprising: (i) applying mechanostimulation to cells from the cell sample from the subject; (ii) measuring the expression level of at least one mechanoresponsive gene. An altered expression level in said mechanoresponsive gene as compared to that of the control sample is indicative of an increased risk of or predisposition to developing a scoliosis.

Cellular mechanostimulation is performed by methods well known in the art (reviewed for example in Thomas D. brown: Techniques for cell and tissue culture mechanostimulation: historical and contemporary design considerations”, Iowa Orthop. J. 1995; 15: 112-117; Cha, B., Geng, X., Mahamud, M. R., Fu, J., Mukherjee, A., Kim, Y & Dixon, J. B. (2016). Mechanotransduction activates canonical Wnt/β-catenin signaling to promote lymphatic vascular patterning and the development of lymphatic and lymphovenous valves. Genes & Development, 30(12), 1454-1469; Zhou X, Liu D, You L, Wang L. Quantifying Fluid Shear Stress in a Rocking Culture Dish. Journal of biomechanics. 2010; 43(8):1598-1602). Such stimulation may be performed in various ways and may include the use of known mechanical devices designed to deliver proper loading, distention, or other mechanical stimuli. In particular embodiments, the mechanostimulation involves the application of fluid sheer stress to the cells. Fluid sheer stress may be defined in terms of the well-known Womersley number (see Example 1 for more details on the calculation of the Womersely number).

Preferably, the mechanical stimulation that is applied in accordance with the present invention is similar to that normally encountered by cells under physiological conditions (i.e., “a physiological mechanostimulation”). For example, in the case of the application of fluid sheer stress, the value of the Womersley number ranges from 5 to 18 in fluid motion of cerebrospinal fluid in the spinal cavity.

In certain embodiments of the methods of the present invention, the mechanostimulation is fluid sheer stress and the level of sheer stress applied is within the range of fluid sheer stress that may be encountered by cells, preferably human cells, under normal conditions. In embodiments, the level of fluid sheer stress applied corresponds to a Womersley number between about 5 and about 18. In particular embodiments the level of fluid sheer stress applied corresponds to a Womersley number of about 8. In embodiments, the frequency of mechanostimulation is between about 1 and 3 hz, preferably, 1 hz.

In another specific embodiment, said mechanostimulation is a pulsative compressive pressure. In another specific embodiment, said pulsative compressive pressure is applied using an inflatable strap. In another specific embodiment, said pulsative compressive pressure is applied using an inflatable cuff. In another specific embodiment, said mechanical stimulus or force is applied for a period of at least about 15 minutes. In another specific embodiment, said mechanical stimulus or force is applied for a period of between about 30 to about 90 minutes. In another specific embodiment, said mechanical stimulus or force is applied for a period of about 90 minutes. In another specific embodiment, the subject is a likely candidate for developing adolescent idiopathic scoliosis. In embodiments, the biological sample is taken from the subject after the end of mechanostimulation at a time which is sufficient for detecting variations in the expression (at the mRNA or protein level) of mechanoresponsive genes (e.g., 15, 20, 30, 45, 60, 90, 120 minutes from end of mechanostimulation). The time necessary to detect variations in gene expression may vary depending on the gene(s) of interest and on whether the variation in expression level is detected at the protein or nucleic acid (mRNA) level. For example, for some genes, a delay of 15 min. from the start of mechanostimulation may be sufficient to detect variations in gene expression. Thus in some embodiments the biological sample is taken from the subject 15, 20, 30, 45, 60, 90, 120 minutes from start of mechanostimulation). As used herein, a “mechanoresponsive gene” is a gene which expression varies in response to mechanostimulation. Non-limiting examples of such gene includes ITGB1, ITGB3, CTNNB1, POC5, BMP2, COX-2 (PTGS2) and RUNX2.

Thus, in embodiments, the methods of the present invention comprise measuring the expression level of at least one of ITGB1, ITGB3, CTNNB1; POC4, BMP2, COX-2 and RUNX2, preferably at least one of ITGB1; CTNNB1; BMP2, COX-2 and RUNX2, and more preferably at least one of ITGB1; CTNNB1; BMP2 and COX-2. An altered expression in at least one of the above mechanoresponsive genes in the cell sample from the subject as compared to that in the control sample is indicative of an increased risk of developing a scoliosis (or predisposition to IS or presence of IS). For example, (i) a decrease in BMP2, POC5, COX-2, ITGB1 (e.g., at 4 h post mechanostimulation) expression; or (ii) an increase in CTNNB1 expression or ITGB3 (e.g., at 8 or 6 h post mechanostimulation) expression in the cell sample from the subject as compared to that of a control sample is indicative of an increased risk of developing IS (or predisposition to IS or presence of IS).

In embodiments, the above method comprises determining in a cell sample from a subject (i) the average length of cilia on the surface of cells; (ii) the number of cells with elongated cilia; (iii) the number of ciliated cells; or (iv) the expression level of mechanoresponsive genes, over time. In embodiments, the determination is made prior to and after applying a mechanical stimuli to the cells (e.g., 1, 2, 4, 6, 8, 10, 12, 16, 24, 48 and/or 72 h following the application of a mechanical stimulation). An altered average length of cilia, an increase in the number of cells with elongated cilia, a reduced number of ciliated cells or an altered expression level in at least one mechanoresponsive gene over at least one point in time is indicative of an increased risk of (or predisposition to) developing a scoliosis.

In a particular aspect, the present invention provides a method of determining the risk of developing a scoliosis (or of detecting a predisposition to IS or the presence of IS) in a subject comprising (i) applying a physiological level of fluid sheer stress to a cell sample from the subject; and (ii) determining the expression level of a mechanoresponsive gene in the cell sample; (iii) comparing the expression level of the mechanoresponsive gene to that in a control sample; and (iv) determining the risk of developing a scoliosis (or predisposition to IS or the presence of IS) based on the results in step (iii), wherein the mechanoresponsive gene is BMP2, COX-2, RUNX2, ITGB1, ITGB3, CTNNB1, POC5 or any combination of at least two of these genes. An altered gene expression in the mechanoresponsive gene in the cell sample from the subject as compared to that in the control sample is indicative of an increased risk of developing a scoliosis (or predisposition to IS or presence of IS). For example, (i) a decrease in BMP2, COX-2, POC5, ITGB3 (e.g., at 4 h post mechanostimulation) or ITGB1 expression; or (ii) an increase in CTNNB1 expression or ITGB3 expression (e.g., at 8 or 16 h post mechanostimulation) in the cell sample from the subject as compared to that of a control sample is indicative of an increased risk of developing IS (or predisposition to IS or presence of IS).

In a related aspect of the present invention, there is provided a method (e.g., an in vitro method) for determining the risk of developing a scoliosis (e.g., an Idiopathic Scoliosis (IS) such as Infantile Idiopathic Scoliosis, Juvenile Idiopathic Scoliosis or Adolescent Idiopathic Scoliosis (AIS)), in a subject said method comprising: (a) measuring a first level of at least one mechanoresponsive gene in a biological sample from said subject; (b) applying a mechanical stimulus or force to one or more members from said subject (e.g., compressive pressure); (c) measuring a second level of the at least one mechanoresponsive gene in a corresponding biological sample from the subject after the application of said mechanostimulation (biomechanical stimulus); (d) determining a variation between the first level of the at least one mechanoresponsive gene and the second level of the at least one mechanoresponsive gene; (e) comparing the variation to a control variation value; and (f) determining whether the subject has a scoliosis or is predisposed to developing a scoliosis based on the comparison.

Any of the above methods may also be used to classify subjects into specific IS groups. Thus, in a further aspect, the present invention provides a method of classifying a subject (e.g., a subject suffering from IS or at risk of developing IS) comprising determining the biomechanical profile of cells in a cell sample from a subject, wherein an altered biomechanical profile in the cell sample as compared to that in a control sample allows classifying the subject into a specific IS group.

In embodiments, determining the biochemical profile comprises (i) determining the average length of cilia on the surface of cells in a cell sample from the subject; (ii) determining the number of cells with elongated cilia in a cell sample from the subject (iii) determining the number of ciliated cells in a cell sample from the subject; or (iv) any combination of (i), (ii) and (iii). Scoliotic subjects may then be classified into specific groups based on for example, the average length of cilia on the surface of their cells, their number of cells having elongated cilia or based on the number of ciliated cells in their cell sample.

In embodiments, determining the biochemical profile comprises measuring a response to mechanostimulation of cells in a cell sample from a subject suffering from a scoliosis, comprising: (i) applying mechanostimulation to cells from the cell sample from the subject; (ii) measuring the expression level of at least one mechanoresponsive gene. An altered expression level in the at least one mechanoresponsive gene as compared to that of a control sample allows classifying the subject into a specific IS group.

Thus, in embodiments, the above classification method comprises measuring the expression level of at least one of the following mechanoresponsive gene: ITGB1, ITGB3, CTNNB1; POC5, BMP2, COX-2, RUNX2 and CTNNB1, preferably of at least one of ITGB1; CTNNB1; BMP2, COX-2 and RUNX2, and more preferably ITGB1; CTNNB1; BMP2 and COX-2. Subjects may be classified for example according to the specific gene or genes which expression is altered. In addition (or alternatively), subjects may be classified according to the level of variation in gene expression detected (overtime or at a single point in time) and/or based on the absence or presence of a variation in gene expression following mechanostimulation (overtime or at a single point in time). Scoliotic subjects may be compared to control non-scoliotic subjects or to each other and classified accordingly.

As noted above, the present invention discloses the presence of certain gene variants (polymorphic markers) in cells from IS subjects (see Table 4). In particular, rare gene variants (e.g., polymorphisms such as SNPs) have been identified in the following genes: FEZF1, CDH13, FBXL2, TRIM13, CD1B, VAX1, CLASP1, SUGT1, MIPEP, FAM188A, TAF6, WHSC1, GPR158, HNRNPD, RUNX1T1, GRIK3, FUZ, LYN, DDX5, PODXL, ATP5B, PIGK, AL159977.1, SEPT9, TMEM87A, CDYL, SPINT3, SERTM1, FOLR3, FCER2, MAEA, PXT1, UVRAG, SPPL3, IGHV3-50, HIVEP1, SMAD5, PPP1R21, SEC62, TOPBP1, HIPK3, KRTAP12-2, FYB, PXDN, CDV3, RP3-344J20.2, RP11-405L18.2, MRPL18, SOD2, FOXP2, REEP1, C1orf106, DNASE1L1, BTN1A1, MLST8, HMP19, OR8B4, AC105901.1, OR5F1, GLE1, OR5P3, SCFD1, CDK11A, HSD17B14, NFU1, GTF2H3, RAB7A, HOXA3, ZC3H4, DDX55, FBXW10, OSBPL2, POLR1A, NOP58, RAB31, EFNB2, ZCCHC14, GLP1R, RNF149, OR1J2, WI2-81516E3.1, GAPDHP27, SFTA3, ACSF3, POU2F2, MIR345, SNPH, MATR3, RP11-73B8.2, SNORA48, PATZ1, RBM5, HMGA1, ATP1A3, ACTG1P1, PAIP1, KCNMA1, PALB2, PLEKHG5, C11orf2, MT1DP, CYC1, DTD1, CREB3L3, RPL23A, CD164L2, PCCB, GIMAP7, AHCYL1, TNNT2, ZNF134, AC079612.1, MTA2, RP11-672F9.1, CLEC5A, C1orf222, CD96, PPFIBP1, ZNF323 and SUPT3H.

Accordingly, in a further aspect, the present invention provides a method of determining the risk of developing a scoliosis (or a predisposition to IS or the presence of IS) in a cell sample from a subject, the method comprising detecting the presence or absence of at least one polymorphic marker (gene variant) in at least one gene allele of FEZF1, CDH13, FBXL2, TRIM13, CD1B, VAX1, CLASP1, SUGT1, MIPEP, FAM188A, TAF6, WHSC1, GPR158, HNRNPD, RUNX1T1, GRIK3, FUZ, LYN, DDX5, PODXL, ATP5B, PIGK, AL159977.1, SEPT9, TMEM87A, CDYL, SPINT3, SERTM1, FOLR3, FCER2, MAEA, PXT1, UVRAG, SPPL3, IGHV3-50, HIVEP1, SMAD5, PPP1R21, SEC62, TOPBP1, HIPK3, KRTAP12-2, FYB, PXDN, CDV3, RP3-344J20.2, RP11-405L18.2, MRPL18, SOD2, FOXP2, REEP1, C1orf106, DNASE1L1, BTN1A1, MLST8, HMP19, OR8B4, AC105901.1, OR5F1, GLE1, OR5P3, SCFD1, CDK11A, HSD17B14, NFU1, GTF2H3, RAB7A, HOXA3, ZC3H4, DDX55, FBXW10, OSBPL2, POLR1A, NOP58, RAB31, EFNB2, ZCCHC14, GLP1R, RNF149, OR1J2, WI2-81516E3.1, GAPDHP27, SFTA3, ACSF3, POU2F2, MIR345, SNPH, MATR3, RP11-73B8.2, SNORA48, PATZ1, RBM5, HMGA1, ATP1A3, ACTG1P1, PAIP1, KCNMA1, PALB2, PLEKHG5, C11orf2, MT1DP, CYC1, DTD1, CREB3L3, RPL23A, CD164L2, PCCB, GIMAP7, AHCYL1, TNNT2, ZNF134, AC079612.1, MTA2, RP11-672F9.1, CLECSA, C1orf222, CD96, PPFIBP1, ZNF323 or SUPT3H.

In another aspect, the present invention provides a method of genotyping a subject (e.g., a subject suffering from a scoliosis or at risk of developing a scoliosis (e.g., Idiopathic scoliosis)) comprising determining in a cell sample from the subject the presence or absence of at least one polymorphic marker in an allele of at least one of FEZF1, CDH13, FBXL2, TRIM13, CD1B, VAX1, CLASP1, SUGT1, MIPEP, FAM188A, TAF6, WHSC1, GPR158, HNRNPD, RUNX1T1, GRIK3, FUZ, LYN, DDX5, PODXL, ATP5B, PIGK, AL159977.1, SEPT9, TMEM87A, CDYL, SPINT3, SERTM1, FOLR3, FCER2, MAEA, PXT1, UVRAG, SPPL3, IGHV3-50, HIVEP1, SMAD5, PPP1R21, SEC62, TOPBP1, HIPK3, KRTAP12-2, FYB, PXDN, CDV3, RP3-344J20.2, RP11-405L18.2, MRPL18, SOD2, FOXP2, REEP1, C1orf106, DNASE1L1, BTN1A1, MLST8, HMP19, OR8B4, AC105901.1, OR5F1, GLE1, OR5P3, SCFD1, CDK11A, HSD17B14, NFU1, GTF2H3, RAB7A, HOXA3, ZC3H4, DDX55, FBXW10, OSBPL2, POLR1A, NOP58, RAB31, EFNB2, ZCCHC14, GLP1R, RNF149, OR1J2, WI2-81516E3.1, GAPDHP27, SFTA3, ACSF3, POU2F2, MIR345, SNPH, MATR3, RP11-73B8.2, SNORA48, PATZ1, RBM5, HMGA1, ATP1A3, ACTG1P1, PAIP1, KCNMAI, PALB2, PLEKHG5, C11orf2, MTIDP, CYC1, DTD1, CREB3L3, RPL23A, CD164L2, PCCB, GIMAP7, AHCYL1, TNNT2, ZNF134, AC079612.1, MTA2, RP11-672F9.1, CLEC5A, C1orf222, CD96, PPFIBP1, ZNF323 or SUPT3H. Such method allows classifying subjects into specific IS groups. Such classification may allow the application of personalized prevention and treatment regimens, based on the specific genotype the subject.

In embodiments, the above methods comprise detecting the presence or absence of at least one polymorphic marker (gene variant) in at least one gene allele of FEZF1, CDH13, FBXL2, TRIM13, CD1B, VAX1, CLASP1, SUGT1, MIPEP, FAM188A, TAF6, WHSC1, GPR158, HNRNPD, RUNX1T1, GRIK3, FUZ, LYN, DDX5, PODXL, ATP5B, PIGK, AL159977.1, BTN1A1, CDK11A, HIVEP1, HSD17B14, KCNMA1, PXDN, RAB31, RBM5, RNF149, SOD2, TOPBP1, ZCCHC14 and ZNF323.

In embodiments, the above methods comprise detecting the presence or absence of at least one polymorphic marker (gene variant) in at least one gene allele of FEZF1, CDH13, FBXL2, TRIM13, CD1B, VAX1, CLASP1, SUGT1, MIPEP, FAM188A, TAF6, WHSC1, GPR158, HNRNPD, RUNX1T1, GRIK3, FUZ, LYN, DDX5, PODXL, ATP5B, PIGK and AL159977.1.

In embodiments, the above methods comprise detecting the presence or absence of at least one polymorphic marker (gene variant) in at least one gene allele of ATP5B, BTN1A1, CD1B, CDK11A, CLASP1, DDX5, FBXL2, HIVEP1, HSD17B14, KCNMA1, PXDN, RAB31, RBM5, RNF149, SOD2, SUGT1, TOPBP1, ZCCHC14 and ZNF323.

In embodiments, the above methods comprise detecting the presence or absence of at least one polymorphic marker (gene variant) in at least one gene allele of HNRNPD, ATP5B, LYN, CD1B, CLASP1, SUGT1 and AL159977.1.

Preferably, the above methods comprise detecting the presence or absence of at least one polymorphic marker (gene variant) in at least one gene allele of CD1B, CDK11A, CLASP1, RNF149 and SUGT1, more preferably, in at least one of CDB1, CLASP1 and SUGT1. In embodiments, the methods comprise detecting the presence or absence of a polymorphic marker in CDB1, CLASP1 and SUGT1.

In a particular aspect, the above-mentioned polymorphic marker is a gene variant (e.g., SNP) as defined in Table 6. In embodiments, the polymorphic marker is a risk variant defined in Table 6.

Methods of the present invention may further comprise detecting the presence or absence of at least polymorphic marker (e.g., risk variant, SNP) in at least one gene listed in Table 2.

In the above methods, detecting the presence of a risk allele (risk variant(s)) in polymorphic markers of one or more of the above genes is indicative of a risk of developing a scoliosis (or predisposition to IS). The level of risk or the likelihood of developing a scoliosis is determined depending on the number of risk-associated variants that are present in cells from a subject. The level of risk is determined by calculating a genetic score (ODD ratio), as well known in the art.

Accordingly, the present invention encompasses detecting the presence or absence of a polymorphic marker (e.g., SNP) in multiple genes listed in Tables 2 and 4-6 (e.g., a combination of 2, 3, 4, 5, 6, 7, 8, 9, 10, 11, 12, 13, 14, 15, 15, 16, 17, 18, 20, 21, 21, 22, 23, 24, 25, 26, 27, 28, 29, 30, 35, 40, 46, 50, 55, 60, 65, 70, 75, 80, 85, 90, 95, 100, 105, 110, 115 and 120 genes).

Alleles for SNP markers as referred to herein refer to the bases A, C, G or T as they occur at the polymorphic site in the SNP assay employed. The person skilled in the art will realize that by assaying or reading the opposite DNA strand, the complementary allele can in each case be measured. Thus, for a polymorphic site (polymorphic marker) characterized by an A/G polymorphism, the assay employed may be designed to specifically detect the presence of one or both of the two bases possible, i.e. A and G. Alternatively, by designing an assay that is designed to detect the opposite strand on the DNA template, the presence of the complementary bases T and C can be measured. Quantitatively (for example, in terms of relative risk), identical results would be obtained from measurement of DNA strands (+ strand or − strand).

Detecting specific gene variants or polymorphic markers and/or haplotypes of the present invention can be accomplished by methods known in the art. Such detection can be made at the nucleic acid or amino acid (protein) level.

For example, standard techniques for genotyping for the presence of gene variants (e.g., SNPs and/or microsatellite markers) can be used, such as sequencing, fluorescence-based techniques (Chen, X. et al., Genome Res. 9(5): 492-98 (1999)), methods utilizing PCR, LCR, Nested PCR and other methods for nucleic acid amplification. Specific methodologies available for SNP genotyping include, but are not limited to, TaqMan™ genotyping assays and SNPlex™ platforms (Applied Biosystems), mass spectrometry (e.g., MassARRAY™ system from Sequenom), minisequencing methods, real-time PCR, Bio-Plex™ system (BioRad), CEQ and SNPstream™ systems (Beckman), Molecular Inversion Probe™ array technology (e.g., Affymetrix GeneChip), and BeadArray™ Technologies (e.g., Illumine GoldenGate and Infinium assays). By these or other methods available to the person skilled in the art, one or more alleles at polymorphic markers, including microsatellites, SNPs or other types of polymorphic markers, can be identified.

In accordance with another aspect of the present invention, there is provided a method of selecting a preventive measure, treatment or follow-up schedule for a subject suffering from IS or at risk of developing IS comprising classifying or genotyping the subject using one or more of the above-noted methods:

### Linkage Disequilibrium

In order to determine the risk of developing a scoliosis (or predisposition to IS) it is also possible to assess the presence of a gene variant (such as a SNP) in linkage disequilibrium with any of the gene variants identified herein (e.g., SNPs/variants listed in Table 6).

Once a first SNP has been identified in a genomic region of interest, the practitioner of ordinary skill in the art can easily identify additional SNPs in linkage disequilibrium with this first SNP. In the context of the invention, the additional SNPs in linkage disequilibrium with a first SNP are within the same gene of said first SNP. Linkage disequilibrium (LD) is defined as the non-random association of alleles at different loci across the genome. Alleles at two or more loci are in LD if their combination occurs more or less frequently than expected by chance in the population.

For example, if a particular genetic element (e.g., an allele of a polymorphic marker, or a haplotype) occurs in a population at a frequency of 0.50 (50%) and another element occurs at a frequency of 0.50 (50%), then the predicted occurrence of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict.

When there is a causal locus in a DNA region, due to LD, one or more SNPs nearby are likely associated with the trait too. Therefore, any SNPs in LD with a first SNP associated with autism or an associated disorder will be associated with this trait. Identification of additional SNPs in linkage disequilibrium with a given SNP involves: (a) amplifying a fragment from the gene comprising a first SNP from a plurality of individuals; (b) identifying of second SNPs in the gene comprising said first SNP; (c) conducting a linkage disequilibrium analysis between said first SNP and second SNPs; and (d) selecting said second SNPs as being in linkage disequilibrium with said first marker. Subcombinations comprising steps (b) and (c) are also contemplated.

Methods to identify SNPs and to conduct linkage disequilibrium analysis can be carried out by the skilled person without undue experimentation by using well-known methods.

Thus, the practitioner of ordinary skill in the art can easily identify SNPs or combination of SNPs within haplotypes in linkage disequilibrium with the at risk gene variant (e.g. risk SNP).

Such markers are mapped and listed in public databases like HapMap as well known to the skilled person. Genomic LD maps have been generated across the genome, and such LD maps have been proposed to serve as framework for mapping disease-genes (Risch et ai, 1996; Maniatis et ai, 2002; Reich et ai, 2001). If all polymorphisms in the genome were independent at the population level (i.e., no LD), then every single one of them would need to be investigated in association studies, to assess all the different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

The two metrics most commonly used to measure LD are D′ and r² and can be written in terms of each other and allele frequencies. Both measures range from 0 (the two alleles are independent or in equilibrium) to 1 (the two allele are completely dependent or in complete disequilibrium), but with different interpretation. D′ is equal to 1 if at most two or three of the possible haplotypes defined by two markers are present, and <1 if all four possible haplotypes are present. r² measures the statistical correlation between two markers and is equal to 1 if only two haplotypes are present.

Most SNPs in humans probably arose by single base modifying events that took place within chromosomes many times ago. A single newly created allele, at its time of origin, would have been surrounded by a series of alleles at other polymorphic loci like SNPs establishing a unique grouping of alleles (i.e. haplotype). If this specific haplotype is transmitted intact to next generations, complete LD exists between the new allele and each of the nearby polymorphisms meaning that these alleles would be 100% predictive of the new allele. Thus, because of complete LD (D′=1 or r²=1) an allele of one polymorphic marker can be used as a surrogate for a specific allele of another. Event like recombination may decrease LD between markers. But, moderate (i.e. 0.5≤r; r²<0.8) to high (i.e. 0.8≤; r²<1) LD conserve the “surrogate” properties of markers. In LD based association studies, when LD exist between markers and an unknown pathogenic allele, then all markers show a similar association with the disease.

It is well known that many SNPs have alleles that show strong LD (or high LD, defined as r2≥0.80) with other nearby SNP alleles and in regions of the genome with strong LD, a selection of evenly spaced SNPs, or those chosen on the basis of their LD with other SNPs (proxy SNPs or Tag SNPs), can capture most of the genetic information of SNPs, which are not genotyped with only slight loss of statistical power. In association studies, this region of LD are adequately covered using few SNPs (Tag SNPs) and a statistical association between a SNP and the phenotype under study means that the SNP is a causal variant or is in LD with a causal variant. It is a general consensus that a proxy (or Tag SNP) is defined as a SNP in LD (r²≥0.8) with one or more other SNPs. The genotype of the proxy SNP could predict the genotype of the other SNP via LD and inversely. In particular, any SNP in LD with one of the SNPs used herein may be replaced by one or more proxy SNPs defined according to their LD as r²≥0.8.

These SNPs in linkage disequilibrium can also be used in the methods according to the present invention, and more particularly in the diagnostic methods according to the present invention. In particular, the presence of SNPs in linkage disequilibrium (LD) with the above identified SNPs may be genotyped, in place of, or in addition to, said identified SNPs. In the context of the present invention, the SNPs in linkage disequilibrium with the above identified SNP are within the same gene of the above identified SNP. Therefore, in the present invention, the presence of SNPs in linkage disequilibrium (LD) with a SNP of interest and located within the same gene as the SNP of interest may be genotyped, in place of, or in addition to, said SNP of interest. Preferably, such an SNP and the SNP of interest have r²≥0.70, preferably r2≥0.75, more preferably r²≥0.80, and/or have D′≥0.60, preferably D′≥0.65, D′≥0.7, D′≥0.75, more preferably D+≥0.80. Most preferably, such an SNP and the SNP of interest have r²≥0.80, which is used as reference value to define “LD” between SNPs.

### Compositions and Kits

Compositions and kits for use in the methods of the present invention (i.e., for determining the risk of developing a scoliosis; for genotyping a subject and for classifying a subject suffering from a scoliosis or at risk of developing a scoliosis) may include for example (i) one or more reagents for detecting (a) the length of cilia at the surface of cells; (b) the number of ciliated cells; (c) the level of expression of at least one mechanoresponsive gene; and/or (d) the presence or absence of a variant (polymorphic marker) in a gene listed in Table 4 or 6 or a substitute marker in linkage disequilibrium therewith.

Compositions and kits can comprise oligonucleotide primers and hybridization probes (e.g., allele-specific oligonucleotide primers and hybridization probes for determining the level of a mechanoresponsive gene or variant in a gene listed in Tables 4-6), restriction enzymes (e.g., for RFLP analysis) and/or antibodies that bind to a mutated polypeptide (polymorphic polypeptide) which is encoded by a nucleic acid comprising a polymorphic marker (e.g., gene variant) of the present invention (e.g., a nucleic acid comprising a variant (polymorphic marker) as defined in Table 6).

The kit (or composition) may also include any necessary buffers, enzymes (e.g., DNA polymerase) and/or reagents necessary for performing the methods of the present invention. The kit may comprise one or more labeled nucleic acids (or labeled antibody) capable of specific detection of one or more polymorphic markers of the present invention (e.g., gene variants defined in Table 6) or any markers in linkage disequilibrium therewith as well as reagents for the detection of the label. The kit may also comprise a device for applying a mechanical stimulus or force on one or more members of the subject (e.g., an inflatable strap or arm cuff).

Reagents may be provided in separate containers or premixed depending on the requirements of the method. Suitable labels are well known in the art and will be chosen according to the specific method used. Non-limiting examples of suitable labels (including non-naturally occurring labels/synthetic labels) include a radioisotope, a fluorescent label, a magnetic label, an enzyme, etc.

In a preferred embodiment, the detection of a polymorphic marker (e.g., gene variant defined in Table 6) in a gene associated with IS in accordance with the present invention is determined by DNA Chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the alteration of the genes, a sample from a test subject is labelled and contacted with the microarray in hybridization conditions, leading to the formation of complexes between target nucleic acids that are complementary to probe sequences attached to the microarray surface. The presence of labelled hybridized complexes is then detected. Many variants of the microarray hybridization technology are available to the man skilled in the art.

In embodiments, there is provided a composition (e.g., a diagnostic composition) which is generated following one or more steps of the methods describe herein and which include a biological sample (e.g., cell sample, blood sample, etc.) from the subject to be tested. The preparation of such composition occurs while testing a subject's biological sample for the risk of developing a scoliosis (including the risk of developing a more severe scoliosis); for aiding in the prevention and treatment of scoliosis including for determining the best treatment regimen; for adapting an undergoing treatment regimen; for selecting a new treatment regimen or for determining the frequency of a specific treatment regimen or follow-up schedule. Such compositions may be prepared using as kits described herein.

In embodiments, compositions and kits of the present invention may thus comprise one or more oligonucleotides probe or amplification primer for the detection (e.g., amplification or hybridization) of a mechanoresponsive gene (e.g., ITGB1, ITGB3, CTNNB1; POC5, BMP2, COX-2, RUNX2 and CTNNB1) or for the detection of a polymorphic marker of the present invention (e.g., a variant or reference sequence defined in Table 6). In embodiments, oligonucleotide probes are provided in the form of a microarray or DNA chip. The kit may further include instructions to use the kit in accordance with the methods of the present invention (e.g., for determining the risk of (or predisposition to) developing a scoliosis; for genotyping a subject or for classifying a subject suffering from a scoliosis or at risk of developing a scoliosis in a specific genetic or functional group).

The present invention is illustrated in further details by the following non-limiting examples.

### Method of Treatment

In certain subjects, scoliosis develops rapidly over a short period of time to the point of requiring a corrective surgery (often when the deformity reaches a Cobb's angle≥45°). Current courses of action available from the moment a scoliosis such as IS is diagnosed (when scoliosis is apparent) include observation (including periodic x-rays, when Cobb's angle is around 10-25°), orthopedic devices (such as bracing, when Cobb's angle is around 25-30°), and surgery (Cobb's angle over 45°). Thus, a more reliable determination of the risk assessment (through using one or more methods of the present invention) could enable to 1) select an appropriate diet to remove certain food products identified as contributors to scoliosis in certain subjects; 2) select the best therapeutic agent or treatment or preventive measure (e.g., neutralizing antibody specific to OPN, long term brace treatment, melatonin, selenium, PROTANDIM; HA supplements or HA-rich diet, antibody against CD44 etc.); 3) select the least invasive available treatment such as postural exercises (e.g., massages, or low intensity pulsed ultrasound (LIPUS), orthopedic device (brace) or other treatment or preventive measure (e.g., accupoint heat sensitive moxibustion, heat therapy with pad, thermal bath, electroacupuncture); or less invasive surgeries or surgeries without fusions (a surgery that does not fuse vertebra and preserves column mobility) and/or 4) the best follow-up schedule (e.g., increasing or decreasing the number of follow-up visit to the doctor during for example a 3, 6 or 12 month period or increasing or decreasing the number of x-rays during for example a 3, 6 or 12 month period).

### EXAMPLE 1

### Methods

Study cohorts. This study was approved by the institutional review boards of The Sainte-Justine University Hospital, The Montreal Children's Hospital, The Shriners Hospital for Children in Montreal and McGill University, as well as the Affluent and Montreal English School Boards. Parents or legal guardians of all the participants gave written informed consent, and minors gave their assent. All the experiments were performed in accordance with the approved guidelines and regulations. All subjects are residents of Quebec, Canada and are of European descent. Each IS patient was clinically assessed by an orthopedic surgeon at the Sainte-Justine Children's Hospital. The inclusion criteria for this study were a minimum Cobb angle of 10 degrees and a diagnosis of idiopathic scoliosis. Cobb angle is the clinical parameter used for quantification of curve magnitude where a larger angle denotes a greater magnitude. For cellular experiments, we used bone samples from a subset of patients who required corrective surgery. Control bone samples were from surgical non-scoliotic trauma patients recruited at the Sainte-Justine University Hospital. All patients used for cellular studies were adolescent females (Table 1). The medical files of controls were reviewed to exclude the possibility of scoliosis. For exome sequencing, control blood samples were collected from non-IS participants that were first screened by an orthopedic surgeon using the forward-bending test (Adam's test).⁶⁷ Any children with apparent spinal curvature were not included in the control cohort.

TABLE 1
Clinical features of patients tested for ciliary morphology.

| Patient ID | Cobb Angle (degree) | Curve Type |
|---|---|---|
| 1 | 42°-66°-38° | ITrTIL |
| 2 | 21°-50°-67°-31° | ITrTITLrL |
| 3 | 50°-56° | ITrL |
| 4 | 50°-89° | rTITL |

All samples (patients and controls) for the cellular assays (Examples 3-5) were from female subjects. Mean age for controls was 15 ± 3, mean age for patients was 15 ± 1. In the above table, Cobb angles are in degrees, multiple angles reflect multiple curves. Curve types-T: Thoracic; L: Lumbar; TL: Thoracolumbar; l: left and r right.

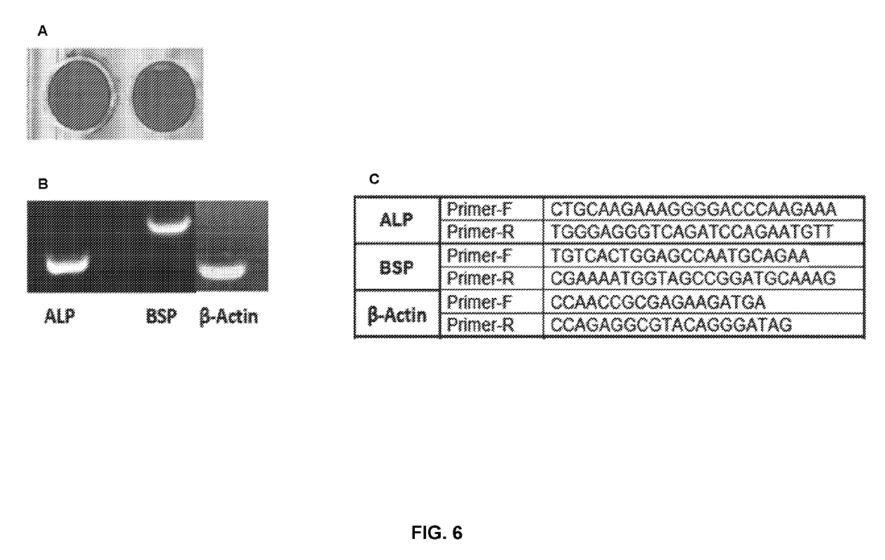

Cell culture. Primary osteoblasts were derived from bone specimens obtained from IS patients and trauma patients (as controls), intraoperatively. For all IS cases, bone specimens were surgically removed from affected vertebrae (the sampled vertebrae varied from T3 to L4) as a part of correctional surgery. For non-scoliotic control cases, bone specimens were obtained from other anatomic sites (tibia or femur) during trauma surgery. Using a cutter, bone fragments were manually reduced to small pieces under sterile conditions. The small bone pieces were incubated in aMEM medium containing 10% fetal bovine serum (FBS; certified FBS, Invitrogen Life Technologies, ON, Canada) and 1% penicillin/streptomycin (Invitrogen) at 37° C. in 5% CO₂, in a 10-cm² culture dish. After one month, osteoblasts emerging from the bone pieces were separated from the remaining bone fragments by trypsinization. Bone cells were characterized using alizarin red (FIG. 6) and ALP staining (data not shown). In addition to the expression of two osteoblast markers (RUNX2 and SPP1) used in the qPCR experiment, the expression of Alkaline Phosphatase and Bone Sialoprotein II was also confirmed using RT-PCR, as osteoblast specific genes in our primary cultures (FIG. 6). For serum starvation and to promote ciliogenesis, cells were washed in PBS and incubated in media supplemented with 1% FBS for the desired time periods (0 h, 24 h, 48 h, and 72 h). For shear stress experiments, cells were washed with PBS after starvation and incubated in regular media right before fluid flow application.

Immunofluorescence. Cells were seeded in 8-well chamber slides (Falcon, Corning Incorporated, AZ, USA) at a density of 9×10⁵ cells per well. Upon reaching 80% confluence, the cells were washed with PBS and starved to induce cilia differentiation. At each time point during starvation, the cells were washed with PBS, fixed with 4% PFA and 3% sucrose in PBS buffer for 10 minutes at room temperature, washed (1% BSA in PBS), and then permeabilized with 0.1% Triton™ X-100 in PBS for 10 min at room temperature. After two washes, the cells were blocked in 5% BSA in PBS for 1 h at room temperature. Mouse anti-acetylated α-tubulin (Invitrogen; 32-2700) diluted (1:1000) in 3% BSA-PBS was the primary antibody to detect cilia. Cells were incubated with this primary antibody overnight at 4° C. The following day, after 3 washes, the cells were incubated for 1 h at room temperature with Alexa Fluor® 488 conjugated goat anti-mouse secondary antibody (Invitrogen; A11029). After 3 washes, 1 μg/ml dilution of Hoechst (Sigma-Aldrich, ON, Canada; 94403) in 1% BSA-PBS was used to stain the nucleus at room temperature for 10 min. Alexa Fluor® 555 Phalloidin dilution (1:40) in 1% BSA-PBS incubation at room temperature for 20 min was used to stain the cytoskeleton. The images were captured on a Leica Confocal TCS-SP8 or Zeiss Confocal 880 using ×63 (oil) objective with 1,024×1,024 pixel resolution. Each sample has been examined in stitched 5×5 tile images, in duplicate (50 fields of view). Maximum projections of the Z-stacks were used for primary cilium measurement and counting was done in Image J (NIH). Two separate double staining with anti-Ninein antibody (Millipore, CA, USA, ABN1720) as the cilia base marker and anti-IFT88 (Proteintech, IL, USA, 13967-1-AP) were performed to co-stain the cilia alongside anti-acetylated α-tubulin to confirm the method.

Proliferation assay. Bone cells acquired from IS patients were cultured, as previously described above for cell culture. Upon reaching 90% confluence, cells were harvested by adding Trypsin-EDTA (0.25%) and phenol red (Thermo Fisher Scientific Inc., NY, USA 25200-072) for subculture (P3 to P5). Cells were washed and counted (Trypan Blue staining of viable cells) using the Vi-cell® XR (Beckman Coulter, Inc., CA, USA) automated cell counter and then seeded in 12 well plates (100,000 per well in triplicate for each sample). Cells were allowed to grow at 37° C. in 5% CO₂ and each well was counted at different time points (24 h, 48 h, 72 h, 96 h and 120 h post culture). Bone cells from age- and gender-matched trauma surgical patients were used as controls.

In vitro fluid flow stimulation. For each sample, the cells were divided equally between 4 vented 75 cm2 tissue culture flasks and cultured in 21 ml medium (αMEM+10% FBS+1% penicillin/streptomycin). Upon reaching 80% confluence, culture medium was removed, the cells were washed with warm PBS and then transferred to starvation medium for 48 h. After 48 h, cells were washed again and transferred back to 20 ml regular medium immediately before they were subjected to oscillatory fluid flow using a double-tier rocking platform, with some modification of the rocker method described by Robin M. Delaine Smith et al.¹⁶ with a maximum tilt angle of ±20 degrees, and a speed of 1 tilt per second (equal to 1 Hz). The entire unit was housed in a cell culture incubator held at 37° C. and 5% CO₂ for the duration of the flow experiments (0 h, 4 h, 8 h, and 16 h). Control, no flow cells were housed in the same incubator and harvested at 8 h.

Fluid shear stress patterns were applied to cells in a predictable, controlled, and physiologically relevant manner through the whole experiment. From a biomechanical point of view, one expects that the cilia-related gene expression to be a function of time elapsed, t, and the shear stress exerted on the cells, which in turn depends on the fluid viscosity, v, the frequency of flow oscillations, f, and the thickness of the fluid film, h. Designing an experiment in which all these parameters match the physiological conditions can be prohibitively challenging, and in fact unnecessary. Fortunately, the design of the experiment can be simplified using the π-Buckingham theorem⁶⁸ that is widely used in engineering and physics. The π-Buckingham theorem states that the dynamics of a problem (e.g. fluid flow) can be completely described and measured by a set of nondimensional quantities.

Here, ϕ is defined as the ratio of gene expression measured at a given time, t, to its expression at 0 h. Noting that ϕ is a nondimensional quantity, one can use the π-Buckingham theorem to show that ϕ is a function of two other nondimensional numbers:

$$φ = φ ( α , {t}^{*} )$$

$$where α is the Womersley number defined as$$

$$α = h \sqrt{( \frac{2 π f}{v} )} , \\ and {t}^{*} is a nondimensionalized time$$

$${t}^{*} = tf .$$

The Womersley number takes into account the effect of viscosity and shear stress exerted on the cell and is widely used in biomechanical studies involving pulsating fluid flow.69 The value of Womersley number ranges from 5 to 18 in fluid motion of cerebrospinal fluid in the spinal cavity.25 We designed our experiment such that the Womersley number experienced by the cells is equal to 8, which is well within the expected range in vivo. This value corresponds to an average shear stress at the center of the dish with a magnitude of 1 [Pa] in our experiment (see Zhou et al. 201070 for details of calculation).

RNA extraction. All RNA was extracted using Trizol™ (Invitrogen-Thermo Fisher Scientific, 15596-026), according to the manufacturer's instructions. Briefly, cell culture dishes containing adherent bone cells (passage 2 or 3) were washed with PBS before trypsinization, then transferred to a 15 ml tube and after centrifugation, the cell pellet was stored immediately at −80° C. All the cells went through RNA extraction the following day and were lysed in 1 ml Trizol™. RNeasy MinElute™ Cleanup Kit (Qiagen Inc., ON, Canada; 74204) was used to purifiy RNA, according to the manufacturer's instructions.

Quantitative RT-PCR. Reverse transcriptase quantitative PCR (RT-qPCR) was used to assay gene expression levels. All primer design, validation, and gene expression were performed at the Genomics core facility of Institut de Recherche en Immunologie et Cancérologie (IRIC), University of Montreal, Quebec. All RNA was run on a bioanalyzer using a Nano RNA chip to verify its integrity. Total RNA was treated with DNase and reverse transcribed using the Maxima™ First Strand cDNA synthesis kit with ds DNase (Thermo Fisher Scientific). Before use, RT samples were diluted 1:5. Gene expression was determined using assays designed with the Universal Probe Library from Roche (www.universalprobelibrary.com). For each qPCR assay, a standard curve was performed to ensure that the efficacy of the assay is between 90% and 110%. Quantitative PCR (qPCR) reactions were performed in triplicate with 2 internal controls (GAPDH and HPRT) using PERFECTA qPCR FASTMIX II™ (Quanta Biosciences, Inc., MD, USA), 2 μM of each primer, and 1 μM of the corresponding UPL probe. The Viia7 qPCR instrument (Thermo Fisher Scientific) was used to detect the amplification level and was programmed with an initial step of 20 sec at 95° C., followed by 40 cycles of: 1 sec at 95° C. and 20 sec at 60° C. Relative expression (RQ=2−ΔΔCT) was calculated using the Expression Suite software (Thermo Fisher Scientific), and normalization was done using both GAPDH and HPRT. The baseline expression level at 0 h (before treatment) of every sample was defined as its own calibrator. The calibrator has a RQ value of 1 because it does not vary compared to itself. For each gene, the two groups (control and IS) were compared at each time point using a pairwise t-test. In this way, we asked one question per gene and we did three comparisons to answer (three comparisons per family of test). After looking at the results of these tests, we asked another question for three of the genes that seemed to show an overall expression profile that was different between IS and controls (ITGB1, CTNNB1 and POC5). For these genes we added three more comparisons: the expression at 0 h for IS was compared to each of the other time points (4 h, 8 h and 16 h) using 3 separate pairwise t-tests. We also examined the base line of gene expression in IS versus control, by comparing the delta Ct mean (expression level normalized with endogenous controls) at 0 h of all samples using t-test.

RT-PCR primer sequences are as follows:
BMP2
(SEQ ID NO: 1303)
F: 5′-cagaccaccggttggaga-3′;
(SEQ ID NO: 1304)
R: 3′-ccactcgtttctggtagttcttc-5′
SPP1
(SEQ ID NO: 1305)
F: 5′-gcttggttgtcagcagca-3′;
(SEQ ID NO: 1306)
R: 3′-tgcaattctcatggtagtgagttt-5′
ITGB3
(SEQ ID NO: 1307)
F: 5′-gggcagtgtcatgttggtag-3′;
(SEQ ID NO: 1308)
R: 3′-cagccccaaagagggataat-5′
PTGS2
(SEQ ID NO: 1309)
F: 5′-gctttatgctgaagccctatga-3′;
(SEQ ID NO: 1310)
R: 3′-tccaactctgcagacatttcc-5′
RUNX2
(SEQ ID NO: 1311)
F: 5′-ggttaatctccgcaggtcac-3′;
(SEQ ID NO: 1312)
R: 3′-ctgcttgcagccttaaatga-5′
ITGB1
(SEQ ID NO: 1313)
F: 5′-cgatgccatcatgcaagt-3′;
(SEQ ID NO: 1314)
R: 3′-acaccagcagccgtgtaac-5′
POC5
(SEQ ID NO: 1315)
F: 5′-aacaactgtgtaatcagatcaatgaa-3′;
(SEQ ID NO: 1316)
R: 3′-tgcctatggcatgagacaag-5′
LBX1
(SEQ ID NO: 1317)
F: 5′-tcgccagcaagacgttta-3′;
(SEQ ID NO: 1318)
R: 3′-gccgcttcttaggggtct-5′
FUZ
(SEQ ID NO: 1319)
F: 5′-tcacctccacgcacttcc-3′;
(SEQ ID NO: 1320)
R: 3′-gggcctggtagacctcatct-5′
GAPDH
(SEQ ID NO: 1321)
F: 5′-agccacatcgctcagacac-3′;
(SEQ ID NO: 1322)
R: 3′-gcccaatacgaccaaatcc-5′
HPRT
(SEQ ID NO: 1323)
F: 5′-tgatagatccattcctatgactgtaga-3′;
(SEQ ID NO: 1324)
R: 3′-caagacattctttccagttaaagttg-5′.

Exome and Sanger sequencing. Genomic DNA was extracted from the whole blood of 73 IS patients and 70 controls using the PureLink® Genomic DNA extraction kit (Thermo Fisher Scientific). Library preparation and exome sequencing was performed at GENESE (Génomique de la Santé de l'Enfant, Sainte-Justine University Hospital Research Center). Selected variants were confirmed using Sanger sequencing technologies at the Genome Quebec Innovation Centre. Samples were barcoded, and captured using libraries of synthetic biotinylated RNA oligonucleotides (baits) targeting 50 Mb of genome (Agilent SureSelect Human All Exon 50 Mb v3), and sequenced on the 5500 SOLiD™ Sequencing System (Thermo Fisher Scientific). Trimmed FASTQ formatted sequences were aligned to the exome target sequence using Bfast+bwa (version 0.7.0a) in the paired-end alignment mode.71 Mapped reads were refined using GATK and Picard program suites³⁴ to improve mapped reads near indels (GATK indel realigner) and improve quality scores (GATK base recalibration) and to remove duplicate reads with the same paired start sites (Picard mark Duplicates). Variants were called using SAMTOOLS batch calling procedure referenced against the UCSC assembly hg19 (NCBI build 37). Variants were additionally filtered to remove variants that are present with minor allele frequencies (MAF)>0.05 (dbSNP, 1000 genomes, ExAC and/or Exome variant server (ESP). Variants were annotated using the GEMINI framework⁷² that provides quality metrics and extensive metadata (e.g. OMIM, clinVar, etc.) to help further prioritize variants. To optimize the querying criteria for the GEMINI database, we performed bidirectional Sanger sequencing for more than 100 different variants. Using an optimized threshold (Coverage DP>10x, Genotype quality GQ>80, Call rate>90%, Alternate quality (QUAL)>50, Map quality>20), the results show 85% genotype correlation between the sequencing methods. This threshold was used to filter our data prior to analysis.

Statistical analyses. To test for accumulation of rare variants in genes associated with IS, we used the Sequence Kernel Association Optimal unified test algorithm SKAT-O.35 SKAT-O is a region-based omnibus test that increases a study's power to detect rare variants. Because there is no model for the genetic basis underlying IS, SKAT-O is optimal over SKAT or burden testing alone, since it is a robust technique to detect variable effect rare polymorphisms.22 Variants that passed our filtering criteria, with a dataset minor allele frequency 5% were analyzed in two different sets. Additionally, high quality variants with membership to the Illumina Human Exome Chip were extracted from the Gemini database for population structure analyses using R package SNPRelate. The top two components were used as covariates in the SKAT-O analysis. The first set used the manual recommended settings for rare variants: SKATBinary with SNP weighting based on Madsen and Browning weights (i.e. less frequent are more impactful) (B1=0.5, B2=0.5). The second set weighted-SNPs are based on Combined Annotation Dependent Depletion (CADD) scores (i.e. functional, deleterious, and disease-causing variants have greater impact).⁷³ For both sets, we generated a null model of no association between genetic variables and outcome phenotype adjusting for covariates (see Tables 4 and 5 in Example 6). Covariate analysis confirmed that there was not population stratification in our dataset. The gene-level significant thresholds were determined by the efficient resampling (ER) method and the conservative minor allele count (MAC) threshold of≤40.74 To examine the number of ciliary genes in our datasets, we used two reference lists that define a ciliary function based on experimental evidence: 1) the SYSCILIA™ gold standard list of genes containing 303 ciliary genes verified by independent publications³⁶; 2) a list of 52 genes (51 novel genes because one is in the SYSCILIA™ list) from a functional genomic screen that used RNA interference to identify genes involved in the regulation of ciliogenesis and cilia length.³⁷

Review of ciliary genes associated with spinal curvature. If idiopathic scoliosis is a genetically heterogeneous ciliopathy-like condition, then we expect a large number of known ciliopathies have spinal curvature as a comorbidity. We reviewed the SYSCILIA™ gold standard gene list and the Kim et al., 2010 list^{36,37} to ascertain how many ciliary genes were associated with spinal curvature phenotypes in either a human syndrome or an animal model. For each of the 303 genes, the search terms in Google included the gene name with “spinal curvature” and “scoliosis”. Additionally, if the gene was known to be associated with a syndrome in the OMIM database, we also searched the syndrome name with “scoliosis”.

### EXAMPLE 2

### Ciliary Genes Associated with Scoliosis Phenotype in Human and/or Animal Models

Ciliopathies comprise a large number of human genetic disorders that are defined by the causative or predisposing gene being related to cilia structure, function, sensory pathways, or localization. To examine whether the IS cilia phenotype is linked to ciliary genes, the inventors reviewed established cilia gene lists for associations to spinal curvature and surveyed well defined IS genes in human and animal studies for a functional link to cilia. From the review using the SYSCILIA gold standard list of 303 verified ciliary genes, 55 genes are associated with a human syndrome having clinical reports of scoliosis were found. Two of these genes, SUFU and Adherens junctions associated protein 1 (AJAP1) are in loci that are associated with IS through linkage studies,^{55,56} and 19 have both clinical (human) and experimental (animal model) associations with scoliosis. Furthermore, an additional 13 published animal model studies were found in which manipulation of the ciliary gene caused spinal curvature. In summary, 22% of these well-established cilia genes are associated with spinal curvature. In addition, from the study by Kim et al. 2010,³⁷ 3 other genes that modulate ciliogenesis or cilia length and feature a clinical syndrome with reported scoliosis were identified. Table 2 below provides a list of ciliary genes that are associated with a scoliosis phenotype.

TABLE 2
IS-associated genes in humans and/or animal models,
which are also associated with cilia.

| Gene | Scoliosis Association | Cilia Association |
|---|---|---|
| TBX6 | PMIDs: 26120555, 20228709 | PMIDs: 18575602 & |
| | (Congenital and | 17765888 (Affects |
| | idiopathic scoliosis in humans) | morphology and motility |
| | | of nodal cilia in mice & |
| | | zebrafish) |
| LBX1 | PMIDs: 26394188, | PMIDs: 18541024 (deleted |
| | 25987191, 25675428, | in a mouse model |
| | 24721834 (Idiopathic | of the primary ciliary |
| | scoliosis association in | dyskinesia gene) |
| | several ethnic groups, | |
| | confirmed using different | |
| | approaches) | |
| GPR126 | PMIDs: 25954032, | PMIDs: 16875686, |
| | 25479386, 23666238 | 24227709 (No direct |
| | (Idiopathic scoliosis in | relation to cilia. Essential |
| | humans and mice) | for the development |
| | | of myelinated axons in |
| | | zebrafish and mice) |
| PAX1 | PMIDs: 25784220, | PMIDs: 19517571, |
| | 19080705, 16093716 | 23907320, 24740182 |
| | (Congenital and idiopathic | (Other family members |
| | scoliosis in humans | are associated with cilia |
| | and mice) | signaling pathways or |
| | | ciliated tissues) |
| POC5 | PMID: 25642776 (Idiopathic | PMID: 23844208, 19349582 |
| | scoliosis in humans) | (interacts with cilia |
| | | and is essential for |
| | | centriole structure in |
| | | humans and Drosophila) |
| KIF6 | PMID: 25283277 | PMID: 16084724 |
| | (idiopathic-type curvature in | (Predicted to be involved in |
| | zebrafish) | ciliary function or structure) |
| PTK7 | PMID: 25182715 | PMID: 20305649 (Role |
| | (idiopathic-type curvature in | in cilia orientation in |
| | zebrafish) | zebrafish) |
| FGF3 | PMID: 25852647, 24864036 | PMID: 26091072 (Affecting |
| | (Idiopathic scoliosis | the organization of |
| | in a KO mouse model; | chondrocyte primary |
| | Scoliosis in a human case | cilia in the growth plate in |
| | report carrying loss-of- | mice) |
| | function mutation in the | |
| | gene) | |

### EXAMPLE 3

### Osteoblasts of IS Patients Have Longer Cilia

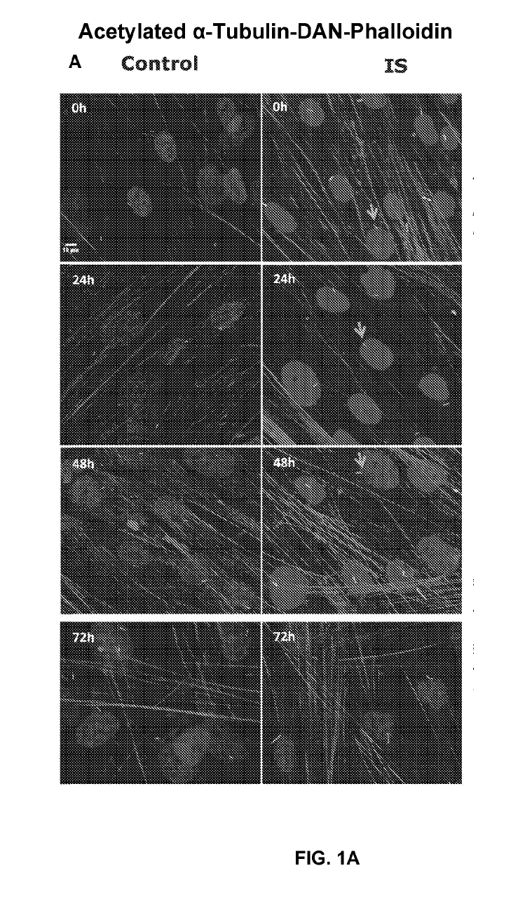

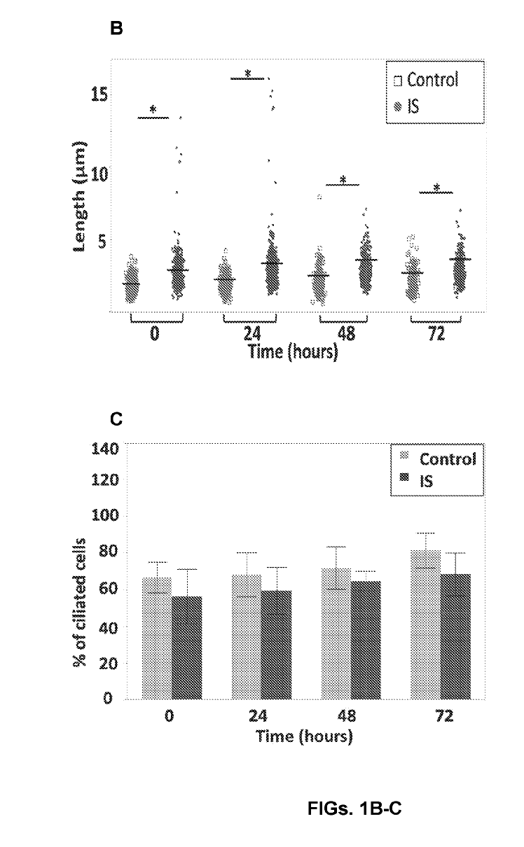

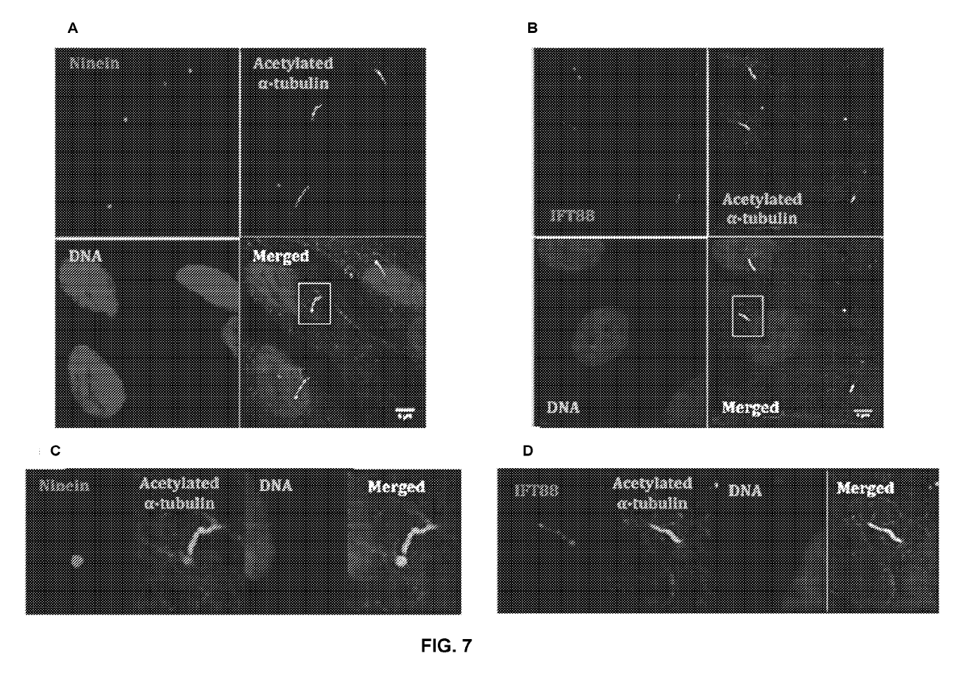

To assess whether there is an observable defect associated with primary cilia in IS patients, osteoblast cells derived from bone specimens obtained during surgery were examined. All samples were from age matched adolescent female subjects. FBS deprivation was used to promote ciliogenesis and differentiation. Cilia morphology was examined using anti-acetylated α-tubulin immunofluorescence staining prior to and after 24, 48, and 72 hours (h) starvation in primary osteoblasts from 4 IS patients and 4 non-scoliotic trauma patients used as controls (FIG. 1A). The fraction of ciliated cells and cilia length were quantified in fixed and stained cells. Measurements were acquired from 5×5 stitched tile images per sample, in duplicate (50 fields). Results have shown that the cilia in IS-derived cells were approximately 30% to 40% longer than cilia in control cells (FIG. 1B, Table 3). IS cells also showed a reduced incidence of ciliated cells compared to controls, although the difference did not reach the statistical significance (FIG. 1C). To validate the staining of cilia, double immunostaining was performed on fixed IS osteoblasts using anti-Ninein, as the basal body marker or anti-IFT88 to stain the length of cilia alongside the anti-acetylated α-Tubulin (FIG. 7).

TABLE 3
The average length of cilia in IS-derived bone cells

| | 0 h | 24 h | 48 h | 72 h |
|---|---|---|---|---|
| Controls | 1.94 ± 0.35 | 1.99 ± 0.32 | 2.05 ± 0.66 | 2.16 ± 0.78 |
| IS | 2.66 ± 1.14 | 2.87 ± 1.84 | 2.82 ± 0.81 | 2.80 ± 0.65 |
| P Value | 2.62632E−22 | 1.00327E−20 | 2.49E−25 | 1.03284E−14 |

The average length of cilia in μm±variance at four starvation time points (0, 24 h, 48 h, 72 h) is shown in Table 3. To compare the length of cilia between IS and non-IS controls, the inventors combined measurements of up to 1000 cilia for each sample (25 fields per sample, in duplicate), at each time point, then used a t-test to compare the mean lengths of cilia in IS vs. control pools. The results show that cells from IS subjects have significantly longer cilia. The difference in length was significant across all the time points for IS patients (P value≤0.005), n=8 (4 IS vs. 4 Controls).

### EXAMPLE 4

### IS and Control Cells Grow at the Same Rate

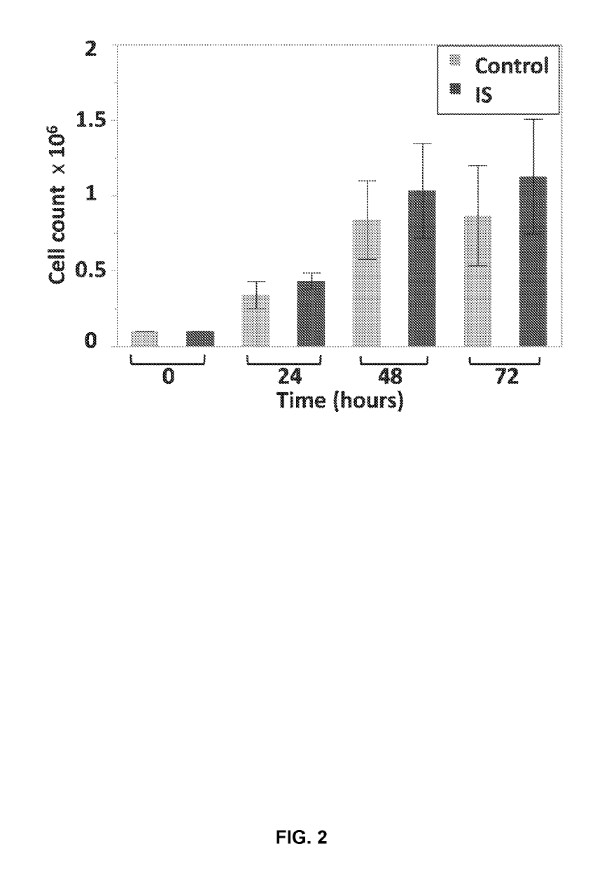

Cilia assembly, disassembly, and length have been associated with cell cycle and control of cell proliferation.²³ To investigate if there is a correlation between longer cilia in IS patients and a differential growth rate, cell proliferation was assayed by counting viable cell (Trypan Blue stained) number as a function of time. Cell proliferation rate varied in all the samples as visible in the error bars (FIG. 2). It seems that IS cells increase in number slightly faster than controls but the difference does not pass the significant threshold at any of the three time points analyzed (24, 48 and 72 h).

As shown in Example 3, cilia in IS cells are significantly longer across all measured time points, but the most conspicuous length differences are visible before starvation and at 24 hours after starvation (FIG. 1B), suggesting an abnormality in cilia formation and early stages of cilia growth. Although no significant differences were seen in the percentage of ciliated cells before starvation (FIG. 1C), long cilia (up to 13 μm) were visible in pre-starved IS bone cells and not controls. This could suggest actin organization impairment, considering that actin polymerization inhibitors induce longer cilia and facilitate ciliogenesis independent of starvation.³⁷ Irregularity in the control of cilia length has been associated with cytoskeletal disruption and actin dynamics, either due to genetic mutation³⁷ or in response to mechanical stress.³⁸ While there is no statistically significant difference in the incidence of cilia between controls and IS, there does seem to be a trend of IS cells having a lower incidence than control at every time point (FIG. 10). This might be an indicator of cell cycle irregularities, considering the tight correlation of cilia differentiation to cell cycle progression. The proliferation assay confirmed that the IS cilia phenotype occurs independent of cell proliferation.

### EXAMPLE 5

### Impaired Biomechanical Response in IS Cells

The functional response of IS cells having long cilia was evaluated by monitoring changes in expression for several mechanoresponsive genes under fluid flow, at four time points (0, 4 h, 8 h, 16 h). A 1 [Pa] shear stress (the magnitude at the center of the dish) in 1 [Hz] frequency was applied, which corresponds to a Womersley number of 8. The biomechanical parameters were chosen to be physiologically relevant based on the reported frequency spectra of forces affecting the human hip during walking, (1-3 Hz),²⁴ and the Womersley number estimated for cerebrospinal fluid motion in the spinal cavity (5-18).²⁵

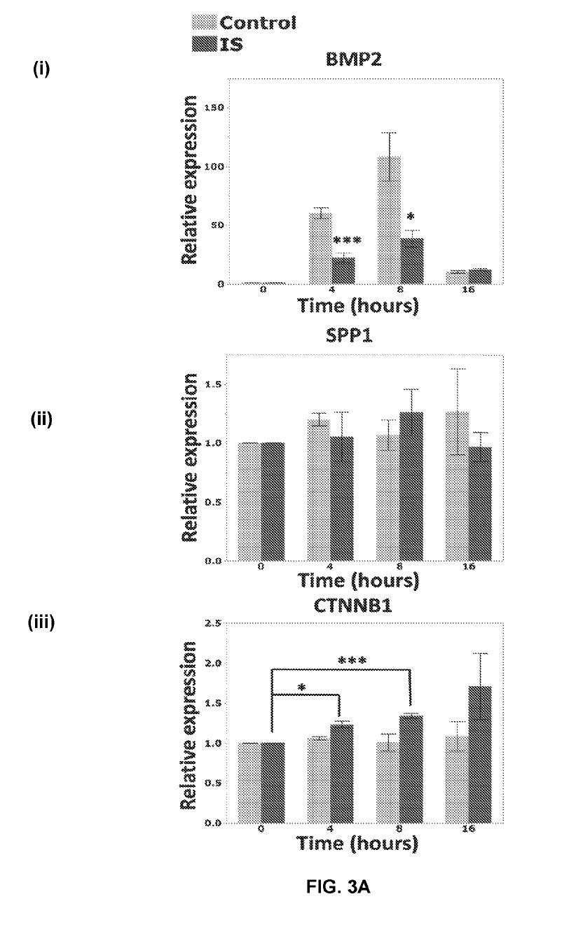

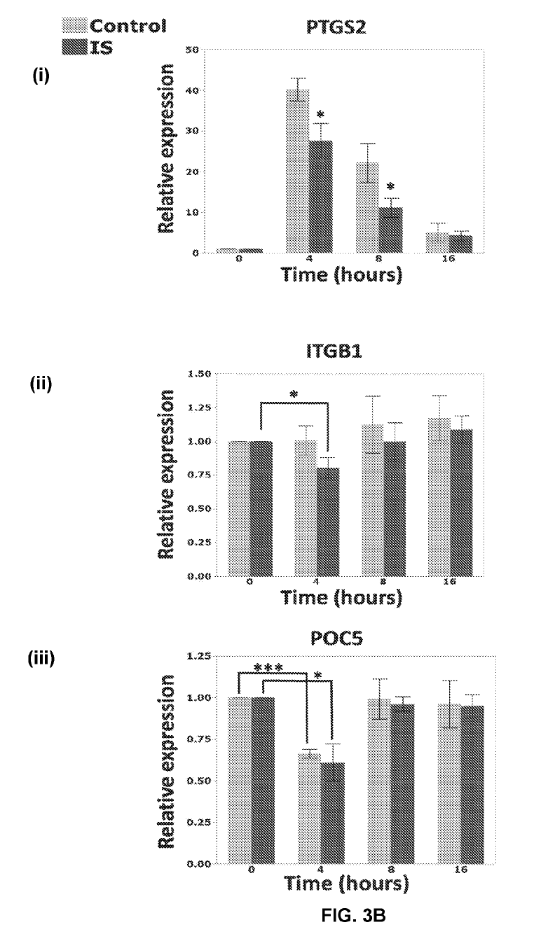

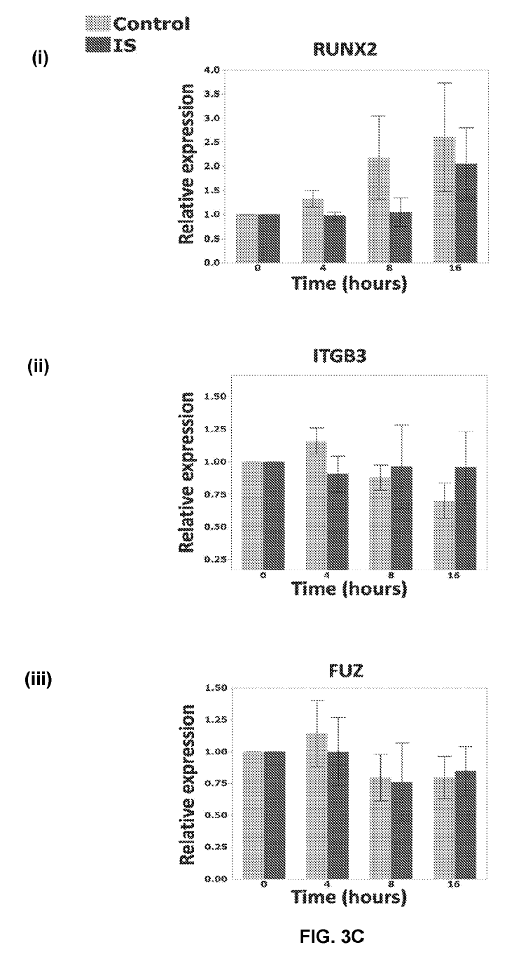

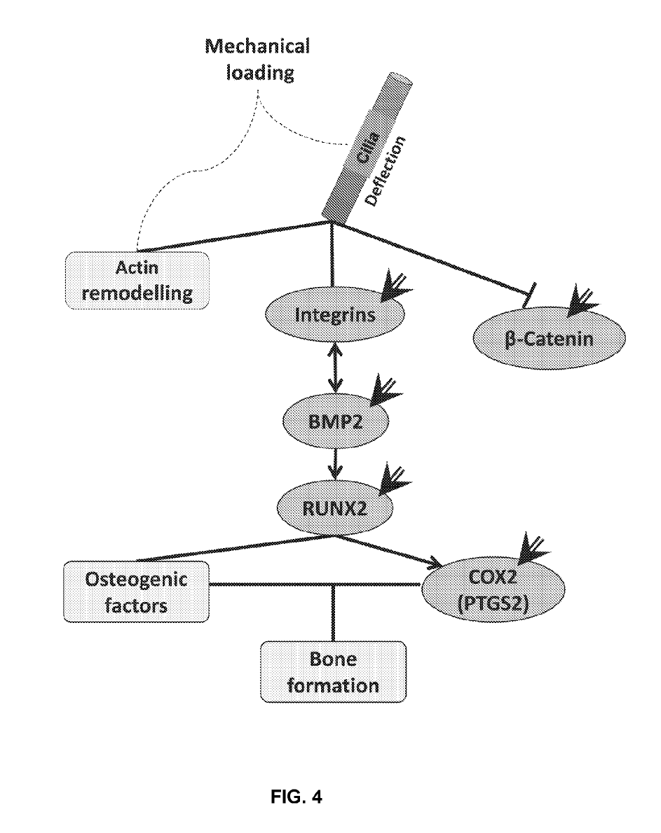

Differential gene expression was compared for IS vs. controls at each time point, and then the whole response profile of each gene was examined. For each gene, after normalization to two endogenous controls (GAPDH and HPRT), the baseline expression level at 0 h (before treatment) of every sample was defined as its own calibrator. The gene expression for all time points for each sample was compared to its own 0 h (which has a RQ value of 1). The results have been shown as fold changes compared to the calibrator. One question per gene was asked: is there any difference between IS and control at each time point? For three genes (ITGB1, CTNNB1 and POC5) that showed a different overall expression pattern in IS vs. controls, a second question was asked: is there a significant difference in gene expression before and after flow? Each gene has been analysed independently using a pair wise t-test for each question followed by a post hoc Bonferroni. Concordant with previous findings regarding biomechanical induction and the expression of osteogenic factors,^{12,17,26} our assay showed a dramatic increase in Bone morphogenetic protein 2 (BMP2) and Cyclooxygenase-2 (COX2) expression in IS and controls, following 4 and 8 hours of fluid flow induction. However, for both genes, the IS response was significantly less than controls (FIG. 3). The response for Runt-Related Transcription Factor 2 (RUNX2) in IS patients, while not significant, is also less than what we observed in controls. We also tested the expression of Secreted Phosphoprotein 1 (SPP1, also known as Osteopontin or OPN) as an osteogenic factor in bone, and did not observe a biomechanical response in IS or control cells (FIG. 3). The modified responses to mechanical stress observed in this study corroborate those previously reported in human mesenchymal stem cells (MSCs),¹⁷ Expression of integrin beta 1 (ITGB1) and integrin beta 3 (ITGB3) were monitored due to their possible role in transmitting mechanical signals in bone.²⁷ The expression of ITGB1 did not notably change during 16 h of flow application in controls, while a significant decrease in expression was observed in IS cells (p=0.025) at 4 hours post flow. ITGB3 expression did not significantly change in IS or control cells. Cilia are well known for their regulatory effect on the Wnt signaling pathway.²⁸ Beta-catenin, a main player in the Wnt pathway,²⁸⁻³⁰ is localized to the cilium.³¹ Results show that the expression of beta-catenin (CTNNB1) did not change in control osteoblasts as it has been shown previously,³⁰ while IS cells showed a significant continual rise in CTNNB1 expression in response to flow application (p at 4 h=0.03, 8 h=0.008).

Finally, Fuzzy planar cell polarity (FUZ), Protein Of Centriole 5 (POC5), and Ladybird homeobox 1 (LBX1) genes were added to the experiment following our exome analysis, and recent published scoliosis genetic studies.^{3,32,33} For FUZ, we did not see any significant differential expression between IS and controls at any time point following flow application. POC5 expression decreased almost by half at the 4 hour point in both IS and controls (p<0.05), suggesting a role in early stages of mechanotransduction response (FIG. 3). No statistically significant difference was detected between patients and controls in the basal level of expression of all 9 genes. LBX1 mRNA was not detected after 35 cycles in two attempts of RT-qPCR (data not shown).

### EXAMPLE 6

### Whole Exome Sequencing (WET) Identifies New Gene Markers for Scoliosis

Whole exome sequencing was performed to test the hypothesis that rare variants in ciliary genes might be causal for IS. Exome sequencing was done on peripheral blood DNA samples from 73 IS and 70 matched controls using the Agilent SureSelect™ Human All Exon 50 Mb v3 capture kit and the Life Technologies 5500 SOLiD™ Sequencing System. Variants were called and annotated using a customized bioinformatics pipeline including SAMTOOLS™, GATK™ and Picard™ program suites.³⁴ To reduce the number of likely variants, we subsequently filtered the total variant set to remove those with a minor allele frequency greater than 5%, as well as variants not in or adjacent to protein-coding exons. After filtering, our dataset included 73 IS patients, 70 controls, 8544 genes, and 16,384 variants. We used SKAT-O to survey our exome data under two different weighting parameters: in favor of lower frequency variants (Madsen Browning weighting, Set I), and in favor of variants with projected deleterious effects and pathogenicity (Combined Annotation Dependent Depletion: CADD weighting, Set II). Since the underlying biology of idiopathic scoliosis is not understood, an omnibus test such as SKAT-O is considered more powerful than a burden test because it does not make assumptions regarding direction or size of variant effect.³⁵ Analysis using Madsen Browning weighting (Set I) identified 259 genes and analysis using CADD weighting (Set II) identified 240 genes that are significant (p≤0.01) after correction for multiple testing. The Sets were compared and genes that were significant in both (n=120; Table 4) were considered candidates for idiopathic scoliosis (i.e., polymorphic/genetic markers comprising risk variants). This list was examined for ciliary genes using the SYSCILIA gold standard list³⁶ and the Kim et al. 2010³⁷ list as references, along with inquiries using Google search engine. Fuzzy planar cell polarity protein (FUZ) is the only known ciliary gene in both data lists. However, there is a greater number of variants in controls compared with cases (12 controls with at least one variant vs 1 patient). Of the candidate genes, the 23 most significant (p<0.001) were further examined to determine the number of patients and controls having at least one variant (Table 5). Seven of these genes have greater variant enrichment among patients: CD1B, CLASP1, SUGT1, HNRNPD, LYN, ATPSB, AL159977.1. Table 6, provides a list of rare variants identified in the 120 genes linked to IS (risk variants) and listed in Tables 4 and 5. The polynucleotide sequence used as a reference for each of these genes was from Ensembl version 70.

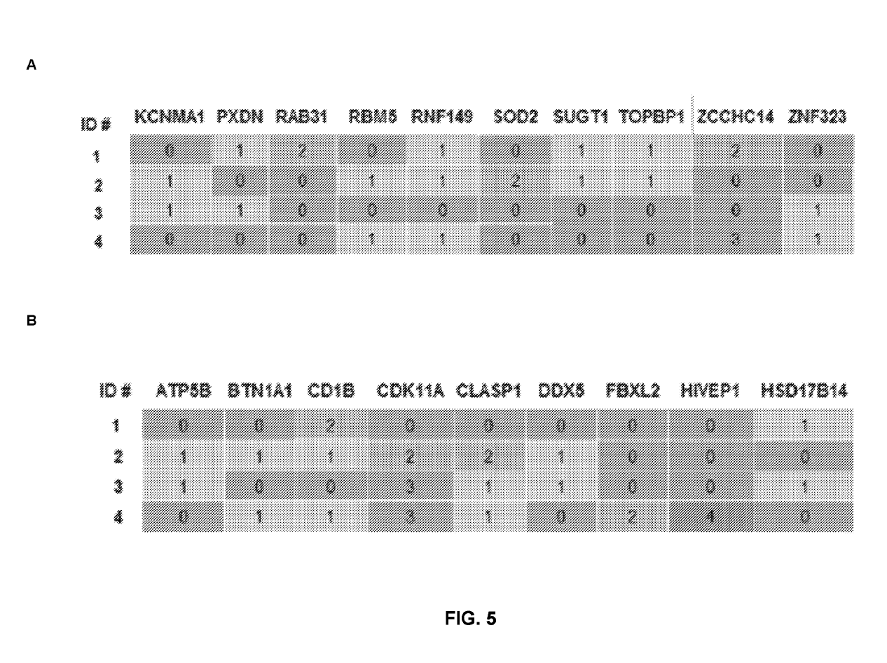

The variant profile for each of the four IS patients used in the cellular analyses (see Examples 3-5) was also analyzed, to see if there are shared genes with variant enrichment. Controls could not be examined because the cohort used for molecular work differs from the genotyped control cohort. Control bone tissue were obtained intraoperatively from non-scoliotic trauma patients whereas the genotyped controls were from a non-surgical cohort. None of the genes identified in our combined SKAT-O table were shared among all the four patients, but all the patients have variants in either CD1B, CLASP1, or SUGT1. The CDK11A gene is represented among three patients, but in the exome cohort, nearly all patients and controls have at least one variant for this gene (FIG. 5).

After analyses by SKAT-O the genes statistically significant (at p<0.01) in Set I and Set II were compared. One hundred and twenty (120) genes were identified by this approach.

TABLE 4
Genes associated with IS

| Gene | Set I p-Value | Set II P-Value |
|---|---|---|
| FEZF1 | 9.14623292262238E−11 | 2.65141767492733E−19 |
| CDH13 | 2.56597266918007E−10 | 3.25195355366862E−17 |
| FBXL2 | 2.55658311855141E−08 | 9.36018198760692E−16 |
| TRIM13 | 1.26269039884992E−15 | 1.39786847792469E−12 |
| CD1B | 3.0115635847658E−10 | 9.56319016750188E−11 |
| VAX1 | 1.87672433282771E−06 | 1.0287163141036E−10 |
| CLASP1 | 8.91363938841512E−11 | 2.39567078392548E−08 |
| SUGT1 | 1.45579966126519E−06 | 0.0000726157171608077 |
| MIPEP | 0.0014093581094382 | 0.00008359177892184 |
| FAM188A | 1.91437820546762E−07 | 0.000142711996677612 |
| TAF6 | 0.00141269941960906 | 0.000219166807116741 |
| WHSC1 | 0.00718326903405001 | 0.000240868230053009 |
| GPR158 | 0.000278262161730402 | 0.000278262161730402 |
| HNRNPD | 0.00175975467919198 | 0.000510922803128836 |
| RUNX1T1 | 0.00235146027422903 | 0.000548781011086469 |
| GRIK3 | 0.00061173751083077 | 0.00061173751083077 |
| FUZ | 0.000807306176520932 | 0.000678191067697156 |
| LYN | 0.000886349660487618 | 0.000745049768714577 |
| DDX5 | 0.000188591327183851 | 0.00109108884771161 |
| PODXL | 0.00136843519158836 | 0.0011242369420949 |
| ATP5B | 0.0000845225124055382 | 0.00115393759263137 |
| PIGK | 0.00136203072056991 | 0.00136203072056991 |
| AL159977.1 | 0.00142441312386281 | 0.00142441312386281 |
| SEPT9 | 0.00228543962790456 | 0.00164370948384936 |
| TMEM87A | 0.00329280831792091 | 0.00173809657273625 |
| CDYL | 0.00240581003554028 | 0.00180243517726981 |
| SPINT3 | 0.00520962403944304 | 0.00197099019153421 |
| SERTM1 | 0.00214019241475247 | 0.00214019241475247 |
| FOLR3 | 0.00661930780286749 | 0.00218827584521567 |
| FCER2 | 0.00896615540398866 | 0.00228065553031091 |
| MAEA | 0.00454245474175253 | 0.00242080457979593 |
| PXT1 | 0.00244475689717455 | 0.00244475689717455 |
| UVRAG | 0.00205781167832715 | 0.0026755250865001 |
| SPPL3 | 0.00345366665266877 | 0.00272879887377066 |
| IGHV3-50 | 0.00283719813492006 | 0.00283719813492006 |
| HIVEP1 | 0.00837400060514892 | 0.00287381720962344 |
| SMAD5 | 0.00298451543444997 | 0.00298451543444997 |
| PPP1R21 | 0.00491566706882474 | 0.00313751151358811 |
| SEC62 | 0.0037165577299206 | 0.0032280762662854 |
| TOPBP1 | 0.00323964254448472 | 0.00333239428269868 |
| HIPK3 | 0.00268675870063158 | 0.00388097152295147 |
| KRTAP12-2 | 0.00658588136575445 | 0.00421381163196263 |
| FYB | 0.00503173910897245 | 0.00423204579453898 |
| PXDN | 0.00650207897956268 | 0.00428663102881143 |
| CDV3 | 0.00338353701138391 | 0.00447605631402648 |
| RP3-344J20.2 | 0.00450584299938946 | 0.00450584299938946 |
| RP11-405L18.2 | 0.00453527085149184 | 0.00453527085149184 |
| MRPL18 | 0.0045443521968739 | 0.0045443521968739 |
| SOD2 | 0.00327227103560483 | 0.00468608259221111 |
| FOXP2 | 0.0121444225604324 | 0.0052679796206819 |
| REEP1 | 0.00772567235504315 | 0.0053911371779884 |
| C1orf106 | 0.0149905704346799 | 0.0055752636254589 |
| DNASE1L1 | 0.00600961915498247 | 0.00600961915498247 |
| BTN1A1 | 0.00389126762824743 | 0.00612221913773433 |
| MLST8 | 0.00613332513213671 | 0.00613332513213671 |
| HMP19 | 0.00614661590113029 | 0.00614661590113029 |
| OR8B4 | 0.00619936434146968 | 0.00619936434146968 |
| AC105901.1 | 0.00619938762158156 | 0.00619938762158156 |
| OR5F1 | 0.00620906939213432 | 0.00620906939213432 |
| GLE1 | 0.00623092834521804 | 0.00623092834521804 |
| OR5P3 | 0.00626875402805009 | 0.00626875402805009 |
| SCFD1 | 0.00467674270335083 | 0.00630325165080644 |
| CDK11A | 0.00700203109325059 | 0.00651030226142316 |
| HSD17B14 | 0.00654775676647322 | 0.00654775676647322 |
| NFU1 | 0.00478000085939227 | 0.00672413614999529 |
| GTF2H3 | 0.00947742559537341 | 0.00674952997447815 |
| RAB7A | 0.0103840301065211 | 0.00678196611652705 |
| HOXA3 | 0.00892854556769163 | 0.00696933833944223 |
| ZC3H4 | 0.0142957400207602 | 0.0078344408913238 |
| DDX55 | 0.00917109676114347 | 0.00786798046240299 |
| FBXW10 | 0.0131410217667573 | 0.00824821539511738 |
| OSBPL2 | 0.0151769502993362 | 0.00848456725608149 |
| POLR1A | 0.00641889074615831 | 0.00849223022283857 |
| NOP58 | 0.00115580967671382 | 0.00855393941227298 |
| RAB31 | 0.00599172034835371 | 0.00859441405981285 |
| EFNB2 | 0.00876644774019711 | 0.00876644774019711 |
| ZCCHC14 | 0.00614735144634448 | 0.00882722290517162 |
| GLP1R | 0.00274244563327707 | 0.00901743918062178 |
| RNF149 | 0.00185729586203136 | 0.00916089980851448 |
| OR1J2 | 0.00306370216065147 | 0.00924745497417392 |
| WI2-81516E3.1 | 0.00927022266477135 | 0.00927022266477135 |
| GAPDHP27 | 0.00940200328461592 | 0.00940200328461592 |
| SFTA3 | 0.00941624925811713 | 0.00941624925811713 |
| ACSF3 | 0.00493488174891588 | 0.0094399413624774 |
| POU2F2 | 0.00945359273444211 | 0.00945359273444211 |
| MIR345 | 0.00955672892878693 | 0.00955672892878693 |
| SNPH | 0.00959288817570845 | 0.00959288817570845 |
| MATR3 | 0.0096761290454168 | 0.0096761290454168 |
| RP11-73B8.2 | 0.00989883201280166 | 0.00989883201280166 |
| SNORA48 | 0.0143541843894121 | 0.0100947394551106 |
| PATZ1 | 0.0112659528970391 | 0.0100976522816034 |
| RBM5 | 0.0129867336054512 | 0.0103482440021942 |
| HMGA1 | 0.0107855712586593 | 0.0107855712586593 |
| ATP1A3 | 0.00727924303341687 | 0.0107874117620093 |
| ACTG1P1 | 0.0110246369078176 | 0.0110246369078176 |
| PAIP1 | 0.00764996842916127 | 0.0117165308629053 |
| KCNMA1 | 0.00614648964331421 | 0.0117955670612322 |
| PALB2 | 0.00707205779157172 | 0.0121181228685785 |
| PLEKHG5 | 0.00302106533164803 | 0.0123984799917172 |
| C11orf2 | 0.00456145115159546 | 0.0124733070513875 |
| MT1DP | 0.0127363013366284 | 0.0127363013366284 |
| CYC1 | 0.0130028970599229 | 0.0130028970599229 |
| DTD1 | 0.013040539461903 | 0.013040539461903 |
| CREB3L3 | 0.0130488318029866 | 0.0130488318029866 |
| RPL23A | 0.0131062187387933 | 0.0131062187387933 |
| CD164L2 | 0.0131437523635148 | 0.0131437523635148 |
| PCCB | 0.0131461931246614 | 0.0131461931246614 |
| GIMAP7 | 0.0131582410488705 | 0.0131582410488705 |
| AHCYL1 | 0.0131585916962087 | 0.0131585916962087 |
| TNNT2 | 0.0131632188456312 | 0.0131632188456312 |
| ZNF134 | 0.0131638270433327 | 0.0131638270433327 |
| AC079612.1 | 0.0131749593116791 | 0.0131749593116791 |
| MTA2 | 0.0132228764053148 | 0.0132228764053148 |
| RP11-672F9.1 | 0.0132262706985138 | 0.0132262706985138 |
| CLEC5A | 0.0132773100436662 | 0.0132773100436662 |
| C1orf222 | 0.0088939638627839 | 0.013916460742146 |
| CD96 | 0.0128160789801138 | 0.0140861326578095 |
| PPFIBP1 | 0.0057784561937857 | 0.0142691077274711 |
| ZNF323 | 0.000983099432403177 | 0.0147700339883208 |
| SUPT3H | 0.0144812651497707 | 0.0152422197922659 |

TABLE 5
Top genes associated with IS

| | | | | | # of | | | |
|---|---|---|---|---|---|---|---|---|
| | | | | | Indi- | | | |
| | | | | | viduals | | | |
| | | | | | who carry | | | |
| | | | | | at least | | | |
| | | | | | 1 risk | | | |
| | | | | | variant | | % of | |
| | | | | Total | in the | | carrier in | |
| | | | | # of | gene | | each cohort | |
| Gene | Ref IDs | Set I p-Value | Set II P-Value | variants | Ctrl | IS | Ctrl | IS |
| FEZF1 | HNGC: 22788; | 9.14623292262238E−11 | 2.65141767492733E−19 | 5 | 66 | 32 | 97.05 | 48.48 |
| | Entrez Gene: | | | | | | | |
| | 389549; | | | | | | | |
| | Ensembl: | | | | | | | |
| | ENSG00000128610; | | | | | | | |
| | OMIM: 613301; | | | | | | | |
| | UniprotKB: | | | | | | | |
| | A0PJY2 | | | | | | | |
| CDH13 | HGNC: 1753 | 2.56597266918007E−10 | 3.25195355366862E−17 | 19 | 60 | 33 | 88.23 | 47.82 |
| | Entrez Gene: 1012 | | | | | | | |
| | Ensembl: | | | | | | | |
| | ENSG00000140945 | | | | | | | |
| | OMIM: 601364 | | | | | | | |
| | UniProtKB: P55290 | | | | | | | |
| FBXL2 | HGNC: 13598 | 2.55658311855141E−08 | 9.36018198760692E−16 | 12 | 61 | 33 | 89.7 | 47.82 |
| | Entrez Gene: 25827 | | | | | | | |
| | Ensembl: | | | | | | | |
| | ENSG00000153558 | | | | | | | |
| | OMIM: 605652 | | | | | | | |
| | UniProtKB: | | | | | | | |
| | Q9UKC9 | | | | | | | |
| TRIM13 | HGNC: 9976 | 1.26269039884992E−15 | 1.39786847792469E−12 | 4 | 66 | 24 | 97.05 | 34.78 |
| | Entrez Gene: 10206 | | | | | | | |
| | Ensembl: | | | | | | | |
| | ENSG00000204977 | | | | | | | |
| | OMIM: 605661 | | | | | | | |
| | UniProtKB: O60858 | | | | | | | |
| CD1B | HGNC: 1635 | 3.0115635847658E−10 | 9.56319016750188E−11 | 8 | 9 | 39 | 13.23 | 56.52 |
| | Entrez Gene: 910 | | | | | | | |
| | Ensembl: | | | | | | | |
| | ENSG00000158485 | | | | | | | |
| | OMIM: 188360 | | | | | | | |
| | UniProtKB: P29016 | | | | | | | |
| VAX1 | HGNC: 12660 | 1.87672433282771E−06 | 1.0287163141036E−10 | 2 | 68 | 33 | 100 | 47.82 |
| | Entrez Gene: 11023 | | | | | | | |
| | Ensembl: | | | | | | | |
| | ENSG00000148704 | | | | | | | |
| | OMIM: 604294 | | | | | | | |
| | UniProtKB: | | | | | | | |
| | Q5SQQ9 | | | | | | | |
| CLASP1 | HGNC: 17088 | 8.91363938841512E−11 | 2.39567078392548E−08 | 20 | 20 | 40 | 29.41 | 57.97 |
| | Entrez Gene: 23332 | | | | | | | |
| | Ensembl: | | | | | | | |
| | ENSG00000074054 | | | | | | | |
| | OMIM: 605852 | | | | | | | |
| | UniProtKB: | | | | | | | |
| | Q7Z460 | | | | | | | |
| SUGT1 | HGNC: 16987 | 1.45579966126519E−06 | 0.0000726157171608077 | 4 | 3 | 24 | 4.41 | 34.78 |
| | Entrez Gene: 10910 | | | | | | | |
| | Ensembl: | | | | | | | |
| | ENSG00000165416 | | | | | | | |
| | OMIM: 604098 | | | | | | | |
| | UniProtKB: | | | | | | | |
| | Q9Y2Z0 | | | | | | | |
| MIPEP | HGNC: 7104 | 0.0014093581094382 | 0.00008359177892184 | 10 | 55 | 15 | 22.05 | 21.73 |
| | Entrez Gene: 4285 | | | | | | | |
| | Ensembl: | | | | | | | |
| | ENSG00000027001 | | | | | | | |
| | OMIM: 602241 | | | | | | | |
| | UniProtKB: Q99797 | | | | | | | |
| FAM188A | HGNC: 23578 | 1.91437820546762E−07 | 0.000142711996677612 | 6 | 55 | 18 | 80.88 | 26.08 |
| | Entrez Gene: 80013 | | | | | | | |
| | Ensembl: | | | | | | | |
| | ENSG00000148481 | | | | | | | |
| | OMIM: 611649 | | | | | | | |
| | UniProtKB: | | | | | | | |
| | Q9H8M7 | | | | | | | |
| TAF6 | HGNC: 11540 | 0.00141269941960906 | 0.000219166807116741 | 5 | 19 | 13 | 27.94 | 18.84 |
| | Entrez Gene: 6878 | | | | | | | |
| | Ensembl: | | | | | | | |
| | ENSG00000106290 | | | | | | | |
| | OMIM: 602955 | | | | | | | |
| | UniProtKB: P49848 | | | | | | | |
| WHSC1 | HGNC: 12766 | 0.00718326903405001 | 0.000240868230053009 | 10 | 13 | 13 | 19.1 | 18.84 |
| | Entrez Gene: 7468 | | | | | | | |
| | Ensembl: | | | | | | | |
| | ENSG00000109685 | | | | | | | |
| | OMIM: 602952 | | | | | | | |
| | UniProtKB: O96028 | | | | | | | |
| GPR158 | HGNC: 23689 | 0.000278262161730402 | 0.000278262161730402 | 6 | 10 | 0 | 14.7 | 0 |
| | Entrez Gene: 57512 | | | | | | | |
| | Ensembl: | | | | | | | |
| | ENSG00000151025 | | | | | | | |
| | OMIM: 614573 | | | | | | | |
| | UniProtKB: | | | | | | | |
| | Q5T848 | | | | | | | |
| HNRNPD | HGNC: 5036 | 0.00175975467919198 | 0.000510922803128836 | 2 | 9 | 27 | 13.23 | 39.13 |
| | Entrez Gene: 3184 | | | | | | | |
| | Ensembl: | | | | | | | |
| | ENSG00000138668 | | | | | | | |
| | OMIM: 601324 | | | | | | | |
| | UniProtKB: | | | | | | | |
| | Q14103 | | | | | | | |
| RUNX1T1 | HGNC: 1535 | 0.00235146027422903 | 0.000548781011086469 | 7 | 9 | 6 | 13.23 | 8.69 |
| | Entrez Gene: 862 | | | | | | | |
| | Ensembl: | | | | | | | |
| | ENSG00000079102 | | | | | | | |
| | OMIM: 133435 | | | | | | | |
| | UniProtKB: | | | | | | | |
| | Q06455 | | | | | | | |
| GRIK3 | HGNC: 4581 | 0.00061173751083077 | 0.00061173751083077 | 8 | 10 | 0 | 6.8 | 0 |
| | Entrez Gene: 2899 | | | | | | | |
| | Ensembl: | | | | | | | |
| | ENSG00000163873 | | | | | | | |
| | OMIM: 138243 | | | | | | | |
| | UniProtKB: | | | | | | | |
| | Q13003 | | | | | | | |
| FUZ | HGNC: 26219 | 0.000807306176520932 | 0.000678191067697156 | 2 | 12 | 1 | 17.64 | 1.44 |
| | Entrez Gene: 80199 | | | | | | | |
| | Ensembl: | | | | | | | |
| | ENSG00000010361 | | | | | | | |
| | OMIM: 610622 | | | | | | | |
| | UniProtKB: | | | | | | | |
| | Q9BT04 | | | | | | | |
| LYN | HGNC: 6735 | 0.000886349660487618 | 0.000745049768714577 | 8 | 15 | 31 | 22.05 | 44.09 |
| | Entrez Gene: 4067 | | | | | | | |
| | Ensembl: | | | | | | | |
| | ENSG00000254087 | | | | | | | |
| | OMIM: 165120 | | | | | | | |
| | UniProtKB: P07948 | | | | | | | |
| DDX5 | HGNC: 2746 | 0.000188591327183851 | 0.00109108884771161 | 10 | 21 | 21 | 30.88 | 30.43 |
| | Entrez Gene: 1655 | | | | | | | |
| | Ensembl: | | | | | | | |
| | ENSG00000108654 | | | | | | | |
| | OMIM: 180630 | | | | | | | |
| | UniProtKB: P17844 | | | | | | | |
| PODXL | HGNC: 9171 | 0.00136843519158836 | 0.0011242369420949 | 8 | 29 | 19 | 42.64 | 27.53 |
| | Entrez Gene: 5420 | | | | | | | |
| | Ensembl: | | | | | | | |
| | ENSG00000128567 | | | | | | | |
| | OMIM: 602632 | | | | | | | |
| | UniProtKB: | | | | | | | |
| | O00592 | | | | | | | |
| ATP5B | HGNC: 830 | 0.0000845225124055382 | 0.00115393759263137 | 2 | 39 | 58 | 57.35 | 84.05 |
| | Entrez Gene: 506 | | | | | | | |
| | Ensembl: | | | | | | | |
| | ENSG00000110955 | | | | | | | |
| | OMIM: 102910 | | | | | | | |
| | UniProtKB: P06576 | | | | | | | |
| PIGK | HGNC: 8965 | 0.00136203072056991 | 0.00136203072056991 | 1 | 8 | 0 | 11.76 | 0 |
| | Entrez Gene: 10026 | | | | | | | |
| | Ensembl: | | | | | | | |
| | ENSG00000142892 | | | | | | | |
| | OMIM: 605087 | | | | | | | |
| | UniProtKB: Q92643 | | | | | | | |
| AL159977.1 | GenBank: | 0.00142441312386281 | 0.00142441312386281 | 1 | 7 | 20 | 10.29 | 28.98 |
| | AL159977.1 | | | | | | | |

TABLE 6
Polymorphisms in genes associated with IS identified in Tables 4 and 5.
“Ref” refers to the “normal” allele in non scoliotic subjects and “Alt” to the
altered nucleotide (risk variant/SNP). The nucleotide sequence surrounding the
variant is provided in the table below.

| | | | | | | | | | SEQ |
|---|---|---|---|---|---|---|---|---|---|
| | | | | | | | | | ID |
| | | | | | | | | | NO. |
| | | | | | Position | | | | of |
| | | | | Ref. | of | | | | Ref/ |
| gene | Chr. | start | end | Sequence | variant | Ref | Alt | Risk variant sequence | Risk |
| CDK11A | chr1 | 1650909 | 1650930 | AACAGCACTGC | 1650920 | G | A | AACAGCACTGCATCATGCTTGA | 1/652 |
| | | | | GTCATGCTTGA | | | | | |
| CDK11A | chr1 | 1650985 | 1651006 | CATGATTCAGA | 1650996 | T | C | CATGATTCAGACAGGAACGAAG | 2/653 |
| | | | | TAGGAACGAAG | | | | | |
| CDK11A | chr1 | 1650992 | 1651013 | CAGATAGGAAC | 1651003 | G | A | CAGATAGGAACAAAGCTGAAAC | 3/654 |
| | | | | GAAGCTGAAAC | | | | | |
| C1orf222 | chr1 | 1890548 | 1890569 | TTTGAACTCAC | 1890559 | C | T | TTTGAACTCACTGAACATTTCT | 4/655 |
| | | | | CGAACATTTCT | | | | | |
| C1orf222 | chr1 | 1900007 | 1900028 | AGCCCTGAGGC | 1900018 | C | G | AGCCCTGAGGCGCCACCTGCCC | 5/656 |
| | | | | CCCACCTGCCC | | | | | |
| C1orf222 | chr1 | 1900145 | 1900166 | TCAGGGTCAGC | 1900156 | C | T | TCAGGGTCAGCTGGTGCCTGGC | 6/657 |
| | | | | CGGTGCCTGGC | | | | | |
| C1orf222 | chr1 | 1900200 | 1900221 | CTCCTCAGCCT | 1900211 | C | T | CTCCTCAGCCTTCTCTTTCAGA | 7/658 |
| | | | | CCTCTTTCAGA | | | | | |
| PLEKHG5 | chr1 | 6527585 | 6527606 | TCTCTTGGTCA | 6527596 | A | G | TCTCTTGGTCAGTGGCACTCTT | 8/659 |
| | | | | ATGGCACTCTT | | | | | |
| PLEKHG5 | chr1 | 6533091 | 6533112 | GCAGGCATTGT | 6533102 | C | T | GCAGGCATTGTTCTCATCCTCG | 9/660 |
| | | | | CCTCATCCTCG | | | | | |
| PLEKHG5 | chr1 | 6579449 | 6579470 | TCACTCTGTGT | 6579460 | C | T | TCACTCTGTGTTCTCAAACCTC | 10/661 |
| | | | | CCTCAAACCTC | | | | | |
| PLEKHG5 | chr1 | 6579510 | 6579531 | CCTGAACAAAG | 6579521 | G | C | CCTGAACAAAGCCTGAGCCAGC | 11/662 |
| | | | | GCTGAGCCAGC | | | | | |
| CD164L2 | chr1 | 27706610 | 27706631 | AACACCAGCAC | 27706621 | G | A | AACACCAGCACAACACCTCCGA | 12/663 |
| | | | | GACACCTCCGA | | | | | |
| GRIK3 | chr1 | 37291287 | 37291308 | GAAGGAGAAGA | 37291298 | C | T | GAAGGAGAAGATGCTGGGGTTG | 13/664 |
| | | | | CGCTGGGGTTG | | | | | |
| GRIK3 | chr1 | 37324746 | 37324767 | CAGGCCTTGTG | 37324757 | C | T | CAGGCCTTGTGTCGATGGCACT | 14/665 |
| | | | | CCGATGGCACT | | | | | |
| GRIK3 | chr1 | 37325587 | 37325608 | CGGTAGGGCTC | 37325598 | C | T | CGGTAGGGCTCTAGGTCTAAAG | 15/666 |
| | | | | CAGGTCTAAAG | | | | | |
| GRIK3 | chr1 | 37335226 | 37335247 | ACCCCTACAGC | 37335237 | C | T | ACCCCTACAGCTTGAGGAAGCT | 16/667 |
| | | | | CTGAGGAAGCT | | | | | |
| GRIK3 | chr1 | 37337948 | 37337969 | GAGCTCCTGCA | 37337959 | G | A | GAGCTCCTGCAATCGGATGAGC | 17/668 |
| | | | | GTCGGATGAGC | | | | | |
| GRIK3 | chr1 | 37346099 | 37346120 | AACCGAGTGGA | 37346110 | A | G | AACCGAGTGGAGCTGGGGTATG | 18/669 |
| | | | | ACTGGGGTATG | | | | | |
| GRIK3 | chr1 | 37346321 | 37346342 | TCGGGGTAGAG | 37346332 | G | A | TCGGGGTAGAGATTCACGTAGA | 19/670 |
| | | | | GTTCACGTAGA | | | | | |
| GRIK3 | chr1 | 37356458 | 37356479 | GCAAATGCAGC | 37356469 | G | A | GCAAATGCAGCACTTCCCCTCC | 20/671 |
| | | | | GCTTCCCCTCC | | | | | |
| PIGK | chr1 | 77558223 | 77558244 | CAGCAATCAAT | 77558234 | A | G | CAGCAATCAATGAAGCAAACAT | 21/672 |
| | | | | AAAGCAAACAT | | | | | |
| AHCYL1 | chr1 | 110557302 | 110557323 | CTCTTCACATG | 110557313 | G | C | CTCTTCACATGCATCTCAGAAA | 22/673 |
| | | | | GATCTCAGAAA | | | | | |
| AHCYL1 | chr1 | 110560028 | 110560049 | GGTTAATTCCT | 110560039 | G | A | GGTTAATTCCTATCTCACAAAT | 23/674 |
| | | | | GTCTCACAAAT | | | | | |
| AHCYL1 | chr1 | 110561013 | 110561034 | CACGGGAGCAC | 110561024 | T | C | CACGGGAGCACCTGGATCGCAT | 24/675 |
| | | | | TTGGATCGCAT | | | | | |
| GAPDHP27 | chr1 | 120102161 | 120102182 | CTTTTGGAGGA | 120102172 | T | A | CTTTTGGAGGAAGGTGGTGGGA | 25/676 |
| | | | | TGGTGGTGGGA | | | | | |
| CD1B | chr1 | 158297853 | 158297874 | AGTTTTAAGTA | 158297864 | C | A | AGTTTTAAGTAATTTTTTGCTG | 26/677 |
| | | | | CTTTTTTGCTG | | | | | |
| CD1B | chr1 | 158298678 | 158298699 | TTAAAAAAAAA | 158298689 | A | C | TTAAAAAAAAACACAACACCAC | 27/678 |
| | | | | AACAACACCAC | | | | | |
| CD1B | chr1 | 158298682 | 158298703 | AAAAAAAAACA | 158298693 | A | C | AAAAAAAAACACCACCACCCAC | 28/679 |
| | | | | ACACCACCCAC | | | | | |
| CD1B | chr1 | 158299677 | 158299698 | ACGCCCAAGAG | 158299688 | A | T | ACGCCCAAGAGTTATCGGGGGC | 29/680 |
| | | | | ATATCGGGGGC | | | | | |
| CD1B | chr1 | 158299745 | 158299766 | TTGTATGATTA | 158299756 | G | A | TTGTATGATTAATGCACAGAAT | 30/681 |
| | | | | GTGCACAGAAT | | | | | |
| CD1B | chr1 | 158299755 | 158299776 | AGTGCACAGAA | 158299766 | T | C | AGTGCACAGAACTTCTGTGCCC | 31/682 |
| | | | | TTTCTGTGCCC | | | | | |
| CD1B | chr1 | 158299829 | 158299850 | ATCCAATCCTC | 158299840 | C | T | ATCCAATCCTCTTAGAGCTCCC | 32/683 |
| | | | | CTAGAGCTCCC | | | | | |
| CD1B | chr1 | 158300593 | 158300614 | CTGGAAATCAC | 158300604 | C | T | CTGGAAATCACTGGCAAAGTCT | 33/684 |
| | | | | CGGCAAAGTCT | | | | | |
| C1orf106 | chr1 | 200867541 | 200867562 | GATGAGGTCAG | 200867552 | C | T | GATGAGGTCAGTGACACCGACA | 34/685 |
| | | | | CGACACCGACA | | | | | |
| C1orf106 | chr1 | 200867561 | 200867582 | CAGTGGCATCA | 200867572 | T | A | CAGTGGCATCAACCTGCAGTCT | 35/686 |
| | | | | TCCTGCAGTCT | | | | | |
| C1orf106 | chr1 | 200878015 | 200878036 | GGACCACCCCT | 200878026 | A | T | GGACCACCCCTTTGAGAAGCCC | 36/687 |
| | | | | ATGAGAAGCCC | | | | | |
| TNNT2 | chr1 | 201330355 | 201330376 | CCAGGAGGAGT | 201330366 | G | C | CCAGGAGGAGTCTGAGATGGAG | 37/688 |
| | | | | GTGAGATGGAG | | | | | |
| TNNT2 | chr1 | 201336017 | 201336038 | ACACAGCCATG | 201336028 | G | C | ACACAGCCATGCGTCAGGGGGC | 38/689 |
| | | | | GGTCAGGGGGC | | | | | |
| TNNT2 | chr1 | 201337475 | 201337496 | TGAATTTGGGG | 201337486 | G | A | TGAATTTGGGGACAACCAACGT | 39/690 |
| | | | | GCAACCAACGT | | | | | |
| TNNT2 | chr1 | 201341214 | 201341235 | TGGGTCAGTTT | 201341225 | C | T | TGGGTCAGTTTTGAACCAGGCT | 40/691 |
| | | | | CGAACCAGGCT | | | | | |
| PXDN | chr2 | 1639140 | 1639161 | GTATACCTTAG | 1639151 | G | A | GTATACCTTAGAACATGAAGAT | 41/692 |
| | | | | GACATGAAGAT | | | | | |
| PXDN | chr2 | 1642473 | 1642494 | ACACCCCCAAG | 1642484 | G | T | ACACCCCCAAGTCTCCAGGGTC | 42/693 |
| | | | | GCTCCAGGGTC | | | | | |
| PXDN | chr2 | 1642537 | 1642558 | TGCTATACCCA | 1642548 | G | A | TGCTATACCCAAAAGGTTCGGG | 43/694 |
| | | | | GAAGGTTCGGG | | | | | |
| PXDN | chr2 | 1647231 | 1647252 | GTCGCTCTGCA | 1647242 | C | A | GTCGCTCTGCAACCGGGTGATG | 44/695 |
| | | | | CCCGGGTGATG | | | | | |
| PXDN | chr2 | 1648559 | 1648580 | CAAAGCTGCAC | 1648570 | G | A | CAAAGCTGCACATGGTAAAAAA | 45/696 |
| | | | | GTGGTAAAAAA | | | | | |
| PXDN | chr2 | 1651990 | 1652011 | GGTCCTCGAAC | 1652001 | G | A | GGTCCTCGAACATGTGTGCCGC | 46/697 |
| | | | | GTGTGTGCCGC | | | | | |
| PXDN | chr2 | 1652343 | 1652364 | AAGGCCGCGGT | 1652354 | G | A | AAGGCCGCGGTAGCGAAGGCGT | 47/698 |
| | | | | GGCGAAGGCGT | | | | | |
| PXDN | chr2 | 1657349 | 1657370 | CAACCCCGTTA | 1657360 | C | T | CAACCCCGTTATTCAGGCCATG | 48/699 |
| | | | | CTCAGGCCATG | | | | | |
| PXDN | chr2 | 1657501 | 1657522 | TAAGGATCCCT | 1657512 | C | G | TAAGGATCCCTGGGATACCGGA | 49/700 |
| | | | | CGGATACCGGA | | | | | |
| PXDN | chr2 | 1657568 | 1657589 | TTTAGGGGGAA | 1657579 | G | A | TTTAGGGGGAAAAAAGGAAGAA | 50/701 |
| | | | | GAAAGGAAGAA | | | | | |
| PXDN | chr2 | 1658140 | 1658161 | TGAGGGTCAGC | 1658151 | G | A | TGAGGGTCAGCATGTTTACAGC | 51/702 |
| | | | | GTGTTTACAGC | | | | | |
| PXDN | chr2 | 1658214 | 1658235 | ACAGTCGCAAT | 1658225 | C | T | ACAGTCGCAATTGCTTCCACGA | 52/703 |
| | | | | CGCTTCCACGA | | | | | |
| PXDN | chr2 | 1658262 | 1658283 | TTTCGACTGAC | 1658273 | G | A | TTTCGACTGACATCAGGAACTA | 53/704 |
| | | | | GTCAGGAACTA | | | | | |
| PXDN | chr2 | 1695760 | 1695781 | TTATTGAGAAG | 1695771 | C | T | TTATTGAGAAGTCTATGAAAGA | 54/705 |
| | | | | CCTATGAAAGA | | | | | |
| PPP1R21 | chr2 | 48678085 | 48678106 | ATTTCCTTGAT | 48678096 | A | G | ATTTCCTTGATGTAACCAATTG | 55/706 |
| | | | | ATAACCAATTG | | | | | |
| PPP1R21 | chr2 | 48678086 | 48678107 | TTTCCTTGATA | 48678097 | T | C | TTTCCTTGATACAACCAATTGC | 56/707 |
| | | | | TAACCAATTGC | | | | | |
| PPP1R21 | chr2 | 48681857 | 48681878 | GAACCACGAGG | 48681868 | C | G | GAACCACGAGGGAAGAAAAACA | 57/708 |
| | | | | CAAGAAAAACA | | | | | |
| PPP1R21 | chr2 | 48681907 | 48681928 | GTGACCTTGTC | 48681918 | G | A | GTGACCTTGTCATTAGTTACTG | 58/709 |
| | | | | GTTAGTTACTG | | | | | |
| PPP1R21 | chr2 | 48685406 | 48685427 | TCTGTGATCTG | 48685417 | T | C | TCTGTGATCTGCTAATGTGGAA | 59/710 |
| | | | | TTAATGTGGAA | | | | | |
| PPP1R21 | chr2 | 48687124 | 48687145 | ACTTAGAGTTA | 48687135 | G | C | ACTTAGAGTTACTCATTTCTGG | 60/711 |
| | | | | GTCATTTCTGG | | | | | |
| PPP1R21 | chr2 | 48698374 | 48698395 | TGTCCAGTAGC | 48698385 | A | C | TGTCCAGTAGCCCTTTTAACCT | 61/712 |
| | | | | ACTTTTAACCT | | | | | |
| PPP1R21 | chr2 | 48707198 | 48707219 | CTTTCTTCGAT | 48707209 | C | G | CTTTCTTCGATGTGCCTGAATA | 62/713 |
| | | | | CTGCCTGAATA | | | | | |
| PPP1R21 | chr2 | 48713961 | 48713982 | CATAGGAAAAT | 48713972 | G | C | CATAGGAAAATCGATCTGTAAA | 63/714 |
| | | | | GGATCTGTAAA | | | | | |
| PPP1R21 | chr2 | 48722886 | 48722907 | AAGTCGAGAAG | 48722897 | G | T | AAGTCGAGAAGTCCTTGCACAG | 64/715 |
| | | | | GCCTTGCACAG | | | | | |
| PPP1R21 | chr2 | 48722983 | 48723004 | ATACGTAGAAT | 48722994 | G | C | ATACGTAGAATCATTCAAAAGT | 65/716 |
| | | | | GATTCAAAAGT | | | | | |
| PPP1R21 | chr2 | 48723093 | 48723114 | TAAGTCATCTT | 48723104 | G | A | TAAGTCATCTTAATTCAGTTGG | 66/717 |
| | | | | GATTCAGTTGG | | | | | |
| PPP1R21 | chr2 | 48725552 | 48725573 | ACATTCTGTAA | 48725563 | G | A | ACATTCTGTAAAATAGTTTTTG | 67/718 |
| | | | | GATAGTTTTTG | | | | | |
| PPP1R21 | chr2 | 48732659 | 48732680 | ATGTACACATT | 48732670 | C | T | ATGTACACATTTTGTTCTAAAA | 68/719 |
| | | | | CTGTTCTAAAA | | | | | |
| PPP1R21 | chr2 | 48737154 | 48737175 | TGCCGAGCACT | 48737165 | G | C | TGCCGAGCACTCTCTAAAAGAC | 69/720 |
| | | | | GTCTAAAAGAC | | | | | |
| PPP1R21 | chr2 | 48738360 | 48738381 | AGTAAATTATT | 48738371 | G | T | AGTAAATTATTTGGAAACTATA | 70/721 |
| | | | | GGGAAACTATA | | | | | |
| PPP1R21 | chr2 | 48738384 | 48738405 | TTCCCTACTCC | 48738395 | A | C | TTCCCTACTCCCCATTTTTCTT | 71/722 |
| | | | | ACATTTTTCTT | | | | | |
| PPP1R21 | chr2 | 48738430 | 48738451 | TGGTACGACTC | 48738441 | G | A | TGGTACGACTCATGGGATGTTG | 72/723 |
| | | | | GTGGGATGTTG | | | | | |
| PPP1R21 | chr2 | 48741785 | 48741806 | GTTTATAAACT | 48741796 | A | G | GTTTATAAACTGTGTGAGTTAT | 73/724 |
| | | | | ATGTGAGTTAT | | | | | |
| NFU1 | chr2 | 69633261 | 69633282 | CCATGCAAGTA | 69633272 | C | T | CCATGCAAGTATGAGTATTAAA | 74/725 |
| | | | | CGAGTATTAAA | | | | | |
| NFU1 | chr2 | 69642379 | 69642400 | ATTGTTGCATA | 69642390 | A | G | ATTGTTGCATAGATATCTGGTT | 75/726 |
| | | | | AATATCTGGTT | | | | | |
| NFU1 | chr2 | 69642461 | 69642482 | TTCCAGGCAAC | 69642472 | G | A | TTCCAGGCAACAGCAAAGACCC | 76/727 |
| | | | | GGCAAAGACCC | | | | | |
| NFU1 | chr2 | 69650719 | 69650740 | CAGAGGGGAGC | 69650730 | G | A | CAGAGGGGAGCAAAATGCTGCA | 77/728 |
| | | | | GAAATGCTGCA | | | | | |
| POLR1A | chr2 | 86255154 | 86255175 | CGAGGGTCGAC | 86255165 | C | T | CGAGGGTCGACTGCGATGCCTG | 78/729 |
| | | | | CGCGATGCCTG | | | | | |
| POLR1A | chr2 | 86255669 | 86255690 | CCTGGCTGGTG | 86255680 | C | T | CCTGGCTGGTGTCCAGACCTCG | 79/730 |
| | | | | CCCAGACCTCG | | | | | |
| POLR1A | chr2 | 86255755 | 86255776 | ACCCGCAGCGC | 86255766 | G | A | ACCCGCAGCGCAGCCTCAATGC | 80/731 |
| | | | | GGCCTCAATGC | | | | | |
| POLR1A | chr2 | 86260774 | 86260795 | ACTCACTCACC | 86260785 | C | T | ACTCACTCACCTGACTCCTCCC | 81/732 |
| | | | | CGACTCCTCCC | | | | | |
| POLR1A | chr2 | 86265729 | 86265750 | AATCTCTAGGA | 86265740 | G | A | AATCTCTAGGAACCTTGTGGCT | 82/733 |
| | | | | GCCTTGTGGCT | | | | | |
| POLR1A | chr2 | 86265823 | 86265844 | CTTGTTTCCAT | 86265834 | G | A | CTTGTTTCCATAAAGCGCAGGA | 83/734 |
| | | | | GAAGCGCAGGA | | | | | |
| POLR1A | chr2 | 86266015 | 86266036 | TCAGGTCCTAG | 86266026 | G | A | TCAGGTCCTAGATGACTGCGCA | 84/735 |
| | | | | GTGACTGCGCA | | | | | |
| POLR1A | chr2 | 86270041 | 86270062 | CCCACTGACTA | 86270052 | A | G | CCCACTGACTAGGCCTGGACGT | 85/736 |
| | | | | AGCCTGGACGT | | | | | |
| POLR1A | chr2 | 86271380 | 86271401 | GTGAGATCATA | 86271391 | C | T | GTGAGATCATATTGCACGACCA | 86/737 |
| | | | | CTGCACGACCA | | | | | |
| POLR1A | chr2 | 86272298 | 86272319 | TTTCATCCAAG | 86272309 | G | A | TTTCATCCAAGAAATCTTAAAT | 87/738 |
| | | | | GAATCTTAAAT | | | | | |
| POLR1A | chr2 | 86272339 | 86272360 | AAAGATGATGG | 86272350 | C | G | AAAGATGATGGGAGGAAGGGCC | 88/739 |
| | | | | CAGGAAGGGCC | | | | | |
| POLR1A | chr2 | 86272632 | 86272653 | CAATAGCCCCC | 86272643 | A | G | CAATAGCCCCCGGTGTCTTCTG | 89/740 |
| | | | | AGTGTCTTCTG | | | | | |
| POLR1A | chr2 | 86276384 | 86276405 | AAGCTTGTAGG | 86276395 | C | G | AAGCTTGTAGGGGACCTCAGGC | 90/741 |
| | | | | CGACCTCAGGC | | | | | |
| POLR1A | chr2 | 86281211 | 86281232 | TGTCTAACCCC | 86281222 | G | A | TGTCTAACCCCAGCACTAGAAG | 91/742 |
| | | | | GGCACTAGAAG | | | | | |
| POLR1A | chr2 | 86281295 | 86281316 | CAGGAACGGAT | 86281306 | C | T | CAGGAACGGATTGAGGAGTTTC | 92/743 |
| | | | | CGAGGAGTTTC | | | | | |
| POLR1A | chr2 | 86292488 | 86292509 | AGCTCCATATA | 86292499 | G | C | AGCTCCATATACTGCTCCCGGG | 93/744 |
| | | | | GTGCTCCCGGG | | | | | |
| POLR1A | chr2 | 86297094 | 86297115 | AAGTCATGCTG | 86297105 | A | G | AAGTCATGCTGGCGATGACCAC | 94/745 |
| | | | | ACGATGACCAC | | | | | |
| POLR1A | chr2 | 86305296 | 86305317 | TATTGACGTGG | 86305307 | C | T | TATTGACGTGGTTCTGAAGGCG | 95/746 |
| | | | | CTCTGAAGGCG | | | | | |
| POLR1A | chr2 | 86305393 | 86305414 | CAAAGAGTCTT | 86305404 | T | C | CAAAGAGTCTTCTTCCTGGAAG | 96/747 |
| | | | | TTTCCTGGAAG | | | | | |
| POLR1A | chr2 | 86305394 | 86305415 | AAAGAGTCTTT | 86305405 | T | C | AAAGAGTCTTTCTCCTGGAAGA | 97/748 |
| | | | | TTCCTGGAAGA | | | | | |
| POLR1A | chr2 | 86308120 | 86308141 | AATAAATGAAT | 86308131 | G | T | AATAAATGAATTTTTAGTGTGA | 98/749 |
| | | | | GTTTAGTGTGA | | | | | |
| POLR1A | chr2 | 86310133 | 86310154 | GGCAGGGTTAA | 86310144 | T | C | GGCAGGGTTAACGAGTCCAGGA | 99/750 |
| | | | | TGAGTCCAGGA | | | | | |
| POLR1A | chr2 | 86315625 | 86315646 | CCCAGGGTTTA | 86315636 | G | A | CCCAGGGTTTAAGACACATCTG | 100/751 |
| | | | | GGACACATCTG | | | | | |
| POLR1A | chr2 | 86316833 | 86316854 | TGCAAACTCAG | 86316844 | G | A | TGCAAACTCAGATCTACTTTGG | 101/752 |
| | | | | GTCTACTTTGG | | | | | |
| POLR1A | chr2 | 86316878 | 86316899 | ACAACACAGAG | 86316889 | G | A | ACAACACAGAGAGAAAGTGATA | 102/753 |
| | | | | GGAAAGTGATA | | | | | |
| POLR1A | chr2 | 86327050 | 86327071 | ACCAGCAGCCC | 86327061 | G | A | ACCAGCAGCCCAGAGACCCACA | 103/754 |
| | | | | GGAGACCCACA | | | | | |
| REEP1 | chr2 | 86444162 | 86444183 | TCACGTGGTTT | 86444173 | C | T | TCACGTGGTTTTGGTGGCCGAG | 104/755 |
| | | | | CGGTGGCCGAG | | | | | |
| REEP1 | chr2 | 86444241 | 86444262 | AGAAAACAGAA | 86444252 | A | C | AGAAAACAGAACGGTGTCCCTC | 105/756 |
| | | | | AGGTGTCCCTC | | | | | |
| RNF149 | chr2 | 101911281 | 101911302 | ATAATATAAGC | 101911292 | G | A | ATAATATAAGCAAAAATCAAGA | 106/757 |
| | | | | GAAAATCAAGA | | | | | |
| RNF149 | chr2 | 101911448 | 101911469 | AGCCAGGCTAA | 101911459 | C | T | AGCCAGGCTAATGAGATAATCA | 107/758 |
| | | | | CGAGATAATCA | | | | | |
| RNF149 | chr2 | 101911640 | 101911661 | GTTCCTGTAGG | 101911651 | A | G | GTTCCTGTAGGGAAGAACAAAG | 108/759 |
| | | | | AAAGAACAAAG | | | | | |
| CLASP1 | chr2 | 122098542 | 122098563 | ACTGAATTAGC | 122098553 | A | G | ACTGAATTAGCGGGAAGAAAAG | 109/760 |
| | | | | AGGAAGAAAAG | | | | | |
| CLASP1 | chr2 | 122120668 | 122120689 | TTCTGATTCCT | 122120679 | C | T | TTCTGATTCCTTTATCTCCATG | 110/761 |
| | | | | CTATCTCCATG | | | | | |
| CLASP1 | chr2 | 122125202 | 122125223 | TCTTTCAGGGC | 122125213 | G | A | TCTTTCAGGGCAGTCTTGTCGT | 111/762 |
| | | | | GGTCTTGTCGT | | | | | |
| CLASP1 | chr2 | 122125362 | 122125383 | CCCGGCCCTCA | 122125373 | G | T | CCCGGCCCTCATTGGCAGGGGA | 112/763 |
| | | | | GTGGCAGGGGA | | | | | |
| CLASP1 | chr2 | 122144817 | 122144838 | AGAATAGGCAT | 122144828 | C | T | AGAATAGGCATTGTTATTCAAG | 113/764 |
| | | | | CGTTATTCAAG | | | | | |
| CLASP1 | chr2 | 122154592 | 122154613 | AACAGCAGAGC | 122154603 | C | T | AACAGCAGAGCTTTAGTGATAT | 114/765 |
| | | | | CTTAGTGATAT | | | | | |
| CLASP1 | chr2 | 122154650 | 122154671 | AAATACATTTC | 122154661 | T | C | AAATACATTTCCCTAACACAAG | 115/766 |
| | | | | TCTAACACAAG | | | | | |
| CLASP1 | chr2 | 122168614 | 122168635 | ACTGCCTCTTC | 122168625 | C | A | ACTGCCTCTTCACACCAAGTAG | 116/767 |
| | | | | CCACCAAGTAG | | | | | |
| CLASP1 | chr2 | 122176264 | 122176285 | ACCCCTGACTC | 122176275 | A | G | ACCCCTGACTCGTGCTGGGTCG | 117/768 |
| | | | | ATGCTGGGTCG | | | | | |
| CLASP1 | chr2 | 122182718 | 122182739 | ATCCTATTCGG | 122182729 | T | C | ATCCTATTCGGCTTGGACTTGT | 118/769 |
| | | | | TTTGGACTTGT | | | | | |
| CLASP1 | chr2 | 122184939 | 122184960 | AACAACAACAA | 122184950 | C | A | AACAACAACAAAAAAAAAAGGC | 119/770 |
| | | | | CAAAAAAAGGC | | | | | |
| CLASP1 | chr2 | 122208631 | 122208652 | GTGGGAGAAAT | 122208642 | A | T | GTGGGAGAAATTACTTCCAAAT | 120/771 |
| | | | | AACTTCCAAAT | | | | | |
| CLASP1 | chr2 | 122220021 | 122220042 | ACAACATTACA | 122220032 | T | C | ACAACATTACACAGGCAGAATT | 121/772 |
| | | | | TAGGCAGAATT | | | | | |
| CLASP1 | chr2 | 122220065 | 122220086 | AGTTGACCCTT | 122220076 | G | C | AGTTGACCCTTCTTTGTAATCA | 122/773 |
| | | | | GTTTGTAATCA | | | | | |
| CLASP1 | chr2 | 122227429 | 122227450 | TTCCCATTCCA | 122227440 | A | G | TTCCCATTCCAGCGTTTCTCCG | 123/774 |
| | | | | ACGTTTCTCCG | | | | | |
| CLASP1 | chr2 | 122247821 | 122247842 | CTTTTCAGAAT | 122247832 | A | G | CTTTTCAGAATGTATTAGAATA | 124/775 |
| | | | | ATATTAGAATA | | | | | |
| CLASP1 | chr2 | 122283524 | 122283545 | TAGTGAGAACT | 122283535 | G | A | TAGTGAGAACTAAGGAAAGAAA | 125/776 |
| | | | | GAGGAAAGAAA | | | | | |
| CLASP1 | chr2 | 122286185 | 122286206 | CACACACCCTC | 122286196 | G | A | CACACACCCTCACACGTGCATG | 126/777 |
| | | | | GCACGTGCATG | | | | | |
| CLASP1 | chr2 | 122286252 | 122286273 | CCTGGGGATTA | 122286263 | G | A | CCTGGGGATTAACAGCTTGATC | 127/778 |
| | | | | GCAGCTTGATC | | | | | |
| CLASP1 | chr2 | 122363450 | 122363471 | AGGACTCCATG | 122363461 | C | A | AGGACTCCATGAGAGGCTCCAT | 128/779 |
| | | | | CGAGGCTCCAT | | | | | |
| NOP58 | chr2 | 203130621 | 203130642 | TACAGCTTCTG | 203130632 | G | C | TACAGCTTCTGCCAGGCCGTGC | 129/780 |
| | | | | GCAGGCCGTGC | | | | | |
| NOP58 | chr2 | 203157527 | 203157548 | CAGCTCTATGA | 203157538 | A | G | CAGCTCTATGAGTATCTACAAA | 130/781 |
| | | | | ATATCTACAAA | | | | | |
| NOP58 | chr2 | 203160650 | 203160671 | TCTTCTATTGT | 203160661 | C | T | TCTTCTATTGTTTCTTTCTTGT | 131/782 |
| | | | | CTCTTTCTTGT | | | | | |
| NOP58 | chr2 | 203164961 | 203164982 | GTGAAGACTTA | 203164972 | C | T | GTGAAGACTTATGATCCTTCTG | 132/783 |
| | | | | CGATCCTTCTG | | | | | |
| NOP58 | chr2 | 203168037 | 203168058 | TTATATTTTCA | 203168048 | A | G | TTATATTTTCAGTGTGATTACT | 133/784 |
| | | | | ATGTGATTACT | | | | | |
| AC | chr2 | 240500448 | 240500469 | AGAGACGCTGT | 240500459 | T | G | AGAGACGCTGTGCCCTTGAGGG | 134/785 |
| 079612.1 | | | | TCCCTTGAGGG | | | | | |
| AC | chr2 | 240500547 | 240500568 | CTCTGGGTTCA | 240500558 | A | G | CTCTGGGTTCAGTTAAGAAGGT | 135/786 |
| 079612.1 | | | | ATTAAGAAGGT | | | | | |
| AC | chr2 | 240500549 | 240500570 | CTGGGTTCAAT | 240500560 | T | C | CTGGGTTCAATCAAGAAGGTTA | 136/787 |
| 079612.1 | | | | TAAGAAGGTTA | | | | | |
| FBXL2 | chr3 | 33339135 | 33339156 | CCTTTTTTTTT | 33339146 | T | C | CCTTTTTTTTTCTCTTTCCAGG | 137/788 |
| | | | | TTCTTTCCAGG | | | | | |
| FBXL2 | chr3 | 33406114 | 33406135 | AAGGGTCGAGT | 33406125 | G | T | AAGGGTCGAGTTGTGGAAAATA | 138/789 |
| | | | | GGTGGAAAATA | | | | | |
| FBXL2 | chr3 | 33406268 | 33406289 | AAATAAACCAA | 33406279 | G | A | AAATAAACCAAACCTATTACAT | 139/790 |
| | | | | GCCTATTACAT | | | | | |
| FBXL2 | chr3 | 33414660 | 33414681 | ATGAAGATTAA | 33414671 | T | G | ATGAAGATTAAGTGGTGACCAA | 140/791 |
| | | | | TTGGTGACCAA | | | | | |
| FBXL2 | chr3 | 33415036 | 33415057 | ACTGCACATAA | 33415047 | G | A | ACTGCACATAAATTTTTGTTTC | 141/792 |
| | | | | GTTTTTGTTTC | | | | | |
| FBXL2 | chr3 | 33415050 | 33415071 | TTTGTTTCTTG | 33415061 | T | G | TTTGTTTCTTGGTCTCTCAGTG | 142/793 |
| | | | | TTCTCTCAGTG | | | | | |
| FBXL2 | chr3 | 33416924 | 33416945 | ACTTTTTGCTT | 33416935 | T | C | ACTTTTTGCTTCGCAGCTCAGA | 143/794 |
| | | | | TGCAGCTCAGA | | | | | |
| FBXL2 | chr3 | 33418677 | 33418698 | AAAAGACTCAA | 33418688 | G | A | AAAAGACTCAAATATGCATCAT | 144/795 |
| | | | | GTATGCATCAT | | | | | |
| FBXL2 | chr3 | 33418723 | 33418744 | TAGTTGGTACC | 33418734 | G | T | TAGTTGGTACCTTTTTCTCCCC | 145/796 |
| | | | | GTTTTCTCCCC | | | | | |
| FBXL2 | chr3 | 33418830 | 33418851 | GGCATAGATTT | 33418841 | A | C | GGCATAGATTTCAAGAATACAA | 146/797 |
| | | | | AAAGAATACAA | | | | | |
| FBXL2 | chr3 | 33420171 | 33420192 | CTCCAGATAAC | 33420182 | C | T | CTCCAGATAACTGACAGCACAC | 147/798 |
| | | | | CGACAGCACAC | | | | | |
| FBXL2 | chr3 | 33426940 | 33426961 | AATCCTAAAAA | 33426951 | T | C | AATCCTAAAAACAGTAATGTGT | 148/799 |
| | | | | TAGTAATGTGT | | | | | |
| AC | chr3 | 36211433 | 36211454 | CACATTAGATG | 36211444 | T | A | CACATTAGATGAAGAACTGTGG | 149/800 |
| 105901.1 | | | | TAGAACTGTGG | | | | | |
| RBM5 | chr3 | 50131129 | 50131150 | GTGATTTTGTT | 50131140 | T | A | GTGATTTTGTTAATTGTAACTC | 150/801 |
| | | | | TATTGTAACTC | | | | | |
| RBM5 | chr3 | 50144375 | 50144396 | GTGCCCCAAGT | 50144386 | A | G | GTGCCCCAAGTGTGTTGAGACA | 151/802 |
| | | | | ATGTTGAGACA | | | | | |
| RBM5 | chr3 | 50145443 | 50145464 | CTGAATTTTTT | 50145454 | T | C | CTGAATTTTTTCCCTTAATGCC | 152/803 |
| | | | | TCCTTAATGCC | | | | | |
| RBM5 | chr3 | 50150759 | 50150780 | AATCGACTGAC | 50150770 | A | G | AATCGACTGACGTAGCAGAAAG | 153/804 |
| | | | | ATAGCAGAAAG | | | | | |
| RBM5 | chr3 | 50151612 | 50151633 | GGGAGCCTTAG | 50151623 | C | T | GGGAGCCTTAGTTGAAAGGCAG | 154/805 |
| | | | | CTGAAAGGCAG | | | | | |
| RBM5 | chr3 | 50154844 | 50154865 | CAGGCTTACAG | 50154855 | G | C | CAGGCTTACAGCCCGGTTCCAG | 155/806 |
| | | | | GCCGGTTCCAG | | | | | |
| CD96 | chr3 | 111263814 | 111263835 | CTGAAGTGACT | 111263825 | A | G | CTGAAGTGACTGGGGTTTTTAA | 156/807 |
| | | | | AGGGTTTTTAA | | | | | |
| CD96 | chr3 | 111264001 | 111264022 | AATGGTCCAAG | 111264012 | G | A | AATGGTCCAAGATCACCAATAA | 157/808 |
| | | | | GTCACCAATAA | | | | | |
| CD96 | chr3 | 111286364 | 111286385 | TTACAGTTACA | 111286375 | G | C | TTACAGTTACACCAGATGAATG | 158/809 |
| | | | | GCAGATGAATG | | | | | |
| CD96 | chr3 | 111298007 | 111298028 | GGCGGAAGTTC | 111298018 | T | C | GGCGGAAGTTCCCTTGCCACAT | 159/810 |
| | | | | TCTTGCCACAT | | | | | |
| CD96 | chr3 | 111298019 | 111298040 | CTTGCCACATT | 111298030 | A | G | CTTGCCACATTGGAGTCGGTCC | 160/811 |
| | | | | AGAGTCGGTCC | | | | | |
| CD96 | chr3 | 111312294 | 111312315 | CACCAAACTAC | 111312305 | T | C | CACCAAACTACCTTGCTTTACA | 161/812 |
| | | | | TTTGCTTTACA | | | | | |
| CD96 | chr3 | 111356081 | 111356102 | CCGTCAGGTGC | 111356092 | A | G | CCGTCAGGTGCGGGCTCAACAC | 162/813 |
| | | | | AGGCTCAACAC | | | | | |
| CD96 | chr3 | 111368603 | 111368624 | CAAGAGCCCAA | 111368614 | C | T | CAAGAGCCCAATGAAAGTGATC | 163/814 |
| | | | | CGAAAGTGATC | | | | | |
| RAB7A | chr3 | 128525242 | 128525263 | TTCCAGTCTCT | 128525253 | C | T | TTCCAGTCTCTTGGTGTGGCCT | 164/815 |
| | | | | CGGTGTGGCCT | | | | | |
| RAB7A | chr3 | 128526398 | 128526419 | CGGGCACAGGC | 128526409 | C | G | CGGGCACAGGCGTGGTGCTACA | 165/816 |
| | | | | CTGGTGCTACA | | | | | |
| RAB7A | chr3 | 128533117 | 128533138 | TTTTCATCTCT | 128533128 | C | G | TTTTCATCTCTGCAGGGGGAAA | 166/817 |
| | | | | CCAGGGGGAAA | | | | | |
| CDV3 | chr3 | 133293802 | 133293823 | ATCCACTTTTC | 133293813 | G | A | ATCCACTTTTCATAGTGTGTTA | 167/818 |
| | | | | GTAGTGTGTTA | | | | | |
| CDV3 | chr3 | 133303068 | 133303089 | AGTGCATGATT | 133303079 | G | A | AGTGCATGATTATGGTAGGGTG | 168/819 |
| | | | | GTGGTAGGGTG | | | | | |
| CDV3 | chr3 | 133306667 | 133306688 | AATTCAAGGAC | 133306678 | G | A | AATTCAAGGACAAATATTTTCA | 169/820 |
| | | | | GAATATTTTCA | | | | | |
| TOPBP1 | chr3 | 133327331 | 133327352 | TAAACGTATCT | 133327342 | C | T | TAAACGTATCTTTGGTAATGAG | 170/821 |
| | | | | CTGGTAATGAG | | | | | |
| TOPBP1 | chr3 | 133327548 | 133327569 | AAAAACAAGGT | 133327559 | A | G | AAAAACAAGGTGGTATCACAAT | 171/822 |
| | | | | AGTATCACAAT | | | | | |
| TOPBP1 | chr3 | 133336988 | 133337009 | CATCTGTTTAT | 133336999 | T | C | CATCTGTTTATCTTTAAGAGGT | 172/823 |
| | | | | TTTTAAGAGGT | | | | | |
| TOPBP1 | chr3 | 133338979 | 133339000 | CATCAGTGACA | 133338990 | G | A | CATCAGTGACAAGTACACACCT | 173/824 |
| | | | | GGTACACACCT | | | | | |
| TOPBP1 | chr3 | 133342071 | 133342092 | CAAGCCACTGT | 133342082 | A | G | CAAGCCACTGTGTAAGGTTTCA | 174/825 |
| | | | | ATAAGGTTTCA | | | | | |
| TOPBP1 | chr3 | 133342181 | 133342202 | CTGAGAGTAGT | 133342192 | C | T | CTGAGAGTAGTTGACTATTACA | 175/826 |
| | | | | CGACTATTACA | | | | | |
| TOPBP1 | chr3 | 133347377 | 133347398 | TCCTTTTAATA | 133347388 | A | G | TCCTTTTAATAGGAGAACTAAT | 176/827 |
| | | | | AGAGAACTAAT | | | | | |
| TOPBP1 | chr3 | 133356666 | 133356687 | AACTTTATAAA | 133356677 | C | T | AACTTTATAAATGGTAAAACTG | 177/828 |
| | | | | CGGTAAAACTG | | | | | |
| TOPBP1 | chr3 | 133358983 | 133359004 | CATTGGATTTG | 133358994 | C | T | CATTGGATTTGTGAACAAAGTA | 178/829 |
| | | | | CGAACAAAGTA | | | | | |
| TOPBP1 | chr3 | 133361929 | 133361950 | CACTCATAAAT | 133361940 | A | G | CACTCATAAATGTAATAAAGAC | 179/830 |
| | | | | ATAATAAAGAC | | | | | |
| TOPBP1 | chr3 | 133362298 | 133362319 | ATTTAGTTTTG | 133362309 | A | G | ATTTAGTTTTGGTAAGAAGAAA | 180/831 |
| | | | | ATAAGAAGAAA | | | | | |
| TOPBP1 | chr3 | 133362819 | 133362840 | CAGATATCCTT | 133362830 | C | T | CAGATATCCTTTGATACTTTAT | 181/832 |
| | | | | CGATACTTTAT | | | | | |
| TOPBP1 | chr3 | 133368494 | 133368515 | TAGTTATGTAT | 133368505 | T | C | TAGTTATGTATCAGTGCAAGCT | 182/833 |
| | | | | TAGTGCAAGCT | | | | | |
| TOPBP1 | chr3 | 133368853 | 133368874 | TATATCTAAAT | 133368864 | C | A | TATATCTAAATACACTGTGTAT | 183/834 |
| | | | | CCACTGTGTAT | | | | | |
| TOPBP1 | chr3 | 133371460 | 133371481 | TGAAAGAGTAC | 133371471 | G | A | TGAAAGAGTACAACCTACATAT | 184/835 |
| | | | | GACCTACATAT | | | | | |
| TOPBP1 | chr3 | 133371562 | 133371583 | GGGCAATAAAC | 133371573 | A | C | GGGCAATAAACCACTTCCAAAG | 185/836 |
| | | | | AACTTCCAAAG | | | | | |
| TOPBP1 | chr3 | 133376813 | 133376834 | GTTAAGAAGGA | 133376824 | A | G | GTTAAGAAGGAGAAGACCACCA | 186/837 |
| | | | | AAAGACCACCA | | | | | |
| PCCB | chr3 | 135974675 | 135974696 | GAGTGATCTTT | 135974686 | G | T | GAGTGATCTTTTTTCCATTGTA | 187/838 |
| | | | | GTTCCATTGTA | | | | | |
| PCCB | chr3 | 136002781 | 136002802 | ATGGTAAAGGT | 136002792 | A | C | ATGGTAAAGGTCAGAAAGAAGG | 188/839 |
| | | | | AAGAAAGAAGG | | | | | |
| PCCB | chr3 | 136045944 | 136045965 | AGGATAACCAT | 136045955 | G | C | AGGATAACCATCTGAGGACTTG | 189/840 |
| | | | | GTGAGGACTTG | | | | | |
| PCCB | chr3 | 136047680 | 136047701 | TTTCCCTGCAG | 136047691 | C | T | TTTCCCTGCAGTAGTGCGAGGT | 190/841 |
| | | | | CAGTGCGAGGT | | | | | |
| ACTG1P1 | chr3 | 139213578 | 139213599 | TGGGAATTGCC | 139213589 | G | A | TGGGAATTGCCAACAGGATGCA | 191/842 |
| | | | | GACAGGATGCA | | | | | |
| SEC62 | chr3 | 169693423 | 169693444 | AAGGCTGTGGC | 169693434 | C | G | AAGGCTGTGGCGAAGTATCTTC | 192/843 |
| | | | | CAAGTATCTTC | | | | | |
| SEC62 | chr3 | 169693442 | 169693463 | TTCGATTCAAC | 169693453 | T | A | TTCGATTCAACAGTCCAACAAA | 193/844 |
| | | | | TGTCCAACAAA | | | | | |
| SEC62 | chr3 | 169700463 | 169700484 | GGAGTGTAATG | 169700474 | C | A | GGAGTGTAATGACATACCTGTT | 194/845 |
| | | | | CCATACCTGTT | | | | | |
| SEC62 | chr3 | 169703660 | 169703681 | AGTGAGATGTT | 169703671 | A | C | AGTGAGATGTTCATAGCTATAA | 195/846 |
| | | | | AATAGCTATAA | | | | | |
| SEC62 | chr3 | 169710603 | 169710624 | AAGGAAGATGA | 169710614 | G | C | AAGGAAGATGACGAGGGGAAAG | 196/847 |
| | | | | GGAGGGGAAAG | | | | | |
| SNORA48 | chr4 | 1112674 | 1112695 | TCCTTGGCCTG | 1112685 | G | A | TCCTTGGCCTGAGTAGAGTGGC | 197/848 |
| | | | | GGTAGAGTGGC | | | | | |
| SNORA48 | chr4 | 1112706 | 1112727 | TGGTGCCCATA | 1112717 | T | C | TGGTGCCCATACTAGCCAGGGA | 198/849 |
| | | | | TTAGCCAGGGA | | | | | |
| MAEA | chr4 | 1305788 | 1305809 | AAACGCTTTCG | 1305799 | C | T | AAACGCTTTCGTGCCGCTCAGA | 199/850 |
| | | | | CGCCGCTCAGA | | | | | |
| MAEA | chr4 | 1305791 | 1305812 | CGCTTTCGCGC | 1305802 | C | T | CGCTTTCGCGCTGCTCAGAAGA | 200/851 |
| | | | | CGCTCAGAAGA | | | | | |
| MAEA | chr4 | 1330796 | 1330817 | CGTGCGCAGTG | 1330807 | C | T | CGTGCGCAGTGTGGTTTGGCCT | 201/852 |
| | | | | CGGTTTGGCCT | | | | | |
| WHSC1 | chr4 | 1902568 | 1902589 | CAGAAGTTTAA | 1902579 | C | T | CAGAAGTTTAATGGCCACGACG | 202/853 |
| | | | | CGGCCACGACG | | | | | |
| WHSC1 | chr4 | 1902751 | 1902772 | ATTAAGCTGAA | 1902762 | A | G | ATTAAGCTGAAGATCACCAAAA | 203/854 |
| | | | | AATCACCAAAA | | | | | |
| WHSC1 | chr4 | 1902946 | 1902967 | GTGTCTAAGAT | 1902957 | C | T | GTGTCTAAGATTTCAAGTCCTT | 204/855 |
| | | | | CTCAAGTCCTT | | | | | |
| WHSC1 | chr4 | 1937005 | 1937026 | GTTTGCTTGAC | 1937016 | C | T | GTTTGCTTGACTTGTCAGAGTG | 205/856 |
| | | | | CTGTCAGAGTG | | | | | |
| WHSC1 | chr4 | 1954838 | 1954859 | CTGGAGCTCAG | 1954849 | A | G | CTGGAGCTCAGGTCGCAGCAAG | 206/857 |
| | | | | ATCGCAGCAAG | | | | | |
| WHSC1 | chr4 | 1955005 | 1955026 | TGTTCTTTGCA | 1955016 | C | T | TGTTCTTTGCATCTCTCTCTCC | 207/858 |
| | | | | CCTCTCTCTCC | | | | | |
| WHSC1 | chr4 | 1957837 | 1957858 | CGAGTGTTCCC | 1957848 | G | A | CGAGTGTTCCCATACATGGAGG | 208/859 |
| | | | | GTACATGGAGG | | | | | |
| WHSC1 | chr4 | 1976519 | 1976540 | TGGTGAAAATT | 1976530 | C | T | TGGTGAAAATTTCCTTTAAAAA | 209/860 |
| | | | | CCCTTTAAAAA | | | | | |
| WHSC1 | chr4 | 1976701 | 1976722 | GGCCTGTTTGC | 1976712 | C | T | GGCCTGTTTGCTGTCTGTGACA | 210/861 |
| | | | | CGTCTGTGACA | | | | | |
| WHSC1 | chr4 | 1980335 | 1980356 | TGTGTGCTCAC | 1980346 | A | T | TGTGTGCTCACTTCTTGTGTTC | 211/862 |
| | | | | ATCTTGTGTTC | | | | | |
| HNRNPD | chr4 | 83276397 | 83276418 | AATATAGATTA | 83276408 | T | C | AATATAGATTACAAAAACTACT | 212/863 |
| | | | | TAAAAACTACT | | | | | |
| HNRNPD | chr4 | 83279720 | 83279741 | CTTTGAAAAGC | 83279731 | C | T | CTTTGAAAAGCTAAGTGACTCT | 213/864 |
| | | | | CAAGTGACTCT | | | | | |
| FYB | chr5 | 39110403 | 39110424 | AAGAAAGGCAC | 39110414 | A | T | AAGAAAGGCACTAAATTCTTTT | 214/865 |
| | | | | AAAATTCTTTT | | | | | |
| FYB | chr5 | 39110452 | 39110473 | GCTTACTTGTC | 39110463 | C | T | GCTTACTTGTCTGCTAGGTAAC | 215/866 |
| | | | | CGCTAGGTAAC | | | | | |
| FYB | chr5 | 39110538 | 39110559 | AAATCTTTAGT | 39110549 | A | T | AAATCTTTAGTTCTTTTTTCAG | 216/867 |
| | | | | ACTTTTTTCAG | | | | | |
| FYB | chr5 | 39124409 | 39124430 | ATCAGACTAAC | 39124420 | A | G | ATCAGACTAACGTGAACACAGA | 217/868 |
| | | | | ATGAACACAGA | | | | | |
| FYB | chr5 | 39134406 | 39134427 | AAAGAATCATA | 39134417 | G | A | AAAGAATCATAATCAATCTCTA | 218/869 |
| | | | | GTCAATCTCTA | | | | | |
| FYB | chr5 | 39153551 | 39153572 | TTAGGTCAAAC | 39153562 | G | A | TTAGGTCAAACAGAGGTTTAAT | 219/870 |
| | | | | GGAGGTTTAAT | | | | | |
| FYB | chr5 | 39201888 | 39201909 | GAGTAAACCAT | 39201899 | A | G | GAGTAAACCATGCTGAATAGCA | 220/871 |
| | | | | ACTGAATAGCA | | | | | |
| FYB | chr5 | 39202642 | 39202663 | TGTGAAGAGAT | 39202653 | G | A | TGTGAAGAGATAGCTTGTTTCC | 221/872 |
| | | | | GGCTTGTTTCC | | | | | |
| PAIP1 | chr5 | 43533952 | 43533973 | TACAATAATGA | 43533963 | A | G | TACAATAATGAGGCATGGCTGA | 222/873 |
| | | | | AGCATGGCTGA | | | | | |
| PAIP1 | chr5 | 43556148 | 43556169 | TACAGCAACTT | 43556159 | G | C | TACAGCAACTTCCTATATACTG | 223/874 |
| | | | | GCTATATACTG | | | | | |
| PAIP1 | chr5 | 43556167 | 43556188 | CTGAAAAACCA | 43556178 | T | A | CTGAAAAACCAACTGAAAAGCG | 224/875 |
| | | | | TCTGAAAAGCG | | | | | |
| SMAD5 | chr5 | 135510006 | 135510027 | TATTTGGTTTC | 135510017 | A | G | TATTTGGTTTCGTTGTAATGAT | 225/876 |
| | | | | ATTGTAATGAT | | | | | |
| SMAD5 | chr5 | 135510067 | 135510088 | GTTCATCTGTA | 135510078 | C | T | GTTCATCTGTATTATGTTGGTG | 226/877 |
| | | | | CTATGTTGGTG | | | | | |
| MATR3 | chr5 | 138655185 | 138655206 | AGTCAGGTAAT | 138655196 | A | G | AGTCAGGTAATGTACATAAGGA | 227/878 |
| | | | | ATACATAAGGA | | | | | |
| HMP19 | chr5 | 173473774 | 173473795 | AGTAACCCCAG | 173473785 | C | T | AGTAACCCCAGTGAGAAGGGAA | 228/879 |
| | | | | CGAGAAGGGAA | | | | | |
| HMP19 | chr5 | 173534272 | 173534293 | AGCAGTTTGCT | 173534283 | G | A | AGCAGTTTGCTAAATGACCCCT | 229/880 |
| | | | | GAATGACCCCT | | | | | |
| HMP19 | chr5 | 173534413 | 173534434 | GTGGCCAAGCA | 173534424 | G | A | GTGGCCAAGCAAAGCACTGCCC | 230/881 |
| | | | | GAGCACTGCCC | | | | | |
| CDYL | chr6 | 4735022 | 4735043 | CCCAGCATCTC | 4735033 | C | T | CCCAGCATCTCTGTGAGCAGTG | 231/882 |
| | | | | CGTGAGCAGTG | | | | | |
| CDYL | chr6 | 4773230 | 4773251 | TGGGAAAAGAG | 4773241 | G | A | TGGGAAAAGAGAAGGATCTCAG | 232/883 |
| | | | | GAGGATCTCAG | | | | | |
| CDYL | chr6 | 4773427 | 4773448 | TTGCTGGTAGA | 4773438 | C | T | TTGCTGGTAGATGTGTCTTTTA | 233/884 |
| | | | | CGTGTCTTTTA | | | | | |
| CDYL | chr6 | 4892070 | 4892091 | GAATACATCCA | 4892081 | C | T | GAATACATCCATGACTTCAACA | 234/885 |
| | | | | CGACTTCAACA | | | | | |
| CDYL | chr6 | 4892370 | 4892391 | AAGAGCAGGAC | 4892381 | C | T | AAGAGCAGGACTGCAGTGGACG | 235/886 |
| | | | | CGCAGTGGACG | | | | | |
| HIVEP1 | chr6 | 12120273 | 12120294 | CATCTTTCGCC | 12120284 | G | A | CATCTTTCGCCATTCTTCATAG | 236/887 |
| | | | | GTTCTTCATAG | | | | | |
| HIVEP1 | chr6 | 12120624 | 12120645 | TACTGAAAGCA | 12120635 | A | G | TACTGAAAGCAGTGGAGCCAGA | 237/888 |
| | | | | ATGGAGCCAGA | | | | | |
| HIVEP1 | chr6 | 12120641 | 12120662 | CCAGAACTGAG | 12120652 | C | T | CCAGAACTGAGTACCTTGTCAC | 238/889 |
| | | | | CACCTTGTCAC | | | | | |
| HIVEP1 | chr6 | 12120833 | 12120854 | GCTCAGAAGAA | 12120844 | T | A | GCTCAGAAGAAAGAGCAAGGGG | 239/890 |
| | | | | TGAGCAAGGGG | | | | | |
| HIVEP1 | chr6 | 12121896 | 12121917 | ACCAGAAAGGC | 12121907 | G | A | ACCAGAAAGGCAACATGAATCC | 240/891 |
| | | | | GACATGAATCC | | | | | |
| HIVEP1 | chr6 | 12122091 | 12122112 | CACCAACTCCC | 12122102 | T | G | CACCAACTCCCGTTGCCAGAAG | 241/892 |
| | | | | TTTGCCAGAAG | | | | | |
| HIVEP1 | chr6 | 12122546 | 12122567 | AATTCCATGCC | 12122557 | G | A | AATTCCATGCCAACCACAGGTT | 242/893 |
| | | | | GACCACAGGTT | | | | | |
| HIVEP1 | chr6 | 12122689 | 12122710 | GCCCCAGAGTG | 12122700 | G | A | GCCCCAGAGTGAGCATCCCCGT | 243/894 |
| | | | | GGCATCCCCGT | | | | | |
| HIVEP1 | chr6 | 12123038 | 12123059 | GGCGGTCTGCA | 12123049 | G | A | GGCGGTCTGCAACCTCAGATTC | 244/895 |
| | | | | GCCTCAGATTC | | | | | |
| HIVEP1 | chr6 | 12123221 | 12123242 | GGCTGTAATCC | 12123232 | C | T | GGCTGTAATCCTAGTTTGCCTA | 245/896 |
| | | | | CAGTTTGCCTA | | | | | |
| HIVEP1 | chr6 | 12123527 | 12123548 | AGAACGGGGAA | 12123538 | G | T | AGAACGGGGAATTCCGGGTCTC | 246/897 |
| | | | | GTCCGGGTCTC | | | | | |
| HIVEP1 | chr6 | 12124181 | 12124202 | AGCAAATCATT | 12124192 | C | T | AGCAAATCATTTGATTGTGGAA | 247/898 |
| | | | | CGATTGTGGAA | | | | | |
| HIVEP1 | chr6 | 12124301 | 12124322 | AGGAGAGGCCC | 12124312 | A | G | AGGAGAGGCCCGCTGGTACGGC | 248/899 |
| | | | | ACTGGTACGGC | | | | | |
| HIVEP1 | chr6 | 12125221 | 12125242 | TGGTCGACTTT | 12125232 | C | T | TGGTCGACTTTTCCCTCAACAA | 249/900 |
| | | | | CCCCTCAACAA | | | | | |
| HIVEP1 | chr6 | 12125445 | 12125466 | AGCCCCTGGAC | 12125456 | G | A | AGCCCCTGGACAGTGTGATGTT | 250/901 |
| | | | | GGTGTGATGTT | | | | | |
| HIVEP1 | chr6 | 12125603 | 12125624 | GAAAACATCAA | 12125614 | G | A | GAAAACATCAAATCATCCACAT | 251/902 |
| | | | | GTCATCCACAT | | | | | |
| HIVEP1 | chr6 | 12125650 | 12125671 | ACCTGCTCCTT | 12125661 | C | T | ACCTGCTCCTTTAGAAAATACT | 252/903 |
| | | | | CAGAAAATACT | | | | | |
| HIVEP1 | chr6 | 12125682 | 12125703 | CTTTGAAATGT | 12125693 | A | G | CTTTGAAATGTGCAGACAATAA | 253/904 |
| | | | | ACAGACAATAA | | | | | |
| HIVEP1 | chr6 | 12126017 | 12126038 | AAATCACTATA | 12126028 | C | T | AAATCACTATATTGTCAAGCAA | 254/905 |
| | | | | CTGTCAAGCAA | | | | | |
| HIVEP1 | chr6 | 12161617 | 12161638 | GCACTTGTGCA | 12161628 | C | A | GCACTTGTGCAATTGGAAAGCA | 255/906 |
| | | | | CTTGGAAAGCA | | | | | |
| HIVEP1 | chr6 | 12161690 | 12161711 | GTTATGAGCGA | 12161701 | T | C | GTTATGAGCGACCTGGATATGA | 256/907 |
| | | | | TCTGGATATGA | | | | | |
| HIVEP1 | chr6 | 12163482 | 12163503 | TGTCATAAAAG | 12163493 | G | C | TGTCATAAAAGCATTGCTCTCT | 257/908 |
| | | | | GATTGCTCTCT | | | | | |
| HIVEP1 | chr6 | 12163510 | 12163531 | ATTTAGAGCCC | 12163521 | A | G | ATTTAGAGCCCGTCATCTGTAA | 258/909 |
| | | | | ATCATCTGTAA | | | | | |
| HIVEP1 | chr6 | 12163857 | 12163878 | TCATAGGAATA | 12163868 | C | T | TCATAGGAATATGGTCACAGAA | 259/910 |
| | | | | CGGTCACAGAA | | | | | |
| HIVEP1 | chr6 | 12164213 | 12164234 | GTCAAGTAGCC | 12164224 | G | A | GTCAAGTAGCCATTGATGCACA | 260/911 |
| | | | | GTTGATGCACA | | | | | |
| HIVEP1 | chr6 | 12164252 | 12164273 | CAGCTTCCCAA | 12164263 | A | G | CAGCTTCCCAAGGCAAAGCATG | 261/912 |
| | | | | AGCAAAGCATG | | | | | |
| HIVEP1 | chr6 | 12164601 | 12164622 | CAGGCCCACAG | 12164612 | C | G | CAGGCCCACAGGACTACCGCGG | 262/913 |
| | | | | CACTACCGCGG | | | | | |
| BTN1A1 | chr6 | 26505257 | 26505278 | AGAACTTCCAA | 26505268 | G | A | AGAACTTCCAAAGGAGAGAAGT | 263/914 |
| | | | | GGGAGAGAAGT | | | | | |
| BTN1A1 | chr6 | 26505507 | 26505528 | CAGATGTGACC | 26505518 | T | G | CAGATGTGACCGCATGGCAGAG | 264/915 |
| | | | | TCATGGCAGAG | | | | | |
| BTN1A1 | chr6 | 26508183 | 26508204 | CTCCTGGAAGA | 26508194 | A | C | CTCCTGGAAGACCTCAGTAAGT | 265/916 |
| | | | | ACTCAGTAAGT | | | | | |
| BTN1A1 | chr6 | 26508279 | 26508300 | TTTGTTGCAGA | 26508290 | A | G | TTTGTTGCAGAGTGGAAAAAGG | 266/917 |
| | | | | ATGGAAAAAGG | | | | | |
| BTN1A1 | chr6 | 26509371 | 26509392 | TCCCTACCCAA | 26509382 | C | T | TCCCTACCCAATCCAGCCAAGG | 267/918 |
| | | | | CCCAGCCAAGG | | | | | |
| BTN1A1 | chr6 | 26509381 | 26509402 | ACCCAGCCAAG | 26509392 | G | A | ACCCAGCCAAGAGGCACCTTAA | 268/919 |
| | | | | GGGCACCTTAA | | | | | |
| ZNF323 | chr6 | 28294297 | 28294318 | GTGGCTCCGCC | 28294308 | G | A | GTGGCTCCGCCAATGTTCATTC | 269/920 |
| | | | | GATGTTCATTC | | | | | |
| ZNF323 | chr6 | 28294488 | 28294509 | CCTTGCTGTCT | 28294499 | C | T | CCTTGCTGTCTTGTTTACAAGT | 270/921 |
| | | | | CGTTTACAAGT | | | | | |
| ZNF323 | chr6 | 28295140 | 28295161 | GGACCTGTCAA | 28295151 | A | T | GGACCTGTCAATATCCTTACCT | 271/922 |
| | | | | AATCCTTACCT | | | | | |
| ZNF323 | chr6 | 28295299 | 28295320 | ATGGTCTGGAG | 28295310 | C | G | ATGGTCTGGAGGCTGAAGGCAG | 272/923 |
| | | | | CCTGAAGGCAG | | | | | |
| ZNF323 | chr6 | 28297268 | 28297289 | GACAGAGTTCT | 28297279 | C | T | GACAGAGTTCTTGGAGCCGGCT | 273/924 |
| | | | | CGGAGCCGGCT | | | | | |
| ZNF323 | chr6 | 28297274 | 28297295 | GTTCTCGGAGC | 28297285 | C | T | GTTCTCGGAGCTGGCTCAGAGC | 274/925 |
| | | | | CGGCTCAGAGC | | | | | |
| ZNF323 | chr6 | 28297357 | 28297378 | TGGCCAGAAAA | 28297368 | G | A | TGGCCAGAAAAATTGTTCCCTC | 275/926 |
| | | | | GTTGTTCCCTC | | | | | |
| ZNF323 | chr6 | 28297372 | 28297393 | TTCCCTCGAAG | 28297383 | G | A | TTCCCTCGAAGATGGGTTTCTT | 276/927 |
| | | | | GTGGGTTTCTT | | | | | |
| HMGA1 | chr6 | 34211227 | 34211248 | TTTTCTCTAAC | 34211238 | C | T | TTTTCTCTAACTCTCTAGAAAA | 277/928 |
| | | | | CCTCTAGAAAA | | | | | |
| PXT1 | chr6 | 36368265 | 36368286 | ATGTGTCTCAG | 36368276 | C | T | ATGTGTCTCAGTTGCATGGCCA | 278/929 |
| | | | | CTGCATGGCCA | | | | | |
| GLP1R | chr6 | 39041513 | 39041534 | GGATCTTCAGG | 39041524 | C | T | GGATCTTCAGGTTCTACGTGAG | 279/930 |
| | | | | CTCTACGTGAG | | | | | |
| GLP1R | chr6 | 39041521 | 39041542 | AGGCTCTACGT | 39041532 | G | C | AGGCTCTACGTCAGCATAGGCT | 280/931 |
| | | | | GAGCATAGGCT | | | | | |
| GLP1R | chr6 | 39041591 | 39041612 | GAGACCTTGAC | 39041602 | C | T | GAGACCTTGACTCCTCTTCTAA | 281/932 |
| | | | | CCCTCTTCTAA | | | | | |
| GLP1R | chr6 | 39046717 | 39046738 | CTCTCTCCCTC | 39046728 | C | T | CTCTCTCCCTCTCCAGCTGCTG | 282/933 |
| | | | | CCCAGCTGCTG | | | | | |
| GLP1R | chr6 | 39046783 | 39046804 | CCATTCTCTTT | 39046794 | G | A | CCATTCTCTTTACCATTGGGGT | 283/934 |
| | | | | GCCATTGGGGT | | | | | |
| GLP1R | chr6 | 39046860 | 39046881 | CCTGCCAATCC | 39046871 | C | T | CCTGCCAATCCTCGGCCCCACC | 284/935 |
| | | | | CCGGCCCCACC | | | | | |
| GLP1R | chr6 | 39047407 | 39047428 | ATGGACGAGCA | 39047418 | C | T | ATGGACGAGCATGCCCGGGGGA | 285/936 |
| | | | | CGCCCGGGGGA | | | | | |
| SUPT3H | chr6 | 44921066 | 44921087 | TGAATGAAGGT | 44921077 | T | C | TGAATGAAGGTCGCAGAAATGG | 286/937 |
| | | | | TGCAGAAATGG | | | | | |
| SUPT3H | chr6 | 45073766 | 45073787 | CCAATATTAAG | 45073777 | G | T | CCAATATTAAGTACTATAAAAC | 287/938 |
| | | | | GACTATAAAAC | | | | | |
| SUPT3H | chr6 | 45290623 | 45290644 | AGTTGAAGAAC | 45290634 | G | A | AGTTGAAGAACATTGTTCCAAA | 288/939 |
| | | | | GTTGTTCCAAA | | | | | |
| SUPT3H | chr6 | 45296328 | 45296349 | ATATAAAGTCT | 45296339 | A | G | ATATAAAGTCTGTGTACTCCAG | 289/940 |
| | | | | ATGTACTCCAG | | | | | |
| SUPT3H | chr6 | 45332881 | 45332902 | TTTCAACATGA | 45332892 | A | G | TTTCAACATGAGTAAATTTAAA | 290/941 |
| | | | | ATAAATTTAAA | | | | | |
| RP3- | chr6 | 100584323 | 100584344 | GGGGCCGCTTT | 100584334 | A | G | GGGGCCGCTTTGTCAGTGATGG | 291/942 |
| 344J20.2 | | | | ATCAGTGATGG | | | | | |
| SOD2 | chr6 | 160134067 | 160134088 | GTCCAATTTCA | 160134078 | A | G | GTCCAATTTCAGTATGTACTCT | 292/943 |
| | | | | ATATGTACTCT | | | | | |
| SOD2 | chr6 | 160134518 | 160134539 | TCTTCAATGCC | 160134529 | G | A | TCTTCAATGCCATCTCAGCTAT | 293/944 |
| | | | | GTCTCAGCTAT | | | | | |
| SOD2 | chr6 | 160134748 | 160134769 | CCATTTCAACA | 160134759 | C | G | CCATTTCAACAGTATTAGACTT | 294/945 |
| | | | | CTATTAGACTT | | | | | |
| SOD2 | chr6 | 160163076 | 160163097 | ACTATTTTATC | 160163087 | G | A | ACTATTTTATCATAACAACTAA | 295/946 |
| | | | | GTAACAACTAA | | | | | |
| SOD2 | chr6 | 160163202 | 160163223 | AAAGACTGAAT | 160163213 | A | C | AAAGACTGAATCATCTCCTTTT | 296/947 |
| | | | | AATCTCCTTTT | | | | | |
| SOD2 | chr6 | 160174529 | 160174550 | CTTATCCAGGA | 160174540 | G | A | CTTATCCAGGAAAATCAAGAGC | 297/948 |
| | | | | GAATCAAGAGC | | | | | |
| SOD2 | chr6 | 160174729 | 160174750 | AGATTCTGTAC | 160174740 | A | G | AGATTCTGTACGTTGTTAACCT | 298/949 |
| | | | | ATTGTTAACCT | | | | | |
| MRPL18 | chr6 | 160219024 | 160219045 | TTCAGCCTACT | 160219035 | C | T | TTCAGCCTACTTGTGCTGCTGA | 299/950 |
| | | | | CGTGCTGCTGA | | | | | |
| HOXA3 | chr7 | 27148046 | 27148067 | CGGCTCCGGGG | 27148057 | G | T | CGGCTCCGGGGTGCACGGGGCT | 300/951 |
| | | | | GGCACGGGGCT | | | | | |
| HOXA3 | chr7 | 27155583 | 27155604 | AAGGTTTTTAT | 27155594 | T | C | AAGGTTTTTATCTGTTGGTTTG | 301/952 |
| | | | | TTGTTGGTTTG | | | | | |
| HOXA3 | chr7 | 27161582 | 27161603 | GAAGAACCGAT | 27161593 | G | C | GAAGAACCGATCATGAGCCCTG | 302/953 |
| | | | | GATGAGCCCTG | | | | | |
| TAF6 | chr7 | 99704881 | 99704902 | CTGAGGGGAGC | 99704892 | C | T | CTGAGGGGAGCTGGAGTTGGGC | 303/954 |
| | | | | CGGAGTTGGGC | | | | | |
| TAF6 | chr7 | 99708734 | 99708755 | AAATGACGCTC | 99708745 | A | C | AAATGACGCTCCAGTTTTCCTG | 304/955 |
| | | | | AAGTTTTCCTG | | | | | |
| TAF6 | chr7 | 99709303 | 99709324 | CAGCAGGGTCC | 99709314 | C | T | CAGCAGGGTCCTTAGTCCCTGG | 305/956 |
| | | | | CTAGTCCCTGG | | | | | |
| TAF6 | chr7 | 99711459 | 99711480 | TCACCCCCCCC | 99711470 | C | A | TCACCCCCCCCACGCCTGTCTC | 306/957 |
| | | | | CCGCCTGTCTC | | | | | |
| TAF6 | chr7 | 99711461 | 99711482 | ACCCCCCCCCC | 99711472 | G | C | ACCCCCCCCCCCCCTGTCTCCC | 307/958 |
| | | | | GCCTGTCTCCC | | | | | |
| FOXP2 | chr7 | 114175609 | 114175630 | TATGGCCCAGC | 114175620 | T | A | TATGGCCCAGCAGTCTGTTTTT | 308/959 |
| | | | | TGTCTGTTTTT | | | | | |
| FOXP2 | chr7 | 114210798 | 114210819 | GTTATTAGCAC | 114210809 | A | T | GTTATTAGCACTAGCCTTAAGA | 309/960 |
| | | | | AAGCCTTAAGA | | | | | |
| FOXP2 | chr7 | 114268461 | 114268482 | GTAAAATGTGA | 114268472 | C | T | GTAAAATGTGATGTAAAAATTA | 310/961 |
| | | | | CGTAAAAATTA | | | | | |
| FOXP2 | chr7 | 114268775 | 114268796 | AGGTGTGCACT | 114268786 | T | C | AGGTGTGCACTCATTTTGAAAG | 311/962 |
| | | | | TATTTTGAAAG | | | | | |
| FOXP2 | chr7 | 114292348 | 114292369 | TTGTTAAATGC | 114292359 | T | A | TTGTTAAATGCACACTTAATGG | 312/963 |
| | | | | TCACTTAATGG | | | | | |
| FOXP2 | chr7 | 114298320 | 114298341 | CAGGTAGGATA | 114298331 | T | C | CAGGTAGGATACGAATGCTCAG | 313/964 |
| | | | | TGAATGCTCAG | | | | | |
| FOXP2 | chr7 | 114299577 | 114299598 | TAGTTAGTAAA | 114299588 | C | T | TAGTTAGTAAATCATTATTTTA | 314/965 |
| | | | | CCATTATTTTA | | | | | |
| FOXP2 | chr7 | 114299747 | 114299768 | GTTTTAAGATG | 114299758 | C | G | GTTTTAAGATGGCTACCACAGT | 315/966 |
| | | | | CCTACCACAGT | | | | | |
| FOXP2 | chr7 | 114299763 | 114299784 | CACAGTTCCTT | 114299774 | A | G | CACAGTTCCTTGCAGATAGCAC | 316/967 |
| | | | | ACAGATAGCAC | | | | | |
| FOXP2 | chr7 | 114302078 | 114302099 | ATATTATTTTT | 114302089 | G | A | ATATTATTTTTACCATTTTTTC | 317/968 |
| | | | | GCCATTTTTTC | | | | | |
| FOXP2 | chr7 | 114302254 | 114302275 | TAGTTTTGTAA | 114302265 | T | C | TAGTTTTGTAACCCTGTATCCT | 318/969 |
| | | | | TCCTGTATCCT | | | | | |
| FOXP2 | chr7 | 114304250 | 114304271 | TTAAAAGAAGA | 114304261 | T | C | TTAAAAGAAGACACATGTTTTA | 319/970 |
| | | | | TACATGTTTTA | | | | | |
| FOXP2 | chr7 | 114330050 | 114330071 | AATATTTGACA | 114330061 | A | G | AATATTTGACAGATTTTTACTG | 320/971 |
| | | | | AATTTTTACTG | | | | | |
| FEZF1 | chr7 | 121942433 | 121942454 | TATGGTTCTGA | 121942444 | A | G | TATGGTTCTGAGAAAAAAAATA | 321/972 |
| | | | | AAAAAAAAATA | | | | | |
| FEZF1 | chr7 | 121942838 | 121942859 | TCCTGGTTTGC | 121942849 | A | G | TCCTGGTTTGCGTACCTTTTTG | 322/973 |
| | | | | ATACCTTTTTG | | | | | |
| FEZF1 | chr7 | 121943377 | 121943398 | ACACACGTAGA | 121943388 | A | C | ACACACGTAGACCAGGTTAGCC | 323/974 |
| | | | | ACAGGTTAGCC | | | | | |
| FEZF1 | chr7 | 121943422 | 121943443 | CCGGGCAGAGA | 121943433 | G | C | CCGGGCAGAGACAGGGTAGGAG | 324/975 |
| | | | | GAGGGTAGGAG | | | | | |
| FEZF1 | chr7 | 121943670 | 121943691 | TCCGTTTTGCA | 121943681 | T | A | TCCGTTTTGCAATGTACTTGCC | 325/976 |
| | | | | TTGTACTTGCC | | | | | |
| PODXL | chr7 | 131189197 | 131189218 | TCCATCACTTC | 131189208 | C | T | TCCATCACTTCTAGTGTTGGGT | 326/977 |
| | | | | CAGTGTTGGGT | | | | | |
| PODXL | chr7 | 131191002 | 131191023 | CTTGGGGGGCC | 131191013 | G | A | CTTGGGGGGCCACTTACCTCCT | 327/978 |
| | | | | GCTTACCTCCT | | | | | |
| PODXL | chr7 | 131191450 | 131191471 | CAGTGAGATCA | 131191461 | A | G | CAGTGAGATCAGTTTCTCATCC | 328/979 |
| | | | | ATTTCTCATCC | | | | | |
| PODXL | chr7 | 131191511 | 131191532 | TCCGCGGGGAG | 131191522 | C | T | TCCGCGGGGAGTGTGACAGCAG | 329/980 |
| | | | | CGTGACAGCAG | | | | | |
| PODXL | chr7 | 131193728 | 131193749 | GAGGTTCAGGA | 131193739 | C | T | GAGGTTCAGGATGAGCTGCTTC | 330/981 |
| | | | | CGAGCTGCTTC | | | | | |
| PODXL | chr7 | 131194244 | 131194265 | CGGGCTCGTGG | 131194255 | G | C | CGGGCTCGTGGCCTGCACTGTC | 331/982 |
| | | | | GCTGCACTGTC | | | | | |
| PODXL | chr7 | 131195520 | 131195541 | TTTCAGAGCCA | 131195531 | T | C | TTTCAGAGCCACCACGCCTGGC | 332/983 |
| | | | | TCACGCCTGGC | | | | | |
| PODXL | chr7 | 131195906 | 131195927 | TGCACTTTTTG | 131195917 | T | G | TGCACTTTTTGGGCTCTTGGGG | 333/984 |
| | | | | TGCTCTTGGGG | | | | | |
| CLEC5A | chr7 | 141631496 | 141631517 | GGAAACTGTGG | 141631507 | C | T | GGAAACTGTGGTATGTTCACGT | 334/985 |
| | | | | CATGTTCACGT | | | | | |
| CLEC5A | chr7 | 141635750 | 141635771 | ACTGAAGAGGT | 141635761 | C | G | ACTGAAGAGGTGCAAGAAAGGA | 335/986 |
| | | | | CCAAGAAAGGA | | | | | |
| CLEC5A | chr7 | 141645137 | 141645158 | TTACCTGTTCC | 141645148 | A | G | TTACCTGTTCCGTAGCTCCTGG | 336/987 |
| | | | | ATAGCTCCTGG | | | | | |
| GIMAP7 | chr7 | 150217093 | 150217114 | ATCGTTCTGGT | 150217104 | A | G | ATCGTTCTGGTGGGGAAAACTG | 337/988 |
| | | | | AGGGAAAACTG | | | | | |
| GIMAP7 | chr7 | 150217139 | 150217160 | ACACCATCCTT | 150217150 | G | C | ACACCATCCTTCGAGAGGAAAT | 338/989 |
| | | | | GGAGAGGAAAT | | | | | |
| GIMAP7 | chr7 | 150217959 | 150217980 | TTCCTAATTTA | 150217970 | C | T | TTCCTAATTTATTGTGATTTGT | 339/990 |
| | | | | CTGTGATTTGT | | | | | |
| LYN | chr8 | 56860102 | 56860123 | TTTGTGCCCCA | 56860113 | T | C | TTTGTGCCCCACGAGGTTTGTT | 340/991 |
| | | | | TGAGGTTTGTT | | | | | |
| LYN | chr8 | 56863195 | 56863216 | TGCCGTGGAAC | 56863206 | A | T | TGCCGTGGAACTTAATATGCAG | 341/992 |
| | | | | ATAATATGCAG | | | | | |
| LYN | chr8 | 56864492 | 56864513 | TATAAACATTT | 56864503 | A | G | TATAAACATTTGCTTACACTTT | 342/993 |
| | | | | ACTTACACTTT | | | | | |
| LYN | chr8 | 56866281 | 56866302 | GACTGCGGCAG | 56866292 | G | A | GACTGCGGCAGATTGGACACTA | 343/994 |
| | | | | GTTGGACACTA | | | | | |
| LYN | chr8 | 56866411 | 56866432 | AGAAGATTGGA | 56866422 | G | A | AGAAGATTGGAAAAGGCTTGTA | 344/995 |
| | | | | GAAGGCTTGTA | | | | | |
| LYN | chr8 | 56866483 | 56866504 | CGGGAGTCCAT | 56866494 | C | T | CGGGAGTCCATTAAGTTGGTGA | 345/996 |
| | | | | CAAGTTGGTGA | | | | | |
| LYN | chr8 | 56866504 | 56866525 | AAAAGGCTTGG | 56866515 | C | T | AAAAGGCTTGGTGCTGGGCAGT | 346/997 |
| | | | | CGCTGGGCAGT | | | | | |
| LYN | chr8 | 56910917 | 56910938 | ATGGCATACAT | 56910928 | C | T | ATGGCATACATTGAGCGGAAGA | 347/998 |
| | | | | CGAGCGGAAGA | | | | | |
| RUNX1T1 | chr8 | 93003829 | 93003850 | CCAATCCCGTA | 93003840 | A | G | CCAATCCCGTAGGAAGTGAACA | 348/999 |
| | | | | AGAAGTGAACA | | | | | |
| RUNX1T1 | chr8 | 93004019 | 93004040 | TTATTTGGACT | 93004030 | G | A | TTATTTGGACTATACCGCTGGC | 349/1000 |
| | | | | GTACCGCTGGC | | | | | |
| RUNX1T1 | chr8 | 93029433 | 93029454 | AAGCCTGAAAT | 93029444 | G | A | AAGCCTGAAATAACTACTTACA | 350/1001 |
| | | | | GACTACTTACA | | | | | |
| RUNX1T1 | chr8 | 93029465 | 93029486 | GTAAATGAACT | 93029476 | G | C | GTAAATGAACTCGTTCTTGGAG | 351/1002 |
| | | | | GGTTCTTGGAG | | | | | |
| RUNX1T1 | chr8 | 93029594 | 93029615 | GGAGGATGCCA | 93029605 | C | T | GGAGGATGCCATCAGGAACATA | 352/1003 |
| | | | | CCAGGAACATA | | | | | |
| RUNX1T1 | chr8 | 93074900 | 93074921 | GGCGGCATCGC | 93074911 | C | T | GGCGGCATCGCTGGAGGCAGGG | 353/1004 |
| | | | | CGGAGGCAGGG | | | | | |
| RUNX1T1 | chr8 | 93088093 | 93088114 | AAAAAGAAAAT | 93088104 | C | A | AAAAAGAAAATATTTACAATTA | 354/1005 |
| | | | | CTTTACAATTA | | | | | |
| CYC1 | chr8 | 145151133 | 145151154 | GCTAAGGAGCT | 145151144 | G | C | GCTAAGGAGCTCGCTGCGGAGG | 355/1006 |
| | | | | GGCTGCGGAGG | | | | | |
| CYC1 | chr8 | 145151220 | 145151241 | GATGAGGCTCT | 145151231 | C | T | GATGAGGCTCTTGGTGGCAGGT | 356/1007 |
| | | | | CGGTGGCAGGT | | | | | |
| CYC1 | chr8 | 145151539 | 145151560 | CCCACCGGGGT | 145151550 | G | A | CCCACCGGGGTATCACTGCGGG | 357/1008 |
| | | | | GTCACTGCGGG | | | | | |
| RP11- | chr9 | 37477395 | 37477416 | CTAGGATTCCA | 37477406 | C | T | CTAGGATTCCATACAGACTGGC | 358/1009 |
| 405L18.2 | | | | CACAGACTGGC | | | | | |
| OR1J2 | chr9 | 125273424 | 125273445 | TTACATCAATG | 125273435 | G | A | TTACATCAATGACATATGACCG | 359/1010 |
| | | | | GCATATGACCG | | | | | |
| OR1J2 | chr9 | 125273567 | 125273588 | CTGACCCGGCT | 125273578 | G | A | CTGACCCGGCTATCTTTCTGTG | 360/1011 |
| | | | | GTCTTTCTGTG | | | | | |
| OR1J2 | chr9 | 125273624 | 125273645 | GCTGCCCTGCT | 125273635 | C | G | GCTGCCCTGCTGAAGCTGTCCT | 361/1012 |
| | | | | CAAGCTGTCCT | | | | | |
| OR1J2 | chr9 | 125273693 | 125273714 | GTGGTCATTAC | 125273704 | C | T | GTGGTCATTACTCTGCCATTCA | 362/1013 |
| | | | | CCTGCCATTCA | | | | | |
| GLE1 | chr9 | 131285078 | 131285099 | GGAAGTAATGG | 131285089 | A | G | GGAAGTAATGGGGAAGAGGTGA | 363/1014 |
| | | | | AGAAGAGGTGA | | | | | |
| GLE1 | chr9 | 131287562 | 131287583 | CCAGAGCCTGC | 131287573 | G | A | CCAGAGCCTGCAAAGACAAGAG | 364/1015 |
| | | | | GAAGACAAGAG | | | | | |
| GLE1 | chr9 | 131295835 | 131295856 | CTCTGGAAAAC | 131295846 | C | G | CTCTGGAAAACGTGTTCAATCT | 365/1016 |
| | | | | CTGTTCAATCT | | | | | |
| GLE1 | chr9 | 131298707 | 131298728 | ATCCGTCTCTA | 131298718 | C | T | ATCCGTCTCTATGCTGCTATCA | 366/1017 |
| | | | | CGCTGCTATCA | | | | | |
| FAM188A | chr10 | 15828641 | 15828662 | ATTTATACTAC | 15828652 | G | A | ATTTATACTACAGGGAGAAAGA | 367/1018 |
| | | | | GGGGAGAAAGA | | | | | |
| FAM188A | chr10 | 15831350 | 15831371 | ATAAGAATTTA | 15831361 | C | G | ATAAGAATTTAGTTCAAGAAAT | 368/1019 |
| | | | | CTTCAAGAAAT | | | | | |
| FAM188A | chr10 | 15858805 | 15858826 | CTTAAAAAAAA | 15858816 | A | G | CTTAAAAAAAAGTCCTGCTATA | 369/1020 |
| | | | | ATCCTGCTATA | | | | | |
| FAM188A | chr10 | 15858933 | 15858954 | AATAAAACAAA | 15858944 | T | C | AATAAAACAAACAAACAAATTA | 370/1021 |
| | | | | TAAACAAATTA | | | | | |
| FAM188A | chr10 | 15883629 | 15883650 | GTTTCCTCTGG | 15883640 | A | G | GTTTCCTCTGGGAAAAAAAAAA | 371/1022 |
| | | | | AAAAAAAAAAA | | | | | |
| FAM188A | chr10 | 15885282 | 15885303 | TTAAAGCAATA | 15885293 | A | G | TTAAAGCAATAGTTAGTGAAAT | 372/1023 |
| | | | | ATTAGTGAAAT | | | | | |
| GPR158 | chr10 | 25684901 | 25684922 | GATTCTATCAT | 25684912 | C | A | GATTCTATCATACTGGAGTCTT | 373/1024 |
| | | | | CCTGGAGTCTT | | | | | |
| GPR158 | chr10 | 25861621 | 25861642 | TATATGACTGG | 25861632 | C | T | TATATGACTGGTGGACGGGTCA | 374/1025 |
| | | | | CGGACGGGTCA | | | | | |
| GPR158 | chr10 | 25861625 | 25861646 | TGACTGGCGGA | 25861636 | C | T | TGACTGGCGGATGGGTCATGAG | 375/1026 |
| | | | | CGGGTCATGAG | | | | | |
| GPR158 | chr10 | 25885506 | 25885527 | AGATATAGATA | 25885517 | T | C | AGATATAGATACATGCAATGCG | 376/1027 |
| | | | | TATGCAATGCG | | | | | |
| GPR158 | chr10 | 25886716 | 25886737 | CTCTATGCCCA | 25886727 | A | G | CTCTATGCCCAGCTGGAAATAT | 377/1028 |
| | | | | ACTGGAAATAT | | | | | |
| GPR158 | chr10 | 25887027 | 25887048 | GAGAGACCAAA | 25887038 | C | G | GAGAGACCAAAGGGAAGAGTCC | 378/1029 |
| | | | | CGGAAGAGTCC | | | | | |
| RP11- | chr10 | 38536871 | 38536892 | ATGTTTGCCCT | 38536882 | G | A | ATGTTTGCCCTAGTGTGCTGCT | 379/1030 |
| 672F9.1 | | | | GGTGTGCTGCT | | | | | |
| KCNMA1 | chr10 | 78637578 | 78637599 | GGTACTCATGG | 78637589 | G | T | GGTACTCATGGTCTTGATTTGA | 380/1031 |
| | | | | GCTTGATTTGA | | | | | |
| KCNMA1 | chr10 | 78643347 | 78643368 | CCCAAAGCAAG | 78643358 | T | C | CCCAAAGCAAGCTGGACTAAAT | 381/1032 |
| | | | | TTGGACTAAAT | | | | | |
| KCNMA1 | chr10 | 78649215 | 78649236 | GTGCACTGACT | 78649226 | G | A | GTGCACTGACTAGGGGTGCTGA | 382/1033 |
| | | | | GGGGGTGCTGA | | | | | |
| KCNMA1 | chr10 | 78649333 | 78649354 | AGACAGCAAAA | 78649344 | G | T | AGACAGCAAAATAACAGAGAGA | 383/1034 |
| | | | | GAACAGAGAGA | | | | | |
| KCNMA1 | chr10 | 78651374 | 78651395 | CCTCTAAGGGC | 78651385 | G | A | CCTCTAAGGGCATTTTCCTCAG | 384/1035 |
| | | | | GTTTTCCTCAG | | | | | |
| KCNMA1 | chr10 | 78651419 | 78651440 | GTGGCTCCTCC | 78651430 | G | A | GTGGCTCCTCCAGTCACCAGGG | 385/1036 |
| | | | | GGTCACCAGGG | | | | | |
| KCNMA1 | chr10 | 78669713 | 78669734 | GGATAACTCAC | 78669724 | C | T | GGATAACTCACTGCGCTCATGA | 386/1037 |
| | | | | CGCGCTCATGA | | | | | |
| KCNMA1 | chr10 | 78674610 | 78674631 | TAAGAAATAAA | 78674621 | C | A | TAAGAAATAAAACAACCTCCTC | 387/1038 |
| | | | | CCAACCTCCTC | | | | | |
| KCNMA1 | chr10 | 78704584 | 78704605 | ATGTTGAGTGA | 78704595 | C | T | ATGTTGAGTGATGCCAAGATGC | 388/1039 |
| | | | | CGCCAAGATGC | | | | | |
| KCNMA1 | chr10 | 78709072 | 78709093 | ATGCAGACCAC | 78709083 | G | A | ATGCAGACCACAACATGGCCAC | 389/1040 |
| | | | | GACATGGCCAC | | | | | |
| KCNMA1 | chr10 | 78713515 | 78713536 | TGGGGCAGAAG | 78713526 | C | T | TGGGGCAGAAGTGGGCAACATC | 390/1041 |
| | | | | CGGGCAACATC | | | | | |
| KCNMA1 | chr10 | 78737284 | 78737305 | GATATTAACTC | 78737295 | G | A | GATATTAACTCACTGACCTTTG | 391/1042 |
| | | | | GCTGACCTTTG | | | | | |
| KCNMA1 | chr10 | 78782783 | 78782804 | CAATTGGCTAG | 78782794 | C | T | CAATTGGCTAGTGGGGCTCAGT | 392/1043 |
| | | | | CGGGGCTCAGT | | | | | |
| KCNMA1 | chr10 | 78833059 | 78833080 | TTGTTTAACAT | 78833070 | T | C | TTGTTTAACATCTCTTCTGGGA | 393/1044 |
| | | | | TTCTTCTGGGA | | | | | |
| KCNMA1 | chr10 | 78839373 | 78839394 | AATTTTACTGT | 78839384 | C | A | AATTTTACTGTATCCAAATGGG | 394/1045 |
| | | | | CTCCAAATGGG | | | | | |
| KCNMA1 | chr10 | 78844329 | 78844350 | CCAAAAGGGCC | 78844340 | G | A | CCAAAAGGGCCATGAACAGCCA | 395/1046 |
| | | | | GTGAACAGCCA | | | | | |
| KCNMA1 | chr10 | 78850309 | 78850330 | ACAGGACCCTG | 78850320 | C | G | ACAGGACCCTGGATCCCACCCC | 396/1047 |
| | | | | CATCCCACCCC | | | | | |
| KCNMA1 | chr10 | 78868193 | 78868214 | AGGATTCTACC | 78868204 | G | A | AGGATTCTACCACAGCAGAGGC | 397/1048 |
| | | | | GCAGCAGAGGC | | | | | |
| KCNMA1 | chr10 | 78872066 | 78872087 | ACTCAGAGAGG | 78872077 | G | T | ACTCAGAGAGGTTCTTGTTGCA | 398/1049 |
| | | | | GTCTTGTTGCA | | | | | |
| KCNMA1 | chr10 | 78872270 | 78872291 | GTAACAGCACC | 78872281 | C | T | GTAACAGCACCTGCTTAGCAGG | 399/1050 |
| | | | | CGCTTAGCAGG | | | | | |
| KCNMA1 | chr10 | 78943080 | 78943101 | CATTGTTTGTA | 78943091 | G | A | CATTGTTTGTAAGGAGACAGCC | 400/1051 |
| | | | | GGGAGACAGCC | | | | | |
| KCNMA1 | chr10 | 78944752 | 78944773 | GTAGTGCTTAG | 78944763 | G | A | GTAGTGCTTAGAGTAGACGATG | 401/1052 |
| | | | | GGTAGACGATG | | | | | |
| VAX1 | chr10 | 118891904 | 118891925 | CAAAACATTCA | 118891915 | G | C | CAAAACATTCACAACAAAGTTA | 402/1053 |
| | | | | GAACAAAGTTA | | | | | |
| VAX1 | chr10 | 118891983 | 118892004 | ATTTGCCTAGA | 118891994 | A | G | ATTTGCCTAGAGAAAAAAAAAA | 403/1054 |
| | | | | AAAAAAAAAAA | | | | | |
| OR5P3 | chr11 | 7846956 | 7846977 | AGCAAGCTTCA | 7846967 | A | G | AGCAAGCTTCAGAAGTGGTGAA | 404/1055 |
| | | | | AAAGTGGTGAA | | | | | |
| OR5P3 | chr11 | 7847118 | 7847139 | GGTAGAGTAGA | 7847129 | G | A | GGTAGAGTAGAACAGGGGTGAG | 405/1056 |
| | | | | GCAGGGGTGAG | | | | | |
| HIPK3 | chr11 | 33308068 | 33308089 | GGAAAGAAACT | 33308079 | A | G | GGAAAGAAACTGTCCACGGACC | 406/1057 |
| | | | | ATCCACGGACC | | | | | |
| HIPK3 | chr11 | 33308090 | 33308111 | TATGTGAATGG | 33308101 | T | C | TATGTGAATGGCAGAAACTTTG | 407/1058 |
| | | | | TAGAAACTTTG | | | | | |
| HIPK3 | chr11 | 33308258 | 33308279 | CAGCAAGCTCA | 33308269 | C | T | CAGCAAGCTCATGTGCAGGCAC | 408/1059 |
| | | | | CGTGCAGGCAC | | | | | |
| HIPK3 | chr11 | 33308278 | 33308299 | ACCTCAGATTG | 33308289 | G | A | ACCTCAGATTGAGGCGTGGCGA | 409/1060 |
| | | | | GGGCGTGGCGA | | | | | |
| HIPK3 | chr11 | 33308458 | 33308479 | AGCTACCACAG | 33308469 | G | A | AGCTACCACAGAATCAAAACAG | 410/1061 |
| | | | | GATCAAAACAG | | | | | |
| HIPK3 | chr11 | 33350070 | 33350091 | TATTGGGGTTG | 33350081 | C | A | TATTGGGGTTGACATTTTGTGA | 411/1062 |
| | | | | CCATTTTGTGA | | | | | |
| HIPK3 | chr11 | 33362676 | 33362697 | AAGAATGTGTA | 33362687 | G | A | AAGAATGTGTAATAATTAATAA | 412/1063 |
| | | | | GTAATTAATAA | | | | | |
| HIPK3 | chr11 | 33363046 | 33363067 | TATTTATACAG | 33363057 | C | T | TATTTATACAGTGTGTATATTT | 413/1064 |
| | | | | CGTGTATATTT | | | | | |
| HIPK3 | chr11 | 33363047 | 33363068 | ATTTATACAGC | 33363058 | G | A | ATTTATACAGCATGTATATTTC | 414/1065 |
| | | | | GTGTATATTTC | | | | | |
| HIPK3 | chr11 | 33369674 | 33369695 | TTACAAACACT | 33369685 | A | G | TTACAAACACTGAGCCAGCTCC | 415/1066 |
| | | | | AAGCCAGCTCC | | | | | |
| HIPK3 | chr11 | 33369790 | 33369811 | TTGCCCTTTTG | 33369801 | A | T | TTGCCCTTTTGTTTTATTATCT | 416/1067 |
| | | | | ATTTATTATCT | | | | | |
| HIPK3 | chr11 | 33370867 | 33370888 | AGTAAGTCTAC | 33370878 | T | A | AGTAAGTCTACAAAAAAGCCTA | 417/1068 |
| | | | | TAAAAAGCCTA | | | | | |
| HIPK3 | chr11 | 33373246 | 33373267 | GCACTTTTGTG | 33373257 | G | A | GCACTTTTGTGAAGGACACTCA | 418/1069 |
| | | | | GAGGACACTCA | | | | | |
| HIPK3 | chr11 | 33373820 | 33373841 | ATTTGTGGATA | 33373831 | T | C | ATTTGTGGATACGTAGGAGTCT | 419/1070 |
| | | | | TGTAGGAGTCT | | | | | |
| HIPK3 | chr11 | 33374836 | 33374857 | TCAGCCACCCT | 33374847 | C | T | TCAGCCACCCTTAGTAGTGCTG | 420/1071 |
| | | | | CAGTAGTGCTG | | | | | |
| OR5F1 | chr11 | 55761240 | 55761261 | GAGGATTCAAC | 55761251 | A | G | GAGGATTCAACGTGGGAATCAC | 421/1072 |
| | | | | ATGGGAATCAC | | | | | |
| OR5F1 | chr11 | 55761856 | 55761877 | CAGCATCTTTG | 55761867 | G | A | CAGCATCTTTGAGGTGATGGTA | 422/1073 |
| | | | | GGGTGATGGTA | | | | | |
| MTA2 | chr11 | 62362040 | 62362061 | AGCTGGTTTCT | 62362051 | G | A | AGCTGGTTTCTATTGATCGGTG | 423/1074 |
| | | | | GTTGATCGGTG | | | | | |
| MTA2 | chr11 | 62363462 | 62363483 | CCAGTGCCCCC | 62363473 | G | A | CCAGTGCCCCCATAACTCACTG | 424/1075 |
| | | | | GTAACTCACTG | | | | | |
| MTA2 | chr11 | 62364269 | 62364290 | TTCCTTTGCAA | 62364280 | G | A | TTCCTTTGCAAAGTATCCATGG | 425/1076 |
| | | | | GGTATCCATGG | | | | | |
| MTA2 | chr11 | 62365460 | 62365481 | AACCTGGTAGC | 62365471 | C | T | AACCTGGTAGCTGTACAGTCTT | 426/1077 |
| | | | | CGTACAGTCTT | | | | | |
| C11orf2 | chr11 | 64876379 | 64876400 | ACTTCCGGGTA | 64876390 | C | T | ACTTCCGGGTATGCCTCCTCTT | 427/1078 |
| | | | | CGCCTCCTCTT | | | | | |
| C11orf2 | chr11 | 64877008 | 64877029 | TGGGAATGCAG | 64877019 | A | G | TGGGAATGCAGGTGGCTGGACA | 428/1079 |
| | | | | ATGGCTGGACA | | | | | |
| FOLR3 | chr11 | 71850119 | 71850140 | CCACCTGCAAG | 71850130 | C | T | CCACCTGCAAGTGCCACTTTAT | 429/1080 |
| | | | | CGCCACTTTAT | | | | | |
| FOLR3 | chr11 | 71850141 | 71850162 | CCAGGACAGCT | 71850152 | G | A | CCAGGACAGCTATCTCTGAGTG | 430/1081 |
| | | | | GTCTCTGAGTG | | | | | |
| FOLR3 | chr11 | 71850183 | 71850204 | GGATCCGGCAG | 71850194 | G | A | GGATCCGGCAGATATGAGTGCT | 431/1082 |
| | | | | GTATGAGTGCT | | | | | |
| FOLR3 | chr11 | 71850720 | 71850741 | CACTCCTTCAA | 71850731 | G | A | CACTCCTTCAAAGTCAGCAACT | 432/1083 |
| | | | | GGTCAGCAACT | | | | | |
| UVRAG | chr11 | 75591155 | 75591176 | TTCTGATTCTG | 75591166 | C | T | TTCTGATTCTGTGTTTCCTATT | 433/1084 |
| | | | | CGTTTCCTATT | | | | | |
| UVRAG | chr11 | 75694538 | 75694559 | AATTGCATTAC | 75694549 | A | C | AATTGCATTACCAGACAAAGGT | 434/1085 |
| | | | | AAGACAAAGGT | | | | | |
| UVRAG | chr11 | 75715133 | 75715154 | GGTAAATGCAC | 75715144 | A | G | GGTAAATGCACGCTGAGAAGAA | 435/1086 |
| | | | | ACTGAGAAGAA | | | | | |
| UVRAG | chr11 | 75851708 | 75851729 | TGAGTTCTGAA | 75851719 | G | A | TGAGTTCTGAAATCCAAAGTAA | 436/1087 |
| | | | | GTCCAAAGTAA | | | | | |
| UVRAG | chr11 | 75851803 | 75851824 | CTCCATATTTG | 75851814 | G | T | CTCCATATTTGTGGGTGCAGAT | 437/1088 |
| | | | | GGGGTGCAGAT | | | | | |
| UVRAG | chr11 | 75851876 | 75851897 | GCCAGCTCTGA | 75851887 | G | A | GCCAGCTCTGAAAATGAGAGAC | 438/1089 |
| | | | | GAATGAGAGAC | | | | | |
| UVRAG | chr11 | 75851926 | 75851947 | CAACTCAGCAT | 75851937 | T | C | CAACTCAGCATCAGCCCAGCCT | 439/1090 |
| | | | | TAGCCCAGCCT | | | | | |
| UVRAG | chr11 | 75852437 | 75852458 | CGCAGGAGTTC | 75852448 | C | T | CGCAGGAGTTCTGATAAGTGAA | 440/1091 |
| | | | | CGATAAGTGAA | | | | | |
| OR8B4 | chr11 | 124294202 | 124294223 | GTGCAGGAGAG | 124294213 | C | A | GTGCAGGAGAGATGCAAGAGGG | 441/1092 |
| | | | | CTGCAAGAGGG | | | | | |
| PPFIBP1 | chr12 | 27746290 | 27746311 | CAGGCCTATCC | 27746301 | C | T | CAGGCCTATCCTTTCCTATCCT | 442/1093 |
| | | | | CTTCCTATCCT | | | | | |
| PPFIBP1 | chr12 | 27802921 | 27802942 | TGTATCTGTTA | 27802932 | A | C | TGTATCTGTTACATTATAATAG | 443/1094 |
| | | | | AATTATAATAG | | | | | |
| PPFIBP1 | chr12 | 27811869 | 27811890 | CTCACCAAAGA | 27811880 | T | C | CTCACCAAAGACGTAAAGTTGC | 444/1095 |
| | | | | TGTAAAGTTGC | | | | | |
| PPFIBP1 | chr12 | 27829336 | 27829357 | TTCACTATTCT | 27829347 | T | C | TTCACTATTCTCATTTGCCTCT | 445/1096 |
| | | | | TATTTGCCTCT | | | | | |
| PPFIBP1 | chr12 | 27830104 | 27830125 | TGATCTCTAGA | 27830115 | A | C | TGATCTCTAGACAGCGATCTGA | 446/1097 |
| | | | | AAGCGATCTGA | | | | | |
| PPFIBP1 | chr12 | 27832541 | 27832562 | AAATCCAGAGG | 27832552 | T | C | AAATCCAGAGGCATCATGAAAC | 447/1098 |
| | | | | TATCATGAAAC | | | | | |
| PPFIBP1 | chr12 | 27832571 | 27832592 | AAGTAAGTAAA | 27832582 | G | A | AAGTAAGTAAAACAGTAAACAA | 448/1099 |
| | | | | GCAGTAAACAA | | | | | |
| PPFIBP1 | chr12 | 27840503 | 27840524 | CGAAACTAAGA | 27840514 | G | A | CGAAACTAAGAACCATTTTTCT | 449/1100 |
| | | | | GCCATTTTTCT | | | | | |
| PPFIBP1 | chr12 | 27841249 | 27841270 | GAAGTTCAGAA | 27841260 | G | C | GAAGTTCAGAACTGGACTAACC | 450/1101 |
| | | | | GTGGACTAACC | | | | | |
| PPFIBP1 | chr12 | 27841925 | 27841946 | CTTTTTTTTTT | 27841936 | T | C | CTTTTTTTTTTCTCTTTAAACA | 451/1102 |
| | | | | TTCTTTAAACA | | | | | |
| PPFIBP1 | chr12 | 27842041 | 27842062 | TTCAACCTTCT | 27842052 | G | A | TTCAACCTTCTAATTGGGGCTG | 452/1103 |
| | | | | GATTGGGGCTG | | | | | |
| RP11- | chr12 | 43963758 | 43963779 | TCCGTTTTCAT | 43963769 | A | G | TCCGTTTTCATGCTGCTCATTC | 453/1104 |
| 73B8.2 | | | | ACTGCTCATTC | | | | | |
| ATP5B | chr12 | 57032214 | 57032235 | GATGGGGGAGA | 57032225 | A | G | GATGGGGGAGAGAAAAAAAAAG | 454/1105 |
| | | | | AAAAAAAAAAG | | | | | |
| ATP5B | chr12 | 57037198 | 57037219 | CATCTTTTAAG | 57037209 | T | C | CATCTTTTAAGCTGATAACACC | 455/1106 |
| | | | | TTGATAACACC | | | | | |
| SPPL3 | chr12 | 121201398 | 121201419 | ACACTAGTTAC | 121201409 | T | G | ACACTAGTTACGCCCAGAAATC | 456/1107 |
| | | | | TCCCAGAAATC | | | | | |
| SPPL3 | chr12 | 121202906 | 121202927 | TTAAACATGAG | 121202917 | G | A | TTAAACATGAGACACACACAGC | 457/1108 |
| | | | | GCACACACAGC | | | | | |
| SPPL3 | chr12 | 121206338 | 121206359 | CTGCTTTCAGC | 121206349 | A | G | CTGCTTTCAGCGTCAGCCCTCC | 458/1109 |
| | | | | ATCAGCCCTCC | | | | | |
| SPPL3 | chr12 | 121221450 | 121221471 | TTTAAGAGGAA | 121221461 | A | G | TTTAAGAGGAAGGTCCCAAGTC | 459/1110 |
| | | | | AGTCCCAAGTC | | | | | |
| SPPL3 | chr12 | 121221507 | 121221528 | TACTGGCACAT | 121221518 | C | T | TACTGGCACATTGGGAGGAGAA | 460/1111 |
| | | | | CGGGAGGAGAA | | | | | |
| SPPL3 | chr12 | 121221564 | 121221585 | AGAAAAGGTAT | 121221575 | A | C | AGAAAAGGTATCATTTTTTAAA | 461/1112 |
| | | | | AATTTTTTAAA | | | | | |
| DDX55 | chr12 | 124090644 | 124090665 | GCTTTTGTCAT | 124090655 | C | T | GCTTTTGTCATTCCCATCCTGG | 462/1113 |
| | | | | CCCCATCCTGG | | | | | |
| DDX55 | chr12 | 124092022 | 124092043 | AAATAGACGAG | 124092033 | G | C | AAATAGACGAGCTCCTGTCGCA | 463/1114 |
| | | | | GTCCTGTCGCA | | | | | |
| DDX55 | chr12 | 124094401 | 124094422 | TTGAATACCTG | 124094412 | T | C | TTGAATACCTGCTTAGTATCGT | 464/1115 |
| | | | | TTTAGTATCGT | | | | | |
| DDX55 | chr12 | 124097872 | 124097893 | TTTTGGATTCC | 124097883 | A | T | TTTTGGATTCCTTCTAGCATGG | 465/1116 |
| | | | | ATCTAGCATGG | | | | | |
| DDX55 | chr12 | 124099617 | 124099638 | AGGGAGGGCTT | 124099628 | G | A | AGGGAGGGCTTATAGTTAGGTT | 466/1117 |
| | | | | GTAGTTAGGTT | | | | | |
| DDX55 | chr12 | 124099702 | 124099723 | ATGACTCTAAG | 124099713 | C | A | ATGACTCTAAGACCTCTGTCCC | 467/1118 |
| | | | | CCCTCTGTCCC | | | | | |
| DDX55 | chr12 | 124102318 | 124102339 | CGCTGCGGTCG | 124102329 | C | G | CGCTGCGGTCGGACAGCTCGCA | 468/1119 |
| | | | | CACAGCTCGCA | | | | | |
| DDX55 | chr12 | 124102345 | 124102366 | CACGGGGGCAG | 124102356 | C | T | CACGGGGGCAGTGCTCTGGTGT | 469/1120 |
| | | | | CGCTCTGGTGT | | | | | |
| DDX55 | chr12 | 124102883 | 124102904 | GAGGCACCTCG | 124102894 | G | A | GAGGCACCTCGATCATGGAGTG | 470/1121 |
| | | | | GTCATGGAGTG | | | | | |
| DDX55 | chr12 | 124104391 | 124104412 | AATTTGCTCTA | 124104402 | T | C | AATTTGCTCTACTTGCAGAAGC | 471/1122 |
| | | | | TTTGCAGAAGC | | | | | |
| DDX55 | chr12 | 124104710 | 124104731 | ACAAGGACATA | 124104721 | G | A | ACAAGGACATAACTGTTCCCTA | 472/1123 |
| | | | | GCTGTTCCCTA | | | | | |
| GTF2H3 | chr12 | 124139553 | 124139574 | GAGAGCCTGCC | 124139564 | G | A | GAGAGCCTGCCATTTAAAGTAT | 473/1124 |
| | | | | GTTTAAAGTAT | | | | | |
| GTF2H3 | chr12 | 124140391 | 124140412 | TTTGTGTCGGT | 124140402 | G | A | TTTGTGTCGGTAGTTATGACAA | 474/1125 |
| | | | | GGTTATGACAA | | | | | |
| GTF2H3 | chr12 | 124144139 | 124144160 | GACAGCAGCGG | 124144150 | C | T | GACAGCAGCGGTGACCCTGATG | 475/1126 |
| | | | | CGACCCTGATG | | | | | |
| GTF2H3 | chr12 | 124144348 | 124144369 | TTTCTTCCCGA | 124144359 | T | C | TTTCTTCCCGACCAAGATCAGA | 476/1127 |
| | | | | TCAAGATCAGA | | | | | |
| GTF2H3 | chr12 | 124144417 | 124144438 | GCTTGCTTCTG | 124144428 | T | C | GCTTGCTTCTGCCATCGAAATC | 477/1128 |
| | | | | TCATCGAAATC | | | | | |
| MIPEP | chr13 | 24304573 | 24304594 | TCTGAAGCATA | 24304584 | C | T | TCTGAAGCATATCTGCAAACAA | 478/1129 |
| | | | | CCTGCAAACAA | | | | | |
| MIPEP | chr13 | 24321676 | 24321697 | AGGGCAGGATA | 24321687 | G | A | AGGGCAGGATAAGAGTAAGAGA | 479/1130 |
| | | | | GGAGTAAGAGA | | | | | |
| MIPEP | chr13 | 24330740 | 24330761 | TAGCGCTCCCC | 24330751 | G | A | TAGCGCTCCCCAGCAGCCCTGG | 480/1131 |
| | | | | GGCAGCCCTGG | | | | | |
| MIPEP | chr13 | 24334356 | 24334377 | CCTGCCAAGAA | 24334367 | C | G | CCTGCCAAGAAGAGAGAGACAC | 481/1132 |
| | | | | CAGAGAGACAC | | | | | |
| MIPEP | chr13 | 24383971 | 24383992 | ACAAAGGTACA | 24383982 | T | C | ACAAAGGTACACACATACCTGA | 482/1133 |
| | | | | TACATACCTGA | | | | | |
| MIPEP | chr13 | 24410499 | 24410520 | AAAAAAAAAAA | 24410510 | A | G | AAAAAAAAAAAGGTTGGCATGA | 483/1134 |
| | | | | AGTTGGCATGA | | | | | |
| MIPEP | chr13 | 24411589 | 24411610 | TAAAATGATCA | 24411600 | C | T | TAAAATGATCATATTTTCCAAA | 484/1135 |
| | | | | CATTTTCCAAA | | | | | |
| MIPEP | chr13 | 24411865 | 24411886 | GTCTGCCTCCA | 24411876 | C | T | GTCTGCCTCCATGGATAGTGAA | 485/1136 |
| | | | | CGGATAGTGAA | | | | | |
| MIPEP | chr13 | 24443634 | 24443655 | GTCCTATGCAA | 24443645 | C | T | GTCCTATGCAATAATAACACAG | 486/1137 |
| | | | | CAATAACACAG | | | | | |
| MIPEP | chr13 | 24448987 | 24449008 | TTTCTTTGTCT | 24448998 | A | G | TTTCTTTGTCTGGATGGATTCC | 487/1138 |
| | | | | AGATGGATTCC | | | | | |
| AL | chr13 | 27894202 | 27894223 | ATGAGTATGTT | 27894213 | C | T | ATGAGTATGTTTCATGCAATAT | 488/1139 |
| 159977.1 | | | | CCATGCAATAT | | | | | |
| SERTM1 | chr13 | 37269188 | 37269209 | CCAGATCACTC | 37269199 | C | T | CCAGATCACTCTTTCACCCTCC | 489/1140 |
| | | | | CTTCACCCTCC | | | | | |
| TRIM13 | chr13 | 50586051 | 50586072 | ATTTTTTTTTT | 50586062 | T | C | ATTTTTTTTTTCTCTGGTAGGA | 490/1141 |
| | | | | TTCTGGTAGGA | | | | | |
| TRIM13 | chr13 | 50587094 | 50587115 | TTATTTGATGA | 50587105 | C | T | TTATTTGATGATCTGGCAACTT | 491/1142 |
| | | | | CCTGGCAACTT | | | | | |
| TRIM13 | chr13 | 50587129 | 50587150 | TTCAAACTTCA | 50587140 | G | C | TTCAAACTTCACTTCCTATCTG | 492/1143 |
| | | | | GTTCCTATCTG | | | | | |
| TRIM13 | chr13 | 50589555 | 50589576 | CTAGCAACATT | 50589566 | T | A | CTAGCAACATTAATGGTTATAG | 493/1144 |
| | | | | TATGGTTATAG | | | | | |
| SUGT1 | chr13 | 53238137 | 53238158 | CTTTCAACTTA | 53238148 | C | T | CTTTCAACTTATCAAAATCAAT | 494/1145 |
| | | | | CCAAAATCAAT | | | | | |
| SUGT1 | chr13 | 53239756 | 53239777 | TTTTTTTTTTT | 53239767 | A | T | TTTTTTTTTTTTATAGGTATGA | 495/1146 |
| | | | | AATAGGTATGA | | | | | |
| SUGT1 | chr13 | 53241080 | 53241101 | TAGTAATATTT | 53241091 | T | G | TAGTAATATTTGCAAAATTATA | 496/1147 |
| | | | | TCAAAATTATA | | | | | |
| SUGT1 | chr13 | 53262010 | 53262031 | TAATGCCCATT | 53262021 | G | A | TAATGCCCATTATGTATTGATA | 497/1148 |
| | | | | GTGTATTGATA | | | | | |
| EFNB2 | chr13 | 107145599 | 107145620 | GTGTGCTGCGG | 107145610 | C | T | GTGTGCTGCGGTGAGTGCTTCC | 498/1149 |
| | | | | CGAGTGCTTCC | | | | | |
| EFNB2 | chr13 | 107147359 | 107147380 | TGATTAATTAC | 107147370 | G | A | TGATTAATTACAGCACAGACAT | 499/1150 |
| | | | | GGCACAGACAT | | | | | |
| EFNB2 | chr13 | 107165064 | 107165085 | ATACTGGCCAA | 107165075 | C | T | ATACTGGCCAATAGTTTTAGAG | 500/1151 |
| | | | | CAGTTTTAGAG | | | | | |
| EFNB2 | chr13 | 107187284 | 107187305 | TTCCACACGGA | 107187295 | G | A | TTCCACACGGAATCCCTTCTCA | 501/1152 |
| | | | | GTCCCTTCTCA | | | | | |
| SCFD1 | chr14 | 31097383 | 31097404 | TGACAATATTT | 31097394 | G | T | TGACAATATTTTATAAAACTTT | 502/1153 |
| | | | | GATAAAACTTT | | | | | |
| SCFD1 | chr14 | 31099748 | 31099769 | AGACATGGGAA | 31099759 | T | C | AGACATGGGAACCACTCTGCAT | 503/1154 |
| | | | | TCACTCTGCAT | | | | | |
| SCFD1 | chr14 | 31099849 | 31099870 | TTTATGTAGTA | 31099860 | T | C | TTTATGTAGTACTGAACATTTT | 504/1155 |
| | | | | TTGAACATTTT | | | | | |
| SCFD1 | chr14 | 31107517 | 31107538 | CGTAGTTGGCA | 31107528 | T | G | CGTAGTTGGCAGAGATATCTAT | 505/1156 |
| | | | | TAGATATCTAT | | | | | |
| SCFD1 | chr14 | 31112716 | 31112737 | TAGAGGTTCTC | 31112727 | G | A | TAGAGGTTCTCAAGTGGAGTAA | 506/1157 |
| | | | | GAGTGGAGTAA | | | | | |
| SCFD1 | chr14 | 31143247 | 31143268 | GATTTTTTTTT | 31143258 | T | A | GATTTTTTTTTAATTTAACTAA | 507/1158 |
| | | | | TATTTAACTAA | | | | | |
| SCFD1 | chr14 | 31152500 | 31152521 | AGCAGGGTGTG | 31152511 | A | G | AGCAGGGTGTGGGGAATTTCAC | 508/1159 |
| | | | | AGGAATTTCAC | | | | | |
| SCFD1 | chr14 | 31164022 | 31164043 | TGAGCAAAACT | 31164033 | A | G | TGAGCAAAACTGCTCTGGATAA | 509/1160 |
| | | | | ACTCTGGATAA | | | | | |
| SCFD1 | chr14 | 31188372 | 31188393 | AATAAAGGTTA | 31188383 | G | C | AATAAAGGTTACCACATAGTAA | 510/1161 |
| | | | | GCACATAGTAA | | | | | |
| SCFD1 | chr14 | 31188386 | 31188407 | CATAGTAAGTG | 31188397 | C | T | CATAGTAAGTGTTCATTAAGTA | 511/1162 |
| | | | | CTCATTAAGTA | | | | | |
| SCFD1 | chr14 | 31188494 | 31188515 | TTCCACAGTCA | 31188505 | T | C | TTCCACAGTCACGGTAAAGTTC | 512/1163 |
| | | | | TGGTAAAGTTC | | | | | |
| SCFD1 | chr14 | 31204698 | 31204719 | AGTGTAATTTA | 31204709 | T | C | AGTGTAATTTACTAAGGGGTTT | 513/1164 |
| | | | | TTAAGGGGTTT | | | | | |
| SCFD1 | chr14 | 31204704 | 31204725 | ATTTATTAAGG | 31204715 | G | A | ATTTATTAAGGAGTTTACAAAT | 514/1165 |
| | | | | GGTTTACAAAT | | | | | |
| SCFD1 | chr14 | 31204710 | 31204731 | TAAGGGGTTTA | 31204721 | C | A | TAAGGGGTTTAAAAATATGTTT | 515/1166 |
| | | | | CAAATATGTTT | | | | | |
| SFTA3 | chr14 | 36946271 | 36946292 | AGGTGGGTATC | 36946282 | C | T | AGGTGGGTATCTGCTTTTCCCT | 516/1167 |
| | | | | CGCTTTTCCCT | | | | | |
| MIR345 | chr14 | 100774192 | 100774213 | AGAGACCCAAA | 100774203 | C | T | AGAGACCCAAATCCTAGGTCTG | 517/1168 |
| | | | | CCCTAGGTCTG | | | | | |
| IGHV3-50 | chr14 | 107022376 | 107022397 | CTGCACCCCAC | 107022387 | A | G | CTGCACCCCACGCTAGACACCT | 518/1169 |
| | | | | ACTAGACACCT | | | | | |
| IGHV3-50 | chr14 | 107022468 | 107022489 | ACTCCATATCT | 107022479 | C | T | ACTCCATATCTTAAGTTCTCCA | 519/1170 |
| | | | | CAAGTTCTCCA | | | | | |
| TMEM87A | chr15 | 42503943 | 42503964 | CGTTCCTAGGG | 42503954 | A | G | CGTTCCTAGGGGAAAAAAAAAA | 520/1171 |
| | | | | AAAAAAAAAAA | | | | | |
| TMEM87A | chr15 | 42528433 | 42528454 | GAACTACTACA | 42528444 | T | C | GAACTACTACACTGGGAACATG | 521/1172 |
| | | | | TTGGGAACATG | | | | | |
| TMEM87A | chr15 | 42529608 | 42529629 | CAAAGATTTTC | 42529619 | T | C | CAAAGATTTTCCGGTTACTTAC | 522/1173 |
| | | | | TGGTTACTTAC | | | | | |
| TMEM87A | chr15 | 42536172 | 42536193 | TAAAAAAGCAT | 42536183 | A | G | TAAAAAAGCATGTGTAAAGTAC | 523/1174 |
| | | | | ATGTAAAGTAC | | | | | |
| TMEM87A | chr15 | 42560307 | 42560328 | CTATTCATTTC | 42560318 | G | A | CTATTCATTTCATACCCTAAAC | 524/1175 |
| | | | | GTACCCTAAAC | | | | | |
| MLST8 | chr16 | 2258756 | 2258777 | CCAGCTTCCTC | 2258767 | G | A | CCAGCTTCCTCAGACAACCTGG | 525/1176 |
| | | | | GGACAACCTGG | | | | | |
| PALB2 | chr16 | 23619224 | 23619245 | CCCACGCTGAG | 23619235 | A | C | CCCACGCTGAGCGTCGTCTTAG | 526/1177 |
| | | | | AGTCGTCTTAG | | | | | |
| PALB2 | chr16 | 23634282 | 23634303 | TTTCTTACCCT | 23634293 | C | T | TTTCTTACCCTTCATCTTCTGC | 527/1178 |
| | | | | CCATCTTCTGC | | | | | |
| PALB2 | chr16 | 23635359 | 23635380 | AGCTACACACA | 23635370 | C | T | AGCTACACACATGAGATTATAC | 528/1179 |
| | | | | CGAGATTATAC | | | | | |
| PALB2 | chr16 | 23635380 | 23635401 | CACATCAGGCA | 23635391 | C | G | CACATCAGGCAGTGGAACTATC | 529/1180 |
| | | | | CTGGAACTATC | | | | | |
| PALB2 | chr16 | 23637745 | 23637766 | AAGTGGCACTC | 23637756 | G | C | AAGTGGCACTCCAGTGCTGTTT | 530/1181 |
| | | | | GAGTGCTGTTT | | | | | |
| PALB2 | chr16 | 23641208 | 23641229 | CAGCAAGTTCG | 23641219 | T | C | CAGCAAGTTCGCCCAGCAACTT | 531/1182 |
| | | | | TCCAGCAACTT | | | | | |
| PALB2 | chr16 | 23641450 | 23641471 | AAGGTCCTCTT | 23641461 | C | G | AAGGTCCTCTTGTAAGTCCTCC | 532/1183 |
| | | | | CTAAGTCCTCC | | | | | |
| PALB2 | chr16 | 23646250 | 23646271 | AACAATCGACA | 23646261 | G | A | AACAATCGACAAGCTAGAAGTT | 533/1184 |
| | | | | GGCTAGAAGTT | | | | | |
| PALB2 | chr16 | 23646284 | 23646305 | TCACAATGATC | 23646295 | T | C | TCACAATGATCCGATGCTGGGG | 534/1185 |
| | | | | TGATGCTGGGG | | | | | |
| PALB2 | chr16 | 23646846 | 23646867 | CATTTGCTGGT | 23646857 | A | G | CATTTGCTGGTGAGTTATTGTA | 535/1186 |
| | | | | AAGTTATTGTA | | | | | |
| PALB2 | chr16 | 23646931 | 23646952 | ACTTTTACTTA | 23646942 | T | C | ACTTTTACTTACAGCTTTATTT | 536/1187 |
| | | | | TAGCTTTATTT | | | | | |
| PALB2 | chr16 | 23646957 | 23646978 | GGAGGTTATCT | 23646968 | G | A | GGAGGTTATCTATAGAGACAGT | 537/1188 |
| | | | | GTAGAGACAGT | | | | | |
| PALB2 | chr16 | 23647135 | 23647156 | CCTGGTGAAAT | 23647146 | T | C | CCTGGTGAAATCAGGTCTTCTT | 538/1189 |
| | | | | TAGGTCTTCTT | | | | | |
| PALB2 | chr16 | 23647227 | 23647248 | TAACTGGTTCT | 23647238 | G | A | TAACTGGTTCTAGAGAATCTGG | 539/1190 |
| | | | | GGAGAATCTGG | | | | | |
| PALB2 | chr16 | 23647512 | 23647533 | GTATAGGTAAT | 23647523 | C | A | GTATAGGTAATACTCCTGGGCC | 540/1191 |
| | | | | CCTCCTGGGCC | | | | | |
| MT1DP | chr16 | 56678566 | 56678587 | GTCGGGGAGAT | 56678577 | C | T | GTCGGGGAGATTCCTGGTCAAG | 541/1192 |
| | | | | CCCTGGTCAAG | | | | | |
| CDH13 | chr16 | 82891891 | 82891912 | GTTGCGGATTT | 82891902 | G | C | GTTGCGGATTTCGCGAAAGTTA | 542/1193 |
| | | | | GGCGAAAGTTA | | | | | |
| CDH13 | chr16 | 82891916 | 82891937 | GGGCAAACACA | 82891927 | T | C | GGGCAAACACACAAGCCGCTAT | 543/1194 |
| | | | | TAAGCCGCTAT | | | | | |
| CDH13 | chr16 | 82892026 | 82892047 | GTTCCATATCA | 82892037 | A | G | GTTCCATATCAGTCAGCCAGCT | 544/1195 |
| | | | | ATCAGCCAGCT | | | | | |
| CDH13 | chr16 | 83065755 | 83065776 | ACCCCCCATGC | 83065766 | G | A | ACCCCCCATGCAGAAGATATGG | 545/1196 |
| | | | | GGAAGATATGG | | | | | |
| CDH13 | chr16 | 83065780 | 83065801 | AACTCGTGATT | 83065791 | G | A | AACTCGTGATTATCGGGGGGAA | 546/1197 |
| | | | | GTCGGGGGGAA | | | | | |
| CDH13 | chr16 | 83065829 | 83065850 | ACATCTGTTTG | 83065840 | A | T | ACATCTGTTTGTGATAACTTGG | 547/1198 |
| | | | | AGATAACTTGG | | | | | |
| CDH13 | chr16 | 83158867 | 83158888 | GGAATACAGTG | 83158878 | A | G | GGAATACAGTGGACACTTTCCA | 548/1199 |
| | | | | AACACTTTCCA | | | | | |
| CDH13 | chr16 | 83159144 | 83159165 | TTATGAAAAGA | 83159155 | T | C | TTATGAAAAGACGAGCACAGCA | 549/1200 |
| | | | | TGAGCACAGCA | | | | | |
| CDH13 | chr16 | 83251099 | 83251120 | TCAAGTGAGTA | 83251110 | C | T | TCAAGTGAGTATCCCTCTCCCA | 550/1201 |
| | | | | CCCCTCTCCCA | | | | | |
| CDH13 | chr16 | 83520153 | 83520174 | AATATCCGTCA | 83520164 | G | A | AATATCCGTCAACAGACGCCTG | 551/1202 |
| | | | | GCAGACGCCTG | | | | | |
| CDH13 | chr16 | 83520287 | 83520308 | TTTCACGAGAA | 83520298 | T | C | TTTCACGAGAACAGAATGTGGC | 552/1203 |
| | | | | TAGAATGTGGC | | | | | |
| CDH13 | chr16 | 83520331 | 83520352 | GGCTCCAGTCA | 83520342 | G | A | GGCTCCAGTCAATGGTTTTTTT | 553/1204 |
| | | | | GTGGTTTTTTT | | | | | |
| CDH13 | chr16 | 83636186 | 83636207 | TCACCAAGAAA | 83636197 | G | C | TCACCAAGAAACAGGTAAACCC | 554/1205 |
| | | | | GAGGTAAACCC | | | | | |
| CDH13 | chr16 | 83704408 | 83704429 | TCGAGGAAGGA | 83704419 | G | A | TCGAGGAAGGAACTGTGGGAGT | 555/1206 |
| | | | | GCTGTGGGAGT | | | | | |
| CDH13 | chr16 | 83711876 | 83711897 | CTCGTACCCGA | 83711887 | C | T | CTCGTACCCGATGTCTCCTACG | 556/1207 |
| | | | | CGTCTCCTACG | | | | | |
| CDH13 | chr16 | 83711933 | 83711954 | CTGGATGTCAA | 83711944 | C | T | CTGGATGTCAATGAGGGCCCAG | 557/1208 |
| | | | | CGAGGGCCCAG | | | | | |
| CDH13 | chr16 | 83781984 | 83782005 | TAAATGTTTAA | 83781995 | A | C | TAAATGTTTAACTATACACATG | 558/1209 |
| | | | | ATATACACATG | | | | | |
| CDH13 | chr16 | 83816860 | 83816881 | TACACACGCCC | 83816871 | T | G | TACACACGCCCGGGTAAGCCTT | 559/1210 |
| | | | | TGGTAAGCCTT | | | | | |
| CDH13 | chr16 | 83828673 | 83828694 | ATAGCAACAGG | 83828684 | A | G | ATAGCAACAGGGAAAAAAAAAA | 560/1211 |
| | | | | AAAAAAAAAAA | | | | | |
| ZCCHC14 | chr16 | 87445100 | 87445121 | CGCGGCCATGG | 87445111 | C | T | CGCGGCCATGGTGCGCGCTTAC | 561/1212 |
| | | | | CGCGCGCTTAC | | | | | |
| ZCCHC14 | chr16 | 87446524 | 87446545 | GTGATTCAGCA | 87446535 | G | C | GTGATTCAGCACCATCACCGGC | 562/1213 |
| | | | | GCATCACCGGC | | | | | |
| ZCCHC14 | chr16 | 87446603 | 87446624 | GGTCAGTGCCA | 87446614 | T | A | GGTCAGTGCCAATCCACAGCTG | 563/1214 |
| | | | | TTCCACAGCTG | | | | | |
| ZCCHC14 | chr16 | 87457515 | 87457536 | TAGAAACAGAC | 87457526 | A | G | TAGAAACAGACGCCACATACTT | 564/1215 |
| | | | | ACCACATACTT | | | | | |
| ZCCHC14 | chr16 | 87457549 | 87457570 | GTGTCCTGGTA | 87457560 | C | T | GTGTCCTGGTATGACTGGGGCA | 565/1216 |
| | | | | CGACTGGGGCA | | | | | |
| ZCCHC14 | chr16 | 87500920 | 87500941 | TGCCCCAAGCG | 87500931 | A | G | TGCCCCAAGCGGAAACAGAAAA | 566/1217 |
| | | | | AAAACAGAAAA | | | | | |
| ZCCHC14 | chr16 | 87501011 | 87501032 | AAGCTCTCCTG | 87501022 | G | A | AAGCTCTCCTGAAGCAAATATG | 567/1218 |
| | | | | GAGCAAATATG | | | | | |
| ACSF3 | chr16 | 89167375 | 89167396 | GAGAGGGTCTC | 89167386 | C | T | GAGAGGGTCTCTTTCCTATGCG | 568/1219 |
| | | | | CTTCCTATGCG | | | | | |
| ACSF3 | chr16 | 89168988 | 89169009 | CAGCTGTGCTC | 89168999 | T | C | CAGCTGTGCTCCCGTCCCCTGC | 569/1220 |
| | | | | TCGTCCCCTGC | | | | | |
| ACSF3 | chr16 | 89178463 | 89178484 | GCTCATCTTCC | 89178474 | T | C | GCTCATCTTCCCACCGAGTGCT | 570/1221 |
| | | | | TACCGAGTGCT | | | | | |
| ACSF3 | chr16 | 89178520 | 89178541 | TTCTGAAACGC | 89178531 | C | T | TTCTGAAACGCTGCGGATCAAT | 571/1222 |
| | | | | CGCGGATCAAT | | | | | |
| ACSF3 | chr16 | 89199511 | 89199532 | AGCTCTGACCT | 89199522 | C | T | AGCTCTGACCTTCATGTTCTTC | 572/1223 |
| | | | | CCATGTTCTTC | | | | | |
| FBXW10 | chr17 | 18675791 | 18675812 | CGTGGAAAAAA | 18675802 | C | T | CGTGGAAAAAATGAAACAAAAG | 573/1224 |
| | | | | CGAAACAAAAG | | | | | |
| FBXW10 | chr17 | 18675927 | 18675948 | ATCCAAGAGCT | 18675938 | C | T | ATCCAAGAGCTTCTACCAGGCA | 574/1225 |
| | | | | CCTACCAGGCA | | | | | |
| RPL23A | chr17 | 27047330 | 27047351 | TCCATGTCCCC | 27047341 | G | A | TCCATGTCCCCAGGCCTGTAAG | 575/1226 |
| | | | | GGGCCTGTAAG | | | | | |
| RPL23A | chr17 | 27047481 | 27047502 | AAGTATCAAGC | 27047492 | G | T | AAGTATCAAGCTTTCATTCAGT | 576/1227 |
| | | | | GTTCATTCAGT | | | | | |
| RPL23A | chr17 | 27050406 | 27050427 | TCCCATAAGAG | 27050417 | A | G | TCCCATAAGAGGATTGGCTTTG | 577/1228 |
| | | | | AATTGGCTTTG | | | | | |
| RPL23A | chr17 | 27050858 | 27050879 | GTAACGAGGCT | 27050869 | C | G | GTAACGAGGCTGCCTTTTGTTT | 578/1229 |
| | | | | CCCTTTTGTTT | | | | | |
| DDX5 | chr17 | 62496250 | 62496271 | CAAAGCTCCCA | 62496261 | T | C | CAAAGCTCCCACTGGTGTAATT | 579/1230 |
| | | | | TTGGTGTAATT | | | | | |
| DDX5 | chr17 | 62496659 | 62496680 | ATCCTTACCTG | 62496670 | A | C | ATCCTTACCTGCACCTCTGTCT | 580/1231 |
| | | | | AACCTCTGTCT | | | | | |
| DDX5 | chr17 | 62498714 | 62498735 | ATTGCTAGGGC | 62498725 | C | G | ATTGCTAGGGCGACACATTTAT | 581/1232 |
| | | | | CACACATTTAT | | | | | |
| DDX5 | chr17 | 62499152 | 62499173 | TCTTCAGCAAG | 62499163 | C | T | TCTTCAGCAAGTTGTCTTACTT | 582/1233 |
| | | | | CTGTCTTACTT | | | | | |
| DDX5 | chr17 | 62499301 | 62499322 | CTTACTCTTAT | 62499312 | T | C | CTTACTCTTATCTGATCCACAA | 583/1234 |
| | | | | TTGATCCACAA | | | | | |
| DDX5 | chr17 | 62499690 | 62499711 | CTAAGGAAAGA | 62499701 | G | C | CTAAGGAAAGACAAACAGCTTT | 584/1235 |
| | | | | GAAACAGCTTT | | | | | |
| DDX5 | chr17 | 62499748 | 62499769 | TTATTATACTA | 62499759 | G | A | TTATTATACTAACAGATCCTTT | 585/1236 |
| | | | | GCAGATCCTTT | | | | | |
| DDX5 | chr17 | 62500276 | 62500297 | AAGAAAAAGAG | 62500287 | G | A | AAGAAAAAGAGAGGGTAGGTGG | 586/1237 |
| | | | | GGGGTAGGTGG | | | | | |
| DDX5 | chr17 | 62500282 | 62500303 | AAGAGGGGGTA | 62500293 | G | A | AAGAGGGGGTAAGTGGAAACAA | 587/1238 |
| | | | | GGTGGAAACAA | | | | | |
| DDX5 | chr17 | 62500457 | 62500478 | GTCTTAAAATT | 62500468 | C | G | GTCTTAAAATTGATGACAACCA | 588/1239 |
| | | | | CATGACAACCA | | | | | |
| 9-Sep | chr17 | 75398254 | 75398275 | CAGGACCTGGG | 75398265 | C | T | CAGGACCTGGGTGTGAAGAACT | 589/1240 |
| | | | | CGTGAAGAACT | | | | | |
| 9-Sep | chr17 | 75425179 | 75425200 | CCGTGTCCTCC | 75425190 | G | A | CCGTGTCCTCCAGTGTGTGTGA | 590/1241 |
| | | | | GGTGTGTGTGA | | | | | |
| 9-Sep | chr17 | 75425195 | 75425216 | GTGTGAGGCCA | 75425206 | A | G | GTGTGAGGCCAGGCTCCTGGGG | 591/1242 |
| | | | | AGCTCCTGGGG | | | | | |
| 9-Sep | chr17 | 75472046 | 75472067 | GGGAAGACAGG | 75472057 | G | A | GGGAAGACAGGAGAATGGCATT | 592/1243 |
| | | | | GGAATGGCATT | | | | | |
| 9-Sep | chr17 | 75483517 | 75483538 | TTGGGTAAATC | 75483528 | C | T | TTGGGTAAATCTACCTTAATCA | 593/1244 |
| | | | | CACCTTAATCA | | | | | |
| 9-Sep | chr17 | 75484307 | 75484328 | CGTGGCTCTGT | 75484318 | G | A | CGTGGCTCTGTACAGATATTGA | 594/1245 |
| | | | | GCAGATATTGA | | | | | |
| 9-Sep | chr17 | 75484793 | 75484814 | CCCATCCCCCA | 75484804 | C | T | CCCATCCCCCATGCAGCTGGCA | 595/1246 |
| | | | | CGCAGCTGGCA | | | | | |
| 9-Sep | chr17 | 75488663 | 75488684 | GCCACAGGGAT | 75488674 | G | A | GCCACAGGGATAGGCCCATCTC | 596/1247 |
| | | | | GGGCCCATCTC | | | | | |
| 9-Sep | chr17 | 75488771 | 75488792 | GGACCGGCTGG | 75488782 | T | C | GGACCGGCTGGCGAACGAGAAG | 597/1248 |
| | | | | TGAACGAGAAG | | | | | |
| RAB31 | chr18 | 9775156 | 9775177 | CAGTCTCATCA | 9775167 | A | G | CAGTCTCATCAGCCAGAAATAG | 598/1249 |
| | | | | ACCAGAAATAG | | | | | |
| RAB31 | chr18 | 9775351 | 9775372 | TTGGGTAAGTT | 9775362 | C | T | TTGGGTAAGTTTCTGTATGTCA | 599/1250 |
| | | | | CCTGTATGTCA | | | | | |
| RAB31 | chr18 | 9787146 | 9787167 | ACCACCCCAAA | 9787157 | G | A | ACCACCCCAAAAAATTCCTTCT | 600/1251 |
| | | | | GAATTCCTTCT | | | | | |
| RAB31 | chr18 | 9815055 | 9815076 | TAGTTGAAATA | 9815066 | T | C | TAGTTGAAATACTATATTGAGG | 601/1252 |
| | | | | TTATATTGAGG | | | | | |
| RAB31 | chr18 | 9815062 | 9815083 | AATATTATATT | 9815073 | G | A | AATATTATATTAAGGGGTCTTT | 602/1253 |
| | | | | GAGGGGTCTTT | | | | | |
| RAB31 | chr18 | 9815088 | 9815109 | TGATTTGTGTA | 9815099 | C | T | TGATTTGTGTATACTGTTGGTT | 603/1254 |
| | | | | CACTGTTGGTT | | | | | |
| RAB31 | chr18 | 9845482 | 9845503 | AAAGGAGCTGT | 9845493 | T | C | AAAGGAGCTGTCGCCGCACAAG | 604/1255 |
| | | | | TGCCGCACAAG | | | | | |
| RAB31 | chr18 | 9859330 | 9859351 | GCCGTGGTCCA | 9859341 | C | T | GCCGTGGTCCATGGTACTTGAA | 605/1256 |
| | | | | CGGTACTTGAA | | | | | |
| CREB3L3 | chr19 | 4159711 | 4159732 | GTGGACCTGTC | 4159722 | C | T | GTGGACCTGTCTCCACGATGCA | 606/1257 |
| | | | | CCCACGATGCA | | | | | |
| CREB3L3 | chr19 | 4164463 | 4164484 | CCTGCACCCGC | 4164474 | C | T | CCTGCACCCGCTGTCATTCCTC | 607/1258 |
| | | | | CGTCATTCCTC | | | | | |
| CREB3L3 | chr19 | 4168426 | 4168447 | AAGGAATATAT | 4168437 | C | A | AAGGAATATATAGATGGCCTGG | 608/1259 |
| | | | | CGATGGCCTGG | | | | | |
| FCER2 | chr19 | 7762165 | 7762186 | TTCCTGTGAAA | 7762176 | T | C | TTCCTGTGAAACCTGCGTGGCT | 609/1260 |
| | | | | TCTGCGTGGCT | | | | | |
| FCER2 | chr19 | 7762429 | 7762450 | CATCTGGTCAC | 7762440 | C | T | CATCTGGTCACTGTGGTGGCTT | 610/1261 |
| | | | | CGTGGTGGCTT | | | | | |
| FCER2 | chr19 | 7764507 | 7764528 | TTAGAAATTCA | 7764518 | C | T | TTAGAAATTCATCCTCTTTCCC | 611/1262 |
| | | | | CCCTCTTTCCC | | | | | |
| ATP1A3 | chr19 | 42473565 | 42473586 | CACAGCACCCT | 42473576 | G | T | CACAGCACCCTTCCCTACTCAC | 612/1263 |
| | | | | GCCCTACTCAC | | | | | |
| ATP1A3 | chr19 | 42474381 | 42474402 | TTGTCCGTCCG | 42474392 | C | T | TTGTCCGTCCGTGGGTTCCTGG | 613/1264 |
| | | | | CGGGTTCCTGG | | | | | |
| ATP1A3 | chr19 | 42474699 | 42474720 | GGCACAGGCAG | 42474710 | G | A | GGCACAGGCAGACTCAGAGCAG | 614/1265 |
| | | | | GCTCAGAGCAG | | | | | |
| ATP1A3 | chr19 | 42485757 | 42485778 | GCAGACTCAGA | 42485768 | C | T | GCAGACTCAGATGCATCCCCAG | 615/1266 |
| | | | | CGCATCCCCAG | | | | | |
| ATP1A3 | chr19 | 42486267 | 42486288 | CGAGCAAGGGC | 42486278 | A | C | CGAGCAAGGGCCGGCAAGTTAC | 616/1267 |
| | | | | AGGCAAGTTAC | | | | | |
| POU2F2 | chr19 | 42626196 | 42626217 | CAGCCCTTGGA | 42626207 | C | T | CAGCCCTTGGATTGAGGCAGGC | 617/1268 |
| | | | | CTGAGGCAGGC | | | | | |
| ZC3H4 | chr19 | 47584858 | 47584879 | TACAGCTTACA | 47584869 | C | T | TACAGCTTACATGGGAAATCAC | 618/1269 |
| | | | | CGGGAAATCAC | | | | | |
| ZC3H4 | chr19 | 47585353 | 47585374 | AGAAGGAAGAT | 47585364 | T | C | AGAAGGAAGATCGGCTGTTACT | 619/1270 |
| | | | | TGGCTGTTACT | | | | | |
| ZC3H4 | chr19 | 47585381 | 47585402 | ATCAAGGAGCA | 47585392 | G | A | ATCAAGGAGCAAAGAAGTTCTG | 620/1271 |
| | | | | GAGAAGTTCTG | | | | | |
| ZC3H4 | chr19 | 47585549 | 47585570 | CTCCCTAGAAG | 47585560 | A | G | CTCCCTAGAAGGGAAGCAACAA | 621/1272 |
| | | | | AGAAGCAACAA | | | | | |
| ZC3H4 | chr19 | 47585581 | 47585602 | TGGTGGCGTGG | 47585592 | G | A | TGGTGGCGTGGAAAGCTCTTGC | 622/1273 |
| | | | | GAAGCTCTTGC | | | | | |
| ZC3H4 | chr19 | 47588270 | 47588291 | GGGACCTTGGC | 47588281 | C | T | GGGACCTTGGCTCAAACATCAG | 623/1274 |
| | | | | CCAAACATCAG | | | | | |
| ZC3H4 | chr19 | 47589573 | 47589594 | GCAGTGCTCAC | 47589584 | G | A | GCAGTGCTCACACCCCGGAAAG | 624/1275 |
| | | | | GCCCCGGAAAG | | | | | |
| ZC3H4 | chr19 | 47593443 | 47593464 | GGGACTGCGTC | 47593454 | C | A | GGGACTGCGTCACAGAGATGGG | 625/1276 |
| | | | | CCAGAGATGGG | | | | | |
| HSD17B14 | chr19 | 49318369 | 49318390 | TTGACTCTTCC | 49318380 | G | A | TTGACTCTTCCACAGGTAGGGG | 626/1277 |
| | | | | GCAGGTAGGGG | | | | | |
| FUZ | chr19 | 50310455 | 50310476 | GCAGCCCATGG | 50310466 | G | A | GCAGCCCATGGATGGGACTCTG | 627/1278 |
| | | | | GTGGGACTCTG | | | | | |
| FUZ | chr19 | 50314727 | 50314748 | AGGAGAGGAAG | 50314738 | A | C | AGGAGAGGAAGCAGGGACCAGC | 628/1279 |
| | | | | AAGGGACCAGC | | | | | |
| ZNF134 | chr19 | 58131801 | 58131822 | CCTTAAGAAGG | 58131812 | G | A | CCTTAAGAAGGAATAAAAGTGA | 629/1280 |
| | | | | GATAAAAGTGA | | | | | |
| ZNF134 | chr19 | 58131856 | 58131877 | AGAACCTCATC | 58131867 | C | T | AGAACCTCATCTGTCAGAGAAG | 630/1281 |
| | | | | CGTCAGAGAAG | | | | | |
| ZNF134 | chr19 | 58132523 | 58132544 | TGCATTGAATG | 58132534 | C | T | TGCATTGAATGTGGGAAATTCT | 631/1282 |
| | | | | CGGGAAATTCT | | | | | |
| ZNF134 | chr19 | 58132855 | 58132876 | GTGCCAGGTAC | 58132866 | G | A | GTGCCAGGTACATGGGAACCTT | 632/1283 |
| | | | | GTGGGAACCTT | | | | | |
| SNPH | chr20 | 1285538 | 1285559 | GACCTGAAGAC | 1285549 | G | A | GACCTGAAGACACAGCTGTCAC | 633/1284 |
| | | | | GCAGCTGTCAC | | | | | |
| DTD1 | chr20 | 18576591 | 18576612 | GAGTGTGTTGG | 18576602 | G | A | GAGTGTGTTGGAGGGCTTGTGA | 634/1285 |
| | | | | GGGGCTTGTGA | | | | | |
| DTD1 | chr20 | 18576641 | 18576662 | TCCCCTTAGGG | 18576652 | T | C | TCCCCTTAGGGCCCGAAAGATT | 635/1286 |
| | | | | TCCGAAAGATT | | | | | |
| DTD1 | chr20 | 18608786 | 18608807 | CCTACATGCAG | 18608797 | G | A | CCTACATGCAGATGCACATTCA | 636/1287 |
| | | | | GTGCACATTCA | | | | | |
| DTD1 | chr20 | 18724832 | 18724853 | GAAAAGAAGAC | 18724843 | C | T | GAAAAGAAGACTGCAGTGCCAG | 637/1288 |
| | | | | CGCAGTGCCAG | | | | | |
| SPINT3 | chr20 | 44141389 | 44141410 | TTTCAAAGTTG | 44141400 | A | G | TTTCAAAGTTGGAAAACCATCG | 638/1289 |
| | | | | AAAAACCATCG | | | | | |
| SPINT3 | chr20 | 44141400 | 44141421 | AAAAACCATCG | 44141411 | C | T | AAAAACCATCGTGTCATGTAGG | 639/1290 |
| | | | | CGTCATGTAGG | | | | | |
| OSBPL2 | chr20 | 60834938 | 60834959 | CTCTGTAGTCA | 60834949 | C | A | CTCTGTAGTCAACTGCTTGCAT | 640/1291 |
| | | | | CCTGCTTGCAT | | | | | |
| OSBPL2 | chr20 | 60835020 | 60835041 | TTTCTTGTCTC | 60835031 | G | A | TTTCTTGTCTCACACAGGCTTT | 641/1292 |
| | | | | GCACAGGCTTT | | | | | |
| OSBPL2 | chr20 | 60856241 | 60856262 | GGGTGAGAGCG | 60856252 | C | T | GGGTGAGAGCGTGAGGCTCCGG | 642/1293 |
| | | | | CGAGGCTCCGG | | | | | |
| OSBPL2 | chr20 | 60868824 | 60868845 | TCCCTTGTATC | 60868835 | C | T | TCCCTTGTATCTGGCAGGTGGT | 643/1294 |
| | | | | CGGCAGGTGGT | | | | | |
| KRTAP12- | chr21 | 46086459 | 46086480 | ATACACGACAG | 46086470 | G | A | ATACACGACAGACCTGCAGCTC | 644/1295 |
| 2 | | | | GCCTGCAGCTC | | | | | |
| KRTAP12- | chr21 | 46086477 | 46086498 | GCTCACAGGCA | 46086488 | C | T | GCTCACAGGCATGCACAGGGAG | 645/1296 |
| 2 | | | | CGCACAGGGAG | | | | | |
| PATZ1 | chr22 | 31731521 | 31731542 | TAGCTAGTTGG | 31731532 | G | A | TAGCTAGTTGGATGATGAAGGT | 646/1297 |
| | | | | GTGATGAAGGT | | | | | |
| PATZ1 | chr22 | 31737425 | 31737446 | CCCTCTGGGGT | 31737436 | G | A | CCCTCTGGGGTAGTCCAGCCCT | 647/1298 |
| | | | | GGTCCAGCCCT | | | | | |
| PATZ1 | chr22 | 31740438 | 31740459 | GAGTAGGGCTT | 31740449 | C | T | GAGTAGGGCTTTTCCCCAGAGT | 648/1299 |
| | | | | CTCCCCAGAGT | | | | | |
| W12- | chr22 | 49290622 | 49290643 | AGCCACCGGCT | 49290633 | C | T | AGCCACCGGCTTCCCAGGCTGA | 649/1300 |
| 81516E3.1 | | | | CCCCAGGCTGA | | | | | |
| W12- | chr22 | 49290699 | 49290720 | AGGGCGATCCC | 49290710 | G | A | AGGGCGATCCCACCTGGATGCG | 650/1301 |
| 81516E3.1 | | | | GCCTGGATGCG | | | | | |
| DNASE1L1 | chrX | 153631900 | 153631921 | CCGGGCAAAGA | 153631911 | C | T | CCGGGCAAAGATGTCATCCTCA | 651/1302 |
| | | | | CGTCATCCTCA | | | | | |

The scope of the claims should not be limited by the preferred embodiments set forth in the examples, but should be given the broadest interpretation consistent with the description as a whole.

### REFERENCES

1. Asher, M. A. & Burton, D. C. Adolescent idiopathic scoliosis: natural history and long term treatment effects. Scoliosis 1, 2 (2006).
2. Miller, N. H. Adolescent Idiopathic Scoliosis: Etiology. Pediatr. Spine 354 (2001).
3. Patten, S. A. et al. Functional variants of POC5 identified in patients with idiopathic scoliosis. J. Clin. Invest. 125, 1124-8 (2015).
4. Baschal, E. E. et al. Exome sequencing identifies a rare HSPG2 variant associated with familial idiopathic scoliosis. G3 (Bethesda). 5, 167-74 (2015).
5. Gorman, K. F., Julien, C. & Moreau, A. The genetic epidemiology of idiopathic scoliosis. Eur. Spine J. 21, 1905-19 (2012).
6. Atzal, S., Ramzan, M., Farooq, M. & Rasul, G. Biomechanics of spinal deformity. JK Pract. 11, 1-10 (2004).
7. Janssen, M. M. A. et al. Sagittal spinal profile and spinopelvic balance in parents of scoliotic children. Spine J. 13, 1789-1800 (2013).
8. Schultz, A. B. Biomechanical factors in the progression of idiopathic scoliosis. Ann. Biomed. Eng. 12, 621-30 (1984).
9. Hefti, F. Pathogenesis and biomechanics of adolescent idiopathic scoliosis (AIS). J. Child. Orthop. 7, 17-24 (2013).
10. Resnick, A. Mechanical properties of a primary cilium as measured by resonant oscillation. Biophys. J. 109, 18-25 (2015).
11. Khayyeri, H., Barreto, S. & Lacroix, D. Primary cilia mechanics affects cell mechanosensation: A computational study. J. Theor. Biol. (2015). doi:10.1016/j.jtbi.2015.04.034
12. Nguyen, A. M. & Jacobs, C. R. Emerging role of primary cilia as mechanosensors in osteocytes. Bone 54, 196-204 (2013).
13. Lee, K. L. et al. The primary cilium functions as a mechanical and calcium signaling nexus. Cilia 4, 7 (2015).
14. Delling, M. et al. Primary cilia are not calcium-responsive mechanosensors. Nature 531, 656-660 (2016).
15. Leucht, P. et al. Primary cilia act as mechanosensors during bone healing around an implant. Med. Eng. Phys. 35, 392-402 (2013).
16. Delaine-Smith, R. M., Sittichokechaiwut, A. & Reilly, G. C. Primary cilia respond to fluid shear stress and mediate flow-induced calcium deposition in osteoblasts. FASEB J. 28, 430-9 (2014).
17. Hoey, D. A., Tormey, S., Ramcharan, S., O'Brien, F. J. & Jacobs, C. R. Primary Cilia-Mediated Mechanotransduction in Human Mesenchymal Stem Cells, Stem Cells 30, 2561-2570 (2012).
18. Ascenzi, M.-G. et al. Effect of localization, length and orientation of chondrocytic primary cilium on murine growth plate organization. J. Theor. Biol. 285, 147-55 (2011).
19. Huber, C. & Cormier-Daire, V. Ciliary disorder of the skeleton. Am. J. Med. Genet. Part C Semin. Med. Genet. 160 C, 165-174 (2012).
20. Kobayashi, D. et al. Characterization of the medaka (Oryzias latipes) primary ciliary dyskinesia mutant, jaodori: Redundant and distinct roles of dynein axonemal intermediate chain 2 (dnai2) in motile cilia. Dev. Biol. 347, 62-70 (2010).
21. Buchan, J. G. et al. Kinesin family member 6 (kif6) is necessary for spine development in zebrafish. Dev. Dyn. 243, 1646-57 (2014).
22. Lee, S. et al. Optimal unified approach for rare-variant association testing with application to small-sample case-control whole-exome sequencing studies. Am. J. Hum. Genet. 91, 224-37 (2012).
23. Seeley, E. S. & Nachury, M. V. The perennial organelle: assembly and disassembly of the primary cilium. J. Cell Sci. 123, 511-518 (2010).
24. Bacabac, R. G. et al. Nitric oxide production by bone cells is fluid shear stress rate dependent. Biochem. Biophys. Res. Commun. 315, 823-9 (2004).
25. Loth, F., Yardimci, M. A. & Alperin, N. Hydrodynamic Modeling of Cerebrospinal Fluid Motion Within the Spinal Cavity. J. Biomech. Eng. 123, 71 (2001).
26. Yuan, X., Serra, R. a & Yang, S. Function and regulation of primary cilia and intraflagellar transport proteins in the skeleton. Ann. N. Y. Acad. Sci. 1335, 78-99 (2014).
27. Vaughan, T. J., Mullen, C. a., Verbruggen, S. W. & McNamara, L. M. Bone cell mechanosensation of fluid flow stimulation: a fluid-structure interaction model characterising the role integrin attachments and primary cilia. Biomech. Model. Mechanobiol. 2, (2014).
28. May-Simera, H. L. & Kelley, M. W. Cilia, Wnt signaling, and the cytoskeleton. Cilia 1, 7 (2012).
29. Kumamoto, N. et al. A role for primary cilia in glutamatergic synaptic integration of adult-born neurons. Nat. Neurosci. 15, 399-405, S1 (2012).
30. Norvell, S. M., Alvarez, M., Bidwell, J. P. & Pavalko, F. M. Fluid shear stress induces beta-catenin signaling in osteoblasts. Calcif. Tissue Int. 75, 396-404 (2004).
31. Corbit, K. C. et al. Kif3a constrains beta-catenin-dependent Wnt signalling through dual ciliary and non-ciliary mechanisms. Nat. Cell Biol. 10, 70-6 (2008).
32. Fan, Y.-H. et al. SNP rs11190870 near LBX1 is associated with adolescent idiopathic scoliosis in southern Chinese. J. Hum. Genet. 57, 244-6 (2012).
33. Jiang, H. et al. Association of rs11190870 near LBX1 with adolescent idiopathic scoliosis susceptibility in a Han Chinese population. Eur. Spine J. 22, 282-6 (2013).
34. McKenna, A. et al. The Genome Analysis Toolkit: a MapReduce framework for analyzing next-generation DNA sequencing data. Genome Res. 20, 1297-303 (2010).
35. Wu, M. C. et al. Rare-variant association testing for sequencing data with the sequence kernel association test. Am. J. Hum. Genet, 89, 82-93 (2011).
36. van Dam, T. J., Wheway, G., Slaats, G. G., Huynen, M. a & Giles, R. H. The SYSCILIA gold standard (SCGSv1) of known ciliary components and its applications within a systems biology consortium. Cilia 2, 7 (2013).
37. Kim, J. et al. Functional genomic screen for modulators of ciliogenesis and cilium length. Nature 464, 1048-1051 (2010).
38. McMurray, R. J., Wann, A. K. T., Thompson, C. L., Connelly, J. T. & Knight, M. M. Surface topography regulates wnt signaling through control of primary cilia structure in mesenchymal stem cells. Sci. Rep. 3, 3545 (2013).
39. Satir, P., Pedersen, L. B. & Christensen, S. T. The primary cilium at a glance. J. Cell Sci. 123, 499-503 (2010).
40. Malone, A. M. D. et al. Primary cilia mediate mechanosensing in bone cells by a calcium-independent mechanism. Proc. Natl. Acad. Sci. U.S.A. 104, 13325-30 (2007).
41. Praetorius, H. A. & Spring, K. R. Removal of the MDCK cell primary cilium abolishes flow sensing. J. Membr. Biol. 191, 69-76 (2003).
42. Wann, A. K. T. et al. Primary cilia mediate mechanotransduction through control of ATP-induced Ca2+ signaling in compressed chondrocytes. FASEB J. 26, 1663-71 (2012).
43. You, J. et al. Osteopontin gene regulation by oscillatory fluid flow via intracellular calcium mobilization and activation of mitogen-activated protein kinase in MC3T3-E1 osteoblasts. J. Biol. Chem. 276, 13365-71 (2001).
44. Zhang, X. et al. Cyclooxygenase-2 regulates mesenchymal cell differentiation into the osteoblast lineage and is critically involved in bone repair. J. Clin. Invest. 109, 1405-15 (2002).
45. Ishida, K. et al. Relationship between bone density and bone metabolism in adolescent idiopathic scoliosis. Scoliosis 10, 9 (2015).
46. Cook, S. D. et al. Trabecular bone mineral density in idiopathic scoliosis. J. Pediatr. Orthop. 7, 168-74
47. Jin, D. et al. Prostaglandin signalling regulates ciliogenesis by modulating intraflagellar transport. Nat. Cell Biol. 16, 841-51 (2014).
48. Litzenberger, J. B., Kim, J.-B., Tummala, P. & Jacobs, C. R. Beta1 integrins mediate mechanosensitive signaling pathways in osteocytes. Calcif. Tissue Int. 86, 325-32 (2010).
49. McGlashan, S. R., Jensen, C. G. & Poole, C. A. Localization of extracellular matrix receptors on the chondrocyte primary cilium. J. Histochem. Cytochem. 54, 1005-14 (2006).
50. Kuo, J.-C. Mechanotransduction at focal adhesions: integrating cytoskeletal mechanics in migrating cells. J. Cell. Mol. Med. 17, 704-12 (2013).
51. Lancaster, M. A., Schroth, J. & Gleeson, J. G. Subcellular spatial regulation of canonical Wnt signalling at the primary cilium. Nat. Cell Biol. 13, 700-7 (2011).
52. Besschetnova, T. Y. et al. Identification of signaling pathways regulating primary cilium length and flow-mediated adaptation. Curr. Biol. 20, 182-7 (2010).
53. Yuan, S. et al. Target-of-rapamycin complex 1 (Torc1) signaling modulates cilia size and function through protein synthesis regulation. Proc. Natl. Acad. Sci. U.S.A. 109, 2021-6 (2012).
54. McGlashan, S. R. et al. Mechanical loading modulates chondrocyte primary cilia incidence and length. Cell Biol. Int. 34, 441-6 (2010).
55. Marosy, B. et al. Identification of susceptibility loci for scoliosis in FIS families with triple curves. Am. J. Med. Genet. A 152A, 846-55 (2010).
56. Zhu, Z. et al. Genome-wide association study identifies new susceptibility loci for adolescent idiopathic scoliosis in Chinese girls. Nat. Commun. 6, 8355 (2015).
57. Azimzadeh, J. et al. hPOC5 is a centrin-binding protein required for assembly of full-length centrioles. J. Cell Biol. 185, 101-114 (2009).
58. Das, A., Dickinson, D. J., Wood, C. C., Goldstein, B. & Slep, K. C. Crescerin uses a TOG domain array to regulate microtubules in the primary cilium. Mol. Biol. Cell 26, 4248-64 (2015).
59. Samora, C. P. et al. MAP4 and CLASP1 operate as a safety mechanism to maintain a stable spindle position in mitosis. Nat. Cell Biol. 13, 1040-50 (2011).
60. Tsvetkov, A. S., Samsonov, A., Akhmanova, A., Galjart, N. & Popov, S. V. Microtubule-binding proteins CLASP1 and CLASP2 interact with actin filaments. Cell Motil. Cytoskeleton 64, 519-30 (2007).
61. Gallo, R. M. et al. Regulation of the actin cytoskeleton by Rho kinase controls antigen presentation by CD1d. J. Immunol. 189, 1689-98 (2012).
62. Suzuki, T. et al. Essential roles of Lyn in fibronectin-mediated filamentous actin assembly and cell motility in mast cells. J. Immunol. 161, 3694-701 (1998).
63. Martins, T., Maia, A. F., Steffensen, S. & Sunkel, C. E. Sgt1, a co-chaperone of Hsp90 stabilizes Polo and is required for centrosome organization. EMBO J. 28, 234-47 (2009).
64. Chen, L.-Y. & Lingner, J. AUF1/HnRNP D RNA binding protein functions in telomere maintenance. Mol. Cell 47, 1-2 (2012).
65. Haller, G. et al. A polygenic burden of rare variants across extracellular matrix genes among individuals with adolescent idiopathic scoliosis. Hum. Mol. Genet. 25, 202-9 (2016).
66. Oliazadeh, N., Franco, A., Wang, D. & Moreau, A. Abnormalities in primary cilium of osteoblasts of adolescent idiopathic scoliosis patients. Cilia 4, P6 (2015).
67. Fairbank, J. Historical perspective: William Adams, the forward bending test, and the spine of Gideon Algernon Mantell. Spine (Phila. Pa. 1976). 29, 1953-5 (2004).
68. M., W. F. Fluid Mechanics. (McGraw-Hill, 2010).
69. Fung, Y. C. Motion. Biomechanics (Springer New York, 1990). doi:10.1007/978-1-4419-6856-2_1
70. Zhou, X., Liu, D., You, L. & Wang, L. Quantifying fluid shear stress in a rocking culture dish. J. Biomech. 43, 1598-1602 (2010).
71. Homer, N., Merriman, B. & Nelson, S. F. BFAST: an alignment tool for large scale genome resequencing. PLoS One 4, e7767 (2009).
72. Paila, U., Chapman, B. A., Kirchner, R. & Quinlan, A. R. GEMINI: integrative exploration of genetic variation and genome annotations. PLoS Comput. Biol. 9, e1003153 (2013).
73. Kircher, M. et al. A general framework for estimating the relative pathogenicity of human genetic variants. Nat. Genet. 46, 310-5 (2014).
74. Lee, S., Fuchsberger, C., Kim, S. & Scott, L. An efficient resampling method for calibrating single and gene-based rare variant association analysis in case-control studies. Biostatistics 17, 1-15 (2016).
75. Sham, P. C. & Purcell, S. M. Statistical power and significance testing in large-scale genetic studies. Nat. Rev. Genet. 15, 335-346 (2014).

1. (canceled)

2. A method of determining the risk of or predisposition to developing a scoliosis in a subject comprising:
(a) (i) determining the average length of cilia on the surface of cells in a cell sample from the subject;
(ii) determining the number of cells with elongated cilia in a cell sample from the subject
(iii) determining the number of ciliated cells in a cell sample from the subject; or
(iv) any combination of one of (i), (ii) and (iii),
wherein an increase in the average length of cilia, an increase in the number of cells having elongated cilia or a decrease in the number of ciliated cells in the cell sample from the subject as compared to that in a control sample is indicative of an increased risk of or predisposition to developing a scoliosis;
(b) determining a cellular response to mechanostimulation of cells in a cell sample from a subject, wherein the determining comprises:
(i) applying mechanostimulation to cells in a cell sample from the subject; and
(ii) measuring the expression level of at least one mechanoresponsive gene, wherein the at least one mechanoresponsive gene is ITGB1; ITGB3, CTNNB1; POC5, BMP2, COX-2, RUNX2, CTNNB1 or any combination thereof;
(iii) comparing the expression level measured in (b)(ii) to that of a control sample, wherein an altered expression level in said mechanoresponsive gene as compared to that of the control sample is indicative of an increased risk of or predisposition to developing a scoliosis; or
(c) a combination of (a) and (b).

3. The method of claim 2, wherein (b) is performed on cells having elongated cilia.

4. The method of claim 2, wherein the mechanostimulation is fluid sheer stress.

5. The method of claim 4, wherein the level of sheer stress applied corresponds to a Womersley number of between about 5 and 18 or of about 8.

6. (canceled)

7. The method of claim 4, wherein said mechanostimulation corresponds to an average sheer stress of about 1 Pa; and/or is applied at a frequency of between about 1 and about 3 Hz.

8. (canceled)

9. The method of claim 2, wherein said determining is over time.

10. A method of (A) determining the risk of developing a scoliosis in a subject; or (B) genotyping a subject suffering from Idiopathic scoliosis or at risk of developing a scoliosis, the method comprising detecting in a cell sample from the subject, the presence or absence of a polymorphic marker in at least one allele of at least one gene listed in Table 4 or substitute marker in linkage disequilibrium with the polymorphic marker.

11. (canceled)

12. The method of claim 10, wherein the at least one gene comprises (i) FEZF1, CDH13, FBXL2, TRIM13, CD1B, VAX1, CLASP1, SUGT1, MIPEP, FAM188A, TAF6, WHSC1, GPR158, HNRNPD, RUNX1T1, GRIK3, FUZ, LYN, DDXS, PODXL, ATPSB, PIGK, AL159977.1, BTN1A1, CDK11A, HIVEP1, HSD17B14, KCNMA1, PXDN, RAB31, RBMS, RNF149, SOD2, TOPBP1, ZCCHC14, ZNF323, or any combination thereof (ii) FEZF1, CDH13, FBXL2, TRIM13, CD1B, VAX1, CLASP1, SUGT1, MIPEP, FAM188A, TAF6, WHSC1, GPR158, HNRNPD, RUNX1T1, GRIK3, FUZ, LYN, DDXS, PODXL, ATPSB, PIGK, AL159977.1, or any combination thereof (iii) ATPSB, BTN1A1, CD1B, CDK11A, CLASP1, DDXS, FBXL2, HIVEP1, HSD17B14, KCNMA1, PXDN, RAB31, RBM5, RNF149, SOD2, SUGT1, TOPBP1, ZCCHC14, ZNF323 or any combination thereof, or (iv) CDB1, CLASP1 and SUGT1.

13.-16. (canceled)

17. The method of claim 10, wherein the polymorphic marker is a polymorphic marker defined in Table 6, and preferably a risk variant defined in Table 6.

18. (canceled)

19. The method of claim 10, wherein said method comprises determining the presence or absence of at least two polymorphic markers and preferably comprises determining the presence or absence of at least two polymorphic markers in at least two genes.

20. (canceled)

21. The method of claim 2, wherein the subject is a female.

22. The method of claim 2, wherein the subject is pre-diagnosed with a scoliosis.

23. The method of claim 2, wherein the cell sample comprises bone cells; or the cell sample comprises mesenchymal stem cells, myoblasts, preosteoblasts, osteoblasts, osteocytes and/or chondrocytes.

24. (canceled)

25. The method of claim 2, wherein the cell sample is a nucleic acid sample or a protein sample.

26. (canceled)

27. The method of claim 2, wherein the subject has a family member which has been diagnosed with a scoliosis.

28. A composition or kit or DNA chip comprising at least one oligonucleotide probe or primer for the specific detection of a polymorphic marker in a gene listed in Table 4.

29. The composition or kit of claim 28, wherein the polymorphic marker is a polymorphic marker defined in Table 6, and preferably a risk variant defined in Table 6.

30.-33. (canceled)

34. Use of the composition or kit of claim 29 or of a DNA chip comprising at least one oligonucleotide for detecting the presence or absence of a polymorphic marker in at least one gene listed in Table 4 and a substrate on which the oligonucleotide is immobilized, for determining the risk of developing a scoliosis or for genotyping a subject.

35. (canceled)

36. The method of claim 2, wherein the subject suffers from a scoliosis or is at risk of developing a scoliosis, and the method further comprises classifying the subject into an IS group.

37. A composition comprising (i) a cell sample from the subject; and (ii) one or more reagent for detecting (a) the length of cilia at the surface of cells; (b) the number of cells with elongated cilia; (c) the number of ciliated cells; (d) the level of expression of at least one mechanoresponsive gene; and/or (e) the presence or absence of a polymorphic marker in at least one gene listed in Table 4 or a substitute marker in linkage disequilibrium therewith.

38. The composition of claim 37, wherein said cell sample is from cells which have been submitted to a mechanostimulation.

39. An oligonucleotide primer or probe for detecting the presence or absence of a reference allele or risk variant allele defined in Table 6, preferably further comprising a label.

40. The oligonucleotide primer or probe of claim 39, further comprising a label.

41. The oligonucleotide primer or probe of claim 39 comprising or consisting of a polynucleotide sequence set forth in Table 6 (SEQ ID NOs: 1 to 1302) or the complement thereof.

42. The oligonucleotide primer or probe of claim 39, consisting of 10 to 60 nucleotides.
